(19) United States
(12) Patent Application Publication
(10) Pub. No.: US 2026/0286345 A1
(43) Pub. Date: Sep. 24, 2026

(54) ON-SUPPORT CIRCULARIZATION AND AMPLIFICATION FOR GENERATING IMMOBILIZED NUCLEIC ACID CONCATEMER MOLECULES

(71) Applicant: Element Biosciences, Inc., San Diego, CA (US)

(72) Inventors: Junhua ZHAO, San Diego, CA (US); Xiaodong QI, San Diego, CA (US); Hua YU, San Diego, CA (US); Kim NGO, San Diego, CA (US); Tyler LOPEZ, Valley Center, CA (US); Kerry GUNNING, San Diego, CA (US); Tuval BEN-YEHEZKEL, Pleasanton, CA (US); Sinan ARSLAN, San Diego, CA (US); Hui Zhen MAH, San Diego, CA (US); Andrew PRICE, San Diego, CA (US); Michael PREVITE, San Diego, CA (US); Kyle METCALFE, San Diego, CA (US); Yu LIU, San Diego, CA (US)

(21) Appl. No.: 19/456,148

(22) Filed: Jan. 22, 2026

Related U.S. Application Data
(63) Continuation of application No. PCT/US2024/039186, filed on Jul. 23, 2024.
(60) Provisional application No. 63/515,328, filed on Jul. 24, 2023.

(51) Int. Cl. C12N 15/10

(52) U.S. Cl. CPC C12N 15/1093

(57) ABSTRACT

The present disclosure provides compositions and methods for generating a plurality of nucleic acid concatemers immobilized to a support by conducting on-support circularization and ligation reactions using a plurality of linear library molecules and a plurality of immobilized splint capture primers. The present disclosure provides methods for seeding and optionally re-seeding the support to increase the density of immobilized nucleic acid concatemers that can be sequenced. In some embodiments, the immobilized concatemers can be used for conducting downstream sequencing workflows including batch sequencing and reiterative sequencing workflows. The present disclosure also provides methods for interrupting an ongoing sequencing run to re-seed the support to generate additional concatemers that can be sequenced.

### CROSS REFERENCE TO RELATED APPLICATIONS

This application is a continuation of International Application No. PCT/US2024/039186, filed on Jul. 23, 2024, which claims priority to, and benefit of, U.S. Provisional Application No. 63/515,328, filed on Jul. 24, 2023, the contents of each of which are incorporated by reference in their entirety herein.

### INCORPORATION BY REFERENCE OF SEQUENCE LISTING

The contents of the electronic sequence listing (ELEM-022_C01US_SeqList_ST26-updated.xml; Size: 158,552 bytes; and Date of Creation: Mar. 5, 2026) are herein incorporated by reference in their entireties.

### TECHNICAL FIELD

The present disclosure is directed to compositions and methods for nucleic acid library preparation for next generation sequencing, and methods of sequencing the libraries prepared using the techniques disclosed herein. The nucleic acid libraries comprise nucleic acid concatemer template molecules that can be generated by hybridizing linear library molecules and a plurality of immobilized splint capture primers, and conducting rolling circle amplification. The resulting libraries can be used with downstream sequencing workflows, including batch sequencing and reiterative sequencing.

### BACKGROUND

Improvements in next generation sequencing technology have greatly increased sequencing speed and data output, resulting in the high sample throughput of current sequencing platforms. Efficient preparation of sequencing libraries suitable for next generation sequencing applications is important for downstream amplification and sequencing workflows. Accordingly, there is a need for alternative methods for producing and sequencing a nucleic acid libraries. Provided herein are compositions, methods and kits addressing this need. The compositions and methods of the present disclosure can be used to generate a plurality of nucleic acid concatemer template molecules immobilized to a support, which are compatible with a variety of downstream sequencing methods. The plurality of nucleic acid concatemer template molecules can be generated using a plurality of linear library molecules and a plurality of immobilized splint capture primers. The present disclosure also provides methods for seeding and re-seeding the support. The immobilized nucleic acid concatemer template molecules can also be used for conducting downstream sequence workflows including batch sequencing and reiterative sequencing workflows.

### SUMMARY

The disclosure provides methods for generating a plurality of nucleic acid concatemer template molecules immobilized to a support, comprising: (a) providing a support having a plurality of splint capture primers (200) and a plurality of pinning primers (500) immobilized thereon, wherein individual splint capture primers (200) in the plurality comprise a first portion (210) which binds a first universal binding site in a linear library molecule (100) and a second portion (220) which binds a second universal binding site in the same linear library molecule (100), wherein the density of the splint capture primers (200 on the support is between 10⁵-10¹⁵ per mm², wherein individual pinning primers (500) in the plurality bind at least a portion of individual concatemer template molecules, and wherein individual pinning primers (500) comprise a terminal 3′ non-extendible end; (b) providing a plurality of linear library molecules (100), wherein individual linear library molecules in the plurality comprise a sequence of interest and any one or combination of two or more adaptor sequences in any order, wherein the adaptor sequences comprise: (i) a first universal binding site (120) for a first portion of a splint capture primer or a complementary sequence thereof, (ii) a universal binding site for a first non-splint capture primer (123) or a complementary sequence thereof; (iii) at least one sample index sequence comprising a left sample index sequence (160) and/or a right sample index sequence (170), wherein the left sample index sequence (160) and/or right sample index sequence (170) which distinguishes sequences of interest obtained from different sample sources in a multiplex assay; (iv) at least one universal binding site for a forward sequencing primer (140) or a complementary sequence thereof; (v) at least one universal binding site for a reverse sequencing primer (150) or a complementary sequence thereof; (vi) at least one universal binding site for a compaction oligonucleotide or a complementary sequence thereof; (vii) at least one unique molecular index sequence (UMI) comprising a left unique molecular index sequence (180) and/or a right unique molecular index sequence (190) which can be used to uniquely identify a linear library molecule (100) to which the unique molecular index sequence is appended; (viii) at least one universal binding site for a pinning primer or a complementary sequence thereof; (ix) at least one batch-specific barcode sequence; (x) a universal binding site for a second non-splint capture primer (133) or a complementary sequence thereof; (xi) at least one short random sequence which is about 3-20 nucleotides in length and provides nucleotide sequence diversity; and/or (xii) a second universal binding site (130) for a second portion of the immobilized splint capture primer or a complementary sequence thereof; (c) contacting the plurality of splint capture primers (200) with the plurality of linear library molecules (100), wherein the contacting is conducted under a condition suitable for hybridizing individual linear library molecules to individual splint capture primers to form individual open circle library molecules (300) having a gap or nick between the terminal 5′ and 3′ ends of the open circle library molecule (300), wherein individual linear library molecules comprise the first universal binding site (120) for the first portion of a splint capture primer which is hybridized to the first portion (210) of a splint capture primer, and wherein the same linear library molecule (100) comprises the second universal binding site (130) for the second portion of the immobilized splint capture primer which is hybridized to the second portion (220) of the same splint capture primer, thereby generating a plurality of open circle library molecules (300) comprising nicks or gaps; (d) enzymatically closing the nicks or gaps in the plurality of open circle library molecules, thereby generating a plurality of covalently closed circular library molecules (400), wherein individual covalently closed circular library molecules are hybridized to the splint capture primers (200); (e) contacting the plurality of covalently closed circular library molecules (400) with a rolling circle amplification reaction mixture and conducting a rolling circle amplification reaction, thereby generating the plurality of concatemer template molecules, wherein the plurality of concatemer template molecules are immobilized on the support, and wherein the density of the concatemer template molecules on the support is between 10⁵-10¹⁵ per mm², wherein the rolling circle amplification reaction mixture comprises a strand displacing polymerase and a mixture of nucleotides comprising dATP, dGTP, dCTP, dTTP and dUTP, wherein the rolling circle amplification reaction mixture comprises a plurality of single-stranded nucleic acid compaction oligonucleotides, wherein the 5′ and 3′ regions of individual single-stranded nucleic acid compaction oligonucleotides hybridize to universal binding sites on an individual concatemer template molecule thereby pulling together distal portions of the individual concatemer template molecule and causing compaction of the concatemer template molecule to form a DNA nanoball, wherein the terminal 3′ ends of individual single-stranded nucleic acid compaction oligonucleotides are non-extendible, and wherein at least one portion of individual concatemer template molecules are hybridized to a pinning primer immobilized on the support; and (f) conducting at least one sequencing reaction to determine the sequence of at least a portion of the plurality of concatemer template molecules.

The disclosure provides methods for generating a plurality of nucleic acid concatemer template molecules immobilized to a support, comprising: (a) providing a support comprising a plurality of splint capture primers (200) and a plurality of pinning primers (500) immobilized thereon, wherein individual splint capture primers (200) in the plurality comprise a first portion (210) which binds a first universal binding site in a linear library molecule (100) and a second portion (220) which binds a second universal binding site in the same linear library molecule (100), wherein the density of the splint capture primers (200) on the support is between 10⁵-10¹⁵ per mm², wherein individual pinning primers (500) in the plurality bind at least a portion of individual concatemer template molecules, and wherein individual pinning primers (500) comprise a terminal 3′ non-extendible end; (b) providing a plurality of linear library molecules (100), wherein individual linear library molecules in the plurality comprise a sequence of interest and any one or any combination of two or more adaptor sequences in any order, wherein the adaptor sequences comprise (i) a first universal binding site (120) for a first portion of a splint capture primer or a complementary sequence thereof; (ii) a universal binding site for a first non-splint capture primer (123) or a complementary sequence thereof; (iii) at least one sample index sequence comprising a left sample index sequence (160) and/or a right sample index sequence (170) which distinguishes sequences of interest obtained from different sample sources in a multiplex assay; (iv) at least one universal binding site for a forward sequencing primer (140) or a complementary sequence thereof, (v) at least one universal binding site for a reverse sequencing primer (150) or a complementary sequence thereof, (vi) at least one universal binding site for a compaction oligonucleotide or a complementary sequence thereof, (vii) at least one unique molecular index sequence (UMI) comprising a left unique molecular index sequence (180) and/or a right unique molecular index sequence (190) which can be used to uniquely identify a linear library molecule (100 to which the unique molecular index sequence is appended; (viii) at least one universal binding site for a pinning primer or a complementary sequence thereof, (ix) at least one batch-specific barcode sequence; (x) a universal binding site for a second non-splint capture primer (133) or a complementary sequence thereof; (xi) at least one short random sequence (132) which is about 3-20 nucleotides in length and provides nucleotide sequence diversity; and/or (xii) a second universal binding site (130) for a second portion of the immobilized splint capture (or a complementary sequence thereof); (c) (i) contacting the plurality of splint capture primers (200) with the plurality of linear library molecules (100), wherein the contacting is conducted under a condition suitable for hybridizing individual linear library molecules (100) to individual splint capture primers (200) to form individual open circle library molecules (300) comprising a 5′ overhang flap structure, and (ii) contacting the 5′ overhang flap structures with a flap cleaving reagent under a condition suitable to cleave the 5′ overhang flap structures to generate a plurality of open circle library molecules each having a newly cleaved 5′ end and a non-cleaved 3′ end and comprising a nick between the terminal 5′ and 3′ ends of the open circle library molecule, wherein an individual linear library molecules comprise at least a portion of the first universal binding site (120) for the first portion of a splint capture primer which hybridized to the first portion (210) of the splint capture primer, wherein the same linear library molecule comprises at least a portion of the second universal binding site (130) for the second portion of the immobilized splint capture primer which is hybridized to the second portion (220) of the same splint capture primer; (d) enzymatically closing the nicks in the plurality of open circle library molecules (300), thereby generating a plurality of covalently closed circular library molecules (400), wherein individual covalently closed circular library molecules are hybridized to splint capture primers (200); (e) contacting the plurality of covalently closed circular library molecules (400) with a rolling circle amplification reaction mixture and conducting a rolling circle amplification reaction, thereby generating a plurality of concatemer template molecules immobilized on the support, wherein the density of the concatemer template molecules on the support is 10⁵-10¹⁵ per mm², wherein the rolling circle amplification reaction mixture comprises a strand displacing polymerase and a mixture of nucleotides comprising dATP, dGTP, dCTP, dTTP and dUTP, wherein the rolling circle amplification reaction mixture comprises a plurality of single-stranded nucleic acid compaction oligonucleotides, wherein the 5′ and 3′ regions of individual single-stranded nucleic acid compaction oligonucleotides hybridize to universal binding sites on a nucleic acid concatemer template molecule to pull together distal portions of the concatemer template molecule thereby causing compaction of the concatemer template molecule to form a DNA nanoball, wherein the terminal 3′ ends of the single-stranded nucleic acid compaction oligonucleotides are non-extendible, and wherein at least one portion of individual concatemer template molecules are hybridized to a pinning primer (500); and (f) conducting at least one sequencing reaction to determine the sequence of at least a portion of the plurality of concatemer template molecules.

In some embodiments, the plurality of splint capture primers (200) of step (a) are located at random and non-predetermined positions on the support. In some embodiments, the plurality of splint capture primers (200) of step (a) include a plurality of nearest neighbor splint capture primers that contact each other and/or overlap each other when the support is viewed from any angle including above, below or from the side. In some embodiments, the plurality of splint capture primers of step (a) comprise at least a first sub-population of splint capture primers having a first sequence and a second sub-population of splint capture primers having a second sequence which differs from the first sequence.

In some embodiments, the plurality of linear library molecules (100) in step (b) comprises at least a first sub-population of linear library molecules and a second sub-population of linear library molecules. In some embodiments, the linear library molecules (100) in the first sub-population comprise a mixture of sequences of interest and the linear library molecules (100) in the second sub-population comprise a mixture of sequences of interest. In some embodiments, the first sub-population of linear library molecules comprise a universal binding site for a first batch-specific forward sequencing primer (140-1) or a complementary sequence thereof, a universal binding site for a first batch-specific reverse sequencing primer (150-1) or a complementary sequence thereof, and a first batch-specific barcode sequence (142); and the second sub-population of linear library molecules comprise a universal binding site for a second batch-specific forward sequencing primer (140-2) or a complementary sequence thereof, a universal binding site for a second batch-specific reverse sequencing primer (150-2) or a complementary sequence thereof, and a second batch-specific barcode sequence (152).

In some embodiments, the plurality of concatemer template molecules of step (e) comprise at least a first sub-population of concatemer template molecules and a second sub-population of concatemer template molecules. In some embodiments, the first and second sub-populations of concatemer template molecules are located at random and non-predetermined positions on the support, and wherein individual concatemer template molecules in the first and second sub-populations of concatemer template molecules include nearest neighbor nucleic acid concatemer template molecules that contact each other or overlap each other when the support is viewed from any angle including above, below or from the side.

In some embodiments, the sequencing of step (f) comprises conducting a first batch reiterative sequencing. In some embodiments, the first batch reiterative sequencing comprises: (a) hybridizing the first sub-population of concatemer template molecules with a plurality of first batch-specific forward sequencing primers and conducting a plurality of sequencing reactions, thereby generating a plurality of first batch sequencing read products, wherein the first batch sequencing read products are no more than 50 bases in length; (b) stopping or blocking the first batch reiterative sequencing of step (a) to inhibit further sequencing reactions; (c) removing the plurality of first batch sequencing read products from the first sub-population of concatemer template molecules and retaining the first sub-population of concatemer template molecules; and (d) reiteratively sequencing the first sub-population of concatemer template molecules by repeating steps (a)-(c) at least once.

In some embodiments, the sequencing of step (f) further comprises conducting a second batch reiterative sequencing. In some embodiments, the second batch reiterative sequencing comprises: (a) hybridizing the second sub-population of concatemer template molecules with a plurality of second batch-specific forward sequencing primers and conducting a plurality of sequencing reactions, thereby generating a plurality of second batch sequencing read products, wherein the second batch sequencing read products are no more than 50 bases in length; (b) stopping or blocking the second batch reiterative sequencing of step (a) to inhibit further sequencing reactions; (c) removing the plurality of second batch sequencing read products from the second sub-population of concatemer template molecules and retaining the second sub-population of concatemer template molecules; and (d) reiteratively sequencing the second sub-population of concatemer template molecules by repeating steps (a)-(c) at least once.

In some embodiments, (i) step (c) comprises distributing onto the support a first sub-population of linear library molecules under a condition suitable for hybridizing individual linear library molecules from the first sub-population to individual splint capture primers (200) to generate a first sub-population of open circle library molecules each having nick or gap, wherein the support comprises an excess of splint capture primers immobilized thereon compared to the first sub-population of linear library molecules; (ii) step (d) comprises enzymatically closing the nick or gap to generate a first sub-population of covalently closed circular library molecules, wherein individual covalently closed circular library molecules are hybridized to a splint capture primer; (iii) step (e) comprises conducting a rolling circle amplification reaction to generate a first sub-population of concatemer template molecules; (iv) step (f) comprises sequencing at least a portion of the first sub-population of concatemer template molecules; (v) wherein the method further comprises halting the sequencing of the first sub-population of concatemer template molecules; (vi) distributing onto the same support a second sub-population of linear library molecules under a condition suitable for hybridizing individual linear library molecules from the second sub-population to individual splint capture primers (200) to generate a second sub-population of open circle library molecules each having nick or gap, and enzymatically closing the nick or gap to generate a second sub-population of covalently closed circular library molecules, wherein individual covalently closed circular library molecules are hybridized to a splint capture primer, conducting a rolling circle amplification reaction to generate a second sub-population of concatemer template molecules; and (vii) continuing the sequencing of at least a portion of the first sub-population of concatemer template molecules or sequencing at least a portion of the second sub-population of concatemer template molecules.

In some embodiments, the sequencing of step (f) comprises pairwise sequencing. In some embodiments, the pairwise sequencing comprises: (a) generating a plurality of extended forward sequencing primer strands by contacting the plurality of concatemer template molecules with a plurality of forward sequencing primers under a condition suitable to hybridize at least one forward sequencing primer to at least one of the universal binding sites for a forward sequencing primer (140) of the concatemer template molecules, and conducting forward sequencing reactions using the hybridized first forward sequencing primers, a plurality of sequencing polymerases, and a plurality of nucleotide reagents; (b) retaining the plurality of concatemer template molecules immobilized on the support and replacing the plurality of extended forward sequencing primer strands with a plurality of forward extension strands that are hybridized to the concatemer template molecules by conducting a primer extension reaction using the concatemer template molecules as a template molecules; (c) removing the concatemer molecules by generating abasic sites in the concatemer template molecules at uridine nucleotides in the concatemer template molecules and generating gaps at the abasic sites to generate a plurality of gap-containing concatemer template molecules, while retaining the plurality of forward extension strands and retaining the plurality of immobilized splint capture primers and pinning primers; and (d) sequencing the plurality of forward extension strands by contacting the plurality of forward extension strands with a plurality of soluble reverse sequencing primers, a plurality of sequencing polymerases, and a plurality of nucleotide reagents, and conducting reverse sequencing reactions thereby generating a plurality of extended reverse sequencing primer strands.

In some embodiments, the sequencing of step (f) comprises chain terminator sequencing. In some embodiments, the chain terminator sequencing comprises: (a) contacting the plurality of concatemer template molecules with a plurality of sequencing polymerases and a plurality of nucleic acid sequencing primers, where the contacting is conducted under a condition suitable to form a plurality of sequencing polymerase complexes comprising a sequencing polymerase bound to a nucleic acid duplex, wherein the nucleic acid duplex comprises a portion of a concatemer template molecule hybridized to a nucleic acid sequencing primer; (b) contacting the plurality of sequencing polymerase complexes with a plurality of nucleotides comprising a detectable label and a blocking moiety at the 2′ or 3′ sugar position, where the contacting is conducted under a condition suitable for binding at least one nucleotide to one of the sequencing polymerase complexes, and the condition is suitable for promoting polymerase-catalyzed nucleotide incorporation; (c) incorporating a nucleotide into the 3′ end of the nucleic acid sequencing primer of at least one sequencing polymerase complex, thereby generating a sequencing polymerase complex comprising an incorporated nucleotide; (d) detecting the incorporated nucleotide; (e) removing the blocking moiety from the incorporated nucleotide; and (f) repeating steps (b)-(e) at least once.

In some embodiments, the sequencing of step (f) comprises: (a) contacting the plurality of concatemer template molecules with a plurality of sequencing polymerases and a plurality of nucleic acid sequencing primers, wherein the contacting is conducted under a condition suitable to form a plurality of sequencing polymerase complexes comprising a sequencing polymerase bound to a nucleic acid duplex, wherein the nucleic acid duplex comprises a portion of a concatemer template molecule hybridized to a nucleic acid sequencing primer; (b) contacting the plurality of sequencing polymerase complexes with a plurality of nucleotides comprising detectable labels attached to a phosphate moiety of the phosphate chain, wherein the contacting is conducted under a condition suitable for binding at least one nucleotide to one of the sequencing polymerase complexes, and the condition is suitable for promoting polymerase-catalyzed nucleotide incorporation; (c) incorporating a nucleotide into the 3′ end of a sequencing primer of at least one sequencing polymerase complex, thereby generating a sequencing polymerase complex comprising an incorporated nucleotide; (d) detecting the incorporated nucleotide; and (e) repeating steps (b)-(d) at least once.

In some embodiments, the sequencing of step (f) comprises: (a) contacting the plurality of concatemer template molecules with a plurality of a first sequencing polymerases and a plurality of nucleic acid sequencing primers, wherein the contacting is conducted under a condition suitable to bind the plurality of first polymerases to the plurality of concatemer template molecules and the plurality of nucleic acid primers, thereby forming a plurality of first polymerase complexes comprising a first sequencing polymerase bound to a nucleic acid duplex, wherein the nucleic acid duplex comprises a concatemer template molecule hybridized to a nucleic acid sequencing primer; (b) contacting the plurality of first polymerase complexes with a plurality of multivalent molecules, wherein the multivalent molecules are detectably labeled, and wherein individual multivalent molecules in the plurality comprise a core attached to multiple nucleotide arms and each nucleotide arm is attached to a nucleotide moiety, wherein the contacting is conducted under a condition suitable for binding complementary nucleotide moieties of the multivalent molecules to at least two of the plurality of first polymerase complexes, thereby forming a plurality of multivalent-binding polymerase complexes, and the condition is suitable for inhibiting incorporation of complementary nucleotide moieties into the nucleic acid sequencing primers of the plurality of multivalent-binding polymerase complexes; (c) detecting the plurality of multivalent-binding polymerase complexes; and (d) identifying the base of the nucleotide moieties in the plurality of multivalent-binding polymerase complexes, thereby determining the sequence of the concatemer template molecules. In some embodiments, the methods further comprise (e) dissociating the plurality of multivalent-binding polymerase complexes by removing the plurality of first nucleic acid sequencing polymerases and bound multivalent molecules, and retaining the nucleic acid duplexes, thereby generating a plurality of retained nucleic acid duplexes; (f) contacting the plurality of the retained nucleic acid duplexes of step (e) with a plurality of a second sequencing polymerases under a condition suitable for binding the plurality of second polymerases to the plurality of the retained nucleic acid duplexes, thereby forming a plurality of second polymerase complexes comprising a second sequencing polymerase bound to a nucleic acid duplex; and (g) contacting the plurality of second polymerase complexes with a plurality of nucleotides, wherein the contacting is conducted under a condition suitable for binding complementary nucleotides from the plurality of nucleotides to at least two of the second polymerase complexes, thereby forming a plurality of nucleotide-polymerase complexes, and the condition is suitable for promoting nucleotide incorporation of the bound complementary nucleotides into the primers of the nucleotide-binding complexes. In some embodiments, nucleotides in the plurality of nucleotides comprise detectable labels, and the method comprises: (h) detecting the complementary nucleotides which are incorporated into the nucleic acid sequencing primers of the nucleotide-polymerase complexes. In some embodiments, the methods comprise (h) detecting the complementary nucleotides which are incorporated into the nucleic acid sequencing primers of the nucleotide-polymerase complexes; and (i) identifying the bases of the complementary nucleotides which are incorporated into the nucleic acid sequencing primers of the nucleotide-polymerase complexes. In some embodiments, the plurality of nucleotides of step (g) comprise a plurality of non-labeled nucleotides and wherein detecting the nucleotide incorporation is omitted. In some embodiments, the contacting the plurality of first polymerase complexes with the plurality of multivalent molecules of step (b) is conducted in the presence of a non-catalytic divalent cation that inhibits polymerase-catalyzed nucleotide incorporation, and wherein the non-catalytic divalent cation comprises strontium, barium or calcium. In some embodiments, the contacting the plurality of second polymerase complexes with the plurality of nucleotides of step (g) is conducted in the presence of a catalytic divalent cation that promotes polymerase-catalyzed nucleotide incorporation, and wherein the catalytic divalent cation comprises magnesium or manganese.

In some embodiments, individual multivalent molecules in the plurality of multivalent molecules comprise: (a) a core; and (b) a plurality of nucleotide arms which comprise (i) a core attachment moiety, (ii) a spacer, (iii) a linker, and (iv) a nucleotide moiety, wherein the core is attached to the plurality of nucleotide arms via their core attachment moiety, wherein the spacer is attached to the linker, and wherein the linker is attached to the nucleotide moiety. In some embodiments, the linker comprises an aliphatic chain having 2-6 subunits or an oligo ethylene glycol chain having 2-6 subunits. In some embodiments, the plurality of nucleotide arms attached to a given core have the same type of nucleotide moieties, and wherein the types of nucleotide moieties comprise dATP, dGTP, dCTP, dTTP or dUTP. In some embodiments, the plurality of multivalent molecules comprise one type of a multivalent molecule wherein each multivalent molecule in the plurality has the same type of nucleotide moiety selected from a group consisting of dATP, dGTP, dCTP, dTTP and dUTP. In some embodiments, the plurality of multivalent molecules comprise a mixture of any combination of two or more types of multivalent molecules each type having nucleotide moieties selected from a group consisting of dATP, dGTP, dCTP, dTTP and/or dUTP.

In some embodiments, individual nucleotides in the plurality of nucleotides in step (g) comprise an aromatic base, a five carbon sugar, and 1-10 phosphate groups. In some embodiments, the plurality of nucleotides of step (g) comprise one type of nucleotide selected from a group consisting of dATP, dGTP, dCTP, dTTP and dUTP, or comprise a mixture of any combination of two or more types of nucleotides selected from a group consisting of dATP, dGTP, dCTP, dTTP and/or dUTP. In some embodiments, at least one of the nucleotides in the plurality of nucleotides in step (g) is labeled with a fluorophore. In some embodiments, the plurality of nucleotides in step (g) lack a fluorophore label. In some embodiments, at least one of the nucleotides in the plurality of nucleotides of step (g) comprises a removable chain terminating moiety attached to the 3′ carbon position of the sugar group, optionally wherein the removable chain terminating moiety comprises an alkyl group, alkenyl group, alkynyl group, allyl group, aryl group, benzyl group, azide group, azido group, 0-azidomethyl group, amine group, amide group, keto group, isocyanate group, phosphate group, thiol group, disulfide group, carbonate group, urea group, or silyl group, and optionally wherein the removable chain terminating moiety is cleavable with a chemical compound to generate an extendible 3′OH moiety on the sugar group.

In some embodiments, the methods comprise forming a plurality of binding complexes, comprising the steps: (a) binding a first nucleic acid sequencing primer, a first sequencing polymerase, and a first multivalent molecule to a first portion of a concatemer template molecule, thereby forming a first binding complex, wherein a first nucleotide moiety of the first multivalent molecule binds to the first polymerase; and (b) binding a second nucleic acid sequencing primer, a second sequencing polymerase, and the first multivalent molecule to a second portion of the same nucleic acid concatemer template molecule thereby forming a second binding complex, wherein a second nucleotide moiety of the first multivalent molecule binds to the second polymerase, and wherein the first and second binding complexes include the same multivalent molecule, thereby forming an avidity complex.

In some embodiments, the methods comprise (a) contacting the plurality of the first sequencing polymerases and the plurality of nucleic acid sequencing primers with different portions of an individual nucleic acid concatemer template molecule to form at least first polymerase complex and second polymerase complex on the same nucleic acid concatemer template molecule; (b) contacting a plurality of multivalent molecules comprising detectable labels with the at least first and second polymerase complexes, under conditions suitable to bind a single multivalent molecule from the plurality to the first and second polymerase complexes, wherein at least a first nucleotide moiety of the single multivalent molecule is bound to the first polymerase complex which includes a first nucleic acid sequencing primer hybridized to a first portion of the concatemer template molecule, thereby forming a first binding complex, and wherein at least a second nucleotide moiety of the single multivalent molecule is bound to the second polymerase complex which includes a second nucleic acid sequencing primer hybridized to a second portion of the concatemer template molecule thereby forming a second binding complex, and wherein the contacting is conducted under a condition suitable to inhibit polymerase-catalyzed incorporation of the bound first and second nucleotide moieties in the first and second binding complexes, and wherein the first and second binding complexes bound to the same multivalent molecule form an avidity complex; (c) detecting the first and second binding complexes on the same concatemer molecule; and (d) identifying the first nucleotide moiety in the first binding complex thereby determining the sequence of the first portion of the concatemer template molecule, and identifying the second nucleotide moiety in the second binding complex thereby determining the sequence of the second portion of the concatemer template molecule.

In some embodiments, the plurality of sequencing polymerases of steps (a) and (d) comprise a plurality of engineered polymerases comprising at least 70%, 75%, 80%, 85%, 90%, 91%, 92%, 93%, 94%, 95%, 96%, 97%, 98%, 99%, or greater than 99% sequence identity to any of SEQ ID NOS:128-146. In some embodiments, the plurality of sequencing polymerases of step (a) comprises a plurality of engineered polymerases comprising at least 70%, 75%, 80%, 85%, 90%, 91%, 92%, 93%, 94%, 95%, 96%, 97%, 98%, 99%, or greater than 99% sequence identity to any of SEQ ID NOS: 128-146. In some embodiments, the plurality of sequencing polymerases comprises a plurality of engineered polymerases comprising at least 70%, 75%, 80%, 85%, 90%, 91%, 92%, 93%, 94%, 95%, 96%, 97%, 98%, 99%, or greater than 99% sequence identity to any of SEQ ID NOS: 128-146. In some embodiments, the plurality of a first sequencing polymerases comprises a plurality of engineered polymerases comprising at least 70%, 75%, 80%, 85%, 90%, 91%, 92%, 93%, 94%, 95%, 96%, 97%, 98%, 99%, or greater than 99% sequence identity to any of SEQ ID NOS: 128-146. In some embodiments, the plurality of a second sequencing polymerases comprises a plurality of engineered polymerases comprising at least 70%, 75%, 80%, 85%, 90%, 91%, 92%, 93%, 94%, 95%, 96%, 97%, 98%, 99%, or greater than 99% sequence identity to any of SEQ ID NOS: 128-146.

In some embodiments, the flap cleaving reagent comprises at least one 5′ flap endonuclease from a eukaryote or archaeal organism. In some embodiments, the flap cleaving reagent comprises at least one archaeal 5′ flap endonuclease selected from a group consisting of: Archaeoglobus fulgidus (Afu FEN1), Methanobacterium thermoautotrophicum (Mth FEN1), Pyrococcus furiosus (Pfu FEN1), Methanococcus jannaschii (Mja FEN1), Pyrococcus woesei (Pwo FEN1), Pyrococcus horikoshii (Pho FEN1), Archaeoglobus veneficus (Ave FEN1), Thermococcus kodakarensis (Tko FEN1), Desulfurococcus amylolyticus (Dam FEN1), Aeropyrum pernix (Ape FEN1), and Sulfolobus solfataricus (Sso FEN1). In some embodiments, the flap cleaving reagent comprises a 5′ flap endonuclease from Thermococcus sp. 9 degrees North (9° N FEN1). In some embodiments, the flap cleaving reagent comprises a 5′ flap endonuclease from murine, yeast or human.

In some embodiments, enzymatically closing the nicks comprises contacting the plurality of open circle library molecules with a DNA ligase comprising a T3 ligase, a T4 ligase, a T7 ligase, a Tfu ligase or a ligase from Thermococcus nautili.

In some embodiments, the flap cleaving reagent comprises a DNA ligase. In some embodiments, the DNA ligase comprises a T3 ligase, a T4 ligase, a T7 ligase, a Tfu ligase or a ligase from Thermococcus nautili.

### BRIEF DESCRIPTION OF THE DRAWINGS

The features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a schematic of various exemplary configurations of multivalent molecules. Left (Class I): schematics of multivalent molecules having a “starburst” or “helter-skelter” configuration. Center (Class II): a schematic of a multivalent molecule having a dendrimer configuration. Right (Class III): a schematic of multiple multivalent molecules formed by reacting streptavidin with 4-arm or 8-arm PEG-NHS with biotin and dNTPs. Nucleotide moieties are designated ‘N’, biotin is designated ‘B’, and streptavidin is designated ‘SA’.

FIG. 2 is a schematic of an exemplary multivalent molecule comprising a generic core attached to a plurality of nucleotide-arms.

FIG. 3 is a schematic of an exemplary multivalent molecule comprising a dendrimer core attached to a plurality of nucleotide-arms.

FIG. 4 is a schematic of an exemplary multivalent molecule comprising a core attached to a plurality of nucleotide arms, where the nucleotide arms comprise biotin, spacer, linker and a nucleotide moiety.

FIG. 5 is a schematic of an exemplary nucleotide arm comprising a core attachment moiety, spacer, linker and nucleotide moiety.

FIG. 6 shows the chemical structure of an exemplary spacer (top), and the chemical structures of various exemplary linkers, including an 11-atom Linker, 16-atom Linker, 23-atom Linker and an N3 Linker (bottom).

FIG. 7 shows the chemical structures of various exemplary linkers, including Linkers 1-9.

FIG. 8 shows the chemical structures of various exemplary linkers joined/attached to nucleotide moieties.

FIG. 9 shows the chemical structures of various exemplary linkers joined/attached to nucleotide moieties.

FIG. 10 shows the chemical structures of various exemplary linkers joined/attached to nucleotide moieties.

FIG. 11 shows the chemical structure of an exemplary biotinylated nucleotide arm. In this example, the nucleotide moiety is connected to the linker via a propargyl amine attachment at the 5 position of a pyrimidine base or the 7 position of a purine base.

FIG. 12 is a schematic of a guanine tetrad (e.g., G-tetrad).

FIG. 13 is a schematic of an exemplary intramolecular G-quadruplex structure.

FIG. 14 is a schematic of an exemplary low binding support comprising a glass substrate and alternating layers of hydrophilic coatings which are covalently or non-covalently adhered to the glass, and which further comprises chemically-reactive functional groups that serve as attachment sites for oligonucleotide primers (e.g., splint capture primers). Alternatively, the support can be made of any material such as glass, plastic or a polymer material.

FIG. 15A is a schematic of an exemplary support having a plurality of splint capture primers (200) arranged on the support in a non-predetermined and random manner. A circular spot represents a splint capture primer immobilized to the support. The plurality of splint capture primers can have the same sequence. The splint capture primers can be attached to the support such that some of the nearest neighbor splint capture primers touch each other and/or overlap each other when viewed from any angle of the support including above, below and/or side views of the support, as shown by the dotted lines that surround the four splint capture primers representing nearest neighbor splint capture primers that touch each other.

FIG. 15B is a schematic of the same support shown in FIG. 15A, where individual splint capture primers (200) are attached to a nucleic acid concatemer template molecule having one of four different batch sequences (e.g. a batch-specific sequencing primer binding sites and/or batch-specific barcode sequences, which are common to a particular batch or sub-population of nucleic acid concatemer template molecules in a plurality of nucleic acid concatemer template molecules). The different batch sequences of the concatemer template molecules are represented by horizontal stripes, vertical dashed, brick or solid black. The concatemer template molecules can attach to the support (e.g., via attachment to the splint capture primers) such that some of the nearest neighbor nucleic acid concatemer molecules touch each other and/or overlap each other when viewed from any angle of the support including above, below or side views of the support. The dotted lines that surround the four concatemer template molecules represent nearest neighbor concatemer template molecules that touch each other.

FIG. 16A is a schematic of an exemplary support having a plurality of concatemer template molecules immobilized to the support (e.g., via attachment to splint capture primers (200)) where the concatemer template molecules are arranged on the support in a predetermined manner. A circular spot represents a concatemer template molecule immobilized to the support. The concatemer template molecule comprises one of four different batch sequences (e.g., batch-specific sequencing primer binding sites and/or batch-specific barcode sequences). The different batch sequences of the nucleic acid concatemer template molecules are represented by horizontal stripes, vertical dashed, brick or solid black. For example, the nucleic acid concatemer template molecules can be immobilized to the support to form spots and arranged in row and columns.

FIG. 16B is a schematic of an exemplary support having a plurality of nucleic acid concatemer template molecules immobilized to the support (e.g., via attachment to splint capture primers (200)) where the concatemer template molecules are arranged on the support in a predetermined manner. The concatemer template molecules can comprise one of four different batch sequences (e.g., batch-specific sequencing primer binding sites and/or batch-specific barcode sequences). The different batch sequences of the concatemer template molecules are represented by horizontal stripes, vertical dashed, brick or solid black. For example, a plurality of concatemer template molecules can be immobilized to the support and arranged to form stripes.

FIG. 17A is a schematic showing a support having an exemplary splint capture primer (200) immobilized thereon, where the splint capture primer can be used to conduct an on-support ligation reaction. The splint capture primer comprises a first portion (210) and a second portion (220). The schematic also shows an exemplary open circle library molecule formed from a linear library molecule comprising a first universal binding site (120) for binding the first portion of the splint capture primer, and a second universal binding site (130) for binding the second portion of the same splint capture primer. In some embodiments, the linear library molecule also includes a sequence of interest and at least one adaptor sequence. The linear library molecule is hybridized to the splint capture primer to form an open circle library molecule (300) having a nick or gap, where the nick or gap is asymmetrically positioned on the splint capture primer.

FIG. 17B is a schematic showing an exemplary covalently closed circular library molecule (400) generated by covalently closing the gap or nick of the open circle library molecule of FIG. 17A. The covalently close circular library molecule of FIG. 17B can be used to conduct a workflow comprising rolling circle amplification and sequencing, where the workflow is shown in FIGS. 29-36.

FIG. 18A is a schematic showing a support having an exemplary splint capture primer (200) immobilized thereon, where the splint capture primer can be used to conduct an on-support ligation reaction. The splint capture primer comprises a first portion (210) and a second portion (220). The schematic also shows an exemplary open circle library molecule (300) formed from a linear library molecule comprising a first universal binding site (120) for binding the first portion of the splint capture primer, and a second universal binding site (130) for binding the second portion of the same splint capture primer. The linear library molecule can also include a sequence of interest and at least one adaptor sequence. The linear library molecule is hybridized to the splint capture primer to form an open circle library molecule (300) having a nick or gap, where the nick or gap is asymmetrically positioned on the splint capture primer.

FIG. 18B is a schematic showing an exemplary covalently closed circular library molecule (400) generated by covalently closing the gap or nick of the open circle library molecule (300) of FIG. 18A. The covalently close circular library molecule of FIG. 18B can be used to conduct a workflow comprising rolling circle amplification and sequencing, where the workflow is shown FIGS. 29-36.

FIG. 19A is a schematic showing a support having an exemplary splint capture primer (200) immobilized thereon, where the splint capture primer can be used to conduct an on-support ligation reaction. The splint capture primer comprises a first portion (210) and a second portion (220). The schematic also shows an exemplary open circle library molecule (300) generated from a linear library molecule comprising a first universal binding site (120) for binding the first portion of the splint capture primer, and the linear library comprises a second universal binding site (130) for binding the second portion of the same splint capture primer. The linear library molecule can also include a sequence of interest and at least one adaptor sequence. The linear library molecule is hybridized to the splint capture primer to form an open circle library molecule (300) having a nick or gap, where the nick or gap is symmetrically positioned on the splint capture primer.

FIG. 19B is a schematic showing an exemplary covalently closed circular library molecule (400) generated by covalently closing the gap or nick of the open circle library molecule (300) of FIG. 19A. The covalently close circular library molecule of FIG. 19B can be used to conduct a workflow comprising rolling circle amplification and sequencing, where the workflow is shown in FIGS. 29-36.

FIGS. 20A-20D are schematics showing various embodiments of linear library molecules (100) comprising (i) a sequence of interest, also referred to herein as an “insert”, and any one or any combination of two or more adaptor sequences which can include (ii) a first universal binding site (120) for a first portion of a splint capture primer (or a complementary sequence thereof), (iii) at least one sample index sequence (e.g., a left sample index sequence (160) and/or a right sample index sequence (170) which can be used to distinguish sequences of interest (110) obtained from different sample sources in a multiplex assay, (iv) a universal binding site for a forward sequencing primer (140) or a complementary sequence thereof, (v) a universal binding site for a reverse sequencing primer (150) or a complementary sequence thereof, (vi) at least one unique molecular index sequence (UMI) (e.g., a left unique molecular index sequence (180) and/or a or a right unique molecular index sequence (190)) which can be used to uniquely identify a nucleic acid molecule (e.g., having a sequence of interest) to which the unique molecular index sequence is appended, and (vii) a second universal binding site (130) for a second portion of the immobilized splint capture primer or a complementary sequence thereof. In some embodiments, the universal binding site for a forward sequencing primer (140) comprises a batch-specific forward sequencing primer binding site which can be employed for batch sequencing. In some embodiments, the universal binding site for a reverse sequencing primer (150) comprises a batch-specific reverse sequencing primer binding site which can be employed for batch sequencing. In some embodiments, the at least one sample index sequence (e.g., (160) and/or (170)) comprises a sample index sequence joined to an optional short random sequence (e.g., NNN), where the short random sequence provides nucleotide sequence diversity and is about 3-20 nucleotides in length. In some embodiments, the sequence of interest (110) and any of the adaptor sequences can be arranged in any order.

FIGS. 21A-21F are schematics showing various embodiments of linear library molecules (100) comprising (i) a sequence of interest (110), also referred to herein as an “insert”, and any one or any combination of two or more adaptor sequences where the adaptor sequences comprise, (ii) a first universal binding site (120) for a first portion of a splint capture primer (or a complementary sequence thereof), (iii) at least one sample index sequence (e.g., (160) and/or (170) which can be used to distinguish sequences of interest obtained from different sample sources in a multiplex assay, (iv) a universal binding site for a forward sequencing primer (140) (or a complementary sequence thereof), (v) a universal binding site for a reverse sequencing primer (150) (or a complementary sequence thereof), and (vi) a second universal binding site (130) for a second portion of the immobilized splint capture (or a complementary sequence thereof). In some embodiments, the universal binding site for a forward sequencing primer (140) comprises a batch-specific forward sequencing primer binding site which can be employed for batch sequencing. In some embodiments, the universal binding site for a reverse sequencing primer (150) comprises a batch-specific reverse sequencing primer binding site which can be employed for batch sequencing. In some embodiments, the at least one sample index sequence (e.g., (160) and/or (170) comprises a sample index sequence joined to a short random sequence (e.g., NNN), where the short random sequence provides nucleotide sequence diversity and is about 3-20 nucleotides in length. In some embodiments, the sequence of interest (110) and any of the adaptor sequences can be arranged in any order.

FIGS. 22A-22C are schematic showing various embodiments of linear library molecules (100) comprising (i) a sequence-of-interest, also referred to as an insert (110), and any one or any combination of two or more adaptor sequences, where the adaptor sequences comprise (ii) a first universal binding site (120) for a first portion of a splint capture primer (or a complementary sequence thereof), (iii) a universal binding site for a first non-splint capture primer (123) (or a complementary sequence thereof), (iv) at least one sample index sequence (e.g., (160) and/or (170) which can be used to distinguish sequences of interest obtained from different sample sources in a multiplex assay, (v) at least one universal binding site for a forward sequencing primer (140) (or a complementary sequence thereof), (vi) at least one universal binding site for a reverse sequencing primer (150) (or a complementary sequence thereof), (vii) at least one unique molecular index sequence (UMI) (e.g., (180) and/or (190)) which can be used to uniquely identify a nucleic acid molecule (e.g., having a sequence of interest) to which the unique molecular index sequence is appended, (viii) a universal binding site for a second non-splint capture primer (133) (or a complementary sequence thereof), (ix) at least one optional short random sequence (e.g., NNNN) (132) which provides nucleotide sequence diversity and is about 3-20 nucleotides in length, and (x) a second universal binding site (130) for a second portion of the immobilized splint capture primer (or a complementary sequence thereof). The universal binding site for a forward sequencing primer (140) can comprise a batch-specific forward sequencing primer binding site which can be employed for batch sequencing. The universal binding site for the reverse sequencing primer (150) can comprise a batch-specific reverse sequencing primer binding site which can be employed for batch sequencing. In some embodiments, the at least one sample index sequence (e.g., (160) and/or (170) comprises a sample index sequence joined to an optional short random sequence (e.g., NNN) (not shown), where the short random sequence provides nucleotide sequence diversity and is about 3-20 nucleotides in length. In some embodiments, the sequence of interest (110) and any of the adaptor sequences can be arranged in any order.

FIGS. 23A-23F are schematics showing various embodiments of linear library molecules (100) comprising (i) a sequence of interest and any one or any combination of two or more adaptor sequences, where the adaptor sequences comprise (ii) a first universal binding site (120) for a first portion of a splint capture primer (or a complementary sequence thereof), (iii) a universal binding site for a first non-splint capture primer (123) (or a complementary sequence thereof), (iv) at least one sample index sequence (e.g., (160) and/or (170) which can be used to distinguish sequences of interest obtained from different sample sources in a multiplex assay, (v) at least one universal binding site for a forward sequencing primer (140) (or a complementary sequence thereof), (vi) at least one universal binding site for a reverse sequencing primer (150) (or a complementary sequence thereof), (vii) a universal binding site for a second non-splint capture primer (133) (or a complementary sequence thereof), and (viii) a second universal binding site (130) for a second portion of the immobilized splint capture primer (or a complementary sequence thereof). The universal binding site for the forward sequencing primer (140) can comprise a batch-specific forward sequencing primer binding site which can be employed for batch sequencing. The universal binding site for the reverse sequencing primer (150) can comprise a batch-specific reverse sequencing primer binding site which can be employed for batch sequencing. The at least one sample index sequence (e.g., (160) and/or (170) can comprise a sample index sequence joined to an optional short random sequence (e.g., NNN) (not shown), where the short random sequence provides nucleotide sequence diversity and is about 3-20 nucleotides in length. In some embodiments, the sequence of interest (110) and any of the adaptor sequences can be arranged in any order.

FIGS. 24A-24B are schematics showing various embodiments of linear library molecules (100) comprising at least one junction adaptor sequence located between any of the universal adaptor sequences described herein.

FIG. 25A is a schematic showing an exemplary splint capture primer (200) immobilized to a support, where the splint capture primer is hybridized to an exemplary open circle library molecule (300) having a nick or gap, which has been generated from linear library molecule.

FIG. 25B is a schematic showing an exemplary covalently closed circular library molecule (400) generated by covalently closing the gap or nick of the open circle library molecule (300) of FIG. 25A. The covalently close circular library molecule of FIG. 25B can be used to conduct a workflow comprising rolling circle amplification and sequencing, where the workflow is shown in FIGS. 29-36.

FIG. 26A is a schematic showing an exemplary splint capture primer (200) immobilized to a support, where the splint capture primer is hybridized to an open circle library (300) having a nick or gap generated from a linear library molecule.

FIG. 26B is a schematic showing an exemplary covalently closed circular library molecule (400) generated by covalently closing the gap or nick of the open circle library molecule (300) of FIG. 26A. The covalently close circular library molecule of FIG. 26B can be used to conduct a workflow comprising rolling circle amplification and sequencing, where the workflow is shown in FIGS. 29-36.

FIG. 27A is a schematic showing a mixture of exemplary splint capture primers (200-A) and (200-B), comprising different sequences, immobilized to the same support, where individual splint capture primers can be hybridized to their cognate linear library molecules to form open circle library molecules (300-A) and (300-B) each having a nick or gap. In this example, the first splint capture primer (200-A) can bind a first linear library molecule (while the second splint capture primer (200-B) can bind a second linear library molecule.

FIG. 27B is a schematic showing exemplary covalently closed circular library molecules (400-A) and (400-B) generated by covalently closing the gap or nick of the open circle library molecules of FIG. 27A. The covalently close circular library molecules of FIG. 27B can be used to conduct a workflow comprising rolling circle amplification and sequencing, where the workflow is shown in FIGS. 29-36.

FIG. 28A is a schematic showing various embodiments of open circle library molecules hybridized to a splint capture primer (200), in which the 5′ ends of the open circle library molecules form 5′ flap structures. FIG. 28A (left) is a schematic showing an open circle library molecule (300) comprising a 5′ overhang flap structure that is 2-10 nucleotides in length and a 3′ overhang flap structure that is 1 nucleotide in length, where the 5′ overhang flap structure is cleavable with an endonuclease (e.g., 5′ flap endonuclease 1, or FEN1). FIG. 28A (middle) is a schematic showing an open circle library molecule (300) comprising a 5′ overhang flap structure that is 2-10 nucleotides in length and lacking a 3′ overhang flap structure, where the 5′ overhang flap structure is cleavable with an endonuclease (e.g., 5′ flap endonuclease 1 or FEN1). FIG. 28A (right) is a schematic showing an open circle library molecule (300) comprising a 5′ overhang flap structure that is 2-10 nucleotides in length and a 3′ overhang flap structure that is 2-10 nucleotides in length, wherein the 5′ overhang flap structure is not cleavable with an endonuclease (e.g., 5′ flap endonuclease 1, or FEN1).

FIG. 28B is a schematic showing exemplary open circle library molecules hybridized to a splint capture primer (200-A or 200-B) where the 5′ end of the open circle library molecules form a 5′ flap structure. The schematic on the left shows the open circle library molecule shown in FIG. 28A (left) where the 5′ end of the open circle library molecule (300-A) forms a 5′ overhang flap structure that is 2-10 nucleotides in length and the 3′ end of the open circle library molecule (300-A) forms a 3′ overhang flap structure that is 1 nucleotide in length. In this example, the 5′ overhang flap structure is cleavable with an endonuclease (e.g. 5′ flap endonuclease 1, or FEN1). The schematic on the right shows the open circle library molecule shown in FIG. 27A (right) where the 5′ end of the open circle library molecule (300-B) forms a 5′ overhang flap structure that is 2-10 nucleotides in length and the 3′ end of the open circle library molecule (300-B) forms a 3′ overhang flap structure that is 1 nucleotide in length, and the 5′ overhang flap structure is cleavable with an endonuclease (e.g., 5′ flap endonuclease 1, or FEN1).

FIG. 29 is a schematic showing an exemplary on-support rolling circle amplification reaction using a covalently closed circular library molecule (400) and a mixture of nucleotides including nucleotides having a scissile moiety that can be cleaved to generate an abasic site. In some embodiments, the 3′ end of an immobilized splint capture primer can be used to initiate the rolling circle amplification reaction. The rolling circle amplification reaction generates an immobilized single stranded concatemer molecule having at least one nucleotide with a scissile moiety which can be cleaved to generate an abasic site in the immobilized concatemer molecule. Any of the linear library molecules shown in FIGS. 20-24, among others, can be used to generate the covalently closed circular library molecule which is hybridized to the immobilized splint capture primer as shown in FIG. 29 to initiate on-support rolling circle amplification.

FIG. 30 is a schematic showing an exemplary immobilized single stranded concatemer molecule having at least one nucleotide with a scissile moiety which can be cleaved to generate an abasic site in the immobilized concatemer template molecule.

FIG. 31 is a schematic showing an exemplary forward sequencing reaction conducted on the immobilized concatemer template molecule shown in FIG. 30. The forward sequencing reaction can be conducted with a plurality of soluble forward sequencing primers and generates a plurality of extended forward sequencing primer strands. The immobilized concatemer template molecule can have two or more extended forward sequencing primer strands hybridized thereon.

FIG. 32 is a schematic showing an exemplary method for replacing the extended forward sequencing primer strands by conducting a primer extension reaction with a strand displacing polymerase in the absence of an additional soluble primer, thereby generating a forward extension strand.

FIG. 33 is a schematic showing an exemplary method for replacing the extended forward sequencing primer strands by conducting a primer extension reaction with a soluble forward sequencing primer thereby generating a forward extension strand.

FIG. 34 is a schematic showing an exemplary method for generating abasic sites in the immobilized single stranded concatemer template molecules at the nucleotides having the scissile moiety, and generating gaps at the abasic sites to generate a plurality of gap-containing concatemer template molecules while retaining the plurality of forward extension strands and retaining the plurality of immobilized splint capture primers. The forward extension strand can be generated by the method depicted in FIG. 32 or 33.

FIG. 35 is a schematic showing an exemplary retained forward extension strand after removal of the gap-containing concatemer template molecule as shown in FIG. 34.

FIG. 36 is a schematic showing an exemplary reverse sequencing reaction conducted on the retained forward extension strand shown in FIG. 35. The reverse sequencing reaction can be conducted with a plurality of soluble reverse sequencing primers. The retained forward extension strand can have two or more extended reverse sequencing primer strands hybridized thereon. The extended reverse sequencing primer strands are not hybridized to the splint capture primer, or covalently joined to the splint capture primer. Therefore, the extended reverse sequencing primer strands are not immobilized to the support.

FIG. 37 is a schematic showing an exemplary support having a splint capture primer (200) and a pinning primer (500) immobilized thereon. The splint capture primer is joined to a concatemer template molecule. For example, an immobilized concatemer can be generated by the workflow shown in FIGS. 29-36. The immobilized concatemer template molecule comprises two or more copies of a universal binding sequence for the immobilized pinning primer. The portion of the immobilized concatemer template molecule that includes the universal binding sequence for an pinning primer is hybridized to the pinning primer.

FIG. 38A is a schematic showing an exemplary batch sequencing workflow. A first covalently closed circular molecule (left) can be generated by hybridizing individual linear library molecules (not shown) from a first sub-population to a splint capture primer (200) immobilized to a support. The hybridized first linear library molecule forms a first open circle library molecule (not shown) having a nick or gap which can be enzymatically closed to form a first covalently closed circular library molecule which is hybridized to the first splint capture primer. The first covalently closed library molecule comprises a first insert sequence (110-1), a first batch barcode sequence (142; BC-1); and a universal binding site for a first batch forward sequencing primer (140-1) which selectively hybridizes to a first batch forward sequencing primer. The universal binding site for a first batch forward sequencing primer (140-1) corresponds to the first insert sequence (110-1). A second covalently closed circular molecule (right) can be generated by hybridizing individual linear library molecules (not shown) from a second sub-population to a splint capture primer immobilized to the same support. The hybridized second linear library molecule forms a second open circle library molecule having a nick or gap (not shown) which can be enzymatically closed to form a second covalently closed circular library molecule which is hybridized to a second splint-capture primer. The second covalently closed library molecule comprises a second insert sequence (110-2) which differs from the first insert sequence (110-1), a second batch barcode sequence (143; BC-2) which differs from the first batch barcode sequence (143; BC-1); and a universal binding site for a second batch-specific forward sequencing primer (140-2) which selectively hybridizes to a second batch forward sequencing primer. The universal binding site for the second batch-specific forward sequencing primer (140-2) corresponds to the second insert sequence (110-2). The first and second covalently closed circular library molecules are subjected to rolling circle amplification (RCA) to generate a first batch concatemer template molecule and a second batch concatemer template molecule immobilized to the same support. The first and second concatemer template molecules are subjected to a first batch sequencing workflow which is repeated at least once (e.g., first batch reiterative sequencing). The first and second concatemer template molecules are subjected to a second batch sequencing workflow which is repeated at least once (e.g., second batch reiterative sequencing).

FIG. 38B is a schematic showing exemplary first and second batch reiterative sequencing workflows. The first and second concatemer template molecules are subjected to a first batch sequencing workflow using first batch-specific sequencing primers (solid arrows), sequencing polymerases, and a plurality of nucleotide reagents to generate a plurality of first sequencing read products (dashed arrows), where the first sequencing read products include the first batch barcode sequence (142; BC-1) and a portion of the first insert sequence (110-1). In this example, first concatemer template molecules undergo first batch reiterative sequencing comprising no more than 200 sequencing cycles, but the second concatemer template molecules do not undergo first batch sequencing because the first batch-specific sequencing primers do not hybridize to the universal binding sites for the second batch-specific forward sequencing primers (140-2).

FIG. 38C is a schematic showing a continuation of the exemplary first and second batch reiterative sequencing workflows described in FIG. 38B. The first and second concatemer template molecules are subjected to a second batch sequencing workflow using second batch-specific sequencing primers (solid arrows), sequencing polymerases, and a plurality of nucleotide reagents to generate a plurality of second sequencing read products (dashed arrows), where the second sequencing read products include the second batch barcode sequence ((143; BC-2) and a portion of the second insert sequence (110-2). In some embodiments, the second concatemer template molecules undergo second batch reiterative sequencing comprising no more than 200 sequencing cycles, but the first concatemers do not undergo second batch sequencing because the second batch-specific sequencing primers do not hybridize to the universal binding sites for the first batch-specific forward sequencing primers (140-1).

FIG. 39A is a schematic showing an exemplary batch sequencing workflow. A first covalently closed circular molecule (left) can be generated by hybridizing individual linear library molecules (not shown) from a first sub-population to a splint capture primer (200) immobilized to a support. The hybridized first linear library molecule forms a first open circle library molecule (not shown) having a nick or gap which can be enzymatically closed to form a first covalently closed circular library molecule which is hybridized to a first splint-capture primer. The first covalently closed library molecule (left) comprises a first insert sequence (110-1), a first batch barcode sequence (142; BC-1); and a universal binding site for a first batch-specific forward sequencing primer (140-1) which selectively hybridizes to a first batch forward sequencing primer. The universal binding site for the first batch-specific forward sequencing primer (140-1) corresponds to the first insert sequence (110-1). A second covalently closed circular molecule (right) can be generated by hybridizing individual linear library molecules (not shown) from a second sub-population to a splint capture primer (200) immobilized to the same support. The hybridized second linear library molecule forms a second open circle library molecule (not shown) having a nick or gap which can be enzymatically closed to form a second covalently closed circular library molecule which is hybridized to a second splint-capture primer. The second covalently closed library molecule (right) comprises a second insert sequence (110-2) which differs from the first insert sequence (110-1), a second batch barcode sequence (143; BC-2) which differs from the first batch barcode sequence (143; BC-1); and a universal binding site for a first batch-specific forward sequencing primer (140-1) which selectively hybridizes to a first batch forward sequencing primer. The universal binding site for a first batch-specific forward sequencing primer (140-1) corresponds to the second insert sequence (110-2). The first and second covalently closed circular library molecules are subjected to rolling circle amplification to generate first and second batch concatemer template molecules immobilized to the same support. The first and second concatemer template molecules are subjected to a batch sequencing workflow which is repeated at least once (e.g., batch reiterative sequencing).

FIG. 39B is a schematic showing an exemplary batch reiterative sequencing workflow in which one type of sequencing primer is used to sequence two different concatemer template molecules each carrying the same universal binding site for the first batch-specific forward sequencing primer (140-1). The first and second concatemer template molecules can be subjected to a batch sequencing workflow using first batch-specific sequencing primers (solid arrows), sequencing polymerases, and a plurality of nucleotide reagents to generate a plurality of first sequencing read products (dashed arrows), where the first sequencing read products include the first batch barcode sequence ((142; BC-1) and a portion of the first insert sequence (110-1). In this example, the first concatemer template molecules undergo reiterative sequencing comprising no more than 200 sequencing cycles. The batch sequencing workflow also generates a plurality of second sequencing read products (dashed arrows), where the second sequencing read products include the second batch barcode sequence ((143; BC-2) and a portion of the second insert sequence (110-2). In some embodiments, the second concatemers undergo reiterative sequencing comprising no more than 200 sequencing cycles.

FIG. 40A is a graph showing the nucleotide base diversity of a right sample index sequence (170) including the 3-mer random sequence (NNN). The graph shows a nucleotide diversity of the 3-mer random sequence (NNN) of approximately 30% for A and T base calls, and approximately 20% for C and G base calls.

FIG. 40B is a graph showing the nucleotide base diversity of a left sample index sequence (160) which lacks a 3-mer random sequence (NNN). The graph shows a nucleotide diversity of approximately 40% for A and T base calls, approximately 15% for C base calls, and approximately 5% for G base calls.

FIG. 41 is the amino acid sequence of a wild DNA polymerase having a backbone sequence from RLI 89578.1 (SEQ ID NO:128).

FIG. 42 is the amino acid sequence of a wild type DNA polymerase having a backbone sequence from KUO 42443.1 (SEQ ID NO:129).

FIG. 43 is the amino acid sequence of a wild type DNA polymerase having a backbone sequence from MBC 7218772.1 (SEQ ID NO:130).

FIG. 44 is the amino acid sequence of a wild DNA polymerase having a backbone sequence from NOZ 58130.1 (SEQ ID NO:131).

FIG. 45 is the amino acid sequence of a wild type DNA polymerase having a backbone sequence from RMF 90817.1 (SEQ ID NO:132).

FIG. 46 is the amino acid sequence of a wild DNA polymerase having a backbone sequence from NOZ 77387.1 (SEQ ID NO:133).

FIG. 47 is the amino acid sequence of a wild DNA polymerase having a backbone sequence from RLF 89458.1 (SEQ ID NO:134).

FIG. 48 is the amino acid sequence of a wild DNA polymerase having a backbone sequence from RLF 78286.1 (SEQ ID NO:135).

FIG. 49 is the amino acid sequence of a wild type DNA polymerase having a backbone sequence from WP 175059460.1 (SEQ ID NO:136).

FIG. 50 is the amino acid sequence of a Phi29 polymerase (SEQ ID NO:137).

FIG. 51 is the amino acid sequence of a wild type DNA polymerase having a backbone sequence from Geobacillus stearothermophilus (Bst polymerase) (SEQ ID NO:138).

FIG. 52 is the amino acid sequence of a 9° N polymerase (SEQ ID NO:139).

FIG. 53 is the amino acid sequence of a 9° N polymerase UniProt Q56366 (SEQ ID NO:140).

FIG. 54 is the amino acid sequence of a VENT® polymerase UniProt P30317 (SEQ ID NO:141).

FIG. 55 is the amino acid sequence of a DEEP VENT® polymerase UniProt Q51334 (SEQ ID NO:142).

FIG. 56 is the amino acid sequence of THERMINATOR™ polymerase (SEQ ID NO:143).

FIG. 57 is the amino acid sequence of a Pfu polymerase UniProt P61875 (SEQ ID NO:144).

FIG. 58 is the amino acid sequence of a Pyrococcus abyssi polymerase UniProt POCL77 (SEQ ID NO:145).

FIG. 59 is the amino acid sequence of an RB69 polymerase (SEQ ID NO:146).

FIG. 60 is the amino acid sequence of a bacteriophage T3 DNA ligase (SEQ ID NO:147).

FIG. 61 is the amino acid sequence of a bacteriophage T4 DNA ligase (SEQ ID NO:148).

FIG. 62 is the amino acid sequence of a bacteriophage T7 DNA ligase (SEQ ID NO:149).

FIG. 63 is the amino acid sequence of a bacteriophage Tfu DNA ligase (SEQ ID NO:150).

FIG. 64 is the amino acid sequence of a thermal stable DNA ligase from Thermococcus nautili (SEQ ID NO:151).

### DETAILED DESCRIPTION

### Introduction

The present disclosure provides compositions and methods for generating a plurality of nucleic acid concatemer template molecules immobilized to a support. The nucleic acid concatemer template molecules can be produced by conducting on-support ligation and circularization reactions using a plurality of linear library molecules and a plurality of immobilized splint capture primers. The present disclosure provides methods for seeding and optionally re-seeding the support to increase the density of immobilized nucleic acid concatemers that can be sequenced. In some embodiments, the immobilized concatemers can be used for conducting downstream sequencing workflows including batch sequencing and reiterative sequence workflows. The present disclosure also provides methods for interrupting an ongoing sequencing run to re-seed the support to generate additional concatemer template molecules that can be sequenced.

The headings provided herein are not limitations of the various aspects of the disclosure, which aspects can be understood by reference to the specification as a whole.

Unless defined otherwise, technical and scientific terms used herein have meanings that are commonly understood by those of ordinary skill in the art unless defined otherwise. Generally, terminologies pertaining to techniques of molecular biology, nucleic acid chemistry, protein chemistry, genetics, microbiology, transgenic cell production, and hybridization described herein are those well-known and commonly used in the art. Techniques and procedures described herein are generally performed according to conventional methods well known in the art and as described in various general and more specific references that are cited and discussed throughout the instant specification. For example, see Sambrook et al., Molecular Cloning: A Laboratory Manual (Third ed., Cold Spring Harbor Laboratory Press, Cold Spring Harbor, N.Y. 2000). See also Ausubel et al., Current Protocols in Molecular Biology, Greene Publishing Associates (1992). The nomenclatures utilized in connection with, and the laboratory procedures and techniques described herein are those well-known and commonly used in the art.

### Definitions

Unless otherwise required by context herein, singular terms shall include pluralities and plural terms shall include the singular. Singular forms “a”, “an” and “the”, and singular use of any word, include plural referents unless expressly and unequivocally limited on one referent.

It is understood the use of the alternative term (e.g., “or”) is taken to mean either one or both or any combination thereof of the alternatives.

The term “and/or” used herein is to be taken mean specific disclosure of each of the specified features or components with or without the other. For example, the term “and/or” as used in a phrase such as “A and/or B” herein is intended to include: “A and B”; “A or B”; “A” (A alone); and “B” (B alone). In a similar manner, the term “and/or” as used in a phrase such as “A, B, and/or C” is intended to encompass each of the following aspects: “A, B, and C”; “A, B, or C”; “A or C”; “A or B”; “B or C”; “A and B”; “B and C”; “A and C”; “A” (A alone); “B” (B alone); and “C” (C alone).

As used herein and in the appended claims, terms “comprising”, “including”, “having” and “containing”, and their grammatical variants, as used herein are intended to be non-limiting so that one item or multiple items in a list do not exclude other items that can be substituted or added to the listed items. It is understood that wherever aspects are described herein with the language “comprising,” otherwise analogous aspects described in terms of “consisting of” and/or “consisting essentially of” are also provided.

As used herein, the terms “about” and “approximately” refer to a value or composition that is within an acceptable error range for the particular value or composition as determined by one of ordinary skill in the art, which will depend in part on how the value or composition is measured or determined, i.e., the limitations of the measurement system. For example, “about” or “approximately” can mean within one or more than one standard deviation per the practice in the art. Alternatively, “about” or “approximately” can mean a range of up to 10% (i.e., ±10%) or more depending on the limitations of the measurement system. For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “approximately” should be assumed to be within an acceptable error range for that particular value or composition. Also, where ranges and/or subranges of values are provided, the ranges and/or subranges can include the endpoints of the ranges and/or subranges.

The term “biological sample” refers to a single cell, a plurality of cells, a tissue, an organ, an organism, or section of any of these biological samples. The biological sample can be extracted (e.g., biopsied) from an organism, or obtained from a cell culture grown in liquid or in a culture dish. The biological sample comprises a sample that is fresh, frozen, fresh frozen, or archived (e.g., formalin-fixed paraffin-embedded; FFPE). The biological sample can be embedded in a wax, resin, epoxy or agar. The biological sample can be fixed, for example in any one or any combination of two or more of acetone, ethanol, methanol, formaldehyde, paraformaldehyde-Triton or glutaraldehyde. The biological sample can be sectioned or non-sectioned. The biological sample can be stained, de-stained or non-stained.

The nucleic acids of interest can be extracted from biological samples using any of a number of techniques known to those of skill in the art. For example, a typical DNA extraction procedure comprises (i) collection of the cell sample or tissue sample from which DNA is to be extracted, (ii) disruption of cell membranes (i.e., cell lysis) to release DNA and other cytoplasmic components, (iii) treatment of the lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis. A variety of suitable commercial nucleic acid extraction and purification kits are consistent with the disclosure herein. Examples include, but are not limited to, the QIAamp kits (for isolation of genomic DNA from human samples) and DNAeasy kits (for isolation of genomic DNA from animal or plant samples) from Qiagen (Germantown, MD), or the Maxwell® and ReliaPrep™ series of kits from Promega (Madison, WI).

The terms “nucleic acid”, “polynucleotide” and “oligonucleotide” and other related terms used herein are used interchangeably and refer to polymers of nucleotides and are not limited to any particular length. Nucleic acids include recombinant and chemically-synthesized forms. Nucleic acids can be isolated. Nucleic acids include DNA molecules (e.g., cDNA or genomic DNA), RNA molecules (e.g., mRNA), analogs of the DNA or RNA generated using nucleotide analogs (e.g., peptide nucleic acids (PNA) and non-naturally occurring nucleotide analogs), and chimeric forms containing DNA and RNA. Nucleic acids can be single-stranded or double-stranded. Nucleic acids comprise polymers of nucleotides, where the nucleotides include natural or non-natural bases and/or sugars. Nucleic acids comprise naturally-occurring internucleosidic linkages, for example phosphdiester linkages. Nucleic acids can lack a phosphate group. Nucleic acids comprise non-natural internucleoside linkages, including phosphorothioate, phosphorothiolate, or peptide nucleic acid (PNA) linkages. Nucleic acid comprise a combination of natural and non-natural internucleoside linkages. In some embodiments, nucleic acids comprise a one type of polynucleotides or a mixture of two or more different types of polynucleotides.

The term “universal sequence”, “universal adaptor sequences” and related terms refers to a sequence in a nucleic acid molecule that is common among two or more polynucleotide molecules. For example, adaptors having the same universal sequence can be joined to a plurality of polynucleotides so that the population of co-joined molecules carry the same universal adaptor sequence. Examples of universal adaptor sequences include an amplification primer sequence, a sequencing primer sequence, a splint capture primer sequence, a pinning primer sequence or a non-splint primer sequence or sequences complementary thereto.

The term “operably linked” and “operably joined” or related terms as used herein refers to juxtaposition of components such that the activity of one component affects the other. The juxtapositioned components can be linked together covalently. For example, two nucleic acid components can be enzymatically ligated together where the linkage that joins together the two components comprises phosphodiester linkage. A first and second nucleic acid component can be linked together, where the first nucleic acid component can confer a function on a second nucleic acid component. For example, linkage between a primer binding sequence and a sequence of interest forms a nucleic acid library molecule having a portion that can bind to a primer. In another example, a transgene (e.g., a nucleic acid encoding a polypeptide or a nucleic acid sequence of interest) can be ligated to a vector where the linkage permits expression or functioning of the transgene sequence contained in the vector. In some embodiments, a transgene is operably linked to a host cell regulatory sequence (e.g., a promoter sequence) that affects expression of the transgene. In some embodiments, the vector comprises at least one host cell regulatory sequence, including a promoter sequence, enhancer, transcription and/or translation initiation sequence, transcription and/or translation termination sequence, polypeptide secretion signal sequences, and the like. In some embodiments, the host cell regulatory sequence controls expression of the level, timing and/or location of the transgene. The person of ordinary skill in the art will appreciate that components need not be directly or indirectly physically linked to be operably linked.

The terms “linked”, “joined”, “attached”, “appended” and variants thereof comprise any type of fusion, bond, adherence or association between any combination of compounds or molecules that is of sufficient stability to withstand use in the particular procedure. The procedure can include but are not limited to: nucleotide binding; nucleotide incorporation; de-blocking (e.g., removal of chain-terminating moiety); washing; removing; flowing; detecting; imaging and/or identifying. Such linkage can comprise, for example, covalent, ionic, hydrogen, dipole-dipole, hydrophilic, hydrophobic, or affinity bonding, bonds or associations involving van der Waals forces, mechanical bonding, and the like. In some embodiments, such linkage occurs intramolecularly, for example linking together the ends of a single-stranded or double-stranded linear nucleic acid molecule to form a circular molecule. In some embodiments, such linkage can occur between a combination of different molecules, or between a molecule and a non-molecule, including but not limited to: linkage between a nucleic acid molecule and a solid surface; linkage between a protein and a detectable reporter moiety; linkage between a nucleotide and detectable reporter moiety; and the like. Some examples of linkages can be found, for example, in Hermanson, G., “Bioconjugate Techniques”, Second Edition (2008); Aslam, M., Dent, A., “Bioconjugation: Protein Coupling Techniques for the Biomedical Sciences”, London: Macmillan (1998); Aslam, M., Dent, A., “Bioconjugation: Protein Coupling Techniques for the Biomedical Sciences”, London: Macmillan (1998).

The term “adaptor” and related terms refers to oligonucleotides that can be operably linked (appended) to a target polynucleotide, where the adaptor confers a function to the co-joined adaptor-target molecule. Adaptors comprise DNA, RNA, chimeric DNA/RNA, or analogs thereof. Adaptors can include at least one ribonucleoside residue. Adaptors can be single-stranded, double-stranded, or have single-stranded and/or double-stranded portions. Adaptors can be configured to be linear, stem-looped, hairpin, or Y-shaped forms. Adaptors can be any length, including 4-100 nucleotides or longer. Adaptors can have blunt ends, overhang ends, or a combination of both. Overhang ends include 5′ overhang and 3′ overhang ends. The 5′ end of a single-stranded adaptor, or one strand of a double-stranded adaptor, can have a 5′ phosphate group or lack a 5′ phosphate group. Adaptors can include a 5′ tail that does not hybridize to a target polynucleotide (e.g., tailed adaptor), or adaptors can be non-tailed. An adaptor can include a sequence that is complementary to at least a portion of a primer, such as an amplification primer, a sequencing primer, a splint capture primer, a pinning primer or a non-splint primer. Adaptors can include a random sequence or degenerate sequence. Adaptors can include at least one inosine residue. Adaptors can include at least one phosphorothioate, phosphorothiolate and/or phosphoramidate linkage. Adaptors can include a barcode sequence which can be used to distinguish polynucleotides (e.g., insert sequences) from different sample sources in a multiplex assay. Adaptors can include a unique identification sequence (e.g., unique molecular index, UMI; or a unique molecular tag) that can be used to uniquely identify a nucleic acid molecule to which the adaptor is appended. In some embodiments, a unique identification sequence can be used to increase error correction and accuracy, reduce the rate of false-positive variant calls and/or increase sensitivity of variant detection. Adaptors can include at least one restriction enzyme recognition sequence, including any one or any combination of two or more selected from a group consisting of type I, type II, type III, type IV, type Hs or type IIB.

The term “nucleic acid template”, “template polynucleotide”, “nucleic acid target” “target polynucleotide”, “template strand” and other variations refer to a nucleic acid strand that serves as the basis nucleic acid molecule for any of the analysis methods describe herein (e.g., primer extension, amplifying and/or sequencing). The template nucleic acid can be single-stranded or double-stranded, or the template nucleic acid can have single-stranded or double-stranded portions. The template nucleic acid can be obtained from a naturally-occurring source, recombinant form, or chemically synthesized to include any type of nucleic acid analog. The template nucleic acid can be linear, circular, or other forms. The template nucleic acids can include an insert region having an insert sequence which is also known as a sequence of interest. The template nucleic acids can also include at least one adaptor sequence. The template nucleic acid can be a concatemer having two or tandem copies of a sequence of interest and at least one adaptor sequence. The insert region can be isolated in any form, including chromosomal, genomic, organellar (e.g., mitochondrial, chloroplast or ribosomal), recombinant molecules, cloned, amplified, cDNA, RNA such as precursor mRNA or mRNA, oligonucleotides, whole genomic DNA, obtained from fresh frozen paraffin embedded tissue, needle biopsies, circulating tumor cells, cell free circulating DNA, or any type of nucleic acid library. The insert region can be isolated from any source including from organisms such as prokaryotes, eukaryotes (e.g., humans, plants and animals), fungus, viruses cells, tissues, normal or diseased cells or tissues, body fluids including blood, urine, serum, lymph, tumor, saliva, anal and vaginal secretions, amniotic samples, perspiration, semen, environmental samples, culture samples, or synthesized nucleic acid molecules prepared using recombinant molecular biology or chemical synthesis methods. The insert region can be isolated from any organ, including head, neck, brain, breast, ovary, cervix, colon, rectum, endometrium, gallbladder, intestines, bladder, prostate, testicles, liver, lung, kidney, esophagus, pancreas, thyroid, pituitary, thymus, skin, heart, larynx, or other organs. The template nucleic acid can be subjected to nucleic acid analysis, including sequencing and composition analysis.

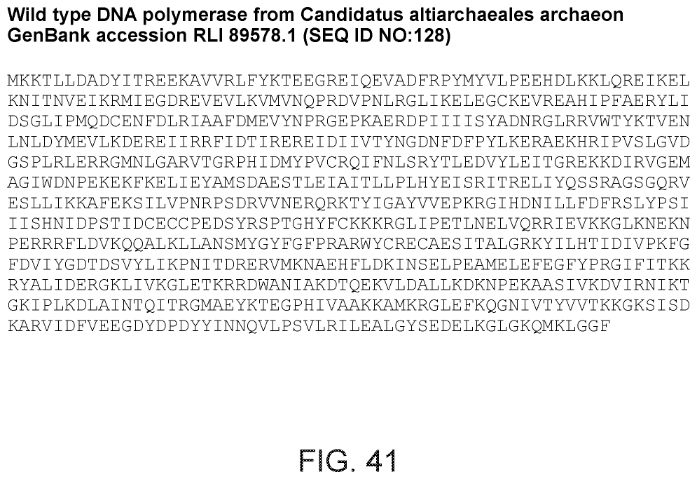

The term “polymerase” and its variants, as used herein, comprises an enzyme comprising a domain that binds a nucleotide (or nucleoside) where the polymerase can form a complex having a template nucleic acid and a complementary nucleotide. The polymerase can have one or more activities including, but not limited to, base analog detection activities, DNA polymerization activity, reverse transcriptase activity, DNA binding, strand displacement activity, and nucleotide binding and recognition. A polymerase can be any enzyme that can catalyze polymerization of nucleotides (including analogs thereof) into a nucleic acid strand. Typically but not necessarily such nucleotide polymerization can occur in a template-dependent fashion. Typically, a polymerase comprises one or more active sites at which nucleotide binding and/or catalysis of nucleotide polymerization can occur. In some embodiments, a polymerase includes other enzymatic activities, such as for example, 3′ to 5′ exonuclease activity or 5′ to 3′ exonuclease activity. In some embodiments, a polymerase has strand displacing activity. A polymerase can include without limitation naturally occurring polymerases and any subunits and truncations thereof, mutant polymerases, variant polymerases, recombinant, fusion or otherwise engineered polymerases, chemically modified polymerases, synthetic molecules or assemblies, and any analogs, derivatives or fragments thereof that retain the ability to catalyze nucleotide polymerization (e.g., catalytically active fragment). The polymerase includes catalytically inactive polymerases, catalytically active polymerases, reverse transcriptases, and other enzymes comprising a nucleotide binding domain. In some embodiments, a polymerase can be isolated from a cell, or generated using recombinant DNA technology or chemical synthesis methods. In some embodiments, a polymerase can be expressed in prokaryote, eukaryote, viral, or phage organisms. In some embodiments, a polymerase can be post-translationally modified proteins or fragments thereof. A polymerase can be derived from a prokaryote, eukaryote, virus or phage. A polymerase comprises DNA-directed DNA polymerase and RNA-directed DNA polymerase. Suitable polymerases are known in the art, and sequences of exemplary suitable polymerases are provided in FIGS. 41-59.

The term “strand displacing” refers to the ability of a polymerase to locally separate strands of double-stranded nucleic acids and synthesize a new strand in a template-based manner. Strand displacing polymerases displace a complementary strand from a template strand and catalyze new strand synthesis. Strand displacing polymerases include mesophilic and thermophilic polymerases. Strand displacing polymerases include wild type enzymes, and variants including exonuclease minus mutants, mutant versions, chimeric enzymes and truncated enzymes. Examples of strand displacing polymerases include phi29 DNA polymerase, large fragment of Bst DNA polymerase, large fragment of Bsu DNA polymerase (exo-), Bca DNA polymerase (exo-), Klenow fragment of E. coli DNA polymerase, T5 polymerase, M-MuLV reverse transcriptase, HIV viral reverse transcriptase, Deep Vent DNA polymerase and KOD DNA polymerase. The phi29 DNA polymerase can be wild type phi29 DNA polymerase (e.g., MagniPhi™ from Expedeon), or variant EquiPhi29™ DNA polymerase (e.g., from Thermo Fisher Scientific, catalog #A39390), or chimeric QualiPhi™ DNA polymerase (e.g., from 4basebio, catalog #510025).

As used herein, the term “fidelity” refers to the accuracy of DNA polymerization by template-dependent DNA polymerase. The fidelity of a DNA polymerase is typically measured by the error rate (the frequency of incorporating an inaccurate nucleotide, i.e., a nucleotide that is not complementary to the template nucleotide). The accuracy or fidelity of DNA polymerization is maintained by both the polymerase activity and the 3′-5′ exonuclease activity of a DNA polymerase.

As used herein, the term “binding complex” refers to a complex formed by binding together a nucleic acid duplex, a polymerase, and a free nucleotide or a nucleotide moiety of a multivalent molecule, where the nucleic acid duplex comprises a nucleic acid template molecule hybridized to a nucleic acid primer. In the binding complex, the free nucleotide or nucleotide moiety may or may not be bound to the 3′ end of the nucleic acid primer at a position that is opposite a complementary nucleotide in the nucleic acid template molecule. A “ternary complex” is an example of a binding complex which is formed by binding together a nucleic acid duplex, a polymerase, and a free nucleotide or nucleotide moiety of a multivalent molecule, where the free nucleotide or nucleotide moiety is bound to the 3′ end of the nucleic acid primer (as part of the nucleic acid duplex) at a position that is opposite a complementary nucleotide in the nucleic acid template molecule. An “avidity complex” refers to complex in which multiple nucleotide-moiety bearing arms of a single multivalent molecule participate in different ternary complexes.

The term “persistence time” and related terms refers to the length of time that a binding complex remains stable without dissociation of any of the components, where the components of the binding complex include a nucleic acid template and nucleic acid primer, a polymerase, a nucleotide moiety of a multivalent molecule or a free (e.g., unconjugated) nucleotide. The nucleotide moiety or the free nucleotide can be complementary or non-complementary to a nucleotide residue in the template molecule. The nucleotide moiety or the free nucleotide can bind to the 3′ end of the nucleic acid primer at a position that is opposite a complementary nucleotide residue in the nucleic acid template molecule. The persistence time is indicative of the stability of the binding complex and strength of the binding interactions. Persistence time can be measured by observing the onset and/or duration of a binding complex, such as by observing a signal from a labeled component of the binding complex. For example, a labeled nucleotide or a labeled reagent comprising one or more nucleotides may be present in a binding complex, thus allowing the signal from the label to be detected during the persistence time of the binding complex. One exemplary label is a fluorescent label. The binding complex (e.g., ternary complex) remains stable until subjected to a condition that causes dissociation of interactions between any of the polymerase, template molecule, primer and/or the nucleotide moiety or the nucleotide. For example, a dissociating condition comprises contacting the binding complex with any one or any combination of a detergent, EDTA and/or water.

The term “primer” and related terms as used herein refers to an oligonucleotide that is capable of hybridizing with a DNA and/or RNA polynucleotide template to form a duplex molecule. Primers comprise natural nucleotides and/or nucleotide analogs. Primers can be recombinant nucleic acid molecules. Primers may have any length, but typically range from 4-50 nucleotides. A typical primer comprises a 5′ end and 3′ end. The 3′ end of the primer can include a 3′ OH moiety which serves as a nucleotide polymerization initiation site in a polymerase-catalyzed primer extension reaction. Alternatively, the 3′ end of the primer can lack a 3′ OH moiety, or can include a terminal 3′ blocking group that inhibits nucleotide polymerization in a polymerase-catalyzed reaction. Any one nucleotide, or more than one nucleotide, along the length of the primer can be labeled with a detectable reporter moiety. A primer can be in solution (e.g., a soluble primer) or can be immobilized to a support (e.g., a splint capture primer or a pinning primer).

When used in reference to nucleic acid molecules, the terms “hybridize” or “hybridizing” or “hybridization” or other related terms refers to hydrogen bonding between two different nucleic acids to form a duplex nucleic acid. Hybridization also includes hydrogen bonding between two different regions of a single nucleic acid molecule to form a self-hybridizing molecule having a duplex region. Hybridization can comprise Watson-Crick or Hoogstein binding to form a duplex double-stranded nucleic acid, or a double-stranded region within a nucleic acid molecule. The double-stranded nucleic acid, or the two different regions of a single nucleic acid, may be wholly complementary, or partially complementary. Complementary nucleic acid strands need not hybridize with each other across their entire length. The complementary base pairing can be the standard A-T or C-G base pairing, or can be other forms of base-pairing interactions. Duplex nucleic acids can include mismatched base-paired nucleotides.

When used in reference to nucleic acids, the terms “extend”, “extending”, “extension” and other variants, refer to incorporation of one or more nucleotides into a nucleic acid molecule. Nucleotide incorporation comprises polymerization of one or more nucleotides into the terminal 3′ OH end of a nucleic acid strand (e.g., a nucleic acid primer), resulting in extension of the nucleic acid strand (e.g., extended primer). Nucleotide incorporation can be conducted with natural nucleotides and/or nucleotide analogs. Typically, but not necessarily, nucleotide incorporation occurs in a template-dependent fashion. Any suitable method of extending a nucleic acid molecule may be used, including primer extension catalyzed by a DNA polymerase or RNA polymerase.

The term “nucleotides” and related terms refers to a molecule comprising an aromatic base, a five carbon sugar (e.g., ribose or deoxyribose), and at least one phosphate group. Canonical or non-canonical nucleotides are consistent with use of the term. The phosphate in some embodiments comprises a monophosphate, diphosphate, or triphosphate, or corresponding phosphate analog. The term “nucleoside” refers to a molecule comprising an aromatic base and a sugar. Nucleotides and nucleosides can be non-labeled or labeled with a detectable reporter moiety.

Nucleotides (and nucleosides) typically comprise a hetero cyclic base including substituted or unsubstituted nitrogen-containing parent heteroaromatic ring which are commonly found in nucleic acids, including naturally-occurring, substituted, modified, or engineered variants, or analogs of the same. The base of a nucleotide (or nucleoside) is capable of forming Watson-Crick and/or Hoogstein hydrogen bonds with an appropriate complementary base. Exemplary bases include, but are not limited to, purines and pyrimidines such as: 2-aminopurine, 2,6-diaminopurine, adenine (A), ethenoadenine, N⁶-Δ²-isopentenyladenine (6iA), N⁶-Δ²-isopentenyl-2-methylthioadenine (2ms6iA), N⁶-methyladenine, guanine (G), isoguanine, N²-dimethylguanine (dmG), 7-methylguanine (7mG), 2-thiopyrimidine, 6-thioguanine (6sG), hypoxanthine and O⁶-methylguanine; 7-deaza-purines such as 7-deazaadenine (7-deaza-A) and 7-deazaguanine (7-deaza-G); pyrimidines such as cytosine (C), 5-propynylcytosine, isocytosine, thymine (T), 4-thiothymine (4sT), 5,6-dihydrothymine, O⁴-methylthymine, uracil (U), 4-thiouracil (4sU) and 5,6-dihydrouracil (dihydrouracil; D); indoles such as nitroindole and 4-methylindole; pyrroles such as nitropyrrole; nebularine; inosines; hydroxymethylcytosines; 5-methycytosines; base (Y); as well as methylated, glycosylated, and acylated base moieties; and the like. Additional exemplary bases can be found in Fasman, 1989, in “Practical Handbook of Biochemistry and Molecular Biology”, pp. 385-394, CRC Press, Boca Raton, Fla.

Nucleotides (and nucleosides) typically comprise a sugar moiety, such as carbocyclic moiety (Ferraro and Gotor 2000 Chem. Rev. 100: 4319-48), acyclic moieties (Martinez, et al., 1999 Nucleic Acids Research 27: 1271-1274; Martinez, et al., 1997 Bioorganic & Medicinal Chemistry Letters vol. 7: 3013-3016), and other sugar moieties (Joeng, et al., 1993 J. Med. Chem. 36: 2627-2638; Kim, et al., 1993 J. Med. Chem. 36: 30-7; Eschenmosser 1999 Science 284:2118-2124; and U.S. Pat. No. 5,558,991). The sugar moiety can comprise ribosyl; 2′-deoxyribosyl; 3′-deoxyribosyl; 2′,3′-dideoxyribosyl; 2′,3′-didehydrodideoxyribosyl; 2′-alkoxyribosyl; 2′-azidoribosyl; 2′-aminoribosyl; 2′-fluororibosyl; 2′-mercaptoriboxyl; 2′-alkylthioribosyl; 3′-alkoxyribosyl; 3′-azidoribosyl; 3′-aminoribosyl; 3′-fluororibosyl; 3′-mercaptoriboxyl; 3′-alkylthioribosyl carbocyclic; acyclic or other modified sugars.

In some embodiments, nucleotides comprise a chain of one, two or three phosphorus atoms where the chain is typically attached to the 5′ carbon of the sugar moiety via an ester or phosphoramide linkage. In some embodiments, the nucleotide is an analog having a phosphorus chain in which the phosphorus atoms are linked together with intervening O, S, NH, methylene or ethylene. In some embodiments, the phosphorus atoms in the chain include substituted side groups including O, S or BH₃. In some embodiments, the chain includes phosphate groups substituted with analogs including phosphoramidate, phosphorothioate, phosphorodithioate, and O-methylphosphoroamidite groups.

The term “reporter moiety”, “reporter moieties” or related terms refers to a compound that generates, or causes to generate, a detectable signal. A reporter moiety is sometimes called a “label”. Any suitable reporter moiety may be used, including luminescent, photoluminescent, electroluminescent, bioluminescent, chemiluminescent, fluorescent, phosphorescent, chromophore, radioisotope, electrochemical, mass spectrometry, Raman, hapten, affinity tag, atom, or an enzyme. A reporter moiety generates a detectable signal resulting from a chemical or physical change (e.g., heat, light, electrical, pH, salt concentration, enzymatic activity, or proximity events). A proximity event includes two reporter moieties approaching each other, or associating with each other, or binding each other. It is well known to one skilled in the art to select reporter moieties so that each absorbs excitation radiation and/or emits fluorescence at a wavelength distinguishable from the other reporter moieties to permit monitoring the presence of different reporter moieties in the same reaction or in different reactions. Two or more different reporter moieties can be selected having spectrally distinct emission profiles, or having minimal overlapping spectral emission profiles. Reporter moieties can be linked (e.g., operably linked) to nucleotides, nucleosides, nucleic acids, enzymes (e.g., polymerases or reverse transcriptases), or support (e.g., surfaces).

A reporter moiety (or label) can comprise a fluorescent label or a fluorophore. Exemplary fluorescent moieties which may serve as fluorescent labels or fluorophores include, but are not limited to fluorescein and fluorescein derivatives such as carboxyfluorescein, tetrachlorofluorescein, hexachlorofluorescein, carboxynapthofluorescein, fluorescein isothiocyanate, NHS-fluorescein, iodoacetamidofluorescein, fluorescein maleimide, SAMSA-fluorescein, fluorescein thiosemicarbazide, carbohydrazinomethylthioacetyl-amino fluorescein, rhodamine and rhodamine derivatives such as TRITC, TMR, lissamine rhodamine, Texas Red, rhodamine B, rhodamine 6G, rhodamine 10, NHS-rhodamine, TMR-iodoacetamide, lissamine rhodamine B sulfonyl chloride, lissamine rhodamine B sulfonyl hydrazine, Texas Red sulfonyl chloride, Texas Red hydrazide, coumarin and coumarin derivatives such as AMCA, AMCA-NHS, AMCA-sulfo-NHS, AMCA-HPDP, DCIA, AMCE-hydrazide, BODIPY® and derivatives such as BODIPY FL C3-SE, BODIPY 530/550 C3, BODIPY 530/550 C3-SE, BODIPY 530/550 C3 hydrazide, BODIPY 493/503 C3 hydrazide, BODIPY FL C3 hydrazide, BODIPY FL IA, BODIPY 530/551 IA, Br-BODIPY 493/503, Cascade® Blue and derivatives such as Cascade Blue acetyl azide, Cascade Blue cadaverine, Cascade Blue ethylenediamine, Cascade Blue hydrazide, Lucifer Yellow and derivatives such as Lucifer Yellow iodoacetamide, Lucifer Yellow CH, cyanine and derivatives such as indolium based cyanine dyes, benzo-indolium based cyanine dyes, pyridium based cyanine dyes, thiozolium based cyanine dyes, quinolinium based cyanine dyes, imidazolium based cyanine dyes, Cy 3, Cy5, lanthanide chelates and derivatives such as BCPDA, TBP, TMT, BHHCT, BCOT, Europium chelates, Terbium chelates, Alexa Fluor® dyes, DyLight dyes, Atto dyes, LightCycler® Red dyes, CAL Flour dyes, JOE and derivatives thereof, Oregon Green dyes, WellRED dyes, IRD dyes, phycoerythrin and phycobilin dyes, Malachite green, stilbene, DEG dyes, NR dyes, near-infrared dyes and others known in the art such as those described in Haugland, Molecular Probes Handbook, (Eugene, Oreg.) 6th Edition; Lakowicz, Principles of Fluorescence Spectroscopy, 2nd Ed., Plenum Press New York (1999), or Hermanson, Bioconjugate Techniques, 2nd Edition, or derivatives thereof, or any combination thereof. Cyanine dyes may exist in either sulfonated or non-sulfonated forms, and consist of two indolenin, benzo-indolium, pyridium, thiozolium, and/or quinolinium groups separated by a polymethine bridge between two nitrogen atoms. Commercially available cyanine fluorophores include, for example, Cy3, (which may comprise 1-[6-(2,5-dioxopyrrolidin-1-yloxy)-6-oxohexyl]-2-(3-{1-[6-(2,5-dioxopyrrolidin-1-yloxy)-6-oxohexyl]-3,3-dimethyl-1,3-dihydro-2H-indol-2-ylidene}prop-1-en-1-yl)-3,3-dimethyl-3H-indolium or 1-[6-(2,5-dioxopyrrolidin-1-yloxy)-6-oxohexyl]-2-(3-{1-[6-(2,5-dioxopyrrolidin-1-yloxy)-6-oxohexyl]-3,3-dimethyl-5-sulfo-1,3-dihydro-2H-indol-2-ylidene}prop-1-en-1-yl)-3,3-dimethyl-3H-indolium-5-sulfonate), Cy5 (which may comprise 1-(6-((2,5-dioxopyrrolidin-1-yl)oxy)-6-oxohexyl)-2-((1E,3E)-5-((E)-1-(6-((2,5-dioxopyrrolidin-1-yl)oxy)-6-oxohexyl)-3,3-dimethyl-5-indolin-2-ylidene)penta-1,3-dien-1-yl)-3,3-dimethyl-3H-indol-1-ium or 1-(6-((2,5-dioxopyrrolidin-1-yl)oxy)-6-oxohexyl)-2-((1E,3E)-5-((E)-1-(6-((2,5-dioxopyrrolidin-1-yl)oxy)-6-oxohexyl)-3,3-dimethyl-5-sulfoindolin-2-ylidene)penta-1,3-dien-1-yl)-3,3-dimethyl-3H-indol-1-ium-5-sulfonate), and Cy7 (which may comprise 1-(5-carboxypentyl)-2-[(1E,3E,5E,7Z)-7-(1-ethyl-1,3-dihydro-2H-indol-2-ylidene)hepta-1,3,5-trien-1-yl]-3H-indolium or 1-(5-carboxypentyl)-2-[(1E,3E,5E,7Z)-7-(1-ethyl-5-sulfo-1,3-dihydro-2H-indol-2-ylidene)hepta-1,3,5-trien-1-yl]-3H-indolium-5-sulfonate), where “Cy” stands for ‘cyanine’, and the first digit identifies the number of carbon atoms between two indolenine groups. Cy2 which is an oxazole derivative rather than indolenin, and the benzo-derivatized Cy3.5, Cy5.5 and Cy7.5 are exceptions to this rule.

In some embodiments, the reporter moiety can be a fluorescence resonance energy transfer (FRET) pair, such that multiple classifications can be performed under a single excitation and imaging step. As used herein, FRET may comprise excitation exchange (Forster) transfers, or electron-exchange (Dexter) transfers.

The disclosure provides supports, for use in the sequencing methods described herein.

The term “support” as used herein refers to a substrate that is designed for deposition of biological molecules or biological samples for assays and/or analyses. Examples of biological molecules to be deposited onto a support include nucleic acids (e.g., DNA, RNA), polypeptides, saccharides, lipids, a single cell or multiple cells. Examples of biological samples include but are not limited to saliva, phlegm, mucus, blood, plasma, serum, urine, stool, sweat, tears and fluids from tissues or organs.

In some embodiments, the support is solid, semi-solid, or a combination of both. In some embodiments, the support is porous, semi-porous, non-porous, or any combination of porosity. In some embodiments, the support can be substantially planar, concave, convex, or any combination thereof. In some embodiments, the support can be cylindrical, for example comprising a capillary or interior surface of a capillary.

In some embodiments, the surface of the support can be substantially smooth. In some embodiments, the support can be regularly or irregularly textured, including bumps, etched, pores, three-dimensional scaffolds, or any combination thereof.

In some embodiments, the support comprises a bead having any shape, including spherical, hemi-spherical, cylindrical, barrel-shaped, toroidal, disc-shaped, rod-like, conical, triangular, cubical, polygonal, tubular or wire-like.

The support can be fabricated from any material, including but not limited to glass, fused-silica, silicon, a polymer (e.g., polystyrene (PS), macroporous polystyrene (MPPS), polymethylmethacrylate (PMMA), polycarbonate (PC), polypropylene (PP), polyethylene (PE), high density polyethylene (HDPE), cyclic olefin polymers (COP), cyclic olefin copolymers (COC), polyethylene terephthalate (PET)), or any combination thereof. Various compositions of both glass and plastic substrates are contemplated.

The support can have a plurality (e.g., two or more) of nucleic acid concatemer template molecules immobilized thereon. The plurality of immobilized nucleic acid concatemer template molecules can have the same sequence or have different sequences. In some embodiments, individual nucleic acid concatemer template molecules in the plurality of nucleic acid templates are immobilized to a different site on the support. In some embodiments, two or more individual nucleic acid concatemer template molecules in the plurality of nucleic acid concatemer templates are immobilized to a site on the support.

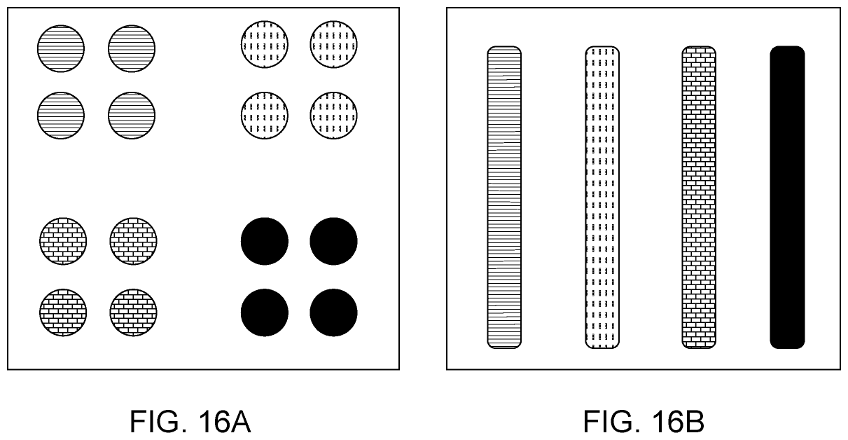

The term “array” refers to a support comprising a plurality of sites located at pre-determined locations on the support to form an array of sites (e.g., FIG. 16). The sites can be discrete and separated by interstitial regions. In some embodiments, the pre-determined sites on the support can be arranged in one dimension in a row or a column, or arranged in two dimensions in rows and columns. In some embodiments, the plurality of pre-determined sites is arranged on the support in an organized fashion. In some embodiments, the plurality of pre-determined sites is arranged in any organized pattern, including rectilinear, hexagonal patterns, grid patterns, patterns having reflective symmetry, patterns having rotational symmetry, or the like. The pitch between different pairs of sites can be that same or can vary. In some embodiments, the support comprises at least 10² sites, at least 10³ sites, at least 10⁴ sites, at least 10⁵ sites, at least 10⁶ sites, at least 10⁷ sites, at least 10⁸ sites, at least 10⁹ sites, at least 10¹⁰ sites, at least 10¹¹ sites, at least 10¹² sites, at least 10¹³ sites, at least 10¹⁴ sites, at least 10¹⁵ sites, or more, where the sites are located at pre-determined locations on the support. In some embodiments, a plurality of pre-determined sites on the support (e.g., 10²-10¹⁵ sites or more) are immobilized with nucleic acid templates to form a nucleic acid template array. In some embodiments, the nucleic acid templates that are immobilized at a plurality of pre-determined sites by hybridization to immobilized capture primers, or the nucleic acid templates are covalently attached to the capture primer. In some embodiments, the nucleic acid templates that are immobilized at a plurality of pre-determined sites, for example immobilized at 10²-10¹⁵ sites or more. In some embodiments, the immobilized nucleic acid templates are clonally-amplified to generate immobilized nucleic acid polonies at the plurality of pre-determined sites. In some embodiments, individual immobilized nucleic acid polonies comprise single-stranded or double-stranded concatemer template molecules.

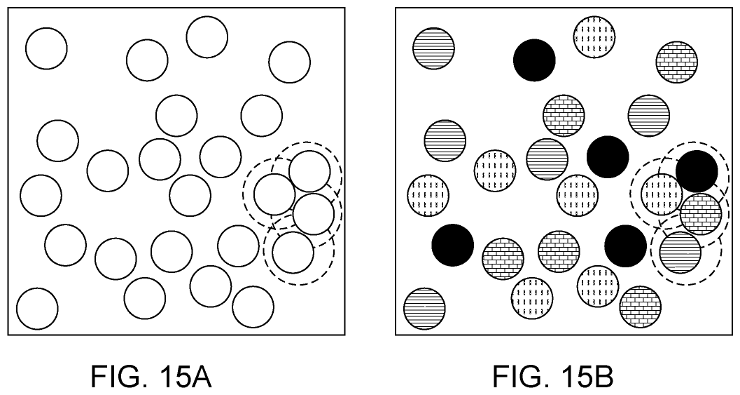

In some embodiments, a support comprising a plurality of sites located at random locations on the support is referred to herein as a support having randomly located sites thereon (e.g., FIG. 15). The location of the randomly located sites on the support are not pre-determined. The plurality of randomly-located sites is arranged on the support in a disordered and/or unpredictable fashion. In some embodiments, the support comprises at least 10² sites, at least 10³ sites, at least 10⁴ sites, at least 10⁵ sites, at least 10⁶ sites, at least 10⁷ sites, at least 10⁸ sites, at least 10⁹ sites, at least 10¹⁰ sites, at least 10¹¹ sites, at least 10¹² sites, at least 10¹³ sites, at least 10¹⁴ sites, at least 10¹⁵ sites, or more, where the sites are randomly located on the support. In some embodiments, a plurality of randomly located sites on the support (e.g., 10²-10¹⁵ sites or more) are immobilized with nucleic acid templates to form a support immobilized with nucleic acid templates. In some embodiments, the nucleic acid templates that are immobilized at a plurality of randomly located sites by hybridization to immobilized capture primers, or the nucleic acid templates are covalently attached to the capture primer. In some embodiments, the nucleic acid templates that are immobilized at a plurality of randomly located sites, for example immobilized at 10²-10¹⁵ sites or more. In some embodiments, the immobilized nucleic acid templates are clonally-amplified to generate immobilized nucleic acid polonies at the plurality of randomly located sites. In some embodiments, individual immobilized nucleic acid polonies comprise single-stranded or double-stranded concatemer template molecules.

When used in reference to a low binding surface coating, one or more layers of a multi-layered surface coating may comprise a branched polymer or may be linear. Examples of suitable branched polymers include, but are not limited to, branched PEG, branched poly(vinyl alcohol) (branched PVA), branched poly(vinyl pyridine), branched poly(vinyl pyrrolidone) (branched PVP), branched), poly(acrylic acid) (branched PAA), branched polyacrylamide, branched poly(N-isopropylacrylamide) (branched PNIPAM), branched poly(methyl methacrylate) (branched PMA), branched poly(2-hydroxylethyl methacrylate) (branched PHEMA), branched poly(oligo(ethylene glycol) methyl ether methacrylate) (branched POEGMA), branched polyglutamic acid (branched PGA), branched poly-lysine, branched poly-glucoside, and dextran.

In some embodiments, the branched polymers used to create one or more layers of any of the multi-layered surfaces disclosed herein may comprise at least 4 branches, at least 5 branches, at least 6 branches, at least 7 branches, at least 8 branches, at least 9 branches, at least 10 branches, at least 12 branches, at least 14 branches, at least 16 branches, at least 18 branches, at least 20 branches, at least 22 branches, at least 24 branches, at least 26 branches, at least 28 branches, at least 30 branches, at least 32 branches, at least 34 branches, at least 36 branches, at least 38 branches, or at least 40 branched.

Linear, branched, or multi-branched polymers used to create one or more layers of any of the multi-layered surfaces disclosed herein may have a molecular weight of at least 500, at least 1,000, at least 2,000, at least 3,000, at least 4,000, at least 5,000, at least 10,000, at least 15,000, at least 20,000, at least 25,000, at least 30,000, at least 35,000, at least 40,000, at least 45,000, or at least 50,000 daltons.

In some embodiments, e.g., wherein at least one layer of a multi-layered surface comprises a branched polymer, the number of covalent bonds between a branched polymer molecule of the layer being deposited and molecules of the previous layer may range from about one covalent linkage per molecule and about 32 covalent linkages per molecule. In some embodiments, the number of covalent bonds between a branched polymer molecule of the new layer and molecules of the previous layer may be at least 1, at least 2, at least 3, at least 4, at least 5, at least 6, at least 7, at least 8, at least 9, at least 10, at least 12, at least 14, at least 16, at least 18, at least 20, at least 22, at least 24, at least 26, at least 28, at least 30, or at least 32 covalent linkages per molecule.

Any reactive functional groups that remain following the coupling of a material layer to the surface may optionally be blocked by coupling a small, inert molecule using a high yield coupling chemistry. For example, in the case that amine coupling chemistry is used to attach a new material layer to the previous one, any residual amine groups may subsequently be acetylated or deactivated by coupling with a small amino acid such as glycine.

The number of layers of low non-specific binding material, e.g., a hydrophilic polymer material, deposited on the surface, may range from 1 to about 10. In some embodiments, the number of layers is at least 1, at least 2, at least 3, at least 4, at least 5, at least 6, at least 7, at least 8, at least 9, or at least 10. In some embodiments, the number of layers may be at most 10, at most 9, at most 8, at most 7, at most 6, at most 5, at most 4, at most 3, at most 2, or at most 1. Any of the lower and upper values described in this paragraph may be combined to form a range included within the present disclosure, for example, in some embodiments the number of layers may range from about 2 to about 4. In some embodiments, all of the layers may comprise the same material. In some embodiments, each layer may comprise a different material. In some embodiments, the plurality of layers may comprise a plurality of materials. In some embodiments at least one layer may comprise a branched polymer. In some embodiment, all of the layers may comprise a branched polymer.

One or more layers of low non-specific binding material may in some cases be deposited on and/or conjugated to the substrate surface using a polar protic solvent, a polar or polar aprotic solvent, a nonpolar solvent, or any combination thereof. In some embodiments the solvent used for layer deposition and/or coupling may comprise an alcohol (e.g., methanol, ethanol, propanol, etc.), another organic solvent (e.g., acetonitrile, dimethyl sulfoxide (DMSO), dimethyl formamide (DMF), etc.), water, an aqueous buffer solution (e.g., phosphate buffer, phosphate buffered saline, 3-(N-morpholino)propanesulfonic acid (MOPS), etc.), or any combination thereof. In some embodiments, an organic component of the solvent mixture used may comprise at least 1%, 5%⁰, 1, 15%, 20%, 25%, 30%, 35%, 40%, 45%, 50%, 55%, 60%, 70%, 75%, 80%, 85%, 90%, 95%, 98%, or 99% of the total, with the balance made up of water or an aqueous buffer solution. In some embodiments, an aqueous component of the solvent mixture used may comprise at least 1%, 5%, 10%, 15%, 20%, 25%, 30%, 35%, 40%, 45%, 50%, 55%, 60%, 70%, 75%, 80%, 85%, 90%, 95%, 98%, or 99% of the total, with the balance made up of an organic solvent. The pH of the solvent mixture used may be less than 6, about 6, 6.5, 7, 7.5, 8, 8.5, 9, or greater than pH 9.

The term “branched polymer” and related terms refers to a polymer having a plurality of functional groups that help conjugate a biologically active molecule such as a nucleotide, and the functional group can be either on the side chain of the polymer or directly attaches to a central core or central backbone of the polymer. The branched polymer can have linear backbone with one or more functional groups coming off the backbone for conjugation. The branched polymer can also be a polymer having one or more sidechains, wherein the side chain has a site suitable for conjugation. Examples of the functional group include but are limited to hydroxyl, ester, amine, carbonate, acetal, aldehyde, aldehyde hydrate, alkenyl, acrylate, methacrylate, acrylamide, active sulfone, hydrazide, thiol, alkanoic acid, acid halide, isocyanate, isothiocyanate, maleimide, vinylsulfone, dithiopyridine, vinylpyridine, iodoacetamide, epoxide, glyoxal, dione, mesylate, tosylate, and tresylate.

When used in reference to immobilized nucleic acids, the term “immobilized” and related terms refer to nucleic acid molecules that are attached to a support through covalent bond or non-covalent interaction, or attached to a coating on the support, or buried within a matrix formed by a coating on the support, where the nucleic acid molecules include splint capture primers (200), pinning primers (500), nucleic acid concatemer template molecules and extension products of capture primers. Extension products of capture primers includes nucleic acid concatemer template molecules that can form nucleic acid polonies.

In some embodiments, one or more nucleic acid concatemer template molecules are immobilized on the support, for example immobilized at the sites on the support. In some embodiments, the one or more nucleic acid templates are clonally-amplified. In some embodiments, the one or more nucleic acid template concatemer template molecules are clonally-amplified off the support (e.g., in-solution) and then deposited onto the support and immobilized on the support. In some embodiments, the clonal amplification reaction of the one or more nucleic acid concatemer template molecules is conducted on the support resulting in immobilization on the support. In some embodiments, the one or more nucleic acid concatemer template molecules are clonally-amplified (e.g., in solution or on the support) using a nucleic acid amplification reaction, including any one or any combination of: polymerase chain reaction (PCR), multiple displacement amplification (MDA), transcription-mediated amplification (TMA), nucleic acid sequence-based amplification (NASBA), strand displacement amplification (SDA), real-time SDA, bridge amplification, isothermal bridge amplification, rolling circle amplification (RCA), circle-to-circle amplification, helicase-dependent amplification, recombinase-dependent amplification, and/or single-stranded binding (SSB) protein-dependent amplification.

The term “capture primer”, “splint capture primer”, “immobilized splint capture primer” and related terms refers to single-stranded oligonucleotides that are immobilized to a support and comprise a sequence that can hybridize to at least a portion of a nucleic acid library molecule. Splint capture primers can be used to immobilize linear library molecules to a support via hybridization. Splint capture primers can be immobilized to a support in a manner that resists primer removal during flowing, washing, aspirating, and changes in temperature, pH, salts, chemical and/or enzymatic conditions. Typically, but not necessarily, the 5′ end of a capture primer can be immobilized to a support. Alternatively, an interior portion or the 3′ end of a capture primer can be immobilized to a support.

The sequence of splint capture primers can be wholly complementary or partially complementary along their length to at least a portion of the linear library molecule. A support can include a plurality of immobilized splint capture primers having the same sequence, or having two or more different sequences. Splint capture primers can be any length, for example 4-50 nucleotides, or 50-100 nucleotides, or 100-150 nucleotides, or longer lengths. A splint capture primer can include a terminal 3′ nucleotide having a sugar 3′ OH moiety which is extendible for nucleotide polymerization (e.g., polymerase catalyzed polymerization). A splint capture primer can include a terminal 3′ nucleotide having a moiety that blocks polymerase-catalyzed extension. A splint capture primer can include a terminal 3′ nucleotide having the 3′ sugar position linked to a chain-terminating moiety that inhibits nucleotide polymerization. The 3′ chain-terminating moiety can be removed (e.g., de-blocked) to convert the 3′ end to an extendible 3′ OH end using a de-blocking agent.

The term “pinning primer”, “immobilized pinning primer” and related terms refers to single-stranded oligonucleotides that are immobilized to a support and comprise a sequence that can hybridize to at least a portion of a nucleic acid concatemer template molecule. Pinning primers can be used to immobilize concatemer template molecules to a support via hybridization. Pinning primers can be immobilized to a support in a manner that resists primer removal during flowing, washing, aspirating, and changes in temperature, pH, salts, chemical and/or enzymatic conditions. Typically, but not necessarily, the 5′ end of a pinning primer can be immobilized to a support. Alternatively, an interior portion or the 3′ end of a pinning primer can be immobilized to a support.

The sequence of pinning primers (500) can be wholly complementary or partially complementary along their length to at least a portion of the concatemer template molecule. A support can include a plurality of immobilized pinning primers having the same sequence, or having two or more different sequences. Pinning primers can be any length, for example 4-50 nucleotides, or 50-100 nucleotides, or 100-150 nucleotides, or longer lengths. A pinning primer can include a terminal 3′ nucleotide having a sugar 3′ OH moiety which is extendible for nucleotide polymerization (e.g., polymerase catalyzed polymerization). A pinning primer can include a terminal 3′ phosphate moiety which blocks polymerase-catalyzed extension. A pinning primer can include a terminal 3′ nucleotide having a moiety that blocks polymerase-catalyzed extension. A pinning primer can include a terminal 3′ nucleotide having the 3′ sugar position linked to a chain-terminating moiety that inhibits nucleotide polymerization. The 3′ chain-terminating moiety can be removed (e.g., de-blocked) to convert the 3′ end to an extendible 3′ OH end using a de-blocking agent.

The splint capture primers (200) and pinning primers (500) can comprise DNA, RNA, or analogs thereof. The capture primers and pinning primers can include a combination of DNA and RNA.

The 3′ terminal end of a splint capture primer (200) or a pinning primer (500) can include a chain terminating moiety. Examples of chain terminating moieties include alkyl group, alkenyl group, alkynyl group, allyl group, aryl group, benzyl group, azide group, amine group, amide group, keto group, isocyanate group, phosphate group, thio group, disulfide group, carbonate group, urea group, acetal group or silyl group. Azide type chain terminating moieties including azide, azido and azidomethyl groups. Examples of de-blocking agents include a phosphine compound, such as Tris(2-carboxyethyl)phosphine (TCEP) and bis-sulfo triphenyl phosphine (BS-TPP), for chain-terminating groups azide, azido and azidomethyl groups. Examples of de-blocking agents include tetrakis(triphenylphosphine)palladium(0) (Pd(PPh₃)₄) with piperidine, or with 2,3-Dichloro-5,6-dicyano-1,4-benzo-quinone (DDQ), for chain-terminating groups alkyl, alkenyl, alkynyl and allyl. Examples of a de-blocking agent includes Pd/C for chain-terminating groups aryl and benzyl. Examples of de-blocking agents include phosphine, beta-mercaptoethanol or dithiothritol (DTT), for chain-terminating groups amine, amide, keto, isocyanate, phosphate, thio and disulfide. Examples of de-blocking agents include potassium carbonate (K₂CO₃) in MeOH, triethylamine in pyridine, and Zn in acetic acid (AcOH), for carbonate chain-terminating groups. Examples of de-blocking agents include tetrabutylammonium fluoride, pyridine-HF, with ammonium fluoride, and triethylamine trihydrofluoride, for chain-terminating groups urea and silyl.

In some embodiments, the plurality of immobilized capture primers (200) and pinning primers (500) on the support are in fluid communication with each other to permit flowing a solution of reagents (e.g., linear library molecules, or covalently closed circular library molecules, soluble primers, enzymes, nucleotides, divalent cations, buffers, reagents and the like) onto the support so that the plurality of immobilized capture primers and pinning primers on the support can be essentially simultaneously reacted with the reagents in a massively parallel manner. In some embodiments, the fluid communication of the plurality of immobilized capture primers and pinning primers can be used to conduct nucleic acid amplification reactions (e.g., RCA, MDA, PCR and bridge amplification) essentially simultaneously on the plurality of immobilized capture primers and pinning primers.

In some embodiment, the plurality of nucleic acid concatemer template molecules immobilized on the support are in fluid communication with each other to permit flowing a solution of reagents (e.g., soluble primers, enzymes, nucleotides, divalent cations, buffers, reagents and the like) onto the support so that the plurality of concatemer template molecules on the support can be essentially simultaneously reacted with the reagents in a massively parallel manner. In some embodiments, the fluid communication of the plurality of concatemer template molecules can be used to conduct nucleotide binding assays and/or conduct nucleotide polymerization reactions (e.g., primer extension or sequencing) essentially simultaneously on the plurality of concatemer template molecules, and optionally to conduct detection and imaging for massively parallel sequencing.

When used in reference to nucleic acids, the terms “amplify”, “amplifying”, “amplification”, and other related terms include producing multiple copies of an original polynucleotide template molecule, where the copies comprise a sequence that is complementary to the template sequence, or the copies comprise a sequence that is the same as the template sequence. In some embodiments, the copies comprise a sequence that is substantially identical to a template sequence, or is substantially identical to a sequence that is complementary to the template sequence.

The present disclosure provides various pH buffering agents. The full name of the pH buffering agents is listed herein. The term “Tris” refers to a pH buffering agent Tris(hydroxymethyl)-aminomethane. The term “Tris-HCl” refers to a pH buffering agent Tris(hydroxymethyl)-aminomethane hydrochloride. The term “Tricine” refers to a pH buffering agent N-[tris(hydroxymethyl)methyl]glycine. The term “Bicine” refers to a pH buffering agent N,N-bis(2-hydroxyethyl)glycine. The term “Bis-Tris propane” refers to a pH buffering agent 1,3 Bis[tris(hydroxymethyl)methylamino]propane. The term “HEPES” refers to a pH buffering agent 4-(2-hydroxyethyl)-1-piperazineethanesulfonic acid. The term “MES” refers to a pH buffering agent 2-(N-morpholino)ethanesulfonic acid). The term “MOPS” refers to a pH buffering agent 3-(N-morpholino)propanesulfonic acid. The term “MOPSO” refers to a pH buffering agent 3-(N-morpholino)-2-hydroxypropanesulfonic acid. The term “BES” refers to a pH buffering agent N,N-bis(2-hydroxyethyl)-2-aminoethanesulfonic acid. The term “TES” refers to a pH buffering agent 2-[(2-Hydroxy-1,1bis(hydroxymethyl)ethyl)amino]ethanesulfonic acid). The term “CAPS” refers to a pH buffering agent 3-(cyclohexylamino)-1-propanesuhinic acid. The term “TAPS” refers to a pH buffering agent N-[Tris(hydroxymethyl)methyl]-3-amino propane sulfonic acid. The term “TAPSO” refers to a pH buffering agent N-[Tris(hydroxymethyl)methyl]-3-amino-2-hyidroxypropansulfonic acid. The term “ACES” refers to a pH buffering agent N-(2-Acetamido)-2-aminoethanesulfonic acid. The term “PIPES” refers to a pH buffering agent piperazine-1,4-bis(2-ethanesulfonic acid.

Throughout this application various publications, patents, and/or patent applications are referenced. The disclosures of the publications, patents and/or patent applications are hereby incorporated by reference in their entireties into this application in order to more fully describe the state of the art to which this disclosure pertains.

### On-Support Circularization and RCA

The present disclosure provides a method for generating a plurality of nucleic acid concatemer template molecules immobilized to a support, comprising step (a): providing a support having a plurality of splint capture primers (200) immobilized thereon. In some embodiments, the support further comprises a plurality of pinning primers (500) immobilized thereon.

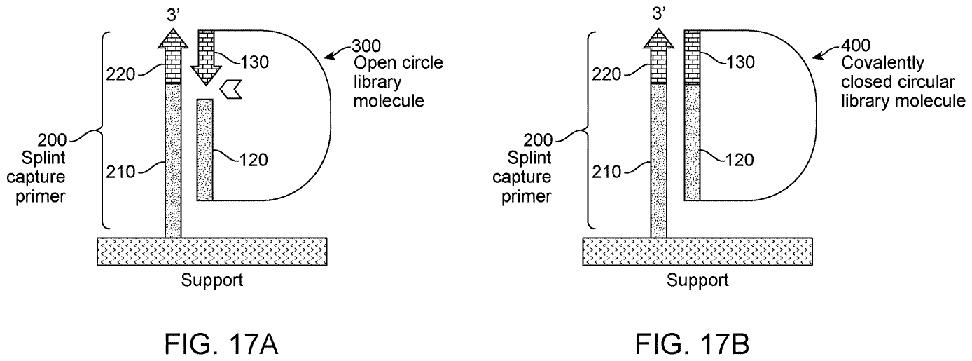

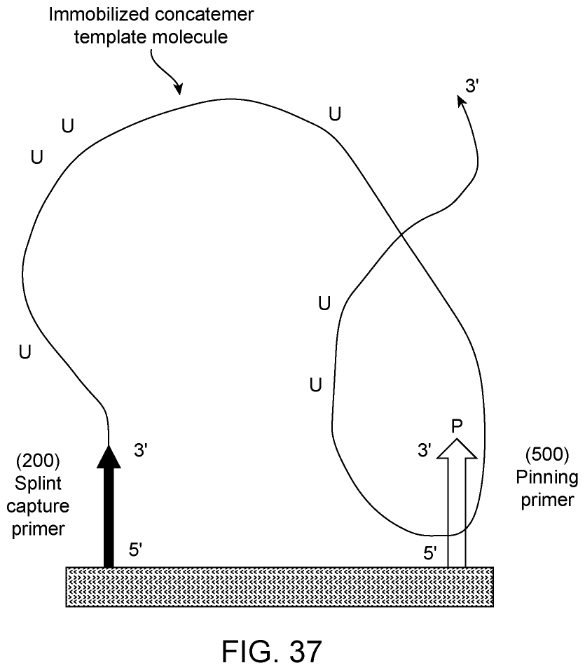

In some embodiments, in step (a), the plurality of immobilized splint capture primers (200) hybridize to portions of linear library molecules (100) and to serve as initiation sites for rolling circle amplification to generate a plurality of immobilized nucleic acid concatemer template molecules (e.g., see FIGS. 17A-17B, 18A-18B, 19A-19B, 25A-25B, 26A-26B and 27A-27B). In some embodiments, the plurality of pinning primers (500) hybridize to portions of a concatemer template molecule and pin down the concatemer template molecule to the support (e.g., see FIG. 37). Together, the splint capture primers and pinning primers generate immobilized concatemer template molecules having a compact shape and size. In some embodiments, the terminal 3′ end of the pinning primers (500) are non-extendible. In some embodiments, the pinning primers (500) include a terminal 3′ blocking group that renders them non-extendible. In some embodiments, the plurality of splint capture primers (200) and pinning primers (500) can be used for batch-specific sequencing (described below) or for non-batch-specific sequencing.

In some embodiments, in step (a), the plurality of splint capture primers (200) comprise the same sequence. In some embodiments, in step (a), the plurality of splint capture primers (200) comprise different sequences. In some embodiments, individual splint capture primers comprise a sequence that is wholly complementary or partially complementary along their lengths to at least a portion of a nucleic acid library molecule (e.g., a linear or circular library molecules). In some embodiments, in step (a), individual splint capture primers comprise a sequence that is complementary to at least a portion of a universal adaptor sequence in a nucleic acid library molecule.

In some embodiments, in step (a), individual splint capture primers (200) in the plurality comprise a first portion (210) which binds a first universal binding site in a linear library molecule, and individual splint capture primers (200) comprise a second portion (220) which binds a second universal binding site in the same linear library molecule (e.g., FIGS. 17A-17B, 18A-18B, 19A-19B, 25A-25B, 26A-26B and 27A-27B). In some embodiments, the first and second portions (e.g., (210) and (220)) of the splint capture primers have the same or different lengths. The first portion (210) of the splint capture primers can be about 4-50 nucleotides, or 50-100 nucleotides, or 100-150 nucleotides, or longer lengths. The second portion (220) of the splint capture primers can be about 4-50 nucleotides, or 50-100 nucleotides, or 100-150 nucleotides, or longer lengths. In some embodiments, the first and second portions (e.g., (210) and (220)) of the splint capture primers have the same sequence. In some embodiments, the first and second portions (e.g., (210) and (220)) of the immobilized splint capture primers have different sequences.

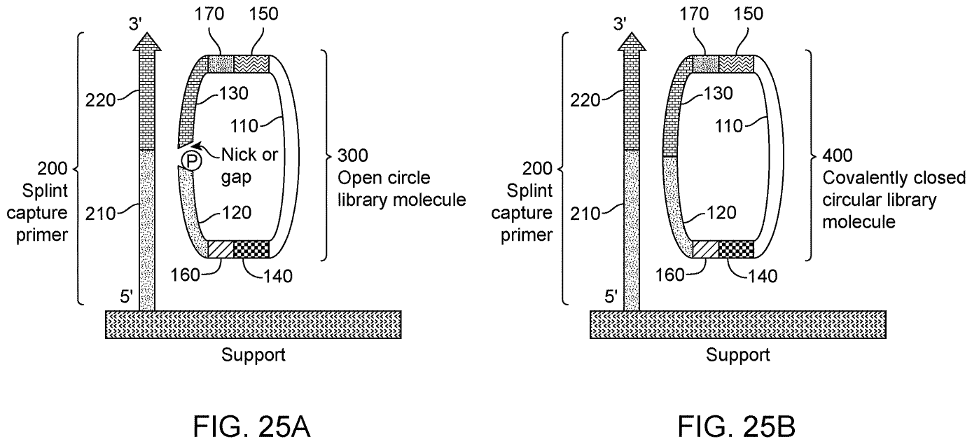

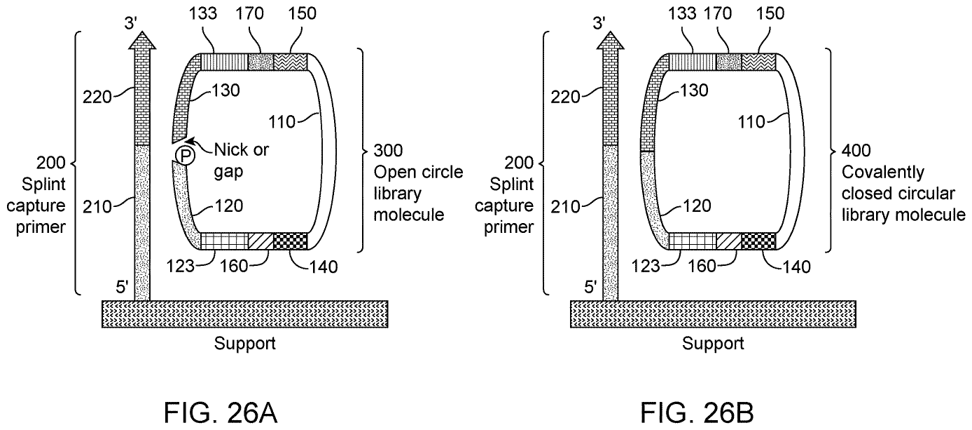

In some embodiments, in step (a), the plurality of splint capture primers (200) that are immobilized to the support comprise one type of splint capture primers having the same sequence. For example, individual immobilized splint capture primers (200) comprise a first portion (210) and a second portion (220), wherein the first portion of the splint capture primer (210) binds a first universal binding site (120) in a linear library molecule and the second portion (220) of the splint capture primer binds a second universal binding site (130) in the same linear library molecule (e.g., FIGS. 25A-25B, 26A-26B).

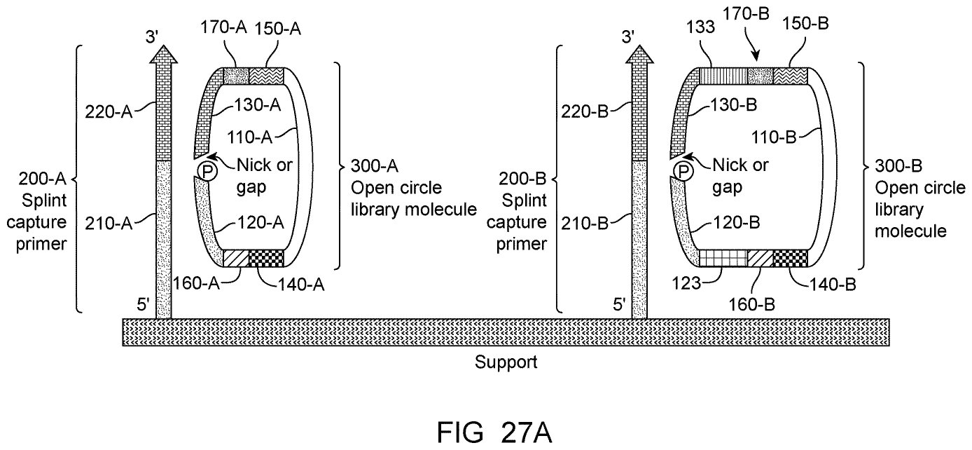

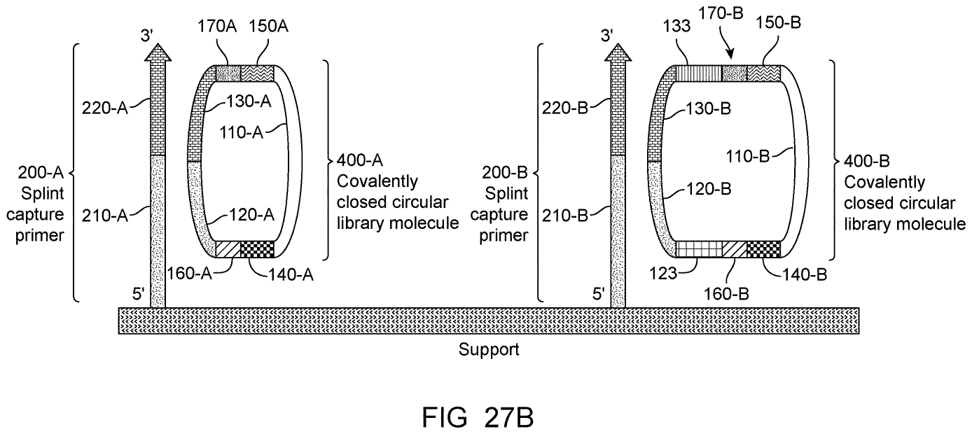

In some embodiments, in step (a), the plurality of splint capture primers (200) that are immobilized to the support comprise a mixture of different types of splint capture primers including at least a first and second sub-population of splint capture primers having different sequences, wherein the different types of splint capture primers bind to different types of linear library molecules (e.g., FIG. 27A-27B). In some embodiments, individual immobilized splint capture primers in the first sub-population (200-A) comprise a first portion (210-A) and a second portion (220-A), wherein the first portion of the splint capture primer (210-A) binds a first universal binding site (120-A) in a first linear library molecule and the second portion of the splint capture primer (220-A) binds a second universal binding site (130-A) in the same linear library molecule (e.g., FIG. 27A, left). In some embodiments, individual immobilized splint capture primers in the second sub-population (200-B) comprise a first portion (210-B) and a second portion (220-B), wherein the first portion of the splint capture primer (210-B) binds a first universal binding site (120-B) in a second linear library molecule and the second portion of the splint capture primer (220-B) binds a second universal binding site (130-B) in the same linear library molecule (e.g., FIG. 27A, right).

In some embodiments of step (a), the immobilized splint capture primers comprise single stranded oligonucleotides comprising DNA, RNA or a combination of DNA and RNA. The immobilized splint capture primers can be any length, for example 4-50 nucleotides, or 50-100 nucleotides, or 100-150 nucleotides, or longer lengths.

In some embodiments of step (a), individual splint capture primers comprise a terminal 3′ extendible end. In some embodiments, individual splint capture primers comprise a terminal 3′ nucleotide having a sugar 3′ OH moiety which is extendible for nucleotide polymerization (e.g., polymerase catalyzed nucleotide polymerization). In some embodiments, individual splint capture primers comprise a 3′ non-extendible end having a blocking moiety which can be removed to generate a 3′ OH moiety.

In some embodiments of step (a), individual splint capture primers lack a nucleotide having a scissile moiety. In some embodiments, individual splint capture primers lack a nucleotide having a scissile moiety that can be cleaved to generate an abasic site in the splint capture primer. For example, the splint capture primers lack uridine, 8-oxo-7,8-dihydroguanine (e.g., 8oxoG) and deoxyinosine.

In some embodiments of step (a), individual splint capture primers include at least one nucleotide having a scissile moiety that can be cleaved to generate an abasic site in the splint capture primer. In some embodiments, the at least one nucleotide having a scissile moiety comprises uridine, 8-oxo-7,8-dihydroguanine (e.g., 8oxoG) or deoxyinosine. In some embodiments, the at least one nucleotide having a scissile moiety comprises a uracil base.

In some embodiments of step (a), individual splint capture primers lack an inosine. In some embodiments of step (a), individual splint capture primers include at least one inosine at any position. In some embodiments, an inosine base in a splint capture primer can hybridize with an adenine, cytosine or uracil in the linear library molecule.

In some embodiments of step (a), individual splint capture primers include at least one nucleotide having a scissile moiety which comprises an endonuclease restriction enzyme recognition sequence which can be cleaved by a restriction enzyme include for example a type I, type II, type IIs, type IIB, type III, and/or type IV restriction enzyme.

In some embodiments, the scissile moiety can be located in the first portion of the splint capture primer (210). In some embodiments, the scissile moiety can be located in the second portion (220) of the splint capture primer.

In any of the embodiments of step (a), the plurality of splint capture primers can be immobilized to the support or immobilized to a coating on the support. The immobilized splint capture primers can be embedded and attached (coupled) to the coating on the support. In some embodiments, the 5′ end of the splint capture primers are immobilized to a support or immobilized to a coating on the support. Alternatively, an interior portion or the 3′ end of the splint capture primers can be immobilized to a support or immobilized to a coating on the support.

In any of the embodiments of step (a), individual splint capture primers comprise at least one phosphorothioate diester bond at their 5′ ends which can render the splint capture primers resistant to exonuclease degradation. In some embodiments, individual splint capture primers comprise 2-5 or more consecutive phosphorothioate diester bonds at their 5′ ends. In some embodiments, individual splint capture primers comprise at least one ribonucleotide and/or at least one 2′-O-methyl or 2′-O-methoxyethyl (MOE) nucleotide which can render the splint capture primers resistant to exonuclease degradation.

In any of the embodiments of step (a), individual splint capture primers comprise at least one locked nucleic acid (LNA) which comprises a methylene bridge bond between a 2′ oxygen and 4′ carbon of the pentose ring. In some embodiments, up to 5 nucleotides at or near the terminal 5′ end comprise a locked nucleic acid (LNA). Immobilized splint capture primers that include at least one LNA can be resistant to nuclease digestions and can exhibit increased melting temperature when hybridized to the forward extension strand.

In some embodiments, in step (a), the plurality of pinning primers (500) that are immobilized to the support comprise one type of pinning primers having the same sequence. For example, individual immobilized pinning primers (500) bind to at least a portion of a nucleic acid concatemer (e.g., FIG. 37)

In some embodiments, in step (a), the plurality of pinning primers (500) that are immobilized to the support comprise a mixture of different types of pinning primers including at least a first and second sub-population of pinning primers having different sequences, wherein the different types of pinning primers bind to different types of nucleic acid concatemer template molecules generated from different types of linear library molecules.

In some embodiments, in step (a), individual immobilized pinning primers (500) in the first sub-population (500-A) bind at least a portion of a universal adaptor sequence in a first nucleic acid concatemer template molecule.

In some embodiments, in step (a), individual immobilized pinning primers (500) in the second sub-population (500-B) bind at least a portion of a universal adaptor sequence in a second nucleic acid concatemer template molecule.

In some embodiments of step (a), the plurality of immobilized pinning primers (500) comprise single stranded oligonucleotides comprising DNA, RNA or a combination of DNA and RNA. The immobilized pinning primers can be any length, for example 4-50 nucleotides, or 50-100 nucleotides, or 100-150 nucleotides, or longer lengths.

In any of the embodiments of step (a), the plurality of immobilized pinning primers (500) comprise a sequence that is wholly complementary or partially complementary along their lengths to at least a portion of a nucleic acid concatemer (e.g., FIG. 37). In some embodiments, the pinning primers comprise a sequence that is complementary to at least a portion of a universal adaptor sequence in a nucleic acid concatemer template molecule. In some embodiments, the sequence of the pinning primers (500) differs from the sequence of the splint capture primers (200).

In some embodiments of step (a), individual pinning primers (500) comprise a terminal 3′ non-extendible end. In some embodiments, individual pinning primers comprise a terminal 3′ blocking moiety that inhibits a polymerase-catalyzed nucleotide polymerization. In some embodiments, the terminal 3′ blocking moiety comprises a phosphate group, a dideoxycytidine group, an inverted dT, or an amino group. In some embodiments, the pinning primers are not extendible in a primer extension reaction. In some embodiments, the 3′ terminal end of the pinning primers comprise an extendible OH moiety.

In some embodiments of step (a), individual pinning primers (500) lack a nucleotide having a scissile moiety that can be cleaved to generate an abasic site in the pinning primer. In some embodiments, individual pinning primers lack a nucleotide having a scissile moiety. For example, the pinning primers lack uridine, 8-oxo-7,8-dihydroguanine (e.g., 8oxoG) and deoxyinosine. In some embodiments, individual pinning primers include a nucleotide having a scissile moiety that can be cleaved to generate an abasic site in the pinning primer. In some embodiments, individual pinning primers include an endonuclease restriction enzyme recognition sequence which can be cleaved by a restriction enzyme include for example a type I, type II, type IIs, type IIB, type III, and/or type IV restriction enzyme.

In some embodiments of step (a), the plurality of pinning primers (500) can be immobilized to the support or immobilized to a coating on the support. The immobilized pinning primers can be embedded and attached (coupled) to the coating on the support. In some embodiments, the 5′ end of the pinning primers are immobilized to a support or immobilized to a coating on the support. Alternatively, an interior portion or the 3′ end of the pinning primers can be immobilized to a support or immobilized to a coating on the support.

In some embodiments of step (a), individual pinning primers (500) comprise at least one phosphorothioate diester bond at their 5′ ends which can render the pinning primers resistant to exonuclease degradation. In some embodiments, individual pinning primers comprise 2-5 or more consecutive phosphorothioate diester bonds at their 5′ ends. In some embodiments, individual pinning primers comprise at least one ribonucleotide and/or at least one 2′-O-methyl or 2′-O-methoxyethyl (MOE) nucleotide which can render the pinning primers resistant to exonuclease degradation.

In some embodiments of step (a), individual pinning primers (500) comprise at least one locked nucleic acid (LNA) which comprises a methylene bridge bond between a 2′ oxygen and 4′ carbon of the pentose ring. Pinning primers that include at least one LNA can be resistant to nuclease digestions and can exhibit increased melting temperature when hybridized to a concatemer.

In some embodiments of step (a), the support comprises about 10²-10¹⁵ immobilized splint capture primers (200) per mm². In some embodiments, the support comprises about 10²-10¹⁵ immobilized pinning primers (500) per mm². In some embodiments, the support comprises about 10²-10¹⁵ immobilized splint capture primers and immobilized pinning primers per mm².

In some embodiments of step (a), the immobilized splint capture primers (200) and pinning primers (500) are in fluid communication with each other to permit flowing various solutions of linear or circular nucleic acid template molecules, soluble primers, enzymes, nucleotides, divalent cations, buffers, reagents, and the like, onto the support so that the plurality of immobilized splint capture and pinning primers (and any primer extension products generated from the immobilized splint capture primers) react with the solutions in a massively parallel manner.

In some embodiments, the method for generating a plurality of nucleic acid concatemers immobilized to a support further comprises step (b): providing a plurality of nucleic acid linear library molecules including at least a first sub-population and a second sub-population of linear library molecules, wherein individual linear library molecules in the plurality comprise a 5′ and 3′ end, and wherein individual linear library molecules in the plurality comprise a sequence of interest and any one or any combination of two or more adaptor sequences in any order, wherein the adaptor sequences comprise: (i) a first universal binding site (120) for a first portion of a splint capture primer (or a complementary sequence thereof); (ii) a universal binding site for a first non-splint capture primer (123) (or a complementary sequence thereof); (iii) at least one sample index sequence (e.g., (160) and/or (170) which can be used to distinguish sequences of interest obtained from different sample sources in a multiplex assay; (iv) at least one universal binding site for a forward sequencing primer (140) (or a complementary sequence thereof); (v) at least one universal binding site for a reverse sequencing primer (150) (or a complementary sequence thereof); (vi) at least one universal binding site for a compaction oligonucleotide (or a complementary sequence thereof); (vii) at least one unique molecular index sequence (UMI) (e.g., a left unique molecular index sequence (180) and/or or a right unique molecular index sequence (190)) which can be used to uniquely identify a nucleic acid molecule (e.g., having a sequence of interest) to which the unique molecular index sequence is appended; (viii) at least one universal binding site for a pinning primer (or a complementary sequence thereof); (ix) at least one batch-specific barcode sequence; (x) a universal binding site for a second non-splint capture primer (133) (or a complementary sequence thereof); (xi) at least one short random sequence (e.g., NNNN) (132) which provides nucleotide sequence diversity and is about 3-20 nucleotides in length; and/or (xii) a second universal binding site (130) for a second portion of the immobilized splint capture (or a complementary sequence thereof) (e.g., see FIGS. 17A-17B, 18A-18B, 19A-19B, 25A-25B, 26A-26B and 27A-27B). In some embodiments, the plurality of linear library molecules comprise single-stranded linear library molecules. In some embodiments, the plurality of linear library molecules comprise double-stranded linear library molecules. In some embodiments, the plurality of linear library molecules comprise a mixture of single-stranded and double-stranded linear library molecules.

In some embodiments, in step (b), individual linear library molecules in the first sub-population comprise a sequence of interest. In some embodiments, the sequences of interest in the linear library molecules of the first sub-population have the same sequence. In some embodiments, the sequences of interest in the linear library molecules of the first sub-population have different sequences.

In some embodiments, in step (b), individual linear library molecules in the second sub-population comprise a sequence of interest. In some embodiments, the sequence of interest in the linear library molecules of the second sub-population have the same sequence. In some embodiments, the sequence of interest in the linear library molecules of the second sub-population have different sequences. In some embodiments, the linear library molecules of the first and second sub-population have the same sequence. In some embodiments, the linear library molecules of the first and second sub-population have different sequences of interest.

In some embodiments, in step (b), individual linear library molecules in the first and second sub-populations comprise a universal binding site for a forward sequencing primer (140) which includes a batch-specific forward sequencing primer binding site which can be employed for forward batch sequencing.

In some embodiments, in step (b), individual linear library molecules in the first and second sub-populations comprise a universal binding site for a reverse sequencing primer (150) which includes a batch-specific reverse sequencing primer binding site which can be employed for reverse batch sequencing.

In some embodiments, in step (b), individual linear library molecules in the first and second sub-populations comprise at least one sample index sequence (e.g., a left sample index sequence (160) and/or a right sample index sequence (170)). In some embodiments, the at least one sample index sequence is joined to an optional short random sequence (e.g., NNN), where the short random sequence provides nucleotide sequence diversity. In some embodiments, the short random sequence is about 3-20 nucleotides in length.

In some embodiments, individual linear library molecules provided in step (b) comprise a 5′ end that is non-phosphorylated or phosphorylated. In some embodiments, the non-phosphorylated 5′ ends can be treated with a polynucleotide kinase (e.g., T4 PNK) to generated 5′ phosphorylated ends that are ligatable.

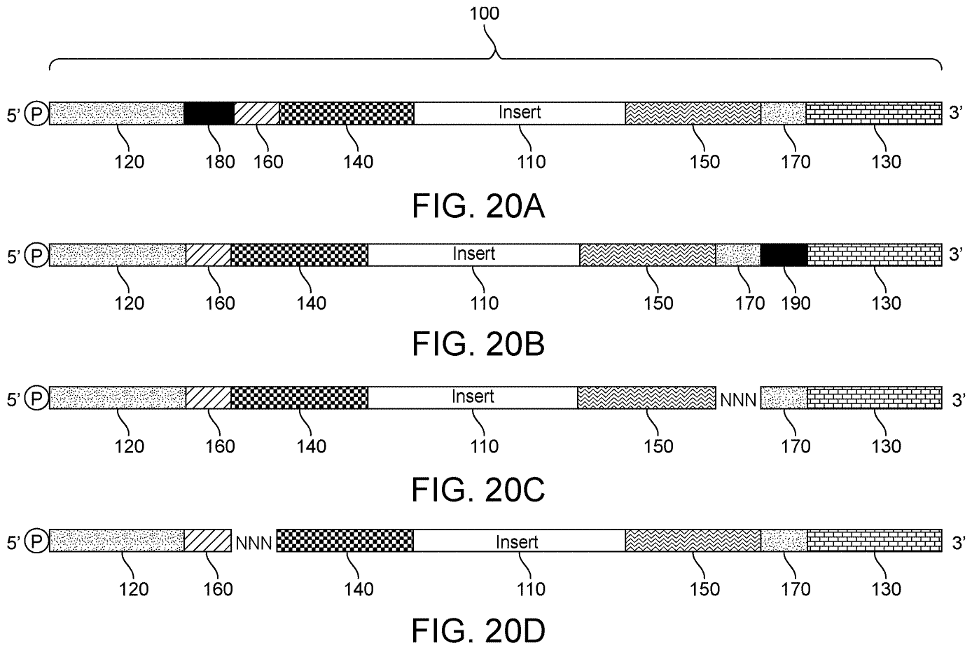

In some embodiments, the plurality of nucleic acid linear library molecules provided in step (b) comprise any of the linear library molecules shown in FIGS. 20-24. The skilled artisan will recognize that linear library molecules having adaptor sequences constructed with other arrangements are possible.

In some embodiments, in step (b), individual linear library molecules in the first and second sub-population comprise the same type of universal binding sites for binding a first portion of a splint capture primer (or complementary sequence thereof), and the same type of universal binding sites for binding a second portion of a splint capture primer (or complementary sequence thereof). In some embodiments, individual linear library molecules in the first and second sub-population of linear library molecules comprise a first universal binding site (120) for binding a first portion (210) of a splint capture primer (or a complementary sequence thereof) (210) and a second universal binding site (130) for binding a second portion (220) of the immobilized splint capture (or a complementary sequence thereof). In some embodiments, linear library molecules in the first and second sub-population comprise the same first and second universal binding sites (120) and (130) (e.g., FIGS. 25-26).

In some embodiments, in step (b), individual linear library molecules in the first and second sub-population comprise different types of universal binding sites for binding a first portion of a splint capture primer (or complementary sequence thereof), and different types of universal binding sites for binding a second portion of a splint capture primer (or complementary sequence thereof). In some embodiments, individual linear library molecules in the first sub-population of linear library molecules comprise a universal binding site (120-A) for binding a first portion of a splint capture primer (or a complementary sequence thereof) (210-A) and a universal binding site (130-A) for binding a second portion of the immobilized splint capture (or a complementary sequence thereof) (220-A). In some embodiments, individual linear library molecules in the second sub-population of linear library molecules comprise a universal binding site (120-B) for binding a first portion of a splint capture primer (or a complementary sequence thereof) (210-B) and a universal binding site (130-B) for binding a second portion of the immobilized splint capture (or a complementary sequence thereof) (220-B). In some embodiments, the universal binding sites (120-A) and (120-B) have different sequences. In some embodiments, the universal binding sites (130-A) and (130-B) have different sequences (e.g., FIG. 27A left and right). In some embodiments, the splint capture primers (220-A and 220-B) that hybridize the first and second sub-populations of linear library molecules comprise different sequences.

In some embodiments, the method for generating a plurality of nucleic acid concatemers immobilized to a support further comprises step (c): contacting the plurality of splint capture primers (200) immobilized on the support with the plurality of linear library molecules (100), wherein the contacting is conducted under a condition suitable for hybridizing individual linear library molecules to individual immobilized splint capture primers (200) to form individual open circle library molecules (300) each having at least a portion of the first terminal region of an individual linear library molecule hybridized to a first portion (210) of a splint capture primer and having at least a portion of the second terminal region of the same linear library molecule hybridized to a second portion (220) of the same splint capture primer, wherein individual open circle library molecules have a gap or nick between the 5′ and 3′ ends of the open circle library molecule (e.g., FIGS. 17A, 18A, 19A, 25A, 26A, 27A left and 27A right). In some embodiments, a first universal binding site (120) for a first portion of a splint capture primer of a given linear library molecule is hybridized to the first portion (210) of a splint capture primer and a second universal binding site (130) for a second portion of the immobilized splint capture of the same given library molecule is hybridized to the second portion (220) of the same splint capture primer. In some embodiments, the contacting of step (c) comprises distributing the plurality of single stranded nucleic acid linear library molecules onto the support having the plurality of immobilized splint capture primers (200) and pinning primers (500). In some embodiments, the contacting of step (c) comprises distributing one type of single stranded nucleic acid linear library molecules onto the support having the plurality of immobilized splint capture primers (200) and pinning primers (500). In some embodiments, the contacting of step (c) comprises distributing a mixture of at least two different types of single stranded nucleic acid linear library molecules onto the support having the plurality of immobilized splint capture primers and pinning primers, wherein the at least two types comprises at least a first and second sub-population of linear library molecules and wherein the support comprises a first and second sub-population of immobilized splint capture primers. In some embodiments, the first universal binding site (120) for a first portion of an immobilized splint capture primer in the linear library molecule) can hybridize to the first portion (210) of the immobilized splint capture primer. In some embodiments, the second universal binding site (130) for a second portion of an immobilized splint capture primer in the linear library molecule can hybridize to the second portion (220) of the immobilized splint capture primer. In some embodiments, the immobilized splint capture primers comprise a first portion (210) and a second portion (220) which hybridize to adaptor sequences, e.g. (120) and (130), in the linear library molecule (100), and the splint capture primers serve as a nucleic acid splint molecule for circularizing the linear library molecules (e.g., FIGS. 17A, 18A, 19A, 25A, 26A, 27A left and 27A right).

In some embodiments, step (c) comprises: contacting the first sub-population of immobilized splint capture primers (200-A) with the first sub-population of linear library molecules (100-A), wherein the contacting is conducted under a condition suitable for hybridizing individual linear library molecules in the first sub-population to individual immobilized splint capture primers in the first sub-population to form individual open circle library molecules each having the first terminal region of a given linear library molecule hybridized to a first portion (210-A) of a splint capture primer and having the second terminal region of the same linear library molecule hybridized to a second portion (220-A) of the same splint capture primer, wherein individual open circle library molecules in the first sub-population have a gap or nick between the 5′ and 3′ ends of the open circle library molecule (e.g., FIG. 27A, left). In some embodiments, the contacting of step (c) comprises distributing the first sub-population of single stranded nucleic acid linear library molecules onto the support having a mixture of first and second sub-populations of immobilized splint capture primers. In some embodiments, the contacting of step (c) comprises distributing the first sub-population of single stranded nucleic acid linear library molecules onto the support having a plurality of pinning primers (500). In some embodiments, the immobilized splint capture primers (200-A) comprise a first portion (210-A) and a second portion (220-A) which hybridize to adaptor sequences (120-A) and (130-A) in the linear library molecules of the first sub-population, and the splint capture primers (200-A) serve as a nucleic acid splint molecule for circularizing the linear library molecules of the first sub-population (e.g., FIG. 27A, left). In some embodiments, individual linear library molecules of the first sub-population comprise a universal binding sequence (120-A) that can hybridize to the first portion (210-A) of individual immobilized splint capture primers in the first sub-population. In some embodiments, individual linear library molecules of the first sub-population comprise a universal binding sequence (130-A) that can hybridize to the second portion (220-A) of individual immobilized splint capture primers in the first sub-population.

In some embodiments, step (c) comprises: contacting the second sub-population of immobilized splint capture primers (200-B) with the second sub-population of linear library molecules (100-B), wherein the contacting is conducted under a condition suitable for hybridizing individual linear library molecules in the second sub-population to individual immobilized splint capture primers in the second sub-population to form individual open circle library molecules each having the first terminal region of a given linear library molecule hybridized to a first portion (210-B) of a splint capture primer and having the second terminal region of the same linear library molecule hybridized to a second portion (220-B) of the same splint capture primer, wherein individual open circle library molecules in the second sub-population have a gap or nick between the 5′ and 3′ ends of the open circle library molecule (e.g., FIG. 27A, right). In some embodiments, the contacting of step (c) comprises distributing the second sub-population of single stranded nucleic acid linear library molecules onto the support having a mixture of first and second sub-populations of immobilized splint capture primers. In some embodiments, the contacting of step (c) comprises distributing the second sub-population of single stranded nucleic acid linear library molecules onto the support having a plurality of pinning primers (500). In some embodiments, the immobilized splint capture primers (200-B) comprise a first portion (210-B) and a second portion (220-B) which hybridize to adaptor sequences (120-B) and (130-B) in the linear library molecules of the second sub-population, and the splint capture primers (200-B) serve as a nucleic acid splint molecule for circularizing the linear library molecules (e.g., FIG. 27A, right). In some embodiments, individual linear library molecules of the second sub-population comprise a universal binding sequence (120-B) that can hybridize to the first portion (210-B) of individual immobilized splint capture primers in the second sub-population. In some embodiments, individual linear library molecules of the second sub-population comprise a universal binding sequence (130-B) that can hybridize to the second portion (220-B) of individual immobilized splint capture primers in the second sub-population.

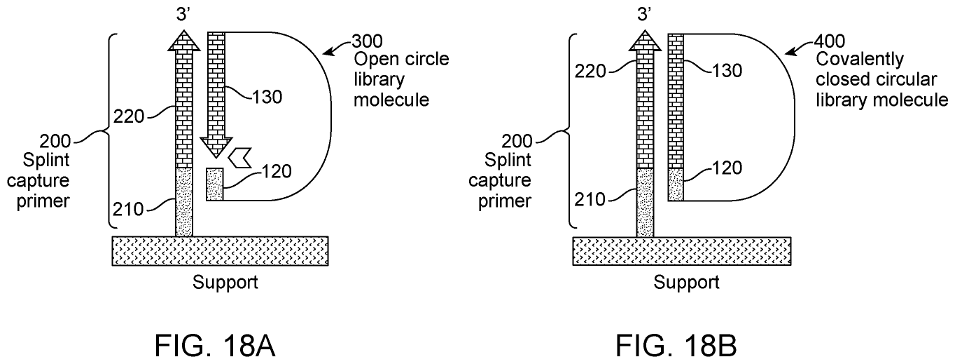

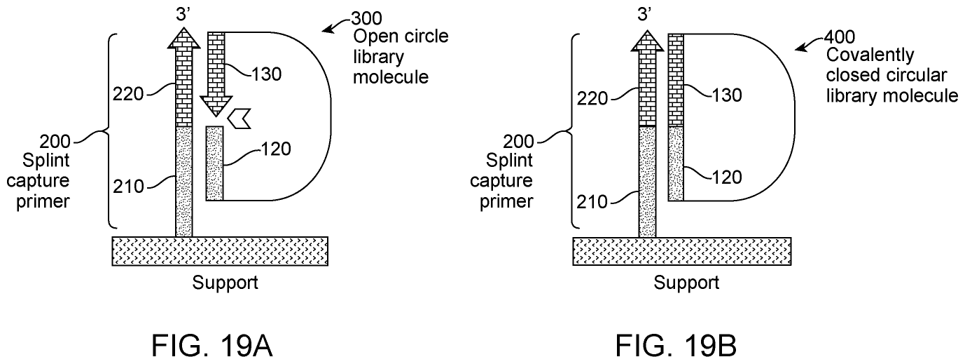

In some embodiments of step (c), the position of the gap or nick in the open circle library molecules can be asymmetrical or symmetrical relative to the duplex formed by hybridizing the 5′ and 3′ ends of the linear library molecule to the immobilized splint capture primers (200). For example, FIG. 17A shows an asymmetrical positioned gap or nick. FIG. 18A shows an asymmetrical positioned gap or nick. FIG. 19A shows a symmetrical positioned gap or nick. An asymmetrical or symmetrical positioned gap/nick can be generated by adjusting the length of the first portion (210) and the second portion (220) in the immobilized splint capture primers. In some embodiments, the length of the first portion (210) can be increased and the length of the second portion (220) can be decreased to improve/increase the percentage of linear library molecules that hybridize to the splint capture primer (200). In some embodiments, the length of the first portion (210) can be decreased and the length of the second portion (220) can be increased to improve/increase the percentage of linear library molecules that hybridize to the splint capture primer (200).

In some embodiments of step (c), when the plurality of linear library molecules comprise double stranded linear library molecules, then the hybridizing conditions of step (c) are suitable for denaturing the double stranded linear library molecules into single stranded linear library molecules that can hybridize to the splint capture primers (200). For example, the hybridizing can be conducted at a temperature of about 35-40° C., or about 40-45° C., or about 45-50° C., or about 50-55° C. In some embodiments, the hybridizing of step (c) can be conducted using a hybridization reagent comprising 3×SSC, formamide and/or a chaotropic agent. In some embodiments, a chaotropic agent can disrupt non-covalent bonds such as hydrogen bonds or van der Waals forces. In some embodiments, the chaotropic agent comprises SDS (sodium dodecyl sulfate), urea, thiourea, guanidinium chloride, guanidine hydrochloride, guanidine thiocyanate, guanidine isothionate, potassium thiocyanate, lithium chloride, sodium iodide or sodium perchlorate.

In some embodiments of step (c), the amount of the plurality of linear library molecules (100) that are contacted with the plurality of immobilized splint capture primers (200) can adjusted to achieve a density of immobilized concatemer template molecules of about 10²-10¹⁵ per mm² where the immobilized concatemer template molecules can be generated in step (e) (described below) by conducting a rolling circle amplification reaction. In some embodiments, the amount of the plurality of linear library molecules (100) that are contacted with the plurality of immobilized splint capture primers (200) can be about 0.1-1 pM, or about 1-5 pM, or about 5-10 pM, or about 10-20 pM, or about 20-30 pM, or about 30-40 pM, or about 40-50 pM.

In some embodiments, the method for generating a plurality of nucleic acid concatemers immobilized to a support further comprises step (d): enzymatically closing the nicks or gaps in the plurality of open circle library molecules thereby generating a plurality of covalently closed circular library molecules (400), wherein individual single stranded covalently closed circular library molecules are hybridized to an immobilized splint capture primer (e.g., FIGS. 17B, 18B, 19B, 25B, 26B, 27B left and 27B right).

In some embodiments, step (d) comprises: enzymatically closing the nicks or gaps in the plurality of open circle library molecules of the first sub-population thereby generating a first sub-population of covalently closed circular library molecules, wherein individual single stranded covalently closed circular library molecules in the first sub-population are hybridized to an immobilized splint capture primer of the first sub-population (e.g., FIG. 27B, left).

In some embodiments, step (d) comprises: enzymatically closing the nicks or gaps in the plurality of open circle library molecules of the second sub-population thereby generating a second sub-population of covalently closed circular library molecules wherein individual single stranded covalently closed circular library molecules in the second sub-population are hybridized to an immobilized splint capture primer of the second sub-population (e.g., FIG. 27B, right).

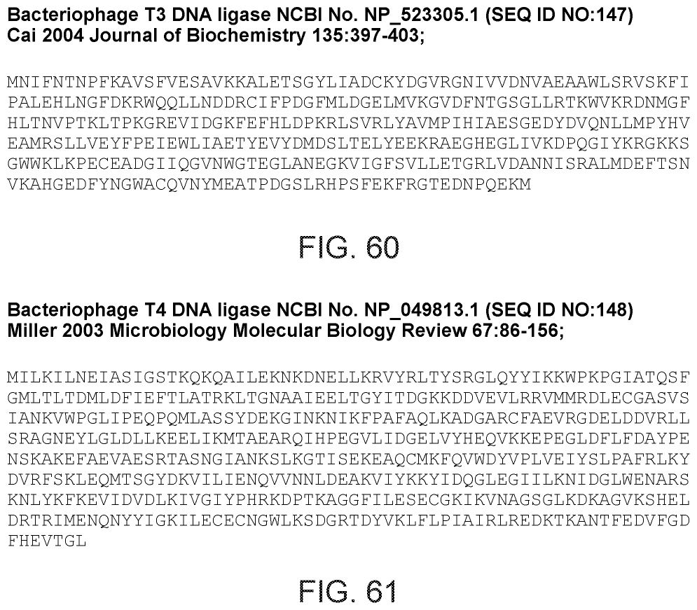

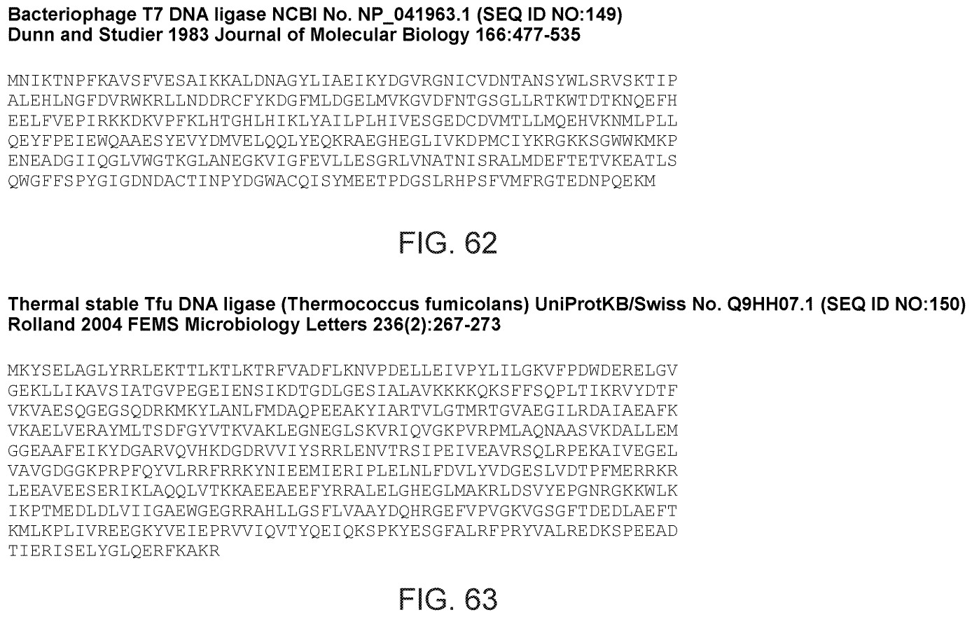

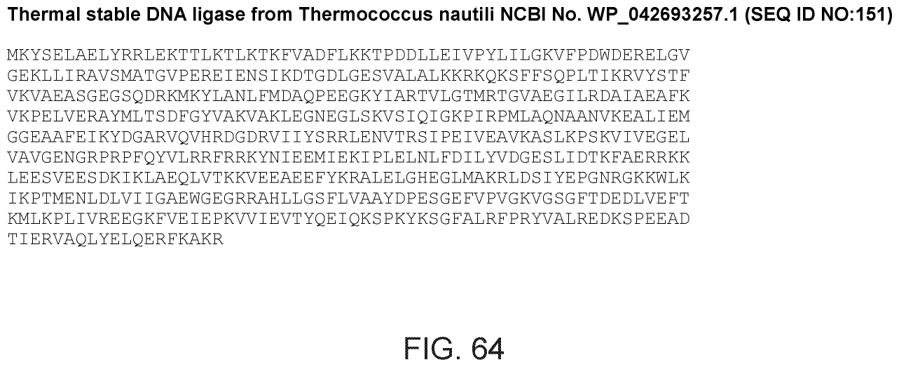

In any of the embodiments of step (d), the nick in individual open circle library molecules can be closed by conducting a ligase-catalyzed ligation reaction to form a single stranded covalently closed circular molecule, wherein individual covalently closed circular molecules are hybridized to individual immobilized splint capture primers. In some embodiments, the ligation reaction can be conducted with a ligase enzyme. In some embodiments, the ligase enzyme comprises a bacteriophage DNA ligase, including a T3 DNA ligase (e.g., FIG. 60), T4 DNA ligase (e.g., FIG. 61) or T7 DNA ligase (e.g., FIG. 62). In some embodiments, the ligase comprises a thermal stable DNA ligase including a Taq DNA ligase, a Tfu DNA ligase (e.g., FIG. 63) or a DNA ligase from Thermococcus nautili (e.g., FIG. 64). In some embodiments, the ligase comprises a recombinant thermal tolerant T4 DNA ligase (e.g., Hi-T4 DNA ligase from New England Biolabs, catalog #M2622S).

In some embodiments of step (d), the gap in individual open circle library molecules can be closed by conducting a polymerase-catalyzed gap fill-in reaction using the 3′ extendible end of the library molecule as an initiation site for the polymerase-catalyzed fill-in reaction and using the immobilized splint capture primer as a template molecule thereby forming covalently closed circularized molecule having a nick. The nick can be closed by conducting an enzymatic ligation reaction to form a single stranded covalently closed circular library molecule, wherein individual covalently closed circular library molecules are hybridized to individual immobilized splint capture primers. In some embodiments, the gap fill-in reaction can be conducting with a plurality of nucleotides and a polymerase that lacks 5′ to 3′ strand displacement activity. The polymerase comprises E. coli DNA polymerase I, Klenow fragment of E. coli DNA polymerase I, T7 DNA polymerase, or T4 DNA polymerase. In some embodiments, after the gap fill-in reaction, the nick can be closed by conducting a ligation reaction using a ligase enzyme. In some embodiments, the ligase enzyme comprises a bacteriophage DNA ligase, including a T3, T4 or T7 DNA ligase. In some embodiments, the ligase comprises a thermal stable DNA ligase including a Taq DNA ligase, a Tfu DNA ligase or a DNA ligase from Thermococcus nautili. In some embodiments, the ligase comprises a recombinant thermal tolerant T4 DNA ligase (e.g., Hi-T4 DNA ligase from New England Biolabs, catalog #M2622S).

In some embodiments of step (d), the ligation reaction can be conducted by contacting the plurality of open circle library molecules (e.g., having a nick) at least once with a ligation reaction mixture comprising one or more DNA ligase(s), a pH buffering agent, and ATP. In some embodiments, the ligation reaction mixture comprises one or more DNA ligase(s), a pH buffering agent, ATP, a plurality of nucleotides, and the ligation reaction mixture lacks or includes a strand displacing polymerase. In any of the embodiments of step (d), the ligation reaction can be conducted using a ligation reaction mixture comprising at least one DNA ligase and a strand displacing polymerase. In some embodiments, the ligation reaction mixture further comprises any combination of magnesium ions, a reducing agent, a detergent, a crowding agent, an amino acid, a phosphine compound, ammonium ions, a salt, a viscosity agent, a plurality of nucleotides and/or a strand displacing polymerase. In some embodiments, the plurality of open circle library molecules (e.g., having a nick) can be contacted with a ligation reaction mixture at least two times, at least three times, at least four times, or up to ten times.

In some embodiments of step (d), the ligation reaction can be conducted at a temperature at which the ligase exhibits activity, for example at about 15-20° C., or about 20-30° C., or about 30-40° C., or about 40-50° C.

In some embodiments of step (d), the ligase enzyme in the ligation reaction mixture comprises a bacteriophage DNA ligase, for example a T3, T4 or T7 DNA ligase. In some embodiments, the ligase enzyme in the ligation reaction mixture comprises a thermal stable DNA ligase including a Taq DNA ligase, a Tfu DNA ligase or a DNA ligase from Thermococcus nautili. In some embodiments, the ligase enzyme in the ligation reaction mixture comprises a recombinant thermal tolerant T4 DNA ligase (e.g., Hi-T4 DNA ligase from New England Biolabs, catalog #M2622S).

In some embodiments of step (d), the ligation reactions can be conducted using a ligase reaction mixture comprising a T3 bacteriophage DNA ligase (e.g., NCBI No. 523305.1), a T4 bacteriophage DNA ligase (e.g., NCBI No. 049813.1), a T7 bacteriophage DNA ligase (e.g., NCBI No. 041963.1), a thermal stable Taq DNA ligase (e.g., from New England Biolabs, catalog No. M0208S), a thermal stable Tfu DNA ligase from Thermococcus fumicolans (e.g., UniProtKB/Swiss No. Q9HH07.1), a thermal stable DNA ligase from Thermococcus nautili (e.g., NCBI No. WP_042693257.1) and/or an engineered thermal tolerant T4 DNA ligase Hi-T4 ligase (from New England Biolabs, catalog No. M2622S).

In some embodiments of step (d), the pH buffering agent in the ligation reaction mixture comprises Tris (e.g., Tris(hydroxymethyl)-aminomethane), Tris-HCL (e.g., Tris(hydroxymethyl)-aminomethane hydrochloride), HEPES (e.g., 4-(2-hydroxyethyl)-1-piperazineethanesulfonic acid) or MOPS (e.g., 3-(N-morpholino)propanesulfonic acid). In some embodiments, the pH buffering agent in the ligation reaction mixture is within a pH range at which a strand displacing polymerase is inactive. For example, the pH buffering agent in the ligation reaction mixture can be a pH range of about 4-9, can be a pH range of about 5-8.5, a pH range of about 5.5-8, a pH range of about 6-7.9, a pH range of about 6.5-7.8, a pH range of about 7-7.9, or a pH range of about 7-7.5.

In some embodiments of step (d), the magnesium ions in the ligation reaction mixture comprises MgCl₂ or MgSO₄.

In some embodiments of step (d), the reducing agent in the ligation reaction mixture comprises DTT (e.g., dithiothreitol), DTE (dithioerythritol), betaine and/or glucuronic acid

In some embodiments of step (d), the detergent in the ligation reaction mixture comprises Tween-20, Tween-80, Triton X-100, Nonidet P-40, CHAPS (e.g., 3-[(3-cholamidopropyl) dimethylammonio]-1-propanesulfonate) or DetX (e.g., N-Dodecyl-N,N-dimethyl-3-amonio-1-propanesulfate).

In some embodiments of step (d), the crowding agent in the ligation reaction mixture comprises PEG (e.g., polyethylene glycol, e.g., 1-50K molecular weight), dextran, dextran sulfate, hydroxypropyl methyl cellulose (HPMC), hydroxyethyl methyl cellulose (HEMC), hydroxybutyl methyl cellulose, hydroxypropyl cellulose, methycellulose, or hydroxyl methyl cellulose.

In some embodiments of step (d), the amino acid in the ligation reaction mixture comprises beta-alanine or beta-valine.

In some embodiments of step (d), the phosphine compound in the ligation reaction mixture comprises a phosphine having a derivatized tri-alkyl phosphine moiety or a derivatized tri-aryl phosphine moiety. In some embodiments, the phosphine compound comprises TCEP (e.g., Tris(2-carboxyethyl)phosphine), BS-TPP (e.g., bis-sulfo triphenyl phosphine), THPP (e.g., Tri(hydroxyproyl)phosphine), or THMP (e.g., Tri(hydroxymethyl)phosphine).

In some embodiments of step (d), the ammonium ions in the ligation reaction mixture comprises ammonium sulfate (e.g., NH₄)₂SO₄) or ammonium acetate.

In some embodiments, the salt in the ligation reaction mixture comprises NaCl, KCl or potassium glutamate.

In some embodiments of step (d), the viscosity agent in the ligation reaction mixture comprises trehalose, sucrose, cellulose, xylitol, mannitol, sorbitol, D-maltose or inositol. In some embodiments, the viscosity agent comprises glycerol or a glycol compound such as ethylene glycol or propylene glycol (e.g., propanediol). In some embodiments, the viscosity agent in the ligation reaction mixture comprises sucrose 50% Brix which comprises 50 grams of sucrose in a total solution of 100 grams.

In some embodiments of step (d), the plurality of nucleotides in the ligation reaction mixture comprises any combination of dATP, dGTP, dCTP, dTTP and/or dUTP.

In some embodiments of step (d), the strand displacing polymerase in the ligation reaction mixture comprises a polymerase that can locally separate strands of double-stranded nucleic acids and synthesize a new strand in a template-based manner. Strand displacing polymerases displace a complementary strand from a template strand and catalyze new strand synthesis. In some embodiments, the strand displacing polymerase comprising a mesophilic or thermophilic polymerase. In some embodiments, the strand displacing polymerase comprises a wild type enzyme, or a variant enzyme including exonuclease minus mutants, mutant versions, chimeric enzymes and truncated enzymes. In some embodiments, the strand displacing polymerases comprises a phi29 DNA polymerase, a large fragment of Bst DNA polymerase, a large fragment of Bsu DNA polymerase (exo-), a Bca DNA polymerase (exo-), a Klenow fragment of E. coli DNA polymerase, a T5 polymerase, an M-MuLV reverse transcriptase, an HIV viral reverse transcriptase, a Deep Vent DNA polymerase or a KOD DNA polymerase. In some embodiments, the phi29 DNA polymerase can be wild type phi29 DNA polymerase (e.g., MagniPhi from Expedeon), or variant EquiPhi29 DNA polymerase (e.g., from Thermo Fisher Scientific, catalog #A39390), or chimeric QualiPhi DNA polymerase (e.g., from 4basebio, catalog #510025).

In some embodiments of step (d), the ligation reaction mixture can include at least one DNA ligase, a strand-displacing polymerase, and a pH buffering agent at a pH at which the strand displacing polymerase is inactive. For example, the pH buffering agent in the ligation reaction mixture can be a pH range of about 6.5-7.8. Thus, the strand-displacing polymerase in the ligation reaction mixture does not catalyze a rolling circle amplification reaction. In some embodiments, the DNA ligase enzyme(s) in the ligation reaction mixture can close the nicks in the open circular library molecules to generate a plurality of covalently closed circular library molecules each hybridized to an immobilized splint capture primer (200) thereby forming a nucleic acid duplex having a splint capture primer with a terminal 3′ end. In some embodiments, the strand displacing polymerases in the ligation reaction mixture can bind the terminal 3′ end of individual splint capture primers that have formed nucleic acid duplexes (e.g., pre-loaded strand displacing polymerases), but the strand displacing polymerases do not initiate primer extension reactions (e.g., rolling circle amplification reactions) because the strand displacing polymerases are not active at the pH of the ligation reaction mixture. In some embodiments, the strand displacing polymerases can be pre-loaded onto the terminal 3′ ends of the splint capture primers during the ligation reaction of step (d), then in step (e) the rolling circle amplification reaction can be initiated essentially simultaneously on the plurality of immobilized covalently closed circular library molecules by changing the pH to a range that permits activity of the pre-loaded strand displacing polymerase.

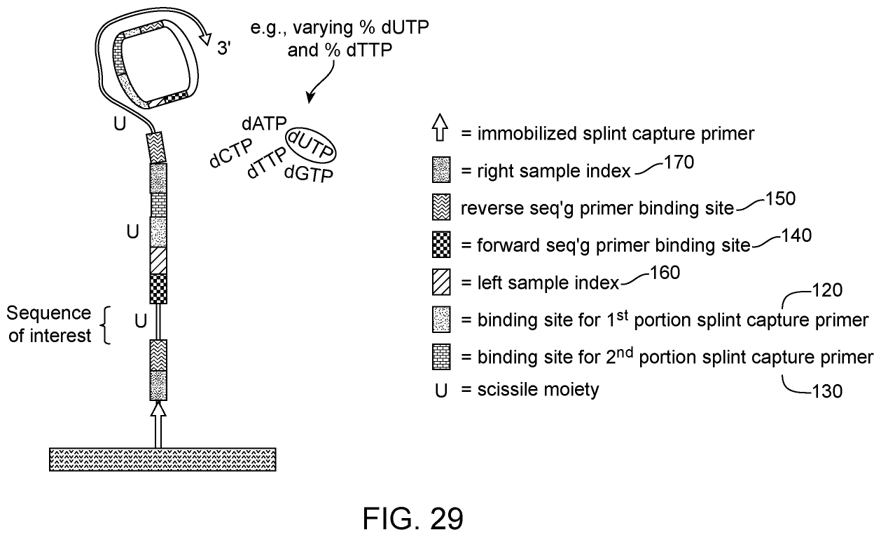

In some embodiments, the method for generating a plurality of nucleic acid concatemers immobilized to a support further comprises step (e): contacting a rolling circle amplification reaction mixture to the plurality of covalently closed circular library molecules which are immobilized to the support and conducting a plurality of rolling circle amplification reaction thereby generating a plurality of immobilized single stranded nucleic acid concatemer template molecules (e.g., FIGS. 29-36). In some embodiments, a single covalently closed circular library molecule can generate a single concatemer template molecule. In some embodiments, the single concatemer template molecule serves as a template molecule for conducting a downstream sequencing workflow.

In some embodiments, step (e) comprises: contacting a rolling circle amplification reaction mixture to the first and second sub-populations of covalently closed circular library molecules which are immobilized to the support and conducting a rolling circle amplification reaction, thereby generating a first and second sub-population of immobilized single stranded nucleic acid concatemer template molecules. In some embodiments, a single covalently closed circular library molecule can generate a single concatemer template molecule. In some embodiments, the single concatemer template molecule serves as a template molecule for conducting a downstream sequencing workflow.

In some embodiments of step (e), the rolling circle amplification reaction is conducted in the presence of a plurality of compaction oligonucleotides. In some embodiments, the plurality of compaction oligonucleotides includes a plurality of compaction oligonucleotides having the same sequence. In some embodiments, the plurality of compaction oligonucleotides includes mixture of compaction oligonucleotides having two or more different sequences. In some embodiments, the rolling circle amplification reaction can be initiated essentially simultaneously on the plurality of covalently closed circular library molecules which are immobilized to the support. In some embodiments, the plurality of covalently closed circular library molecules can be contacted with the rolling circle amplification reaction mixture at least once, at least two times, at least three times, at least four times, or up to ten times.

In some embodiments of step (e), the rolling circle amplification reaction can be conducted on the support which generates a plurality of immobilized concatemer template molecules wherein individual concatemer template molecules are covalently joined to an immobilized splint capture primer (e.g., FIGS. 29-36). In some embodiments, individual concatemer template molecules comprise two or more tandem repeat units wherein a unit comprises a complementary sequence of a given covalently closed circular library molecule which was generated in step (d) above. In some embodiments of step (e), at least one portion of individual concatemer template molecules can be hybridized to an immobilized pinning primer (e.g., FIG. 37). In some embodiments of step (e), the immobilized pinning primers comprise 3′ terminal ends having a non-extendible moiety. Thus, the plurality of immobilized pinning primers comprise terminal 3′ ends that do not initiate a rolling circle amplification reaction in step (e).

In some embodiments of step (e), the rolling circle amplification reaction mixture comprises any combination of magnesium ions, a reducing agent, a detergent, a crowding agent, an amino acid, a phosphine compound, ammonium ions, a salt, a viscosity agent, a plurality of nucleotides and/or a plurality of compaction oligonucleotides. In some embodiments of step (e), the rolling circle amplification reaction mixture lacks a strand displacing polymerase. In some embodiments of step (e), the rolling circle amplification reaction mixture includes a strand displacing polymerase. In some embodiments, when the rolling circle amplification reaction mixture lacks strand displacing polymerases, then the rolling circle amplification reaction is catalyzed by the strand displacing polymerase present in the ligation reaction mixture of step (d).

In some embodiments of step (e), the rolling circle amplification reaction mixture comprises a pH buffering agent, magnesium ions, a reducing agent, a detergent, a crowding agent, an amino acid, a phosphine compound, ammonium ions, a salt, a viscosity agent, a plurality of nucleotides, or a combination thereof. In some embodiments of step (e), the rolling circle amplification reaction mixture comprises a strand displacing polymerase.

In some embodiments of step (e), the pH buffering agent in the rolling circle amplification reaction mixture comprises Tris (e.g., Tris(hydroxymethyl)-aminomethane), Tris-HCL (e.g., Tris(hydroxymethyl)-aminomethane hydrochloride), HEPES (e.g., 4-(2-hydroxyethyl)-1-piperazineethanesulfonic acid) or MOPS (e.g., 3-(N-morpholino)propanesulfonic acid). In some embodiments of step (e), the pH buffering agent in the rolling circle amplification reaction mixture is within a pH range at which a strand displacing polymerase is active. For example, the pH buffering agent in the rolling circle amplification reaction mixture can be a pH range of about 7-9, a pH range of about 7.5-9, a pH range of about 8-9, a pH range of about 8.1-8.9, a pH range of about 8.2-8.8, a pH range of about 8.3-8.7, or a pH range of about 8.4-8.6.

In some embodiments of step (e), the magnesium ions in the rolling circle amplification reaction mixture comprises MgCl₂ or MgSO₄.

In some embodiments of step (e), the reducing agent in the rolling circle amplification reaction mixture comprises DTT (e.g., dithiothritol) and/or betaine.

In some embodiments of step (e), the detergent in the rolling circle amplification reaction mixture comprises Tween-20, Tween-80, Triton X-100, Nonidet P-40, CHAPS (e.g., 3-[(3-cholamidopropyl) dimethylammonio]-1-propanesulfonate) or DetX (e.g., N-Dodecyl-N,N-dimethyl-3-amonio-1-propanesulfate).

In some embodiments of step (e), the crowding agent in the rolling circle amplification reaction mixture comprises PEG (e.g., polyethylene glycol, e.g., 1-50K molecular weight), dextran, dextran sulfate, hydroxypropyl methyl cellulose (HPMC), hydroxyethyl methyl cellulose (HEMC), hydroxybutyl methyl cellulose, hydroxypropyl cellulose, methycellulose, or hydroxyl methyl cellulose.

In some embodiments of step (e), the amino acid in the rolling circle amplification reaction mixture comprises beta-alanine or beta-valine.

In some embodiments of step (e), the phosphine compound in the rolling circle amplification reaction mixture comprises a phosphine having a derivatized tri-alkyl phosphine moiety or a derivatized tri-aryl phosphine moiety. In any of the embodiments of step (e), the phosphine compound comprises TCEP (e.g., Tris(2-carboxyethyl)phosphine), BS-TPP (e.g., bis-sulfo triphenyl phosphine), THPP (e.g., Tri(hydroxyproyl)phosphine), or THMP (e.g., Tri(hydroxymethyl)phosphine).

In some embodiments of step (e), the ammonium ions in the rolling circle amplification reaction mixture comprises ammonium sulfate (e.g., NH₄)₂SO₄) or ammonium acetate.

In some embodiments of step (e), the salt in the rolling circle amplification reaction mixture comprises NaCl, KCl or potassium glutamate.

In some embodiments of step (e), the viscosity agent in the rolling circle amplification reaction mixture comprises trehalose, sucrose, cellulose, xylitol, mannitol, sorbitol, D-maltose or inositol. In some embodiments, the viscosity agent comprises glycerol or a glycol compound such as ethylene glycol or propylene glycol (e.g., propanediol).

In some embodiments of step (e), the plurality of nucleotides in the rolling circle amplification reaction mixture comprises any combination of dATP, dGTP, dCTP, dTTP and/or dUTP.

In some embodiments of step (e), the strand displacing polymerase, if present in the rolling circle amplification reaction mixture, comprises a polymerase that can locally separate strands of double-stranded nucleic acids and synthesize a new strand in a template-based manner. Strand displacing polymerases displace a complementary strand from a template strand and catalyze new strand synthesis. In some embodiments, the strand displacing polymerase comprising a mesophilic or thermophilic polymerase. In some embodiments, the strand displacing polymerase comprises a wild type enzyme, or a variant enzyme including exonuclease minus mutants, mutant versions, chimeric enzymes and truncated enzymes. In some embodiments, the strand displacing polymerases comprises a phi29 DNA polymerase, a large fragment of Bst DNA polymerase, a large fragment of Bsu DNA polymerase (exo-), a Bca DNA polymerase (exo-), a Klenow fragment of E. coli DNA polymerase, a T5 polymerase, an M-MuLV reverse transcriptase, an HIV viral reverse transcriptase, a Deep Vent DNA polymerase or a KOD DNA polymerase. In any of the embodiments of step (e), the phi29 DNA polymerase can be wild type phi29 DNA polymerase (e.g., MagniPhi from Expedeon), or variant EquiPhi29 DNA polymerase (e.g., from Thermo Fisher Scientific, catalog #A39390), or chimeric QualiPhi DNA polymerase (e.g., from 4basebio, catalog #510025) or any suitable polymerase described herein.

In some embodiments of step (e), the rolling circle amplification reaction mixture comprises a pH buffering agent and lacks a strand-displacing polymerase. In some embodiments, the pH buffering agent in the rolling circle amplification reaction mixture is within a pH range at which a strand displacing polymerase is active. For example, the pH buffering agent in the rolling circle amplification reaction mixture can be about pH 8-9. Thus, contacting the covalently closed circular molecules with the rolling circle amplification reaction mixture (e.g., which lacks strand displacing polymerase) initiates a rolling circle amplification reaction.

In some embodiments of step (e), the rolling circle amplification reaction can be conducted under isothermal amplification conditions at a constant temperature such as, for example about 20° C., about 25° C., about 30° C., about 35° C., about 37° C., about 40° C., about 42° C., about 50° C., about 60° C., about 65° C., about 70° C., about 75° C. or at a higher temperature, or within a temperature range defined by any two of the foregoing temperatures.

In some embodiments of step (e), the rolling circle amplification reaction mixture comprises a plurality of nucleotides including dATP, dCTP, dGTP and dTTP. In some embodiments, the plurality of nucleotides further comprises a nucleotide having a scissile moiety, wherein the rolling circle amplification reaction generates a plurality of immobilized single stranded nucleic acid concatemer template molecules each having at least one nucleotide with a scissile moiety (e.g., FIG. 29). In some embodiments, the concatemer template molecule having at least one incorporated nucleotide with a scissile moiety can be cleaved at the scissile moiety to generate an abasic site in the concatemer template molecule. In some embodiments, in the plurality of nucleotides, the nucleotide having the scissile moiety comprises deoxyuridine, 8-oxo-7,8-dihydroguanine (e.g., 8oxoG), deoxyinosine, thymine glycol, 3-methyladenine, 7-methylguanine, deoxyxanthosine, 5-hydroxyuridine, 5-hydroxymethyluridine, 5-formyluridine, cyclobutene pyrimidine dimers, 5 methyl cytosine, 5-hydroxymethylcytosine, 5-formylcytosine, 5-carboxylcytosine, N⁶-methyladenine, 5-methylcytosine, 5-hydroxymethylcytosine, 5-formylcytosine or 5-carboxylcytosine. In some embodiments, the plurality of nucleotides in a rolling circle amplification reaction lacks a nucleotide having a scissile moiety.

In some embodiments of step (e), the plurality of nucleotides in the rolling circle amplification mixture can include an amount of dUTP so that a target percent of the thymidine in the resulting concatemer template molecules are replaced with dUTP. For example, when 30% of dTTP in the concatemer template molecules are to be replaced with dUTP (e.g., 30% is the target percent) then the nucleotide mixture can contain 7.5% dUTP (e.g., 30/4=7.5%), 17.5% dTTP, and 25% each for dATP, dCTP and dGTP. The target percent of dTTP to be replaced by dUTP can be about 0.1-1%, or about 1-5%, or about 5-10%, or about 10-20%, or about 20-30%, or about 30-45%, or about 45-50%, or a higher percent of the dTTP in the immobilized concatemer template molecules are replaced with nucleotides having a scissile moiety.

In some embodiments of step (e), the plurality of nucleotides in the rolling circle amplification reaction mixture can include an amount of deoxyinosine so that a target percent of the guanosine in the resulting concatemer template molecules are replaced with deoxyinosine. For example, when 30% of dGTP in the concatemer template molecules are to be replaced with deoxyinosine (e.g., 30% is the target percent) then the nucleotide mixture can contain 7.5% deoxyinosine (e.g., 30/4=7.5%), 17.5% dGTP, and 25% each for dATP, dCTP and dTTP. The target percent of dGTP to be replaced by deoxyinosine can be about 0.1-1%, or about 1-5%, or about 5-10%, or about 10-20%, or about 20-30%, or about 30-45%, or about 45-50%, or a higher percent of the dGTP in the immobilized concatemer template molecules are replaced with nucleotides having a scissile moiety.

In some embodiments of step (e), the plurality of nucleotides in the rolling circle amplification mixture can include an amount of 8oxoG so that a target percent of the guanosine in the resulting concatemer template molecules are replaced with 8oxoG. For example, when 30% of dGTP in the concatemer template molecules are to be replaced with 8oxoG (e.g., 30% is the target percent) then the nucleotide mixture can contain 7.5% 8oxoG (e.g., 30/4=7.5%), 17.5% dGTP, and 25% each for dATP, dCTP and dTTP. The target percent of dGTP to be replaced by 8oxoG can be about 0.1-1%, or about 1-5%, or about 5-10%, or about 10-20%, or about 20-30%, or about 30-45%, or about 45-50%, or a higher percent of the dGTP in the immobilized concatemer template molecules are replaced with nucleotides having a scissile moiety.

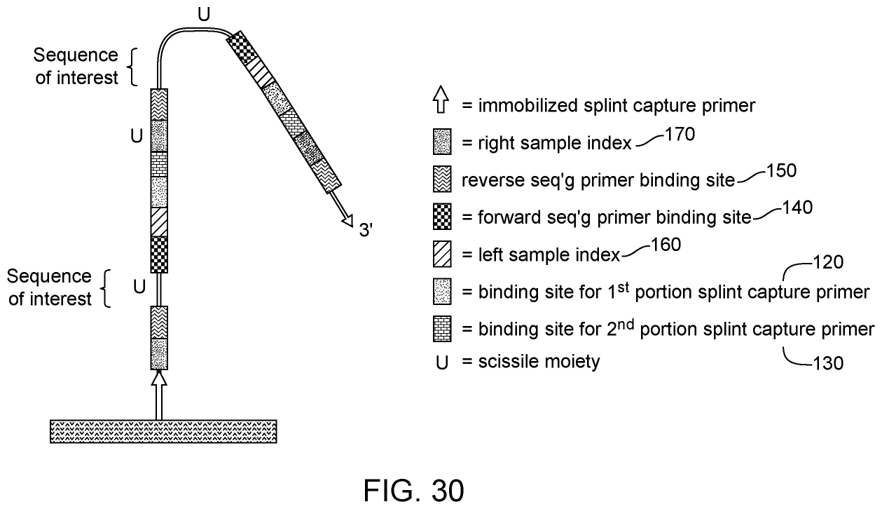

In some embodiments of step (e), the rolling circle amplification reaction generates immobilized concatemer template molecules with incorporated nucleotides having a scissile moiety that are distributed at random positions along individual immobilized concatemer template molecules (e.g., FIGS. 29-30). In some embodiments, the nucleotides having a scissile moiety are distributed at different positions in the different immobilized concatemer template molecules.

In some embodiments of step (e), individual immobilized concatemer template molecules generated by the rolling circle amplification reaction comprise two or more tandem repeat unit wherein a unit comprises a complementary sequence of the covalently closed circular library molecule (e.g., FIG. 29). In some embodiments, a repeat unit of an individual concatemer template molecules comprises any one or any combination of two or more of the following arranged in any order: (i) a sequence of interest; (ii) a first universal binding site (120) for a first portion of a splint capture primer (or a complementary sequence thereof); (iii) a universal binding site for a first non-splint capture primer (123) (or a complementary sequence thereof); (iv) at least one sample index sequence (e.g., (160) and/or (170) which can be used to distinguish sequences of interest obtained from different sample sources in a multiplex assay; (v) at least one universal binding site for a forward sequencing primer (140) (or a complementary sequence thereof); (vi) at least one universal binding site for a reverse sequencing primer (150) (or a complementary sequence thereof); (vii) at least one universal binding site for a compaction oligonucleotide (or a complementary sequence thereof); (viii) at least one unique molecular index sequence (UMI) (e.g., (180) and/or (190)) which can be used to uniquely identify a nucleic acid molecule (e.g., having a sequence of interest) to which the unique molecular index sequence is appended; (ix) at least one universal binding site for a pinning primer (or a complementary sequence thereof); (xi) at least one batch-specific barcode sequence; (xii) a universal binding site for a second non-splint capture primer (133) (or a complementary sequence thereof); (xiii) at least one short random sequence (e.g., NNNN) (132) which provides nucleotide sequence diversity and is about 3-20 nucleotides in length; and/or (xiv) a second universal binding site (130) for a second portion of the immobilized splint capture (or a complementary sequence thereof).

In some embodiments of step (e), the universal binding site for a forward sequencing primer (140) comprises a batch-specific forward sequencing primer binding site which can be employed for forward batch sequencing.

In some embodiments of step (e), the universal binding site for a reverse sequencing primer (150) comprises a batch-specific reverse sequencing primer binding site which can be employed for reverse batch sequencing.

In some embodiments of step (e), the at least one sample index sequence (e.g., (160) and/or (170) comprises a sample index sequence joined to an optional short random sequence (e.g., NNN), where the short random sequence provides nucleotide sequence diversity and is about 3-20 nucleotides in length.

In some embodiments of step (e), the rolling circle amplification reaction can be conducted in the presence, or in the absence, of a plurality of compaction oligonucleotides.

The concatemer template molecule can self-collapse to form a DNA nanoball, sometimes called a polony. The shape and size of the DNA nanoball can be further compacted by including a pair of inverted repeat sequences in the covalently closed circular library molecules, or by conducting the rolling circle amplification reaction in the presence of a plurality of compaction oligonucleotides.

In some embodiments of step (e), rolling circle amplification (RCA) can be conducted with compaction oligonucleotides to generate single stranded concatemer template molecules having multiple copies of a repeat unit arranged in tandem, where each repeat unit comprises a sequence of interest and at least one binding site for a compaction oligonucleotide. Individual immobilized concatemer template molecules can be hybridized to at least one compaction oligonucleotide which can collapse individual concatemer template molecules into a DNA nanoball having a compact shape and size compared to a concatemer template molecules that is not hybridized to a compaction oligonucleotide.

In some embodiments of step (e), the compaction oligonucleotides comprise single-stranded nucleic acid oligonucleotides comprising DNA, RNA, or a combination of DNA and RNA. The compaction oligonucleotides can be any length, including 20-150 nucleotides, or 30-100 nucleotides, or 40-80 nucleotides in length. The compaction oligonucleotides can include a 5′ region, an optional internal region (intervening region), and a 3′ region. The 5′ and 3′ regions of the compaction oligonucleotide can hybridize to binding sites in the concatemer to pull together distal portions of the concatemer causing compaction of the concatemer to form a DNA nanoball (e.g., a polony). For example, the 5′ region of the compaction oligonucleotide is designed to hybridize to a first portion of the concatemer template molecule (e.g., a universal binding site for a compaction oligonucleotide), and the 3′ region of the compaction oligonucleotide is designed to hybridized to a second portion of the same concatemer template molecule (e.g., a universal binding site for a compaction oligonucleotide). The 5′ and 3′ regions of the compaction oligonucleotide can hybridize to regions of the concatemer template molecules having universal sites for binding a compaction oligonucleotide. The 5′ and 3′ regions of the compaction oligonucleotide can hybridize to regions in the concatemer template molecules which overlap with any of a splint capture primer binding site, a non-splint primer binding sites, a pinning primer binding site, a forward sequencing primer binding site and/or a reverse sequencing primer binding site. The intervening region can be any length, for example about 2-20 nucleotides in length. The intervening region can include a homopolymer region having consecutive identical bases (e.g., AAA, GGG, CCC, TTT or UUU). In some embodiments, the intervening region comprises a non-homopolymer sequence.

Inclusion of compaction oligonucleotides during RCA can promote formation of DNA nanoballs having tighter size and shape compared to concatemers generated in the absence of the compaction oligonucleotides. The DNA nanoballs are stable and retain their compact size and shape during multiple reagent flows for example during multiple sequencing cycles. The stable characteristics of the DNA nanoballs improves sequencing accuracy by increasing signal intensity during multiple sequencing cycles.

The DNA nanoballs can be imaged and a FWHM (full width half maximum) measurement can be obtained to determine the shape/size of the nanoballs. A spot image of a DNA nanoball can be represented as a Gaussian spot and the size can be measured as a FWHM. A smaller spot size as indicated by a smaller FWHM typically correlates with an improved image of the spot. In some embodiments, the FWHM of a nanoball spot can be about 10 um or smaller. In some embodiments, the spot image of a DNA nanoball remains a discrete spot during multiple sequencing cycles.

In some of step (e), after conducting the rolling circle amplification reaction, the covalently closed circular library molecules can optionally be removed from the concatemer template molecules with at least one washing step which is conducted under a condition suitable to retain the concatemer template molecules immobilized to the support, where individual concatemer template molecules are operably joined to an immobilized splint capture primer (200).

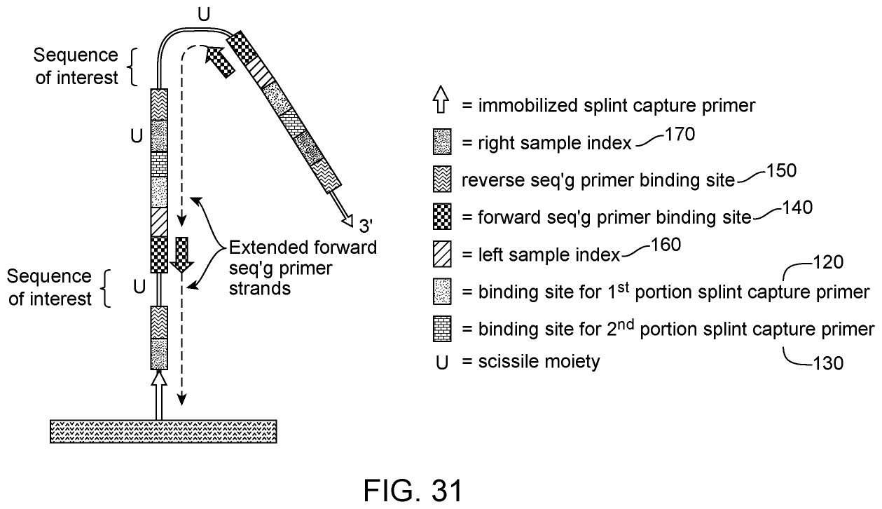

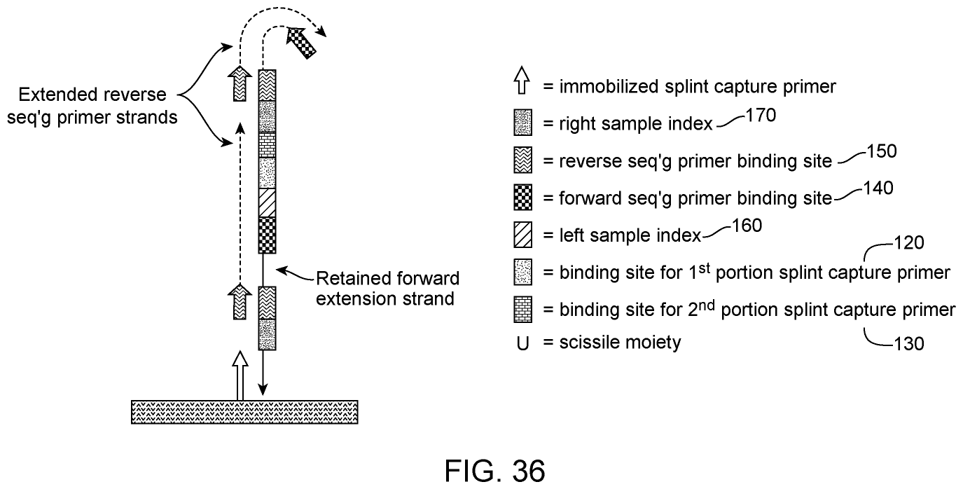

In some embodiments, the method for generating a plurality of nucleic acid concatemer template molecules immobilized to a support further comprises step (f): conducting at least one sequencing reaction to determine the sequence of at least a portion of the concatemer template molecules. In some embodiments, the concatemer template molecules serve as nucleic acid template molecules to be sequenced (e.g., concatemer template molecules). In some embodiments, the concatemer template molecules can be sequenced using any sequencing method. For example, a sequencing method can employ a plurality of sequencing primers, a plurality of sequencing polymerases, and at least one nucleotide reagent (e.g., FIGS. 31 and 36).

In some embodiments, the plurality of sequencing polymerases of step (f) comprise engineered polymerases comprising at least 70%, 75%, 80%, 85%, 90%, 91%, 92%, 93%, 94%, 95%, 96%, 97%, 98%, 99%, or greater than 99% sequence identity to any of SEQ ID NOS: 128-146 (e.g., FIGS. 41-59 respectively).

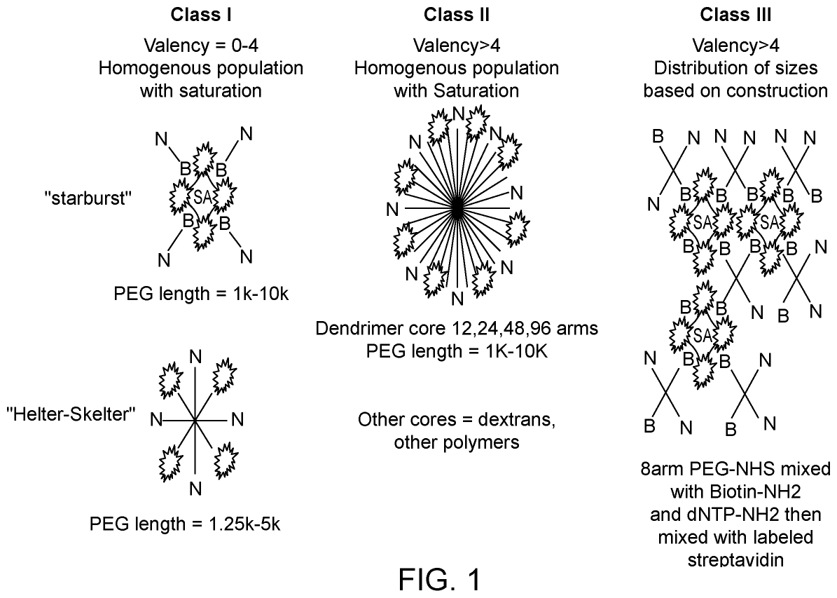

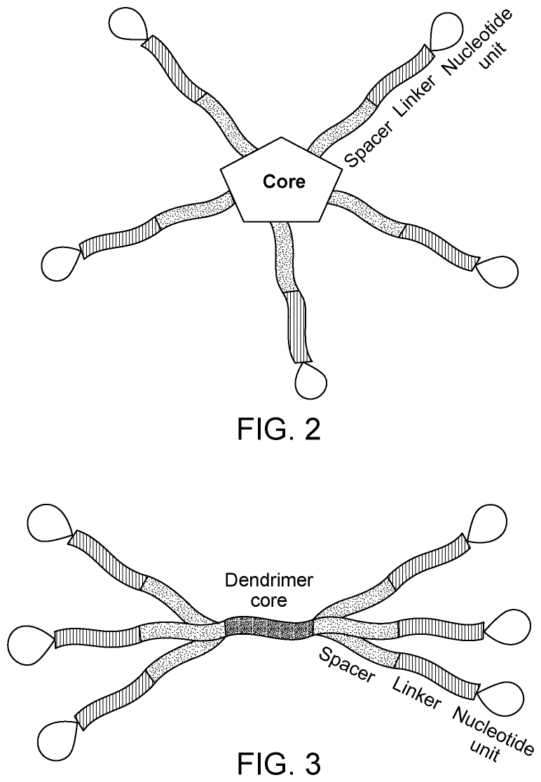

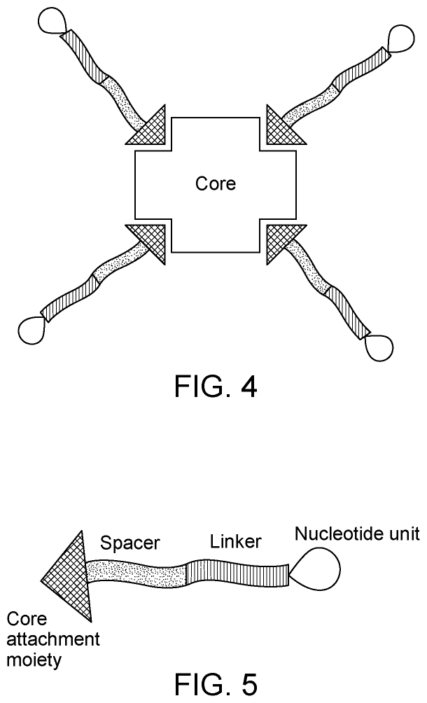

In some embodiments, the nucleotide reagent comprises any one or any combination of nucleotides and/or multivalent molecules. In some embodiments, the nucleotide reagents comprise canonical nucleotides. In some embodiments, the nucleotide reagents comprise nucleotide analogs. In some embodiments, the nucleotide analogs comprise detectably labeled nucleotides. For example, the detectably labeled nucleotides can be labeled at the nucleo-base and/or the phosphate chain. In some embodiments, the nucleotide reagents comprise nucleotides carrying a removable or non-removable chain terminating moiety. In some embodiments, the nucleotide reagents comprise multivalent molecules each comprising a central core attached to multiple polymer arms each having a nucleotide moiety at the end of the arms (e.g., FIGS. 1-4).

In some embodiments, the sequencing reactions employ binding non-labeled nucleotides without incorporation. In some embodiments, the sequencing reactions employ incorporating non-labeled nucleotide analogs. In some embodiments, the sequencing reactions employ incorporating detectably labeled nucleotides having removable chain terminating moiety. In some embodiments, the sequencing reactions employ a two-stage sequencing reaction comprising binding detectably labeled multivalent molecules without incorporation, and incorporating nucleotides or nucleotide analogs. In some embodiments, the sequence reactions employ incorporating a nucleotide moiety from an arm of a multivalent molecule. An exemplary nucleotide arm is shown in FIG. 5, and exemplary multivalent molecules are shown in FIGS. 1-4. In some embodiments, any of the detectably labeled nucleotide reagents comprise at least one fluorophore.

In some embodiments, the sequencing of step (f) comprises generating a plurality of extended forward sequencing primer strands by contacting the plurality of immobilized concatemer template molecules with a plurality of soluble forward sequencing primers under a condition suitable to hybridize at least one forward sequencing primer to at least one of the universal forward sequencing primer binding sites of the immobilized concatemer template molecules, and conducting forward sequencing reactions using the hybridized first forward sequencing primers, one or more types of sequencing polymerases, and the nucleotide reagent (e.g., FIG. 31). In some embodiments, the soluble forward sequencing primers comprise 3′ OH extendible ends. In some embodiments, the soluble forward sequencing primers comprise a 3′ blocking moiety which can be removed to generate a 3′ OH extendible end. In some embodiments, the soluble forward sequencing primers lack a nucleotide having a scissile moiety. The forward sequencing reactions can generate a plurality of extended forward sequencing primer strands. In some embodiments, individual immobilized concatemer template molecules have multiple copies of the universal forward sequencing primer binding sites, wherein each forward sequencing primer binding site is capable of hybridizing to a first forward sequencing primer. Individual forward sequencing primer binding sites in a given immobilized concatemer template molecule can be hybridized to a forward sequencing primer and can undergo a sequencing reaction. Individual immobilized concatemer template molecules can undergo two or more sequence reactions, where each sequencing reaction is initiated from a first forward sequencing primer that is hybridized to a universal forward sequencing primer binding site (e.g., FIG. 31).

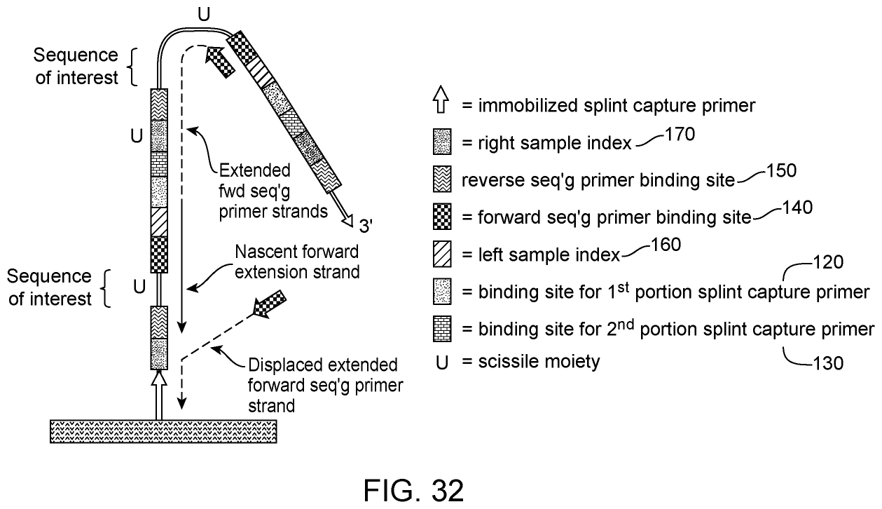

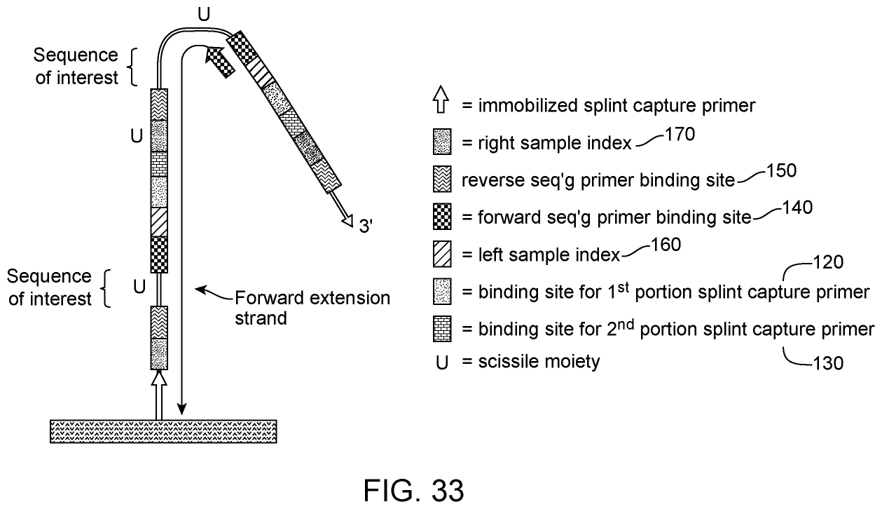

In some embodiments, the sequencing method further comprises step (g): retaining the plurality of immobilized concatemer template molecules and replacing the plurality of extended forward sequencing primer strands with a plurality of forward extension strands that are hybridized to the retained immobilized single stranded nucleic acid concatemer template molecules. The plurality of extended forward sequencing primer strands can be removed and replaced with a plurality of forward extension strands by conducting a primer extension reaction (e.g., FIGS. 32 and 33). In some embodiments, the plurality of forward extension strands can be generated by different workflows which are described below in steps (g1), (g2) and (g3).

In some embodiments, methods for replacing the plurality of extended forward sequencing primer strands with a plurality of forward extension strands of step (g1) comprises contacting at least one extended forward sequencing primer strand with a plurality of strand displacing polymerases and a plurality of nucleotides and in the absence of additional soluble amplification primers, under a condition suitable to conduct a strand displacing primer extension reaction using the at least one extended forward sequencing primers strand to initiate the primer extension reaction thereby generating a forward extension strand that is covalently joined to the extended forward sequencing primers strand, wherein the forward extension strand is hybridized to the immobilized concatemer template molecule (e.g., FIG. 32). For example, one of the extended forward sequencing primer strands can serve as a primer for the strand displacing polymerase. The strand displacing polymerase can extend the extended forward sequencing primer strand, and displace downstream extended forward sequencing primer strands while synthesizing an extended strand that replaces the downstream extended forward sequencing primer strands. The newly extended strand is covalently joined to an extended forward sequencing primer strand. The immobilized concatemer template molecules are retained (e.g., FIG. 32). The primer extension reaction of step (g1) can optionally include a plurality of compaction oligonucleotides and/or hexamine (e.g., cobalt hexamine III) to generate forward extension strands. Individual forward extension strands can collapse into a nanoball having a more compact size and/or shape compared to a nanoball generated from a primer extension reaction conducted without compaction oligonucleotides and/or hexamine (e.g., cobalt hexamine III). Inclusion of compaction oligonucleotides and/or hexamine (e.g., cobalt hexamine III) in the primer extension reaction can improve FWHM (full width half maximum) of a spot image of the nanoball. The spot image can be represented as a Gaussian spot and the size can be measured as a FWHM. A smaller spot size as indicated by a smaller FWHM typically correlates with an improved image of the spot. In some embodiments, the FWHM of a nanoball spot can be about 10 um or smaller. In some embodiments, the plurality of compaction oligonucleotides in step (e) and step (g1) have the same sequence or different sequences.

Examples of strand displacing polymerases include phi29 DNA polymerase, large fragment of Bst DNA polymerase, large fragment of Bsu DNA polymerase (exo-), Bca DNA polymerase (exo-), Klenow fragment of E. coli DNA polymerase, T5 polymerase, M-MuLV reverse transcriptase, HIV viral reverse transcriptase, Deep Vent DNA polymerase and KOD DNA polymerase. The phi29 DNA polymerase can be wild type phi29 DNA polymerase (e.g., MagniPhi from Expedeon), or variant EquiPhi29 DNA polymerase (e.g., from Thermo Fisher Scientific, catalog #A39390), or chimeric QualiPhi DNA polymerase (e.g., from 4basebio, catalog #510025).

In some embodiments, methods for replacing the plurality of extended forward sequencing primer strands with a plurality of forward extension strands of step (g2) comprises: (i) removing the plurality of extended forward sequencing primer strand while retaining the immobilized concatemer template molecules; and (ii) contacting the plurality of retained immobilized concatemer template molecules with a plurality of soluble forward sequencing primers (e.g., a second plurality of soluble forward sequencing primers), a plurality of nucleotides (e.g., a second plurality of nucleotides) and a plurality of primer extension polymerases, under a condition suitable to hybridize the plurality of soluble forward sequencing primers to the plurality of retained immobilized concatemer template molecules and suitable for conducting polymerase-catalyzed primer extension reactions thereby generating a plurality of forward extension strands, wherein the soluble sequencing primers hybridize with the forward sequencing primer binding sequence in the retained immobilized concatemer template molecules (e.g., FIG. 33). The primer extension reaction of step (g2) can optionally include a plurality of compaction oligonucleotides and/or hexamine (e.g., cobalt hexamine III) to generate forward extension strands. Individual forward extension strands can collapse into a nanoball having a more compact size and/or shape compared to a nanoball generated from a primer extension reaction conducted without compaction oligonucleotides and/or hexamine (e.g., cobalt hexamine III). Inclusion of compaction oligonucleotides and/or hexamine (e.g., cobalt hexamine III) in the primer extension reaction can improve FWHM (full width half maximum) of a spot image of the nanoball. The spot image can be represented as a Gaussian spot and the size can be measured as a FWHM. A smaller spot size as indicated by a smaller FWHM typically correlates with an improved image of the spot. In some embodiments, the FWHM of a nanoball spot can be about 10 um or smaller. In some embodiments, the plurality of compaction oligonucleotides in step (e) and step (g2) have the same sequence or different sequences.

In some embodiments, in step (g2), the condition suitable to hybridize the plurality of soluble forward sequencing primers to the plurality of retained immobilized single stranded nucleic acid concatemer template molecules comprises hybridizing retained immobilized concatemer template molecules with the soluble primers in the presence of a primer extension polymerase, a plurality of nucleotides, and a high efficiency hybridization buffer. In some embodiment, the high efficiency hybridization buffer comprises: (i) a first polar aprotic solvent having a dielectric constant that is no greater than 40 and having a polarity index of 4-9; (ii) a second polar aprotic solvent having a dielectric constant that is no greater than 115 and is present in the hybridization buffer formulation in an amount effective to denature double-stranded nucleic acids; (iii) a pH buffer system that maintains the pH of the hybridization buffer formulation in a range of about 4-8; and (iv) a crowding agent in an amount sufficient to enhance or facilitate molecular crowding. In some embodiments, the high efficiency hybridization buffer comprises: (i) the first polar aprotic solvent comprises acetonitrile at 25-50% by volume of the hybridization buffer; (ii) the second polar aprotic solvent comprises formamide at 5-10% by volume of the hybridization buffer; (iii) the pH buffer system comprises 2-(N-morpholino)ethanesulfonic acid (MES) at a pH of 5-6.5; and (iv) the crowding agent comprises polyethylene glycol (PEG) at 5-35% by volume of the hybridization buffer. In some embodiments, the high efficiency hybridization buffer further comprises betaine.

In some embodiments, methods for replacing the plurality of extended forward sequencing primer strands with a plurality of forward extension strands of step (g3) comprises: (i) removing the plurality of extended forward sequencing primer strand while retaining the immobilized concatemer template molecules; and (ii) contacting the plurality of retained immobilized concatemer template molecules with a plurality of soluble amplification primers, a plurality of nucleotides (e.g., a second plurality of nucleotides) and a plurality of primer extension polymerases, under a condition suitable to hybridize the plurality of soluble amplification primers to the plurality of retained immobilized concatemer template molecules and suitable for conducting polymerase-catalyzed primer extension reactions thereby generating a plurality of forward extension strands, wherein the soluble amplification primers hybridize with the soluble amplification primer binding sequence in the retained immobilized concatemer template molecules. The primer extension reaction of step (g3) can optionally include a plurality of compaction oligonucleotides and/or hexamine (e.g., cobalt hexamine III) to generate forward extension strands. Individual forward extension strands can collapse into a nanoball having a more compact size and/or shape compared to a nanoball generated from a primer extension reaction conducted without compaction oligonucleotides and/or hexamine (e.g., cobalt hexamine III). Inclusion of compaction oligonucleotides and/or hexamine (e.g., cobalt hexamine III) in the primer extension reaction can improve FWHM (full width half maximum) of a spot image of the nanoball. The spot image can be represented as a Gaussian spot and the size can be measured as a FWHM. A smaller spot size as indicated by a smaller FWHM typically correlates with an improved image of the spot. In some embodiments, the FWHM of a nanoball spot can be about 10 um or smaller. In some embodiments, the plurality of compaction oligonucleotides in step (e) and step (g3) have the same sequence or different sequences.

In some embodiments, in step (g3), the condition suitable to hybridize the plurality of soluble amplification primers to the plurality of retained immobilized single stranded nucleic acid concatemer template molecules comprises hybridizing retained immobilized concatemer template molecules with the soluble primers in the presence of a primer extension polymerase, a plurality of nucleotides, and a high efficiency hybridization buffer. In some embodiment, the high efficiency hybridization buffer comprises: (i) a first polar aprotic solvent having a dielectric constant that is no greater than 40 and having a polarity index of 4-9; (ii) a second polar aprotic solvent having a dielectric constant that is no greater than 115 and is present in the hybridization buffer formulation in an amount effective to denature double-stranded nucleic acids; (iii) a pH buffer system that maintains the pH of the hybridization buffer formulation in a range of about 4-8; and (iv) a crowding agent in an amount sufficient to enhance or facilitate molecular crowding. In some embodiments, the high efficiency hybridization buffer comprises: (i) the first polar aprotic solvent comprises acetonitrile at 25-50% by volume of the hybridization buffer; (ii) the second polar aprotic solvent comprises formamide at 5-10% by volume of the hybridization buffer; (iii) the pH buffer system comprises 2-(N-morpholino)ethanesulfonic acid (MES) at a pH of 5-6.5; and (iv) the crowding agent comprises polyethylene glycol (PEG) at 5-35% by volume of the hybridization buffer. In some embodiments, the high efficiency hybridization buffer further comprises betaine.

In some embodiments, in steps (g2) and/or (g3), the plurality of extended forward sequencing primer strands can be removed using an enzyme or a chemical reagent. For example, the plurality of extended forward sequencing primer strands can be enzymatically degraded using a 5′ to 3′ double-stranded DNA exonuclease, including T7 exonuclease (e.g., from New England Biolabs, catalog #M0263S). In some embodiments, the plurality of extended forward sequencing primer strands can be removed with a temperature that favors nucleic acid denaturation.

In some embodiments, in steps (g2) and/or (g3), a denaturation reagent can be used to remove the plurality of extended forward sequencing primer strands, wherein the denaturation reagent comprises any one or any combination of compounds such as formamide, acetonitrile, guanidinium chloride and/or a buffering agent (e.g., Tris-HCl, MES, HEPES, or the like).

In some embodiments, in steps (g2) and/or (g3), the plurality of extended forward sequencing primer strands can be removed using an elevated temperature (e.g., heat) with or without a nucleic acid denaturation reagent. The plurality of extended forward sequencing primer strands can be subjected to a temperature of about 45-50° C., or about 50-60° C., or about 60-70° C., or about 70-80° C., or about 80-90° C., or about 90-95° C., or higher temperature.

In some embodiments, in steps (g2) and/or (g3), the plurality of extended forward sequencing primer strands can be removed using 100% formamide at a temperature of about 65° C. for about 3 minutes, and washing with a reagent comprising about 50 mM NaCl or equivalent ionic strength and having a pH of about 6.5-8.5.

In some embodiments, the primer extension polymerase of any of steps (g2) and/or (g3) comprises a high fidelity polymerase. In some embodiments, the primer extension polymerase comprises a DNA polymerase capable of catalyzing a primer extension reaction using a uracil-containing template molecule (e.g., a uracil-tolerant polymerase). Exemplary polymerases include, but are not limited to, Q5U Hot Start high-fidelity DNA polymerase (e.g., catalog #M0515S from New England Biolabs), Taq DNA polymerase, One Taq DNA polymerase (e.g., mixture of Taq and Deep Vent DNA polymerases, catalog #M0480S from New England Biolabs), LongAmp Taq DNA polymerase (e.g., catalog #M0323S from New England Biolabs), Epimark Hot Start Taq DNA polymerase (e.g., catalog #M0490S from New England Biolabs), Bst DNA polymerase (e.g., large fragment, catalog #M0275S from New England Biolabs), Bsu DNA polymerase (e.g., large fragment, catalog #M0330S from New England Biolabs), Phi29 DNA polymerase (e.g., catalog #M0269S from New England Biolabs), E. coli DNA polymerase (e.g., catalog #M0209S from New England Biolabs), Therminator DNA polymerase (e.g., catalog #M0261S from New England Biolabs), Vent DNA polymerase and Deep Vent DNA polymerase.

The sequencing methods described herein can provide increased accuracy in a downstream sequencing reaction because steps (g1), (g2) and (g3) replaces the extended forward sequencing primer strands that were generated in step (f) with forward extension strands having reduced base errors. The extended forward sequencing primer strands are generated in step (e) and may or may not contain erroneously incorporated nucleotides due to polymerase-catalyzed mis-paired bases. When steps (g1), (g2) and (g3) are conducted with a high fidelity DNA polymerase, the resulting forward extension strands may have reduced base errors compared to the extended forward sequencing primer strands. The forward extension strands can be used as a nucleic acid template for a downstream sequencing step (e.g., see step (i) below). Thus, steps (g1), (g2) and (g3) can increase the sequencing accuracy of the downstream step (i) and therefore increase the overall sequencing accuracy of the sequencing workflow.

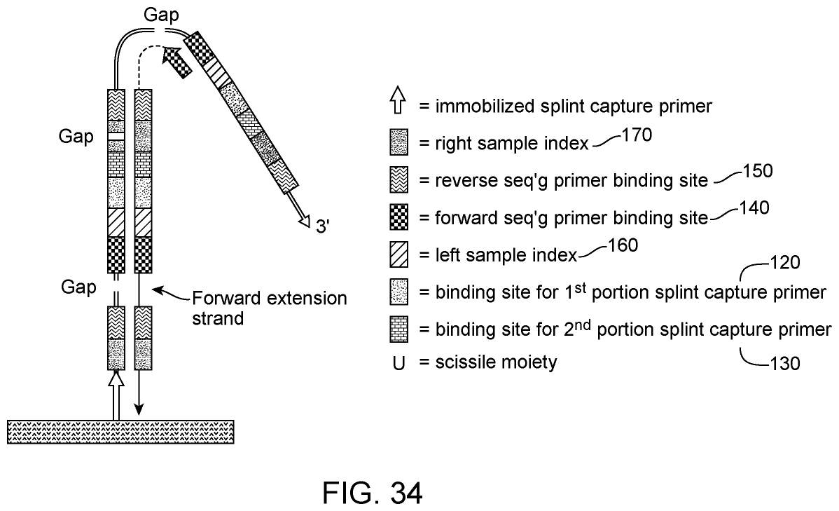

In some embodiments, the sequencing method further comprises step (h): removing the retained immobilized concatemer template molecules by generating abasic sites in the immobilized single stranded concatemer template molecules at the nucleotide(s) having the scissile moiety and generating gaps at the abasic sites to generate a plurality of gap-containing single stranded nucleic acid concatemer template molecules while retaining the plurality of forward extension strands and retaining the plurality of immobilized splint capture primers (200) and pinning primers (500) as shown in e.g., FIG. 34.

The abasic sites are generated on the retained concatemer template molecule strands that contain nucleotides having scissile moieties. In some embodiments, the scissile moieties in the retained concatemer template molecules comprises uridine, 8-oxo-7,8-dihydroguanine (e.g., 8oxoG) or deoxyinosine. The abasic sites can be removed to generate a plurality of single stranded nucleic acid template molecules having gaps while retaining the plurality of forward extension strands. The abasic sites can be generated by contacting the immobilized concatemer template molecules with an enzyme that removes the nucleo-base at the nucleotide having the scissile moiety. The uracil in the retained concatemer template strands can be converted to an abasic site using uracil DNA glycosylase (UDG). The 8oxoG in the retained concatemer template strands can be converted to an abasic site using FPG glycosylase. The deoxyinosine in the retained concatemer template strands can be converted to an abasic site using AlkA glycosylase.

In some embodiments, in step (h), the gaps can be generated by contacting the abasic sites in the immobilized concatemer template molecules with an enzyme or a mixture of enzymes having lyase activity that breaks the phosphodiester backbone at the 5′ and 3′ sides of the abasic site to release the base-free deoxyribose and generate a gap (FIG. 34). The abasic sites can be removed using AP lyase, Endo IV endonuclease, FPG glycosylase/AP lyase, Endo VIII glycosylase/AP lyase. In some embodiments, generating the abasic sites and removal of the abasic sites to generate gaps can be achieved using a mixture of uracil DNA glycosylase and DNA glycosylase-lyase endonuclease VIII, for example USER (Uracil-Specific Excision Reagent Enzyme from New England Biolabs, catalog #M5509) or thermolabile USER (also from New England Biolabs, catalog #M5508).

In some embodiments, in step (h), the concatemer template molecule carrying at least one scissile nucleotide can be reacted with at least one enzyme to convert the scissile nucleotide into an abasic site. In some embodiments, deoxyuridine can be converted to an abasic site using uracil DNA glycosylase (UDG), 8oxoG can be converted to an abasic site using FPG glycosylase, and deoxyinosine can be converted to an abasic site using AlkA glycosylase. Other exemplary enzymes that can convert an scissile nucleotide into an abasic site in a concatemer include single-strand-selective monofunctional uracil DNA glycosylase 1 (SMUG1), methyl-binding domain glycosylase 4 (MBD4), thymine DNA glycosylase (TDG), mutY homolog DNA glycosylase (MYH), alkylpurine glycosylase C (AlkC), alkylpurine glycosylase D (AlkD), 8-oxo-guanine glycosylase 1 (OGG1) without the abasic site lyase activity, endonuclease III-like 1 (NTHL1) without the abasic site lyase activity, endonuclease VIII-like glycosylase 1 (NEIL1) without the abasic site lyase activity, endonuclease VIII-like glycosylase 2 (NEIL2) without the abasic site lyase activity, endonuclease VIII-like glycosylase 3 (NEIL3) without the abasic site lyase activity, and enzymatically active fragments thereof.

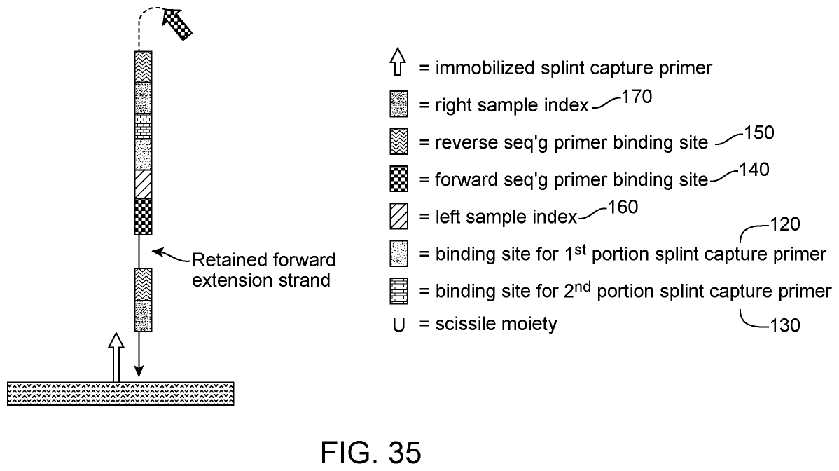

In some embodiments, in step (h), the plurality of gap-containing concatemer template molecules can be removed using an enzyme, chemical compound and/or heat. After the gap-removal procedure, the plurality of retained forward extension strands are hybridized to the retained immobilized splint capture primers as shown in e.g., FIG. 35.

For example, the plurality of gap-containing concatemer template molecules can be enzymatically degraded using a 5′ to 3′ double-stranded DNA exonuclease, including T7 exonuclease (e.g., from New England Biolabs, catalog #M0263S). When a 5′ to 3′ double-stranded DNA exonuclease is used for removing gap-containing concatemer template molecules, then the plurality of soluble amplification primers in step (g3) can comprise at least one phosphorothioate diester bond at their 5′ ends which can render the soluble amplification primers resistant to exonuclease degradation. In some embodiments, the plurality of soluble amplification primers in step (g3) comprise 2-5 or more consecutive phosphorothioate diester bonds at their 5′ ends. In some embodiments, the plurality soluble amplification primers in step (g3) comprise at least one ribonucleotide and/or at least one 2′-O-methyl or 2′-O-methoxyethyl (MOE) nucleotide which can render the forward sequencing primers resistant to exonuclease degradation.

In some embodiments, in step (h), the plurality of gap-containing concatemer template molecules can be removed using a chemical reagent that favors nucleic acid denaturation. The denaturation reagent can include any one or any combination of compounds such as formamide, acetonitrile, guanidinium chloride and/or a buffering agent (e.g., Tris-HCl, MES, HEPES, or the like).

In some embodiments, in step (h), the plurality of gap-containing concatemer template molecules can be removed using an elevated temperature (e.g., heat) with or without a nucleic acid denaturation reagent. The gap-containing template molecules can be subjected to a temperature of about 45-50° C., or about 50-60° C., or about 60-70° C., or about 70-80° C., or about 80-90° C., or about 90-95° C., or higher temperature.

In some embodiments, in step (h), the plurality of gap-containing concatemer template molecules can be removed using 100% formamide at a temperature of about 65° C. for about 3 minutes, and washing with a reagent comprising about 50 mM NaCl or equivalent ionic strength and having a pH of about 6.5-8.5.

In some embodiments, the sequencing method further comprises step (i): sequencing the plurality of retained forward extension strands thereby generating a plurality of extended reverse sequencing primer strands. In some embodiments, the sequencing of step (i) comprises contacting the plurality of retained forward extension strands with a plurality of soluble reverse sequencing primers under a condition suitable to hybridize the reverse sequencing primers to the reverse sequencing primer binding site of the retained forward extension strands, and conducting sequencing reactions using the hybridized reverse sequencing primers wherein the forward sequencing reactions generates a plurality of extended reverse sequencing primer strands (e.g., FIG. 36). The extended reverse sequencing primer strands can be hybridized to the retained forward extension strand. The retained forward extension strand can be hybridized to the splint capture primer. Thus, the retained forward extension strands can be immobilized to the support. The extended reverse sequencing primer strands are not hybridized to the splint capture primer, or covalently joined to the splint capture primer.

In some embodiments, in step (i), the immobilized retained forward extension strands serve as nucleic acid template molecules to be sequenced (e.g., FIG. 36). In some embodiments, the retained forward extension strands can be sequenced using any sequencing method. For example, a sequencing method can employ a plurality of sequencing primers, a plurality of sequencing polymerases, and at least one nucleotide reagent.

In some embodiments, the plurality of sequencing polymerases of step (i) comprise engineered polymerases comprising at least 70%, 75%, 80%, 85%, 90%, 91%, 92%, 93%, 94%, 95%, 96%, 97%, 98%, 99%, or greater than 99% sequence identity to any of SEQ ID NOS:128-146 (e.g., FIGS. 41-59 respectively), or any suitable polymerase described herein.

In some embodiments, in step (i) the nucleotide reagent comprises any one or any combination of nucleotides and/or multivalent molecules. In some embodiments, the nucleotide reagents comprise canonical nucleotides. In some embodiments, the nucleotide reagents comprise nucleotide analogs. In some embodiments, the nucleotide analogs comprise detectably labeled nucleotides. For example, the detectably labeled nucleotides can be labeled at the nucleo-base and/or the phosphate chain. In some embodiments, the nucleotide reagents comprise nucleotides carrying a removable or non-removable chain terminating moiety. In some embodiments, the nucleotide reagents comprise multivalent molecules each comprising a central core attached to multiple polymer arms each having a nucleotide moiety at the end of the arms (e.g., FIGS. 1-4).

In some embodiments, in step (i) the sequencing reactions employ binding non-labeled nucleotides without incorporation. In some embodiments, the sequencing reactions employ incorporating non-labeled nucleotide analogs. In some embodiments, the sequencing reactions employ incorporating detectably labeled nucleotides having removable chain terminating moiety. In some embodiments, the sequencing reactions employ a two-stage sequencing reaction comprising binding detectably labeled multivalent molecules without incorporation, and incorporating nucleotides or nucleotide analogs. In some embodiments, the sequence reactions employ incorporating a nucleotide moiety from an arm of a multivalent molecule. An exemplary nucleotide arm is shown in FIG. 5, and exemplary multivalent molecules are shown in FIGS. 1-4. In some embodiments, any of the detectably labeled nucleotide reagents comprise at least one fluorophore.

In some embodiments, in step (i), the condition suitable to hybridize the reverse sequencing primers to the reverse sequencing primer binding sequences of the retained forward extension strands comprises contacting the plurality of soluble reverse sequencing primers and the retained forward extension strands with a high efficiency hybridization buffer. In some embodiments, the high efficiency hybridization buffer comprises: (i) a first polar aprotic solvent having a dielectric constant that is no greater than 40 and having a polarity index of 4-9; (ii) a second polar aprotic solvent having a dielectric constant that is no greater than 115 and is present in the hybridization buffer formulation in an amount effective to denature double-stranded nucleic acids; (iii) a pH buffer system that maintains the pH of the hybridization buffer formulation in a range of about 4-8; and (iv) a crowding agent in an amount sufficient to enhance or facilitate molecular crowding. In some embodiments, the high efficiency hybridization buffer comprises: (i) the first polar aprotic solvent comprises acetonitrile at 25-50% by volume of the hybridization buffer; (ii) the second polar aprotic solvent comprises formamide at 5-10% by volume of the hybridization buffer; (iii) the pH buffer system comprises 2-(N-morpholino)ethanesulfonic acid (MES) at a pH of 5-6.5; and (iv) the crowding agent comprises polyethylene glycol (PEG) at 5-35% by volume of the hybridization buffer. In some embodiments, the high efficiency hybridization buffer further comprises betaine.

In an alternative embodiment, the sequencing of step (i) comprises using the immobilized splint capture primer, e.g. (200), as a sequencing primer and conducting sequencing reactions to generate a plurality of reverse sequencing strands.

In some embodiments, the reverse sequencing reactions of step (i) comprises contacting the plurality of soluble reverse sequencing primers with the reverse sequencing primer binding sequences of the retained forward extension strands, one or more types of sequencing polymerases, and a plurality of nucleotides and/or a plurality of multivalent molecules (e.g., FIG. 36). In some embodiments, the soluble reverse sequencing primers comprise 3′ OH extendible ends. In some embodiments, the soluble reverse sequencing primers comprise a 3′ blocking moiety which can be removed to generate a 3′ OH extendible end. In some embodiments, the soluble reverse sequencing primers lack a nucleotide having a scissile moiety. The sequencing reactions that employ nucleotides and/or multivalent molecules is described in more detail below. The reverse sequencing reactions can generate a plurality of extended reverse sequencing primer strands. In some embodiments, individual retained forward extension strands have multiple copies of the reverse sequencing primer binding sequences/sites, wherein each reverse sequencing primer binding site is capable of hybridizing to a reverse sequencing primer. Individual reverse sequencing primer binding sites in a given retained forward extension strand can be hybridized to a reverse sequencing primer and can undergo a sequencing reaction. Thus, an individual retained forward extension strand can undergo two or more sequence reactions, where each sequencing reaction is initiated from a reverse sequencing primer that is hybridized to a reverse sequencing primer binding site. In some embodiments, the sequencing reactions comprise a plurality of nucleotides (or analogs thereof) labeled with a detectable reporter moiety. In some embodiments, the sequencing reaction comprise a plurality of multivalent molecules having nucleotide moieties, where the multivalent molecules are labeled with a detectable reporter moiety. In some embodiments, the detectable reporter moiety comprises a fluorophore.

In some embodiments, at least one washing step can be conducted after any of the sequencing steps (a)-(i). The washing step can be conducted with a wash buffer comprising a pH buffering agent, a metal chelating agent, a salt, and a detergent.

In some embodiments, the pH buffering compound in the wash buffer comprises any one or any combination of two or more of Tris, Tris-HCl, Tricine, Bicine, Bis-Tris propane, HEPES, MES, MOPS, MOPSO, BES, TES, CAPS, TAPS, TAPSO, ACES, PIPES, ethanolamine (a.k.a 2-amino methanol; MEA), a citrate compound, a citrate mixture, NaOH and/or KOH. In some embodiments, the pH buffering agent can be present in the wash buffer at a concentration of about 1-100 mM, or about 10-50 mM, or about 10-25 mM. In some embodiments, the pH of the pH buffering agent which is present in any of the reagents described here in can be adjusted to a pH of about 4-9, or a pH of about 5-9, or a pH of about 5-8.

In some embodiments, the metal chelating agent in the wash buffer comprises EDTA (ethylenediaminetetraacetic acid), EGTA (ethylene glycol tetraacetic acid), HEDTA (hydroxyethylethylenediaminetriacetic acid), DPTA (diethylene triamine pentaacetic acid), NTA (N,N-bis(carboxymethyl)glycine), citrate anhydrous, sodium citrate, calcium citrate, ammonium citrate, ammonium bicitrate, citric acid, potassium citrate, or magnesium citrate. In some embodiments, the wash buffer comprises a chelating agent at a concentration of about 0.01-50 mM, or about 0.1-20 mM, or about 0.2-10 mM.

In some embodiments, the salt in the wash buffer comprises NaCl, KCl, NH₂SO₄ or potassium glutamate. In some embodiments, the detergent comprises an ionic detergent such as SDS (sodium dodecyl sulfate). The wash buffer can include a monovalent salt at a concentration of about 25-500 mM, or about 50-250 mM, or about 100-200 mM.

In some embodiments, the detergent in the wash buffer comprises a non-ionic detergent such as Triton X-100, Tween 20, Tween 80 or Nonidet P-40. In some embodiments, the detergent comprises a zwitterionic detergent such as CHAPS (3-[(3-cholamidopropyl) dimethylammonio]-1-propanesulfonate) or N-Dodecyl-N,N-dimethyl-3-amonio-1-propanesulfate (DetX). In some embodiments, the detergent comprises LDS (lithium dodecyl sulfate), sodium taurodeoxycholate, sodium taurocholate, sodium glycocholate, sodium deoxycholate or sodium cholate. In some embodiments, the detergent is included in the wash buffer at a concentration of about 0.01-0.05%, or about 0.05-0.1%, or about 0.1-0.15%, or about 0.15-0.2%, or about 0.2-0.25%.

Forming Immobilized Open Circular Library Molecules with 5′ Flaps

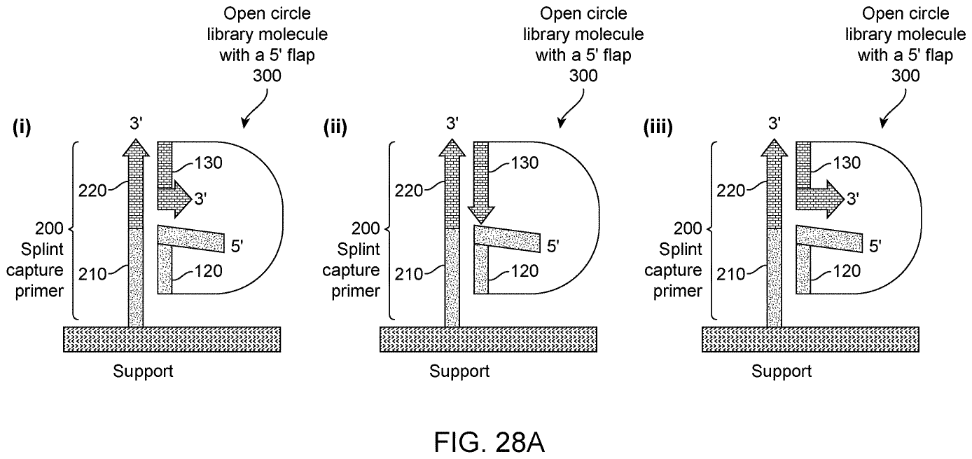

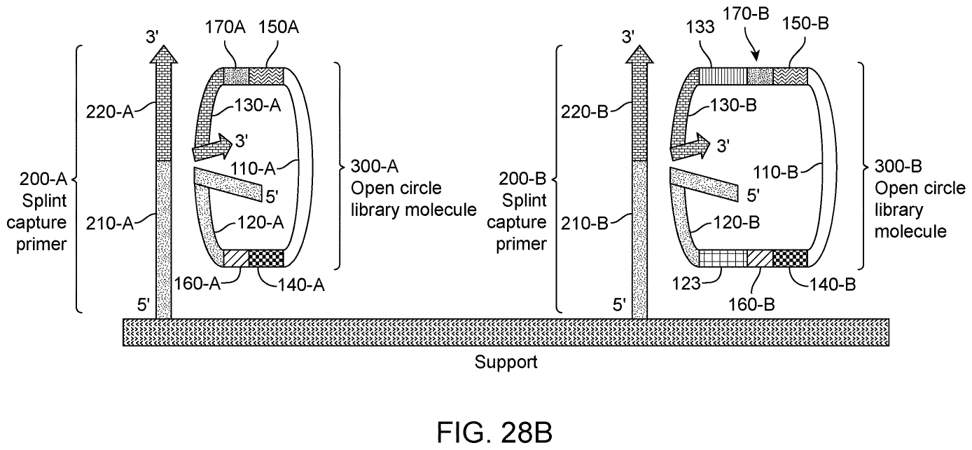

In some embodiments, in the method for generating a plurality of nucleic acid concatemers immobilized to a support as described above, step (c) comprises: (i) forming a plurality of open circle library molecules each having a 5′ overhang flap, by contacting the plurality of immobilized splint capture primers (200) with the plurality of linear library molecules (100), wherein the contacting is conducted under a condition suitable for hybridizing individual linear library molecules to individual immobilized splint capture primers to form individual open circle library molecules each having at least a portion of the first terminal region of a given linear library molecule hybridized to a first portion (210) of a splint capture primer and having at least a portion of the second terminal region of the same linear library molecule hybridized to a second portion (220) of the same splint capture primer, wherein the terminal 5′ end of individual open circle library molecules form a 5′ overhang flap structure that is cleavable with a structure specific 5′ flap endonuclease (e.g., FIGS. 28A(i), 28A(ii), 28B left and 28B right), and step (c) comprises (ii) cleaving the 5′ overhang flap structures by contacting the plurality of open circle library molecules with a flap cleaving reagent under a condition suitable for cleaving the 5′ overhang flap structures thereby forming a plurality of cleavage products. In some embodiments, individual cleavage products comprise an open circle library molecule with a newly cleaved 5′ end and a non-cleaved 3′ end, wherein the newly cleaved 5′ end and the non-cleaved 3′ end of the same open circle library molecule forms a nick while being hybridized to the first portion (210) and the second portion (220) of the same splint capture primer, wherein the nick is enzymatically ligatable. In some embodiments, the flap cleaving reagent cleaves the 5′ flap and the 3′ flap thereby generating a plurality of cleavage products, wherein individual cleavage products comprise an open circle library molecule with a newly cleaved 5′ end and a newly cleaved 3′ end, wherein the newly cleaved 5′ and 3′ ends of the same open circle library molecule form a nick while being hybridized to the first portion (210) and the second portion (220) of the same splint capture primer, wherein the nick is enzymatically ligatable. In some embodiments, the flap cleaving reagent cleaves the 5′ flap and the 3′ flap thereby generating a plurality of cleavage products, wherein individual cleavage products comprise an open circle library molecule with a newly cleaved 5′ end and a newly cleaved 3′ end, wherein the newly cleaved 5′ and 3′ ends of the same open circle library molecule form a gap while being hybridized to the first portion (210) and the second portion (220) of the same splint capture primer. In some embodiments, the gap can be subjected to a polymerase-catalyzed fill-in reaction to generate a nick, wherein the nick is enzymatically ligatable. In some embodiments, the gap in individual open circle library molecules can be closed by conducting a polymerase-catalyzed gap fill-in reaction using the newly-cleaved 3′ end of the library molecule as an initiation site for the polymerase-catalyzed fill-in reaction and using the immobilized splint capture primer as a template molecule thereby forming an open circle library molecule having a nick. The nick can be closed by conducting an enzymatic ligation reaction to form a single stranded covalently closed circular library molecule, wherein individual covalently closed circular library molecules are hybridized to an immobilized splint capture primer.

In some embodiments, the method for generating a plurality of nucleic acid concatemer template molecules immobilized to a support further comprises step (d): enzymatically closing the nicks in the plurality of open circle library molecules thereby generating a plurality of covalently closed circular library molecules (400) wherein individual covalently closed circular library molecules are hybridized to an immobilized splint capture primer (200).

In some embodiments, the method for generating a plurality of nucleic acid concatemer template molecules immobilized to a support further comprises step (e): contacting the plurality of covalently closed circular library molecules with a rolling circle amplification reaction mixture and conducting a plurality of rolling circle amplification reaction thereby generating a plurality of immobilized nucleic acid concatemer template molecules, wherein the density of the immobilized concatemer template molecules is 10⁵-10¹⁵ per mm².

In some embodiments, the method for generating a plurality of nucleic acid concatemer template molecules immobilized to a support further comprises step (f): conducting at least one sequencing reaction to determine the sequence of at least a portion of the plurality of immobilized concatemer template molecules.

In some embodiments, the contacting of step (c) comprises distributing the plurality of single stranded nucleic acid linear library molecules onto the support having the plurality of immobilized splint capture primers (200) and pinning primers (500). In some embodiments, the contacting of step (c) comprises distributing one type of single stranded nucleic acid linear library molecules onto the support having the plurality of immobilized splint capture and pinning primers. In some embodiments, the contacting of step (c) comprises distributing a mixture of at least two different types of single stranded nucleic acid linear library molecules onto the support having the plurality of immobilized splint capture and pinning primers, wherein the at least two types comprises at least a first and second sub-population of linear library molecules and wherein the support comprises a first and second sub-population of immobilized splint capture primers (200), e.g. FIG. 28B left and 28B right. In some embodiments, the universal binding sequence for a first portion of an immobilized splint capture primer in the linear library molecule (100) can hybridize to the first portion (210) of the immobilized splint capture primer. In some embodiments, the second universal binding site (130) for a second portion of an immobilized splint capture primer in the linear library molecule (100) can hybridize to the second portion (220) of the immobilized splint capture primer. In some embodiments, the immobilized splint capture primers comprise a first portion (210) and a second portion (220) which hybridize to adaptor sequences, e.g. (120) and (130), in the linear library molecule, and the splint capture primers serve as a nucleic acid splint molecule for circularizing the linear library molecules (e.g., FIGS. 28A(i), 28A(ii), 28A(iii), 28B left and 28B right).

In some embodiments, step (c) comprises: (i) forming a first sub-population of open circle library molecules, wherein individual open circle library molecules have a 5′ overhang flap, by contacting a first sub-population of immobilized splint capture primers (200-A) with a first sub-population of nucleic acid linear library molecules, wherein the contacting is conducted under a condition suitable for hybridizing individual linear library molecules of the first sub-population to individual immobilized splint capture primers to form individual open circle library molecules (300-A) each having at least a portion of the first terminal region of a given linear library molecule hybridized to a first portion (210-A) of a splint capture primer and having at least a portion of the second terminal region of the same linear library molecule hybridized to a second portion (220-A) of the same splint capture primer, wherein the terminal 5′ end of individual open circle library molecules form a 5′ overhang flap structure that is cleavable with a structure specific 5′ flap endonuclease (e.g., FIG. 28B left), and (ii) cleaving the 5′ overhang flap structures by contacting the first sub-population of open circle library molecules with a flap cleaving reagent under a condition suitable for cleaving the 5′ overhang flap structures thereby forming a first sub-population of cleavage products, wherein individual cleavage products comprise an open circle library molecule with a newly cleaved 5′ end and a non-cleaved 3′ end, wherein the newly cleaved 5′ end and the non-cleaved 3′ end of the same open circle library molecule forms a nick while being hybridized to the first portion (210-A) and the second portion (220-A) of the same splint capture primer. In some embodiments, the contacting of step (c) comprises distributing the first sub-population of linear library molecules onto the support having a mixture of first and second sub-populations of immobilized splint capture primers. In some embodiments, the contacting of step (c) comprises distributing the first sub-population of linear library molecules onto the support having a plurality of pinning primers (500). In some embodiments, the immobilized splint capture primers (200-A) comprise a first portion (210-A) and a second portion (220-A) which hybridize to adaptor sequences (120-A) and (130-A) in the linear library molecules of the first sub-population, and the splint capture primers (200-A) serve as a nucleic acid splint molecule for circularizing the linear library molecules (e.g., FIG. 28B left). In some embodiments, individual linear library molecules of the first sub-population comprise a universal binding sequence (120-A) that can hybridize to the first portion (210-A) of individual immobilized splint capture primers in the first sub-population. In some embodiments, individual linear library molecules of the first sub-population comprise a universal binding sequence (130-A) that can hybridize to the second portion (220-A) of individual immobilized splint capture primers in the first sub-population. In some embodiments, the linear library molecules in the first sub-population are single stranded.

In some embodiments, step (c) comprises: (i) forming a second sub-population of open circle library molecules wherein individual open circle library molecules have a 5′ overhang flap, by contacting the second sub-population of immobilized splint capture primers (200-A) with the second sub-population of nucleic acid linear library molecules, wherein the contacting is conducted under a condition suitable for hybridizing individual linear library molecules of the second sub-population to individual immobilized splint capture primers to form individual open circle library molecules (300-B) each having at least a portion of the first terminal region of a given linear library molecule hybridized to a first portion (210-B) of a splint capture primer and having at least a portion of the second terminal region of the same linear library molecule hybridized to a second portion (220-B) of the same splint capture primer, wherein the terminal 5′ end of individual open circle library molecules form a 5′ overhang flap structure that is cleavable with a structure specific 5′ flap endonuclease (e.g., FIG. 28B right), and (ii) cleaving the 5′ overhang flap structures by contacting the second sub-population of open circle library molecules with a flap cleaving reagent under a condition suitable for cleaving the 5′ overhang flap structures thereby forming a second sub-population of cleavage products, wherein individual cleavage products comprise an open circle library molecule with a newly cleaved 5′ end and a non-cleaved 3′ end, wherein the newly cleaved 5′ end and the non-cleaved 3′ end of the same open circle library molecule forms a nick while being hybridized to the first portion (210-B) and the second portion (220-B) of the same splint capture primer. In some embodiments, the contacting of step (c) comprises distributing the second sub-population of linear library molecules onto the support having a mixture of first and second sub-populations of immobilized splint capture primers. In some embodiments, the contacting of step (c) comprises distributing the second sub-population of linear library molecules onto the support having a plurality of pinning primers (500). In some embodiments, the immobilized splint capture primers (200-B) comprise a first portion (210-B) and a second portion (220-B) which hybridize to adaptor sequences (120-B) and (130-B) in the linear library molecules of the second sub-population, and the splint capture primers (200-B) serve as a nucleic acid splint molecule for circularizing the linear library molecules (e.g., FIG. 28B right). In some embodiments, individual linear library molecules of the second sub-population comprise a universal binding sequence (120-B) that can hybridize to the first portion (210-B) of individual immobilized splint capture primers in the second sub-population. In some embodiments, individual linear library molecules of the second sub-population comprise a universal binding sequence (130-B) that can hybridize to the second portion (220-B) of individual immobilized splint capture primers in the second sub-population. In some embodiments, the linear library molecules in the second sub-population are single stranded.

In some embodiments, contacting the first sub-population of immobilized splint capture primers (200-A) with the first sub-population of nucleic acid linear library molecules and contacting the second sub-population of immobilized splint capture primers (200-A) with the second sub-population of nucleic acid linear library molecules happens simultaneously. In some embodiments, contacting the first sub-population of immobilized splint capture primers (200-A) with the first sub-population of nucleic acid linear library molecules and contacting the second sub-population of immobilized splint capture primers (200-A) with the second sub-population of nucleic acid linear library molecules happens sequentially.

In some embodiments, step (c) comprises: contacting a support comprising a plurality of a first sub-population of immobilized splint capture primers (200-A) and a plurality of a second sub-population of immobilized splint capture primers (200-B) with the first sub-population of nucleic acid linear library molecules or the second sub-population of nucleic acid library molecules.

In some embodiments, step (c) comprises: contacting a support comprising a plurality of a first sub-population of immobilized splint capture primers (200-A) and a plurality of a second sub-population of immobilized splint capture primers (200-B) with the first sub-population of nucleic acid linear library molecules and the second sub-population of nucleic acid library molecules essentially simultaneously or separately in any order.

In some embodiments of step (c), the 5′ flap endonuclease comprises a structure-specific 5′ flap endonuclease which can cleave off the 5′ flap structure of single-stranded DNA or RNA. The structure-specific 5′ flap endonuclease does not cleave a specific sequence, but instead cleaves a 5′ overhang flap structure. The structure specific 5′ flap endonuclease catalyzes hydrolytic cleavage of the phosphodiester bond at the junction of single stranded and double stranded DNA, releasing the 5′ overhang flap.

In some embodiments of step (c), the 5′ overhang flap structure comprises a nucleic acid sequence that is not complementary to the first portion (210) of the splint capture primer.

In some embodiments of step (c), the 5′ overhang flap structure is at least 2 nucleotides in length. In any of the embodiments of step (c), the 5′ overhang flap structure is 2-10 nucleotides in length.

In some embodiments of step (c), cleavage at any position of the 5′ flap structure generates a cleavage product. The cleavage product can be 2-10 nucleotides in length.

In some embodiments of step (c), the structure-specific 5′ flap endonuclease comprises a Flap Endonuclease 1 (FEN1), a RAD2 endonuclease or an XPG endonuclease.

In some embodiments of step (c), individual open circle library molecules lack a 3′ overhang flap structure. In some embodiments, individual open circle library molecules further comprise a 3′ overhang flap structure. In some embodiments, the 3′ overhang flap structure comprises a nucleic acid sequence that is complementary to the second portion (220) of the splint capture primers. In some embodiments, the 3′ overhang flap structure is 1 nucleotide in length. In some embodiments, the 3′ overhang flap structure is 2-10 nucleotides in length. In some embodiments, the 5′ flap endonuclease does not cleave the 3′ overhang flap structure.

In some embodiments of step (c), individual open circle library molecules comprise a 5′ overhang flap structure that is 2-10 nucleotides in length and a 3′ overhang flap structure that is 1 nucleotide in length, wherein the 5′ overhang flap structure is cleavable with a 5′ flap endonuclease (e.g., FIG. 28A (left)). In some embodiments, the 5′ flap endonuclease can cleave the 5′ overhang flap and the 3′ overhang flap. In some embodiments, the 5′ flap endonuclease comprises FEN1.

In some embodiments of step (c), individual open circle library molecules comprise a 5′ overhang flap structure that is 2-10 nucleotides in length and lack a 3′ overhang flap structure, wherein the 5′ overhang flap structure is cleavable with a 5′ flap endonuclease (e.g., FIG. 28A (middle)). In some embodiments, the 5′ flap endonuclease comprises FEN1.

In some embodiments of step (c), individual open circle library molecules comprise a 5′ overhang flap structure that is 2-10 nucleotides in length and a 3′ overhang flap structure that is 2-10 nucleotides in length, wherein the 5′ overhang flap structure is not cleavable with a 5′ flap endonuclease (e.g., FIG. 28A (right)).

In some embodiments of step (c), the flap cleaving reagent comprises at least one 5′ flap endonuclease that originates from a thermophilic organism, a eukaryotic organism or an archaeal organism. In some embodiments, the 5′ flap endonuclease comprises a thermostable enzyme. In some embodiments, the 5′ flap endonuclease comprises FEN-1.

In some embodiments of step (c), the flap cleaving reagent comprises at least one 5′ flap endonuclease that originates from an Archaebacterial species including, without limitation, Archaeoglobus fulgidus (Afu FEN1; Chapados et al., 2004 Cell 116:39-50; Hosfield et al., 1998 J. Biol. Chem. 273:27154-27161; Hosfield 1998 Cell 95; 135-146; Allawi 2003 J. Mol. Biol. 328:537-554), Methanobacterium thermoautotrophicum (Mth FEN1), Pyrococcus furiosus (Pfu FEN1; Kaiser et al., 1999 J. Biol. Chem. 274:21387-21394), Methanococcus jannaschii (Mja FEN1; Hosfield et al., 1998 J. Biol. Chem. 273:27154-27161; Hwang 1998 Nature Struct. Biol. 5:707-713; Rao 1998 J. Bacteriol. 180:5406-5412; Bae 1999 Mol. Cells 9:45-48), Pyrococcus woesei (Pwo FEN1), Pyrococcus horikoshii (Pho FEN1; Matsui et al., 1999 J. Biol. Chem. 274:18297-18309; Matsui 2002 J. Biol. Chem. 277:37840-37847; Matsui 2014 Extremophiles 18:415-427), Archaeoglobus veneficus (Ave FEN1), Thermococcus kodakarensis (Tko FEN1; Burkhart 2017 J. Bacteriol. 199:e00141-17; Muzzamal 2020 Folia Microbiol (Praha) 62:407-415), Desulfurococcus amylolyticus (Dam FEN1; Mase 2011 Acta. Crystallogr. Sect. F. Struct. Biol. Cryst. Commun. 67:209-213), Aeropyrum pernix (Ape FEN1; Collins 2004 Acta. Crystallogr. D. Biol. Crystallogr. 60:1674-1678), Sulfolobus tokodaii (Sto FEN1; Horie 2007 Biosci. Biotechnol. Biochem. 71:855-865), or Sulfolobus solfataricus (Sso FEN1; Beattie and Bell 2012 EMBO. J. 31:1556-1567). The contents of these references are hereby expressly incorporated by reference in their entireties.

In some embodiments of step (c), the flap cleaving reagent comprises a 5′ flap endonuclease from Thermococcus sp. 9 degrees North (9° N FEN-1) (e.g., from New England Biolabs, catalog #M0645S).

In some embodiments of step (c), the flap cleaving reagent comprises at least one 5′ flap endonuclease that originates from a eukaryotic organism, including without limitation murine FEN-1 (Harrington and Lieber 1994 EMBO J. 13:1235-1246), yeast FEN1 (Harrington and Lieber 1994 Genes Dev. 8:1344-1355), and human FEN1 (Hiraoka et al., 1995 Genomics 25:220-225). The contents of these references are hereby expressly incorporated by reference in their entireties.

In some embodiments of step (c), the flap cleaving reagent comprises at least one Family A DNA polymerase from E. coli (DNA polymerase I), Taq DNA polymerase and/or Bst DNA polymerase, all of which exhibit 5′ flap endonuclease activity.

In some embodiments of step (c), the flap cleaving reagent comprises one type of 5′ flap endonuclease for example selected from any of the 5′ flap endonucleases described above.

In some embodiments of step (c), the flap cleaving reagent comprises a mixture of two or more different types of 5′ flap endonucleases for example, selected from any of the 5′ flap endonucleases described above. In some embodiments of step (c), the flap cleaving reagent comprises a mixture of a 5′ flap endonucleases for example, selected from any of the 5′ flap endonucleases described above, and a DNA polymerase which exhibits 5′ flap endonuclease activity.

In some embodiments of step (c), the flap cleaving reagent comprises at least one fusion enzyme comprising a portion of at least one 5′ flap endonuclease, for example selected from any of the 5′ flap endonucleases described above.

In some embodiments of step (c), the flap cleaving reagent comprises a structure specific 5′ flap endonuclease and a solvent. In some embodiments, the solvent comprises any one or any combination of two or more of the following: a pH buffering agent, a viscosity compound, ammonium ions, a salt, magnesium ions, detergent, a reducing compound and/or a nucleotide.

In some embodiments of step (c), the cleaving reagent further comprises a ligase enzyme. In some embodiments, the ligase enzyme comprises a bacteriophage DNA ligase, including a T3, T4 or T7 DNA ligase. In some embodiments, the ligase enzyme comprises a thermal stable DNA ligase including a Taq DNA ligase, a Tfu DNA ligase or a DNA ligase from Thermococcus nautili. In some embodiments, the ligase enzyme comprises a recombinant thermal tolerant T4 DNA ligase (e.g., Hi-T4 DNA ligase from New England Biolabs, catalog #M2622S).

In some embodiments of step (c), the flap cleaving reaction can be conducted at a temperature of about 45-50° C., or about 50-55° C., or about 55-60° C., or about 60-65° C., or about 65-70° C.

In some embodiments of step (c), the flap cleaving reaction can be conducted at a pH of about 6.5-7, or a pH of about 7-7.5, or a pH of about 7.5-8, or a pH of about 8-8.5, or a pH of about 8.5-9.

In some embodiments, the flap cleaving reagent comprises a solvent comprising water or an aqueous buffer.

In some embodiments, the flap cleaving reagent comprises at least one pH buffering agent comprising Tris (e.g., Tris(hydroxymethyl)-aminomethane), Tris-HCL (e.g., Tris(hydroxymethyl)-aminomethane hydrochloride), HEPES (e.g., 4-(2-hydroxyethyl)-1-piperazineethanesulfonic acid) or MOPS (e.g., 3-(N-morpholino)propanesulfonic acid).

In some embodiments, the flap cleaving reagent comprises at least one viscosity compound comprising trehalose, sucrose, cellulose, xylitol, mannitol, sorbitol, D-maltose or inositol. In some embodiments, the viscosity agent comprises glycerol or a glycol compound such as ethylene glycol or propylene glycol (e.g., propanediol).

In some embodiments, the flap cleaving reagent comprises at least one source of ammonium ions comprising ammonium sulfate (e.g., NH₄)₂SO₄) and/or ammonium acetate.

In some embodiments, the flap cleaving reagent comprises at least one salt comprising NaCl, KCl or potassium glutamate.

In some embodiments, the flap cleaving reagent comprises at least one source of magnesium ions comprising MgCl₂ and/or MgSO₄.

In some embodiments, the flap cleaving reagent comprises at least one detergent comprising Tween-20, Tween-80, Triton X-100, Nonidet P-40, CHAPS (e.g., 3-[(3-cholamidopropyl) dimethylammonio]-1-propanesulfonate) and/or DetX (e.g., N-Dodecyl-N,N-dimethyl-3-amonio-1-propanesulfate).

In some embodiments, the flap cleaving reagent comprises at least one reducing compound comprising DTT (dithiothreitol), 2-beta mercaptoethanol, TCEP, (tris(2-carboxyethyl)phosphine), formamide, DMSO (dimethylsulfoxide), sodium dithionite (Na₂S₂O₄), glutathione, methionine, betaine, Tris(3-hydroxypropyl)phosphine (THPP) and/or N-acetyl cysteine.

In some embodiments, the flap cleaving reagent comprises at least one nucleotide. In some embodiments, the at least one nucleotide comprises ATP.

In some embodiments, the flap cleaving reagent comprises at least one ligase enzyme. In some embodiments, the ligase enzyme comprises a bacteriophage DNA ligase, including a T3 DNA ligase (e.g., SEQ ID NO:147, FIG. 60), T4 DNA ligase (e.g., SEQ ID NO:148, FIG. 61) or T7 DNA ligase (e.g., SEQ ID NO:149, FIG. 62). In some embodiments, the ligase enzyme comprises a thermal stable DNA ligase including a Taq DNA ligase, a Tfu DNA ligase (e.g., SEQ ID NO:150, FIG. 63) or a DNA ligase from Thermococcus nautili (e.g., SEQ ID NO:151, FIG. 64). In some embodiments, the ligase enzyme comprises a recombinant thermal tolerant T4 DNA ligase (e.g., Hi-T4 DNA ligase from New England Biolabs, catalog #M2622S).

Seeding the Immobilized Splint Capture Primers with Fresh Linear Library Molecules

In some embodiments, in the method for generating a plurality of nucleic acid concatemers immobilized to a support at step (c) as described above, the support can be seeded at least once. In some embodiments, the support can be seeded multiple times with a plurality of fresh linear library molecules to generate a support having a plurality of immobilized concatemer template molecules. In some embodiments, seeding the support multiple times can generate a plurality of immobilized open circle library molecules at a density of about 10²-10¹⁵ per mm².

In some embodiments, in the method for generating a plurality of nucleic acid concatemers immobilized to a support at step (c) as described above, the method comprises contacting the plurality of immobilized splint capture primers (200) with a first flow of reagents comprising a first plurality of linear library molecules, wherein the contacting the first flow of reagents is conducted under a condition suitable for hybridizing individual linear library molecules in the first plurality to individual immobilized splint capture primers to form a first plurality of open circle library molecules, where individual open circle library molecules have the first terminal region of a linear library molecule hybridized to a first portion (210) of a splint capture primer and a second terminal region of the same linear library molecule hybridized to a second portion (220) of the same splint capture primer, and wherein individual open circle library molecules have a gap or nick between the 5′ and 3′ ends of the open circle library molecule. In some embodiments, after the first flow contacting some of the immobilized splint capture primers are seeded since they are hybridized to an open circle library molecule. In some embodiments, some of the immobilized splint capture primers are un-seeded since they are not hybridized to an open circle library molecule. In some embodiments, the linear library molecules are single stranded. In some embodiments, it is desirable to increase the percent of immobilized splint capture primers that are seeded and hybridized to an open circle library molecule by conducting another flow.

In some embodiments, the method further comprises step (c1): contacting the plurality of immobilized splint capture primers (200) with a second flow of reagents comprising a second plurality of linear library molecules, wherein contacting the second flow is conducted under a condition suitable for hybridizing individual linear library molecules in the second plurality to individual free immobilized splint capture primers to form a second plurality of open circle library molecules. In some embodiments, after the second flow of reagents is contacted with the splint capture primers, some of the immobilized splint capture primers are seeded since they are hybridized to an open circle library molecule. In some embodiments, some of the immobilized splint capture primers are un-seeded since they are not hybridized to an open circle library molecule. In some embodiments, it is desirable to increase the percent of immobilized splint capture primers that are seeded and hybridized to an open circle library molecule by conducting yet another flow of reagents. In some embodiments, at least a third flow of reagents is conducted, at least a fourth flow of reagents is conducted, or at least a fifth flow or reagents is conducted. In some embodiments, the flows of reagents comprise pluralities of linear library molecules. In some embodiments, up to ten flows of reagents can be conducted to increase the percent of immobilized splint capture primers that are seeded and hybridized to an open circle library molecule. In some embodiments, the linear library molecules are single stranded. In some embodiments, the linear library molecules are re-cycled, i.e. had been contacted with the capture primers in a previous flow of reagents. In some embodiments, two or more seeding flows can be conducted to generate a plurality of open circle library molecules (e.g., hybridized to immobilized splint capture primers) at a density of about 10²-10¹⁵ per mm².

In some embodiments, the method further comprises conducting step (d) which comprises enzymatically closing the nick and/or gap formed by the immobilized open circle library molecules using a ligation reaction mixture. Embodiments of step (d) are described above.

In some embodiments, the method further comprises conducting step (e) which comprises conducting a rolling circle reaction using a rolling circle reaction mixture to generate a plurality of immobilized concatemers. Embodiments of step (e) are described above.

In some embodiments, the method further comprises conducting step (f) which comprises sequencing the immobilized concatemer template molecules. Embodiments of step (f) are described above.

In some embodiments, the method further comprises conducting step (g) which comprises replacing the extended forward sequencing primers strands with forward extension strands. Embodiments of step (g) are described above.

In some embodiments, the method further comprises conducting step (h) which comprises removing the retained immobilized concatemer template molecules by generating abasic sites in the immobilized single stranded concatemer template molecules at the nucleotide(s) having the scissile moiety and generating gaps at the abasic sites to generate a plurality of gap-containing single stranded nucleic acid concatemer template molecules while retaining the plurality of immobilized forward extension strands. Embodiments of step (h) are described above.

In some embodiments, the method further comprises conducting step (i) which comprises sequencing the forward extension strands. Embodiments of step (i) are described above.

Seeding the Immobilized Splint Capture Primers with Recycled Linear Library Molecules

In some embodiments, in the method for generating a plurality of nucleic acid concatemer template molecules immobilized to a support at step (c) as described above, the support can be seeded at least once with a plurality of fresh linear library molecules and then seeded again with a plurality of recycled linear library molecules to generate a support having a plurality of immobilized concatemer template molecules. In some embodiments, seeding the support multiple times can generate a plurality of immobilized open circle library molecules at a density of about 10²-10¹⁵ per mm².

In some embodiments, in the method for generating a plurality of nucleic acid concatemers immobilized to a support at step (c1), the method comprises contacting the plurality of immobilized splint capture primers (200) with a first flow of reagents comprising a first plurality of nucleic acid linear library molecules, wherein the first flow contacting is conducted under a condition suitable for hybridizing individual linear library molecules in the first plurality to individual immobilized splint capture primers to form a first plurality of open circle library molecules, where individual open circle library molecules have the first terminal region of an individual linear library molecule hybridized to a first portion (210) of a splint capture primer and have the second terminal region of the same individual linear library molecule hybridized to a second portion (220) of the same splint capture primer, wherein individual open circle library molecules have a gap or nick between the 5′ and 3′ ends of the open circle library molecule. In some embodiments, the linear library molecules are single stranded.

In some embodiments, a first sub-population of the first plurality of linear library molecules hybridizes to the immobilized splint capture primers, and a second sub-population of the first plurality of single stranded nucleic acid linear library molecules is not hybridized to the immobilized splint capture primers. In some embodiments, the second sub-population of the first plurality of single stranded nucleic acid linear library molecules (e.g., the un-hybridized linear library molecules) can be collected and can be re-flowed onto the immobilized splint capture primers (recycled linear library molecules). In some embodiments, after the first flow contacting some of the immobilized splint capture primers are seeded since they are hybridized to an open circle library molecule. In some embodiments, some of the immobilized splint capture primers are un-seeded since they are not hybridized to an open circle library molecule. In some embodiments, it is desirable to increase the percent of immobilized splint capture primers that are seeded and hybridized to an open circle library molecule by conducting another flow. In some embodiments, the linear library molecules are single stranded.

In some embodiments, the method further comprises step (c2): conducting a recycling flow by contacting the plurality of immobilized splint capture primers (200) with a second flow comprising the un-hybridized linear library molecules from the first plurality of linear library molecules, i.e. recycled linear library molecules, wherein the second flow contacting is conducted under a condition suitable for hybridizing individual linear library molecules in the second flow to individual free immobilized splint capture primers, i.e. splint capture primers not already hybridized to a linear library molecule, to form a second plurality of open circle library molecules. In some embodiments, after the second flow contacting, some of the immobilized splint capture primers are seeded since they are hybridized to an open circle library molecule. In some embodiments, some of the immobilized splint capture primers are un-seeded since they are not hybridized to an open circle library molecule. It can be desirable to increase the percent of immobilized splint capture primers that are seeded and hybridized to an open circle library molecule by conducting yet another recycling flow of reagents. In some embodiments, at least a third recycling flow is conducted, at least a fourth recycling flow is conducted, or at least a fifth recycling flow is conducted. In some embodiments, up to ten recycling flows can be conducted to increase the percent of immobilized splint capture primers that are seeded and hybridized to an open circle library molecule.

In some embodiments, the percent of immobilized splint capture primers (200) that are hybridized to linear library molecules (e.g., seeded splint capture primers) can be increased by conducting any number of flows of reagents with fresh linear library molecules and/or any number of recycling flows of reagents with recycled linear library molecules. In some embodiments, the flows with fresh linear library molecules and/or the recycling flows with recycled linear library molecules can be conducted in any order and in any combination.

In some embodiments, the method further comprises conducting step (d) which comprises enzymatically closing the nick and/or gap formed by the immobilized open circle library molecules using a ligation reaction mixture. Embodiments of step (d) are described above.

In some embodiments, the method further comprises conducting step (e) which comprises conducting a rolling circle reaction using a rolling circle reaction mixture to generate a plurality of immobilized concatemers. Embodiments of step (e) are described above.

In some embodiments, the method further comprises conducting step (f) which comprises sequencing the immobilized concatemers. Embodiments of step (f) are described above.

In some embodiments, the method further comprises conducting step (g) which comprises replacing the extended forward sequencing primers strands with forward extension strands. Embodiments of step (g) are described above.

In some embodiments, the method further comprises conducting step (h) which comprises removing the retained immobilized concatemer template molecules by generating abasic sites in the immobilized single stranded concatemer template molecules at the nucleotide(s) having the scissile moiety and generating gaps at the abasic sites to generate a plurality of gap-containing single stranded nucleic acid concatemer template molecules while retaining the plurality of immobilized forward extension strands. Embodiments of step (h) are described above.

In some embodiments, the method further comprises conducting step (i) which comprises sequencing the forward extension strands. Embodiments of step (i) are described above.

Batch Sequencing and Re-Seeding with Interrupted Sequencing

For massively parallel nucleic acid sequencing, the limit of optical resolution impedes the ability to perform highly multiplex sequencing. Batch-specific sequencing enables sequencing a desired subset (e.g., a batch) of the template molecules immobilized to the same flow cell using selected batch-specific sequencing primers to reduce over-crowding signals and images. The use of batch-specific sequencing primers produces optical images that are intense and resolvable. The batch-specific sequencing methods described herein have many uses. For example, the number of spots that are imaged and associated with sequencing can be counted. The counted spots can be used as a measure for target nucleic acid levels in a sample.

The term “batch-specific sequencing primer binding site” and other related terms refers to a pre-determined sequencing primer binding site that is linked to an insert region (e.g., sequence of interest) in a library molecule. Alternatively, a batch-specific sequencing primer binding site can be linked to a batch-specific barcode sequence which is proximal to an insert region. The library molecule can undergo rolling circle amplification to generate a concatemer template molecule carrying complementary sequences of the library molecule. The concatemer template molecule can serve as nucleic acid template molecule to be sequenced. In a mixture of different concatemer template molecules, the batch-specific sequencing primer binding sites facilitate sequencing a sub-population of concatemer template molecules. For example, a mixture of different sub-populations of concatemer template molecules that are immobilized to the same support can be sequenced separately at different times using different batch-specific sequencing primers that hybridize to their cognate batch-specific sequencing primer binding sites. In some embodiments, the mixture of concatemer template molecules comprises at least a first and second sub-population of concatemer template molecules.

The concatemer template molecules of the first sub-population can share the same first batch-specific sequencing primer binding sequence. The first batch-specific sequencing primer binding site can selectively hybridize to its cognate first batch sequencing primer for sequencing the first sub-population of concatemer template molecules that carry the first batch-specific sequencing primer binding site. In some embodiments, the first batch sequencing primer can be used to sequence the insert region only. In some embodiments, the first batch sequencing primer can be used to sequence the batch-specific barcode sequence only. In some embodiments, the first batch sequencing primer can be used to sequence the batch-specific barcode sequence and the insert region.

The concatemer template molecules of the second sub-population can share the same second batch-specific sequencing primer binding sequence. The second batch-specific sequencing primer binding site can selectively hybridize to its cognate second batch sequencing primer for sequencing the second sub-population of concatemer template molecules that carry the second batch-specific sequencing primer binding site. In some embodiments, the second batch sequencing primer can be used to sequence the insert region only. In some embodiments, the second batch sequencing primer can be used to sequence the batch-specific barcode sequence only. In some embodiments, the second batch sequencing primer can be used to sequence the batch-specific barcode sequence and the insert region. The present disclosure provides methods for generating a plurality of nucleic acid concatemers immobilized to a support and conducting separate batches of sequencing on the support. In some embodiments, the separate sequencing batches can be conducted using any massively parallel sequencing technology. In some embodiments, a plurality of sub-populations of concatemer template molecules are immobilized to the support including at least a first and second sub-population. In some embodiments, the first sub-population of concatemer template molecules undergo first sequencing reactions (e.g., first batch sequencing) and a region of the support is imaged to detect the first sequencing reactions, wherein the second sub-population of template molecules do not undergo sequencing reactions. In some embodiments, the second sub-population of concatemer template molecules undergo second sequencing reactions (e.g., second batch sequencing) and the same region of the support is imaged to detect the second sequencing reactions, wherein the first sub-population of template molecules do not undergo sequencing reactions. Thus, the first and second sub-populations of concatemer template molecules undergo batch sequencing. In some embodiments, the first and second sub-populations of concatemer template molecules are distributed over the same area of the support, and this area is imaged in both the first batch and second batch sequencing.

In some embodiments, the plurality of sub-populations of concatemer template molecules are immobilized to the support at a high density where at least some of the immobilized concatemer template molecules in the first and second sub-populations comprise nearest neighbor concatemer template molecules that touch each other and/or overlap each other when viewed from any angle of the support including above, below or side views of the support.

In some embodiments, the support comprises a plurality of concatemer template molecules immobilized at pre-determined positions on the support (e.g., a patterned support). In some embodiments, the support comprises a plurality of concatemer template molecules immobilized at random and non-pre-determined positions on the support. In some embodiments, the support comprises a mixture of at least two sub-populations of concatemer template molecules immobilized at random and non-pre-determined positions on the support. In some embodiments, the support lacks any contours (e.g., wells, protrusions, and the like) arranged in a pre-determined pattern. In some embodiments, the support lacks contours which include features as sites for attachment of the nucleic acid concatemer template molecules. In some embodiments, the support lacks interstitial regions arranged in a pre-determined pattern where the interstitial regions are sites designed to have no attached concatemer template molecules. In some embodiments, the support lacks features that can be prepared using photo-chemical, photo-lithography, or micron-scale or nano-scale printing.

In some embodiments, individual concatemer template molecules in a given sub-population of concatemer template molecules comprise a sequence of interest and a batch-specific sequencing primer binding site sequence that corresponds to the sequence of interest or corresponds to the concatemer template molecules in the given sub-population. In some embodiments, individual concatemer template molecules in a given sub-population of concatemer template molecules further comprise a batch barcode sequence that corresponds to the sequence of interest or corresponds to the concatemer template molecules in the given sub-population. In some embodiments, a pre-determined batch sequencing primer binding site sequence can be linked to a given sequence of interest, thus the pre-determined batch sequencing primer binding site sequence corresponds to a given sequence of interest. In some embodiments, a pre-determined batch barcode sequence can be linked to a given sequence of interest, thus the pre-determined batch barcode sequence corresponds to a given sequence of interest. In some embodiments, concatemer template molecules within a given sub-population have the same sequence of interest. In some embodiments, concatemer template molecules within a given sub-population have different sequences of interest. In some embodiments, concatemer template molecules within a given sub-population have the same batch barcode sequence. In some embodiments, concatemer template molecules within a given sub-population have the same sequencing primer binding site sequence. Thus, the different sub-populations of concatemer template molecules can undergo batch sequencing using a batch-specific sequencing primer.

In some embodiments, the sequence of interest is sequenced. In some embodiments, the sequence of interest need not undergo sequencing. Instead, the target barcode can be sequenced by conducting a small number of sequencing cycles to reveal the target barcode which corresponds to its sequence of interest.

In some embodiments, individual concatemer template molecules in a given sub-population of concatemer template molecules further comprise a sample index sequence that can be used to distinguish sequences of interest obtained from different sample sources in a multiplex assay. In some embodiments, concatemer template molecules within a given sub-population have the same or different sample index sequences.

In some embodiments, the sequence of interest need not undergo sequencing. Instead, the target barcode and/or the sample index can be sequenced by conducting a small number of sequencing cycles to reveal the target barcode which corresponds to its sequence of interest and to reveal the sample index which corresponds to the sample source of the sequence of interest. In some embodiments, the concatemer template molecules lack a sample index and the target barcode can serve as a sample index. In some embodiments, batch-specific sequencing comprises conducting no more than 200 sequencing cycles, conducting no more than 150 sequencing cycles, conducting no more than 100 sequencing cycles, conducting no more than 50 sequencing cycles, conducting no more than 25 sequencing cycles, or conducting no more than 10 sequencing cycles.

In some embodiments, the same portion of individual concatemer template molecules can be re-sequenced (e.g., reiterative sequencing) from the same start position to generate overlapping sequencing reads that can be aligned to a reference sequence. For example, the same portion of individual concatemer template molecules can be sequenced at least two, three, four, five, or up to 50 times. The start sequencing site can be any location of the concatemer template molecule and is dictated by the sequencing primers which are designed to anneal to a selected position within the concatemer template molecule. In some embodiments, the target barcodes (or the target barcodes and sample indexes) can be reiteratively sequenced by repeatedly conducting a short number of sequencing cycles of the target barcode region (or the target barcode and sample index regions) of a given concatemer template molecule. The reiterative sequencing reads increase the redundancy of sequencing information for individual bases in the concatemer template molecule. Reiteratively sequencing one strand of the concatemer template molecule provides enough base coverage so that sequencing the complementary strand is not necessary.

In some embodiments, after sequencing the first and/or second sub-populations of concatemer template molecules, the support can be re-seeded at least once with additional sub-population of linear library molecules (e.g., a third sub-population) which can be used to produce a third batch of concatemer template molecules which undergo additional batch sequencing. In some embodiments, an ongoing batch sequencing run can be stopped prior to completion (e.g., interrupted) to permit re-seeding the support with an additional sub-population of linear library molecules or concatemer template molecules (e.g., third sub-population) and then the interrupted batch sequencing can be resumed. Thus, the support can be re-seeded any time and/or before a previous sequencing batch is completed.

In some embodiments, the support comprises a plurality of concatemer template molecules immobilized at an initial low density where most of the nearest neighbor concatemer template molecules do not touch each other and/or do not overlap each other. In some embodiments, the initial low density support comprises a plurality of immobilized concatemer template molecules having interstitial space between the immobilized template molecules.

In some embodiments, the same support can undergo a first re-seeding with additional linear library molecules where the re-seeded linear library molecules undergo amplification to generate additional concatemer template molecules so that the first re-seeded density has some nearest neighbor concatemer template molecules (e.g., 10-30% of the first immobilized re-seeded template molecules) that touch each other and/or overlap each other. In some embodiments, the resulting first re-seeded support comprises a plurality of immobilized concatemer template molecules having a reduced number of interstitial space (and/or having a reduced size of interstitial space) between the immobilized concatemer template molecules compared to the initial low density support.

In some embodiments, the same support can undergo a second re-seeding with additional linear library molecules which undergo amplification the generate yet more concatemer template molecules so that the second re-seeded density has an increase in nearest neighbor concatemer template molecules (e.g., 25-50% or more of the first immobilized re-seeded template molecules) that touch each other and/or overlap each other. In some embodiments, the resulting second re-seeded support comprises a plurality of immobilized concatemer template molecules having a further reduced number of interstitial space (and/or having a further reduced size of interstitial space) between the immobilized concatemer template molecules compared to the first re-seeded density support. In some embodiments, the support can undergo multiple re-seeding workflows to generate increasing nearest neighbor concatemer template molecules that touch each other and/or overlap each other.

The methods described herein employ batch sequencing on high density immobilized concatemer template molecules which offers the advantage of maximizing space on a support (e.g., flow cell). Furthermore, the same seeded support can be re-used by re-seeding the support to produce additional immobilized concatemer template molecules and conducting additional sequencing reactions on the re-seeded concatemer template molecules.

Batch sequencing can be conducted using concatemer template molecules arranged in a pre-determined manner on the support (e.g., a patterned support). Alternatively, batch sequencing can be conducted using concatemer template molecules arranged in a random manner on the support which obviates the need to fabricate a support having organized and pre-determined features for attaching concatemer template molecules (e.g., fabrication via lithography is not needed).

By conducting short sequencing reads of the target barcode regions of the concatemer template molecules, batch sequencing also significantly reduces sequencing run times, reagent use, and reagent costs.

When short sequencing reads of the target barcode regions are conducted in a reiterative manner, it is not necessary to assemble the sequencing reads or to obtain a full length sequence of the sequence of interest which reduces the need for long assembly computations. Also, the redundant sequencing information obtained from the short sequencing reads obviates the need to sequence the complementary strand of the concatemer template molecules, thus pairwise sequencing is not necessary.

Batch sequencing also offers the flexibility of re-seeding the support any time between sequencing different batches, or an ongoing sequencing batch can be interrupted to permit re-seeding then the ongoing batch sequencing can be resumed. The ability to re-seed the support any time increases throughput and efficiency.

Conducting batch sequencing with immobilized concatemer template molecule offers advantages over one-copy template molecules (e.g., one-copy template molecule generated via bridge amplification). For example concatemer template molecules carry multiple sequencing primer binding sites along the same concatemer template molecule. The multiple sequencing primer binding sites can be used to generate multiple sequencing reads for increased sequencing depth. Together, reiteratively sequencing one strand of the concatemer templates increases sequencing base coverage and sequencing depth compared to sequencing a one-copy template molecule.

Batch sequencing has many uses including but not limited to detecting specific nucleic acids of interest, mutant nucleic acid sequences, splice variants, and their abundance levels thereof.

### Batch Sequencing

In some embodiments, in the method for generating a plurality of nucleic acid concatemers immobilized to a support as described above, step (a) comprises: providing a support having a plurality of splint capture primers (200) immobilized thereon and a plurality of pinning primers (500) immobilized thereon. In some embodiments, the plurality of immobilized splint capture primers and pinning primers can be used to conduct batch sequencing as described below.

In some embodiments, in step (a), the plurality of splint capture primers (200) comprise the same sequence. In some embodiments, the plurality of splint capture primers having the same sequence can hybridize/capture different linear library molecules carrying the same splint capture primer binding site sequences.

In some embodiments, in step (a), the plurality of splint capture primers (200) comprise a plurality of sub-populations of splint capture primers including at least a first and second sub-population of splint capture primers. In some embodiments, the splint capture primers in the at least first and second sub-population have different sequences. In some embodiments, the splint capture primers in the at least first and second sub-population can hybridize/capture different linear library molecules carrying different splint capture primer binding site sequences.

In some embodiments, in step (a), the plurality of splint capture primers (200) that are immobilized to the support comprise different types of splint capture primers including at least a first and second sub-population of splint capture primers having different sequences, wherein the different types of splint capture primers bind to different types of linear library molecules.

In some embodiments, individual immobilized splint capture primers in the first sub-population (200-A) comprise a first portion (210-A) and a second portion (220-A), wherein the first portion of the splint capture primer (210-A) binds a first universal binding site (120-A) in a first linear library molecule and the second portion of the splint capture primer (220-A) binds a second universal binding site (130-A) in the same linear library molecule.

In some embodiments, individual immobilized splint capture primers in the second sub-population (200-B) comprise a first portion (210-B) and a second portion (220-B), wherein the first portion of the splint capture primer (210-B) binds a first universal binding site (120-B) in a second linear library molecule and the second portion of the splint capture primer (220-B) binds a second universal binding site (130-B) in the same second linear library molecule.

In some embodiments, in step (a), the support further comprises a plurality of features on the support that are located in a random and non-pre-determined manner (e.g., FIG. 15A), where the features are sites for attachment of the plurality of splint capture primers (200) and the plurality of pinning primers (500).

In some embodiments, in step (a), the support further comprises features on the support that are located at pre-determined positions on the support (e.g., FIGS. 16A and 16B, where the features are sites for attachment of the plurality of splint capture primers and the plurality of pinning primers.

In some embodiments, in step (a), the support is passivated with at least one polymer coating layer comprising a plurality of splint capture primers (200) and pinning primers (500) that are covalently tethered to the at least one polymer layer.

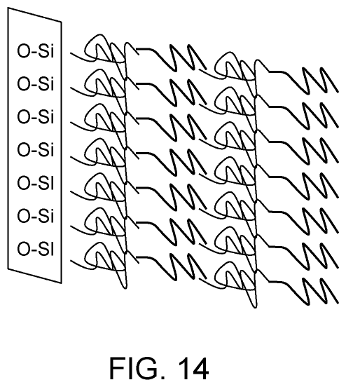

In some embodiments, in step (a), the plurality of plurality of splint capture primers (200) and the plurality of pinning primers (500) are randomly distributed throughout and embedded within the at least one polymer layer (e.g., FIGS. 14 and 15A).

In some embodiments, in step (a), the support lacks any contours (e.g., wells, protrusions, and the like) arranged in a pre-determined pattern where the contours have features that are sites for attachment of the plurality of splint capture primers (200) and the plurality of pinning primers (500). In some embodiments, the support lacks interstitial regions arranged in a pre-determined pattern where the interstitial regions are sites designed to have no attached splint capture primers or pinning primers.

In some embodiments, in step (a), the plurality of splint capture primers (200) and the plurality of pinning primers (500) are located at pre-determined positions within the at least one polymer layer (e.g., FIGS. 14, 16A and 16B).

In some embodiments, in step (a), the support further comprises contours (e.g., wells, protrusions, and the like) arranged in a pre-determined pattern where the contours have features that are sites for attachment of the plurality of splint capture primers (200) and the plurality of pinning primers (500). In some embodiments, the support further comprises interstitial regions arranged in a pre-determined pattern where the interstitial regions are sites designed to have no attached splint capture primers or pinning primers.

In some embodiments, in step (a), the support lacks partitions/barriers that would create separate regions of the support. Thus, the immobilized splint capture primers (200) and pinning primers (500) are in fluid communication with each other in a massively parallel manner with no barriers to physically separate different batches of concatemer template molecules.

In some embodiments, in step (a), the support includes partitions/barriers that would create separate regions of the support. Thus, the immobilized splint capture primers (200) and pinning primers (500) have barriers to physically separate different batches of concatemer template molecules.

In some embodiments, in the method for generating a plurality of nucleic acid concatemers immobilized to a support comprises step (b): providing a plurality of linear library molecules including at least a first sub-population of linear library molecules and a second sub-population of linear library molecules, wherein individual linear library molecules in the plurality comprise a 5′ and 3′ end, and wherein individual linear library molecules in the plurality comprise a sequence of interest and any one or any combination of two or more adaptor sequences in any order, wherein the adaptor sequences comprise: (i) a first universal binding site (120) for a first portion of a splint capture primer (or a complementary sequence thereof); (ii) a universal binding site for a first non-splint capture primer (123) (or a complementary sequence thereof); (iii) at least one sample index sequence (e.g., (160) and/or (170) which can be used to distinguish sequences of interest obtained from different sample sources in a multiplex assay; (iv) at least one universal binding site for a batch-specific forward sequencing primer (140) (or a complementary sequence thereof); (v) at least one universal binding site for a batch-specific reverse sequencing primer (150) (or a complementary sequence thereof); (vi) at least one universal binding site for a compaction oligonucleotide (or a complementary sequence thereof); (vii) at least one unique molecular index sequence (UMI) (e.g., (180) and/or (190)) which can be used to uniquely identify a nucleic acid molecule (e.g., having a sequence of interest) to which the unique molecular index sequence is appended; (viii) at least one universal binding site for a pinning primer (or a complementary sequence thereof); (ix) at least one batch-specific barcode sequence (e.g., a first batch-specific barcode sequence (142) and/or a second batch-specific barcode sequence (152); (x) a universal binding site for a second non-splint capture primer (133) (or a complementary sequence thereof); (xi) at least one short random sequence (e.g., NNNN) (132) which provides nucleotide sequence diversity and is about 3-20 nucleotides in length; and/or (xii) a second universal binding site (130) for a second portion of the immobilized splint capture (or a complementary sequence thereof). In some embodiments, the linear library molecules are single stranded.

In some embodiments, in step (b), the at least one sample index sequence (e.g., (160) and/or (170) comprises a sample index sequence joined to a short random sequence (e.g., NNN), where the short random sequence provides nucleotide sequence diversity and is about 3-20 nucleotides in length.

In some embodiments, in step (b), individual linear library molecules in the first sub-population comprise the same sequence of interest. In some embodiments, individual linear library molecules in the first sub-population comprise a mixture of different sequences of interest. In some embodiments, individual linear library molecules in the second sub-population comprise the same sequence of interest. In some embodiments, individual linear library molecules in the second sub-population comprise a mixture of different sequences of interest.

In some embodiments, in step (b), individual linear library molecules in the first and second sub-population comprise the same first universal binding site (120) for a first portion of a splint capture primer (or a complementary sequence thereof). In some embodiments, individual linear library molecules in the first and second sub-population comprise the same second universal binding site (130) for a second portion of the immobilized splint capture (or a complementary sequence thereof).

In some embodiments, in step (b), individual linear library molecules in the first and second sub-population comprise different types of universal binding sites for binding a first portion of a splint capture primer (or complementary sequence thereof), and different types of universal binding sites for binding a second portion of a splint capture primer (or complementary sequence thereof). In some embodiments, individual linear library molecules in the first sub-population of linear library molecules comprise a universal binding site (120-A) for binding a first portion of a splint capture primer (or a complementary sequence thereof) (210-A) and a universal binding site (130-A) for binding a second portion of the immobilized splint capture (or a complementary sequence thereof) (220-A). In some embodiments, individual linear library molecules in the second sub-population of linear library molecules comprise a universal binding site (120-B) for binding a first portion of a splint capture primer (or a complementary sequence thereof) (210-B) and a universal binding site (130-B) for binding a second portion of the immobilized splint capture (or a complementary sequence thereof) (220-B). In some embodiments, the universal binding sites (120-A) and (120-B) have different sequences. In some embodiments, the universal binding sites (130-A) and (130-B) have different sequences.

Exemplary linear library molecules are shown in FIGS. 20-24. The skilled artisan will recognize that linear library molecules having adaptor sequences constructed with other arrangements are possible.

In some embodiments, in step (b), the amount of the plurality of linear library molecules (100) that are contacted with the plurality of immobilized splint capture primers (200) can adjusted to achieve a density of immobilized concatemer template molecules of about 10²-10¹⁵ per mm² where the immobilized concatemer template molecules will be generated in step (e) by conducting a rolling circle amplification reaction (e.g., described below). In some embodiments, the amount of the plurality of linear library molecules (100) that are contacted with the plurality of immobilized splint capture primers (200) can be about 0.1-1 pM, or about 1-5 pM, or about 5-10 pM, or about 10-20 pM, or about 20-30 pM, or about 30-40 pM, or about 40-50 pM.

In some embodiments, in step (b), individual linear library molecules in the first sub-population comprise a first batch sequencing primer binding site and a sequence of interest. For example, the first batch sequencing primer binding site comprises a first batch forward sequencing primer binding site. In some embodiments, linear library molecules within the first sub-population have the same first batch sequencing primer binding site and have the same first sequence of interest. In some embodiments, the sequence of the first batch sequencing primer binding site corresponds to the first sequence of interest, which are the same sequence in the first sub-population. In some embodiments, a pre-determined first batch sequencing primer binding site sequence can be linked to a first sequence of interest in the first sub-population, thus the pre-determined first batch sequencing primer binding site sequence corresponds to the first sequence of interest in the first sub-population.

In some embodiments, in step (b), linear library molecules within the first sub-population have the same first batch sequencing primer binding site and have different sequences of interest. For example, the first batch sequencing primer binding site comprises a first batch forward sequencing primer binding site. In some embodiments, the sequence of the first batch sequencing primer binding site sequence corresponds to the different sequences of interest in the first sub-population, for example, and without limitation, sequences of interest from a single sample. In some embodiments, a pre-determined first batch sequencing primer binding site sequence can be linked to different sequences of interest in a first sub-population, thus the pre-determined first batch sequencing primer binding site sequence corresponds to a plurality of sequences of interest in the first sub-population.

In some embodiments, in step (b), the sequences of interest in the first sub-population are about 50-250 bases in length, or about 250-500 bases in length, or about 500-800 bases in length, or about 800-1200 bases in length, or up to 2000 bases in length.

In some embodiments, in step (b), individual linear library molecules in the second sub-population comprise a second batch sequencing primer binding site and a sequence of interest. For example, the second batch sequencing primer binding site comprises a second batch forward sequencing primer binding site. In some embodiments, linear library molecules within the second sub-population have the same second batch sequencing primer binding site and have the same second sequence of interest. In some embodiments, the sequence of the second batch sequencing primer binding site corresponds to the second sequence of interest which are the same sequence in the second sub-population. In some embodiments, a pre-determined second batch sequencing primer binding site sequence can be linked to a second sequence of interest in the second sub-population, thus the pre-determined second batch sequencing primer binding site sequence corresponds to the second sequence of interest in the second sub-population.

In some embodiments, in step (b), linear library molecules within the second sub-population have the same second batch sequencing primer binding site and have different sequences of interest. For example, the second batch sequencing primer binding site comprises a second batch forward sequencing primer binding site. In some embodiments, the sequence of the second batch sequencing primer binding site sequence corresponds to the different sequences of interest in the second sub-population, for example, and without limitation, sequences of interest from a single sample. In some embodiments, a pre-determined second batch sequencing primer binding site sequence can be linked to different sequences of interest in a second sub-population, thus the pre-determined second batch sequencing primer binding site sequence corresponds to a plurality sequences of interest in the second sub-population.

In some embodiments, in step (b), the sequences of interest in the second sub-population are about 50-250 bases in length, or about 250-500 bases in length, or about 500-800 bases in length, or about 800-1200 bases in length, or up to 2000 bases in length.

In some embodiments, in step (b), individual linear library molecules in the first and second sub-populations can bind the same splint capture primers (200) since they carry that same sequences for a first universal binding site (120) for a first portion of a splint capture primer (or a complementary sequence thereof) and a second universal binding site (130) for a second portion of a splint capture primer (or a complementary sequence thereof).

In some embodiments, in step (b), individual linear library molecules in the first and second sub-populations can bind different splint capture primers (200) since they carry different universal binding sites for binding either a first or second splint capture primer. In some embodiments, in step (b), individual linear library molecules in the first sub-population can bind a first splint capture primer and individual linear library molecules in the second sub-population can bind a second splint capture primer, and the first and second splint capture primers have different sequences. In some embodiments, individual linear library molecules in the first sub-population carry a first universal binding site (120-1) for a first portion of a first splint capture primer (or a complementary sequence thereof) and a first universal binding site (130-1) for a second portion of a first splint capture primer (or a complementary sequence thereof). In some embodiments, individual linear library molecules in the second sub-population carry a second universal binding site (120-2) for a first portion of a second splint capture primer (or a complementary sequence thereof), and a second universal binding site (130-2) for a second portion of a second splint capture primer (or a complementary sequence thereof).

In some embodiments, in step (b), individual linear library molecules in the first sub-population comprise a first batch barcode sequence which corresponds to the first sequence of interest, or the first batch barcode sequence corresponds to one of the first sequences of interest in the first sub-population. In some embodiments, a pre-determined first batch barcode sequence can be linked to a given sequence of interest in the first sub-population (or can be linked to different sequences of interest in a first sub-population), thus the pre-determined first batch barcode sequence corresponds to a given sequence of interest in the first sub-population.

In some embodiments, in step (b), individual linear library molecules in the second sub-population comprise a second batch barcode sequence which corresponds to the second sequence of interest, or the second batch barcode sequence corresponds to one of the second sequences of interest in the second sub-population. In some embodiments, a pre-determined second batch barcode sequence can be linked to a given sequence of interest in the second sub-population (or can be linked to different sequences of interest in a second sub-population), thus the pre-determined second batch barcode sequence corresponds to a given sequence of interest in the second sub-population.

In some embodiments, in the method for generating a plurality of nucleic acid concatemer template molecules immobilized to a support as described above, step (c1) comprises: hybridizing individual linear library molecules of the first sub-population to individual first splint capture primers (120-1) to generate individual open circle library molecules of the first sub-population each having a nick or gap, and hybridizing individual linear library molecules of the second sub-population to individual second splint capture primers (120-2) to generate individual open circle library molecules of the second sub-population each having a nick or gap.

In some embodiments, in step (c1), in the first sub-population of linear library molecules, the universal binding sequence for a first portion of an immobilized first splint capture primer (120-1) can hybridize to the first portion of the immobilized first splint capture primer (210-1). In some embodiments, in the first sub-population of linear library molecules, the universal binding sequence for a second portion of an immobilized first splint capture primer (130-1) can hybridize to the second portion of the immobilized first splint capture primer (220-1). In some embodiments, the immobilized first splint capture primers comprise a first portion (210-1) and a second portion (220-1) which hybridize to adaptor sequences (120-1) and (130-1) in the first sub-population of linear library molecules, and the first splint capture primers serve as a nucleic acid splint molecule for generating a first sub-population of open circle library molecules each having a nick or gap.

In some embodiments, in step (c1), in the second sub-population of linear library molecules, the universal binding sequence for a first portion of an immobilized second splint capture primer (120-2) can hybridize to the first portion of the immobilized second splint capture primer (210-2). In some embodiments, in the second sub-population of linear library molecules, the universal binding sequence for a second portion of an immobilized second splint capture primer (130-2) can hybridize to the second portion of the immobilized second splint capture primer (220-2). In some embodiments, the immobilized second splint capture primers comprise a first portion (210-2) and a second portion (220-2) which hybridize to adaptor sequences (120-2) and (130-2) in the second sub-population of linear library molecules, and the second splint capture primers serve as a nucleic acid splint molecule for generating a second sub-population of open circle library molecules each having a nick or gap.

In some embodiments, in the method for generating a plurality of nucleic acid concatemers immobilized to a support as described above, step (c2) comprises: (i) hybridizing individual linear library molecules of the first sub-population to individual first splint capture primers (120-1) to generate individual open circle library molecules of the first sub-population each having 5′ overhang flap structure that is cleavable with a structure specific 5′ flap endonuclease (e.g., FIGS. 28A(i), 28A(ii), 28B left and 28B right), and hybridizing individual linear library molecules of the second sub-population to individual second splint capture primers (120-2) to generate individual open circle library molecules of the second sub-population each having 5′ overhang flap structure that is cleavable with a structure specific 5′ flap endonuclease (e.g., FIGS. 28A(i), 28A(ii), 28B left and 28B right); and (ii) cleaving the 5′ overhang flap structures by contacting the first and second sub-population of open circle library molecules with a flap cleaving reagent under a condition suitable for cleaving the 5′ overhang flap structures thereby forming a first and second sub-population of cleavage products. In some embodiments, the flap cleaving reagent cleaves the 5′ overhang flap structures thereby forming a plurality of cleavage products, wherein individual cleavage products comprise an open circle library molecule with a newly cleaved 5′ end and a non-cleaved 3′ end, wherein the newly cleaved 5′ end and the non-cleaved 3′ end of the same open circle library molecule forms a nick while being hybridized to the first portion (210) and the second portion (220) of the same splint capture primer, wherein the nick is enzymatically ligatable. In some embodiments, the flap cleaving reagent cleaves the 5′ flap and the 3′ flap thereby generating a plurality of cleavage products, wherein individual cleavage products comprise an open circle library molecule with a newly cleaved 5′ end and a newly cleaved 3′ end, wherein the newly cleaved 5′ and 3′ ends of the same open circle library molecule form a nick while being hybridized to the first portion (210) and the second portion (220) of the same splint capture primer, wherein the nick is enzymatically ligatable. In some embodiments, the flap cleaving reagent cleaves the 5′ flap and the 3′ flap thereby generating a plurality of cleavage products, wherein individual cleavage products comprise an open circle library molecule with a newly cleaved 5′ end and a newly cleaved 3′ end, wherein the newly cleaved 5′ and 3′ ends of the same open circle library molecule form a gap while being hybridized to the first portion (210) and the second portion (220) of the same splint capture primer. In some embodiments, the gap can be subjected to a polymerase-catalyzed fill-in reaction to generate a nick, wherein the nick is enzymatically ligatable. In some embodiments, the gap in individual open circle library molecules can be closed by conducting a polymerase-catalyzed gap fill-in reaction using the newly-cleaved 3′ end of the library molecule as an initiation site for the polymerase-catalyzed fill-in reaction and using the immobilized splint capture primer as a template molecule thereby forming covalently closed circularized molecule having a nick. The nick can be closed by conducting an enzymatic ligation reaction to form a single stranded covalently closed circular library molecule, wherein individual covalently closed circular library molecules are hybridized to an immobilized splint capture primer.

In some embodiments, in step (c2), individual cleavage products in the first sub-population comprise an open circle library molecule with a newly cleaved 5′ end and a non-cleaved 3′ end, wherein the newly cleaved 5′ end and the non-cleaved 3′ end of the same open circle library molecule forms a nick while being hybridized to the first portion (210-1) and the second portion (220-1) of the same splint capture primer, wherein the nick is enzymatically ligatable. In some embodiments, the flap cleaving reagent cleaves the 5′ flap and the 3′ flap thereby generating a first sub-population of cleavage products, wherein individual cleavage products comprise an open circle library molecule with a newly cleaved 5′ end and a newly cleaved 3′ end, wherein the newly cleaved 5′ and 3′ ends of the same open circle library molecule form a nick while being hybridized to the first portion (210-1) and the second portion (220-1) of the same splint capture primer, wherein the nick is enzymatically ligatable.

In some embodiments, in step (c2), individual cleavage products in the second sub-population comprise an open circle library molecule with a newly cleaved 5′ end and a non-cleaved 3′ end, wherein the newly cleaved 5′ end and the non-cleaved 3′ end of the same open circle library molecule forms a nick while being hybridized to the first portion (210-2) and the second portion (220-2) of the same splint capture primer, wherein the nick is enzymatically ligatable. In some embodiments, the flap cleaving reagent cleaves the 5′ flap and the 3′ flap thereby generating a second sub-population of cleavage products, wherein individual cleavage products comprise an open circle library molecule with a newly cleaved 5′ end and a newly cleaved 3′ end, wherein the newly cleaved 5′ and 3′ ends of the same open circle library molecule form a nick while being hybridized to the first portion (210-2) and the second portion (220-2) of the same splint capture primer, wherein the nick is enzymatically ligatable.

In some embodiments, in the method for generating a plurality of nucleic acid concatemers immobilized to a support as described above, step (d) comprises: enzymatically closing the nick or gap in the first sub-population of open circle library molecules thereby generating a first sub-population of covalently closed circular library molecules wherein individual covalently closed circular library molecules in the first sub-population are hybridized to immobilized first splint capture primers. Step (d) can be conducted after step (c1) or (c2) described above.

In some embodiments, step (d) comprises enzymatically closing the nick or gap in the second sub-population of open circle library molecules thereby generating a second sub-population of covalently closed circular library molecules wherein individual covalently closed circular library molecules in the second sub-population are hybridized to immobilized second splint capture primers. Step (d) can be conducted after step (c1) or (c2) described above.

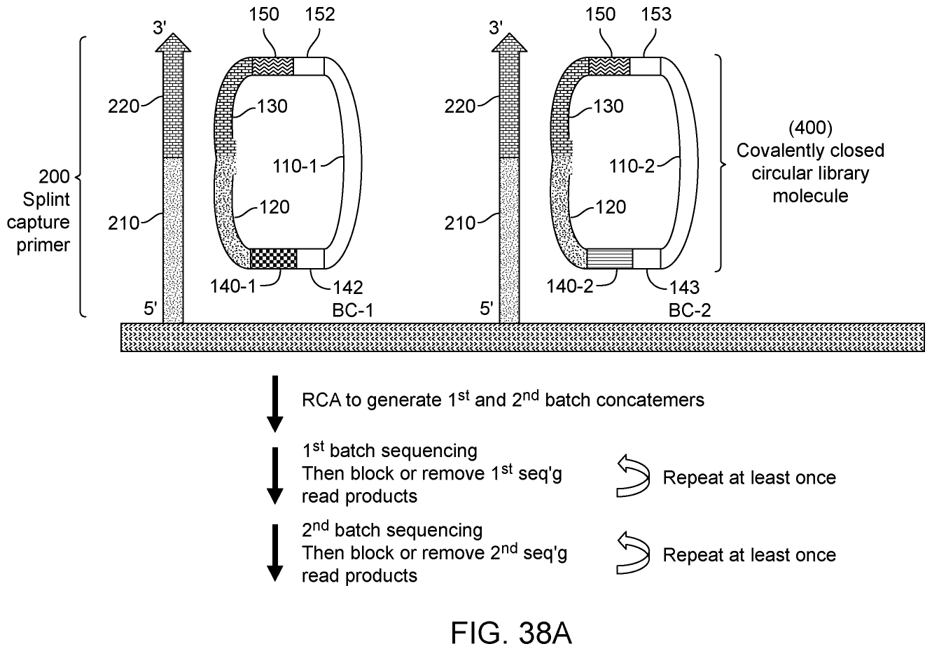

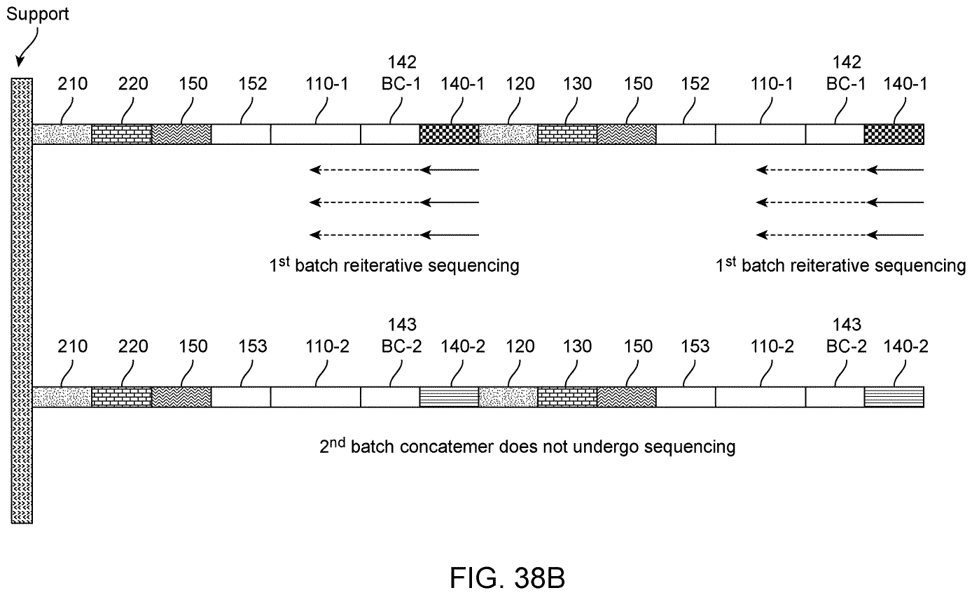

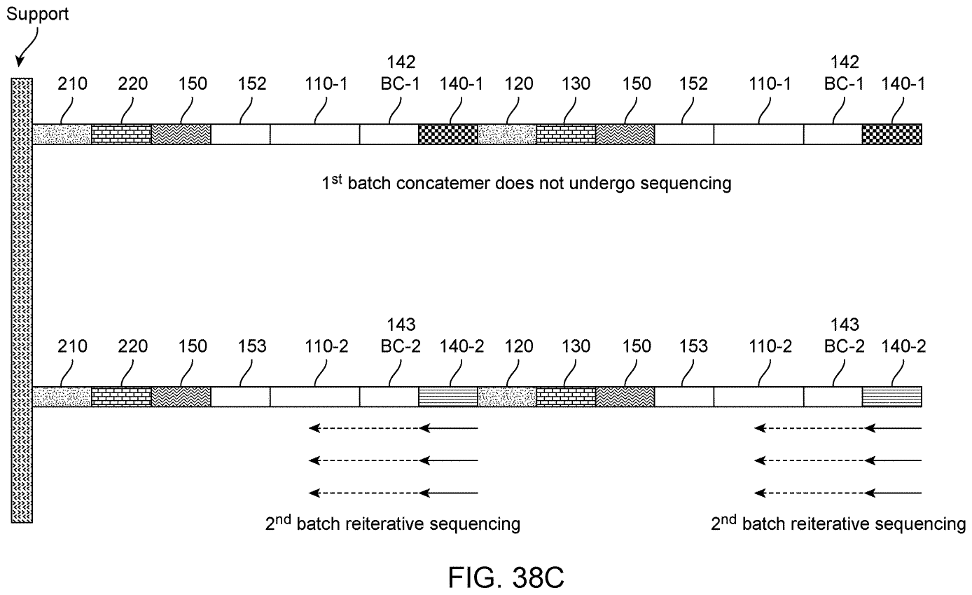

For example, FIG. 38A shows a first covalently closed circular library molecule from a first sub-population hybridized to a first splint capture primer immobilized to a support (FIG. 38A, left), and a second covalently closed circular library molecule from a second sub-population hybridized to a second splint capture primer immobilized to the same support (FIG. 38A, right). The first covalently closed library molecule comprises a first insert sequence (110-1), a first batch barcode sequence (142; BC-1); and a universal binding site for a first batch-specific forward sequencing primer (140-1) which selectively hybridizes to a first batch forward sequencing primer. The universal binding site for the first batch-specific forward sequencing primer (140-1) corresponds to the first insert sequence (110-1). A second covalently closed circular molecule (FIG. 38A, right) can be generated by hybridizing individual linear library molecules from a second sub-population to a splint capture primer (200) immobilized to the same support. The hybridized second linear library molecule forms a second open circle library molecule having a nick or gap which can be enzymatically closed to form a second covalently closed circular library molecule which is hybridized to a second splint-capture primer. The second covalently closed library molecule comprises a second insert sequence (110-2) which differs from the first insert sequence (110-1), a second batch barcode sequence (143; BC-2) which differs from the first batch barcode sequence (143; BC-1); and a universal binding site for a second batch-specific forward sequencing primer (140-2) which selectively hybridizes to a second batch forward sequencing primer. The universal binding site for the second batch-specific forward sequencing primer (140-2) corresponds to the second insert sequence (110-2).

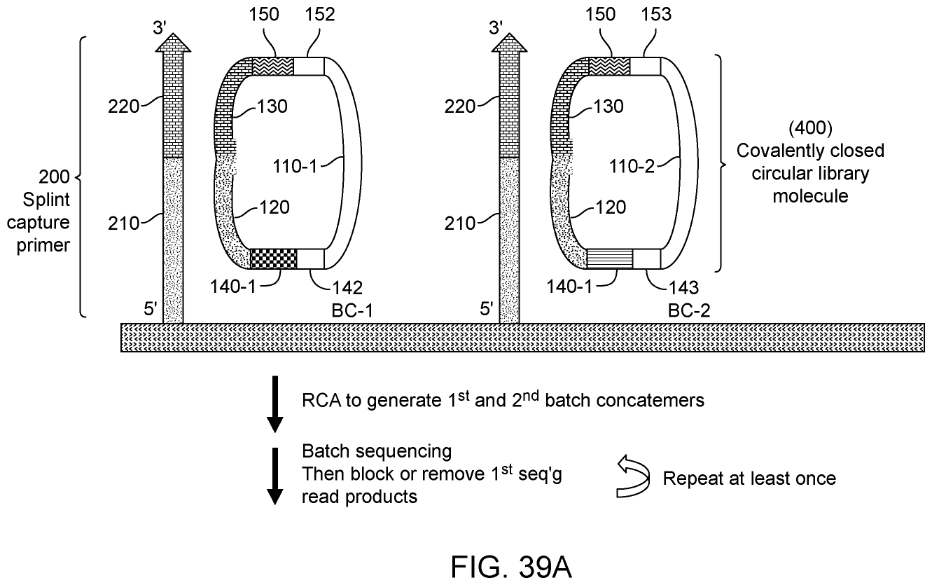

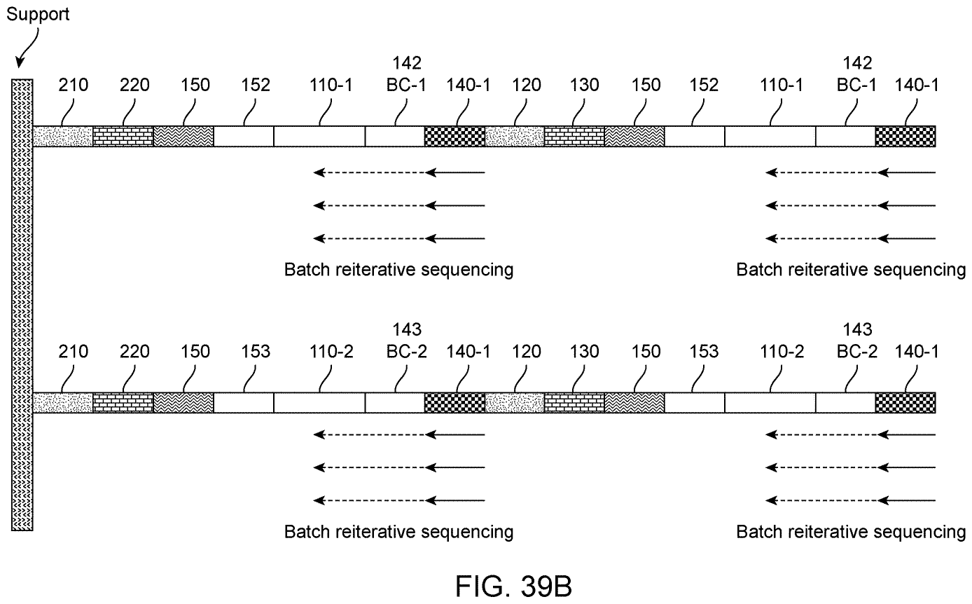

In another example, FIG. 39A shows a first covalently closed circular library molecule from a first sub-population hybridized to a first splint capture primer immobilized to a support (FIG. 39A, left), and a second covalently closed circular library molecule from a second sub-population hybridized to a second splint capture primer immobilized to the same support (FIG. 39A, right). The first covalently closed library molecule (left) comprises a first insert sequence (110-1), a first batch barcode sequence (142; BC-1); and a universal binding site for a first batch-specific forward sequencing primer (140-1) which selectively hybridizes to a first batch forward sequencing primer. The universal binding site for the first batch-specific forward sequencing primer (140-1) corresponds to the first insert sequence (110-1). A second covalently closed circular molecule (right) can be generated by hybridizing individual linear library molecules from a second sub-population to splint capture primers (200) immobilized to the same support. The hybridized second linear library molecule forms a second open circle library molecule having a nick or gap which can be enzymatically closed to form a second covalently closed circular library molecule which is hybridized to a second splint-capture primer. The second covalently closed library molecule (FIG. 39A, right) comprises a second insert sequence (110-2) which differs from the first insert sequence (110-1), a second batch barcode sequence (143; BC-2) which differs from the first batch barcode sequence (143; BC-1); and a universal binding site for a first batch-specific forward sequencing primer (140-1) which selectively hybridizes to a first batch forward sequencing primer. The universal binding site for a first batch-specific forward sequencing primer (140-1) corresponds to the second insert sequence (110-2).

In some embodiments, the method for generating a plurality of nucleic acid concatemers immobilized to a support for batch-specific sequencing comprises step (e) as described above.

In some embodiments, step (e) comprises contacting the immobilized first and second sub-populations of covalently close circular library molecules with a rolling circle amplification reaction mixture and conducting a rolling circle amplification reaction thereby generating a plurality of immobilized single stranded nucleic acid concatemer template molecules including at least a first sub-population and a second sub-population of single stranded nucleic acid concatemer template molecules (e.g., FIGS. 38A and 39A). In some embodiments, the rolling circle amplification reaction is conducted in the presence of a plurality of compaction oligonucleotides. The plurality of compaction oligonucleotides can have the same sequence or can comprise a mixture of compaction oligonucleotides having two or more different sequences.

In some embodiments, in step (e), the rolling circle amplification reaction generates a support comprising a plurality of concatemer template molecules immobilized thereon at a density of about 10²-10¹⁵ concatemer template molecules per mm², wherein the immobilized concatemer template molecules comprise a mixture of at least two sub-populations of concatemer template molecules including at least a first and second sub-population of concatemer template molecules. In some embodiments, the plurality of sub-populations of concatemer template molecules are immobilized to the support at a high density where at least some of the immobilized concatemer template molecules in the first and second sub-populations comprise nearest neighbor concatemer template molecules that touch each other and/or overlap each other when viewed from any angle of the support including above, below or side views of the support (e.g., FIG. 15B). In some embodiments, the support comprises up to 500 million concatemer template molecules immobilized thereon, or up to 1 billion concatemer template molecules immobilized thereon, up to 2 billion concatemer template molecules immobilized thereon, up to 5 billion concatemer template molecules immobilized thereon, or up to 10 billion concatemer template molecules immobilized thereon, or up to 20 billion concatemer template molecules immobilized thereon.

In some embodiments, the method for generating a plurality of nucleic acid concatemer template molecules immobilized to a support comprises step (f) as described above: conducting two or more sequencing reactions to determine the sequences of at least a portion of the immobilized concatemer template molecules of the first and second sub-populations. In some embodiments, the sequencing further comprises step (f1) conducting batch sequencing of the first sub-population of concatemer template molecules and step (f2) conducing batch sequencing of the second sub-population of concatemer template molecules (e.g., FIG. 38A). In some embodiments, the immobilized concatemer template molecules serve as nucleic acid template molecules to be sequenced (e.g., concatemer template molecules). In some embodiments, the concatemer template molecules can be sequenced using any sequencing method. For example, a sequencing method can employ a plurality of sequencing primers, a plurality of sequencing polymerases, and at least one nucleotide reagent.

In some embodiments, step (f1) further comprises: sequencing the first sub-population of concatemer template molecules using a plurality of first batch sequencing primers thereby generating a plurality of first batch sequencing read products. In some embodiments, the batch sequencing of step (f1) comprises hybridizing the first sub-population of concatemer template molecules with a plurality of first batch sequencing primers and conducting at least one sequencing reaction thereby generating a plurality of first batch sequencing read products (e.g., FIG. 38B). In some embodiments, the at least one sequencing reaction employs a plurality of sequencing primers, a plurality of sequencing polymerases, and a plurality of nucleotide reagents. In some embodiments, the sequencing of step (f1) comprises imaging a region of the support to detect the sequencing reactions of the first sub-population of concatemer template molecules.

In some embodiments, step (f1-i) comprises: conducting short read sequencing by performing no more than 200 sequencing cycles of the first sub-population of concatemer template molecules to generate a plurality of first batch sequencing read products that comprise no more than 200 bases in length. In some embodiments, batch-specific sequencing comprises conducting no more than 200 sequencing cycles, conducting no more than 150 sequencing cycles, conducting no more than 100 sequencing cycles, conducting no more than 50 sequencing cycles, conducting no more than 25 sequencing cycles, or conducting no more than 10 sequencing cycles. In some embodiments, the first batch sequencing read products comprise at least a portion of a sequence of interest from the first sub-population of concatemer template molecules. In some embodiments, the first batch sequencing read products comprise the first batch barcode sequence. In some embodiments, the first batch sequencing read products comprise the first batch barcode sequence and the sample index sequence. In some embodiments, the first batch sequencing read products comprise the first batch barcode sequence and at least a portion of a sequence of interest from the first sub-population. In some embodiments, the first batch sequencing read products comprise the first batch barcode sequence, the sample index sequence, and at least a portion of a sequence of interest from the first sub-population. In some embodiments, the short read sequencing comprises hybridizing sequencing primers to sequencing primer binding sites on concatemer template molecules and conducting up to 50 cycles of polymerase-catalyzed sequencing reactions using nucleotide reagents (e.g., FIG. 38B).

In some embodiments, step (f1-ii) comprises: stopping or blocking the short read sequencing of step (f1-ii). In some embodiments, the stopping or blocking comprises incorporating a chain terminating nucleotide to the 3′ terminal end of the first batch sequencing read products to inhibit further sequencing reactions. Exemplary chain terminating nucleotides include dideoxynucleotide or a nucleotide having a 2′ or 3′ chain terminating moiety.

In some embodiments, step (f1-iii) comprises: removing the plurality of first batch sequencing read products from the concatemer template molecules of the first sub-population, and retaining the concatemer template molecules of the first sub-population.

In some embodiments, step (f1-iv) comprises: reiteratively sequencing the concatemer template molecules of the first sub-population by repeating steps (f1-i)-(f1-iii) at least once. In some embodiments, the reiterative sequencing can be conducted 1-10 times, or 10-25 times, or 25-50 times or more. For example, the reiterative sequencing can be conducted up to 100 times (e.g., FIG. 38B).

In some embodiments, when the first and second sub-populations of concatemer template molecules carry a first batch sequencing primer binding site that binds the same first batch sequencing primers, then the first and second sub-populations of concatemer template molecules can be reiteratively sequenced together up to 100 times (e.g., FIG. 39B).

In some embodiments, the sequences of all or substantially all of the first batch sequencing read products can be determined and aligned with a first reference sequence to confirm the presence of the first sequence of interest. The first reference sequence can be the first batch barcode and/or a sequence of interest from the first sub-population.

In some embodiments, hybridizing the first batch sequencing primers to the concatemer template molecules of step (f1-i) can be conducted with a hybridization reagent comprising an SSC buffer (e.g., 2× saline-sodium citrate) buffer with formamide (e.g., 10-20% formamide).

In some embodiments, in step (f1-iii) the plurality of plurality of first batch sequencing read products can be removed from the concatemer template molecules and the plurality of concatemer template molecules can be retained using a de-hybridization reagent comprising an SSC buffer (e.g., saline-sodium citrate) buffer, with or without formamide, at a temperature that promotes nucleic acid denaturation.

In some embodiments, the methods for batch sequencing further comprise step (f2): sequencing the second sub-population of concatemer template molecules using a plurality of second batch sequencing primers thereby generating a plurality of second batch sequencing read products. In some embodiments, the batch sequencing of step (f2) comprises hybridizing the second sub-population of concatemer template molecules with a plurality of second batch sequencing primers and conducting at least one sequencing reaction thereby generating a plurality of second batch sequencing read products (e.g., FIG. 38C). In some embodiments, the at least one sequencing reaction employs a plurality of sequencing primers, a plurality of sequencing polymerases, and a plurality of nucleotide reagents. In some embodiments, the sequencing of step (f2) comprising imaging the same region of the support to detect the sequencing reactions of the second sub-population of concatemer template molecules.

In some embodiments, the sequencing reactions of the first sub-population of concatemer template molecules is stopped before initiating the sequencing reactions of the second sub-population of concatemer template molecules.

In some embodiments, the methods for batch sequencing further comprise step (f2-i): conducting short read sequencing by performing no more than 200 sequencing cycles of the second sub-population of concatemer template molecules to generate a plurality of second batch sequencing read products that comprise no more than 200 bases in length. In some embodiments, the second batch sequencing read products comprise at least a portion of a sequence of interest from the second sub-population. In some embodiments, the second batch sequencing read products comprise the second batch barcode sequence. In some embodiments, the second batch sequencing read products comprise the second batch barcode sequence and a sample index sequence. In some embodiments, the second batch sequencing read products comprise the second batch barcode sequence and at least a portion of a sequence of interest from the second sub-population. In some embodiments, the second batch sequencing read products comprise the second batch barcode sequence, the sample index sequence, and at least a portion of a second sequence of interest from the second sub-population. In some embodiments, the short read sequencing comprises hybridizing sequencing primers to sequencing primer binding sites on concatemer template molecules and conducting up to 50 cycles of polymerase-catalyzed sequencing reactions using nucleotide reagents (e.g., FIG. 38C).

In some embodiments, the methods for batch sequencing further comprise step (f2-ii): stopping or blocking the short read sequencing of step (f2-i). In some embodiments, the stopping or blocking comprises incorporating a chain terminating nucleotide to the 3′ terminal end of the first batch sequencing read products to inhibit further sequencing reactions. Exemplary chain terminating nucleotides include dideoxynucleotide or a nucleotide having a 2′ or 3′ chain terminating moiety.

In some embodiments, the methods for batch sequencing further comprise step (f2-iii): removing the plurality of second batch sequencing read products from the concatemer template molecules of the second sub-population, and retaining the concatemer template molecules of the second sub-population.

In some embodiments, the methods for batch sequencing further comprise step (f2-iv): reiteratively sequencing the concatemer template molecules of the second sub-population by repeating steps (f2-i)-(f2-iii) at least once. In some embodiments, the reiterative sequencing can be conducted 1-10 times, or 10-25 times, or 25-50 times or more. For example, the reiterative sequencing can be conducted up to 100 times (e.g., FIG. 38C).

In some embodiments, the sequences of all or substantially all of the second batch sequencing read products can be determined and aligned with a second reference sequence to confirm the presence of the second sequence of interest. The second reference sequence can be the second batch barcode and/or a sequence of interest from the second sub-population.

In some embodiments, hybridizing the second batch sequencing primers to the concatemer template molecules of step (f2-i) can be conducted with a hybridization reagent comprising an SSC buffer (e.g., 2× saline-sodium citrate) buffer with formamide (e.g., 10-20% formamide).

In some embodiments, in step (f2-iii) the plurality of plurality of second batch sequencing read products can be removed from the concatemer template molecules and the plurality of concatemer template molecules can be retained using a de-hybridization reagent comprising an SSC buffer (e.g., saline-sodium citrate) buffer, with or without formamide, at a temperature that promotes nucleic acid denaturation.

Re-Seeding a Support with Interrupted Sequencing

The present disclosure provides methods for re-seeding a support comprising step (a): providing a support comprising a plurality of splint capture primers (200) immobilized to the support. In some embodiments, the plurality of immobilized splint capture primers have the same sequence. In some embodiments, the plurality of immobilized splint capture primers comprise at least two sub-populations of splint capture primers including at least a first sub-population of splint capture primers having a first sequence and a second sub-population of splint capture primers having a second sequence which differs from the first sequence. In some embodiments, the plurality of splint capture primers comprise single-stranded oligonucleotides. In some embodiments, the plurality of splint capture primers can be used to generate nucleic acid concatemer template molecules immobilized to the support. In some embodiments, the density of the plurality of splint capture primers is about 10²-10¹⁵ per um².

In some embodiments, in step (a), the plurality of splint capture primers (200) can be immobilized to the support at random and non-pre-determined positions. In some embodiments, the plurality of splint capture primers can be immobilized to the support at pre-determined positions (e.g., a patterned support).

In some embodiments, in step (a), the support is passivated with at least one polymer coating layer comprising a plurality of splint capture primers (200) covalently tethered to the at least one polymer layer. In some embodiments, the plurality of splint capture primers are randomly distributed throughout and embedded within the at least one polymer layer.

In some embodiments, in step (a), the support lacks any contours (e.g., wells, protrusions, and the like) arranged in a pre-determined pattern where the contours have features that are sites for attachment (e.g., immobilization) of the nucleic acid template molecules.

In some embodiments, in step (a), the support lacks partitions/barriers that would create separate regions of the support.

In some embodiments, the methods for re-seeding a support further comprise step (b): distributing on the support a first plurality of linear nucleic acid library molecules under a condition suitable for hybridizing individual linear library molecules to individual splint capture primers (200) to generate a first plurality of open circle library molecules each having a nick or gap, or having a 5′ overhang flap structure. In some embodiments, the first plurality of open circle library molecules having a nick or gap are reacted with one or more enzymatic reagents to close the nick or gap to generate a first plurality of covalently closed circular library molecules. In some embodiments, the first plurality of open circle library molecules having a 5′ overhang flap structure are reacted with a flap cleaving reagent to cleave the 5′ flap and ligate the newly cleaved 5′ end to the 3′ end to generate a first plurality of covalently closed circular library molecules.

In some embodiments, in step (b), the first plurality of covalently closed circular library molecules are subjected to a rolling circle amplification reaction, in a template-dependent manner using individual covalently closed circular library molecules in the first plurality, thereby generating a first plurality of nucleic acid concatemer template molecules immobilized to the support, wherein a subset of the splint capture primers hybridize to individual linear library molecules to generate the first plurality of concatemer template molecules. In some embodiments, the number of splint capture primers immobilized to the support exceeds the number of first plurality of linear nucleic acid library molecules distributed onto the support. In some embodiments, individual concatemer template molecules in the first plurality comprise a plurality of tandem copies of a polynucleotide unit, where each polynucleotide unit comprises a sequence of interest and a batch seeding sequencing primer binding site sequence.

In some embodiments, in the methods for re-seeding a support of step (b), filling the gap or nick in the open circle library molecules. In some embodiments, the gap can be filled-in using a non-strand displacing polymerase and a plurality of nucleotides to form open circle library molecules having a nick. In some embodiments, open circle library molecules having a nick can be reacted with a ligation reaction mixture comprising one or more DNA ligase(s), a pH buffering agent, and ATP. In some embodiments, the ligation reaction mixture comprises one or more DNA ligase(s), a pH buffering agent, ATP, a plurality of nucleotides, and the ligation reaction mixture lacks or includes a strand displacing polymerase. In some embodiments, the ligation reaction can be conducted using a ligation reaction mixture comprising at least one DNA ligase and a strand displacing polymerase. In some embodiments, the pH buffering agent in the ligation reaction mixture is within a pH range at which a strand displacing polymerase is inactive. For example, the pH buffering agent in the ligation reaction mixture can be a pH range of about 4-9, a pH range of about 5-8.5, a pH range of about 5.5-8, a pH range of about 6-7.9, a pH range of about 6.5-7.8, a pH range of about 7-7.9, or a pH range of about 7-7.5.

In some embodiments, in the methods for re-seeding a support of step (b), the open circle library molecules comprise a 5′ overhang flap. In some embodiments, the 5′ overhang flap can be cleaved with a flap cleaving reagent comprising at least one 5′ flap endonuclease. In some embodiments, the 5′ flap endonuclease originates from a thermophilic organism, a eukaryote organism or an archaeal organism. In some embodiments, the 5′ flap endonuclease comprises a thermostable enzyme. In some embodiments, the 5′ flap endonuclease comprises FEN1. Any suitable 5′ flap endonuclease described herein or known in the art can be used.

In some embodiments, in the methods for re-seeding a support of step (b), individual linear library molecules in the first plurality comprise a sequence of interest and a seeding batch sequencing primer binding site sequence which corresponds to the sequence of interest. In some embodiments, individual linear library molecules in the first plurality further comprise any one or any combination of two or adaptor sequences arranged in any order: (i) a first universal binding site (120) for a first portion of a splint capture primer (or a complementary sequence thereof); (ii) a universal binding site for a first non-splint capture primer (123) (or a complementary sequence thereof); (iii) at least one sample index sequence (e.g., (160) and/or (170) which can be used to distinguish sequences of interest obtained from different sample sources in a multiplex assay; (iv) at least one universal binding site for a forward sequencing primer (140) (or a complementary sequence thereof); (v) at least one universal binding site for a reverse sequencing primer (150) (or a complementary sequence thereof); (vi) at least one universal binding site for a compaction oligonucleotide (or a complementary sequence thereof); (vii) at least one unique molecular index sequence (UMI) (e.g., (180) and/or (190)) which can be used to uniquely identify a nucleic acid molecule (e.g., having a sequence of interest) to which the unique molecular index sequence is appended; (viii) at least one universal binding site for a pinning primer (or a complementary sequence thereof); (ix) at least one batch-specific barcode sequence; (x) a universal binding site for a second non-splint capture primer (133) (or a complementary sequence thereof); (xi) at least one short random sequence (e.g., NNNN) (132) which provides nucleotide sequence diversity and is about 3-20 nucleotides in length; and/or (xii) a second universal binding site (130) for a second portion of the immobilized splint capture (or a complementary sequence thereof). In some embodiments, the universal binding site for a forward sequencing primer (140) comprises a seeding batch forward sequencing primer binding site which can be employed for re-seeding batch sequencing. In some embodiments, the universal binding site for a reverse sequencing primer (150) comprises a seeding batch reverse sequencing primer binding site which can be employed for re-seeding batch sequencing. In some embodiments, the at least one sample index sequence (e.g., (160) and/or (170) comprises a sample index sequence joined to a short random sequence (e.g., NNN), where the short random sequence provides nucleotide sequence diversity and is about 3-20 nucleotides in length.

Exemplary linear library molecules are shown in FIGS. 20-24. The skilled artisan will recognize that linear library molecules having adaptor sequences constructed with other arrangements are possible.

In some embodiments, in step (b), a pre-determined first seeding batch sequencing primer binding site sequence can be linked to a given sequence of interest in the first plurality of linear library molecules (or can be linked to different sequences of interest in a first plurality of linear library molecules), thus the pre-determined first seeding batch sequencing primer binding site sequence corresponds to a given sequence of interest in the first plurality of linear library molecules.

In some embodiments, in step (b), individual linear library molecules in the first plurality further comprise a seeding batch barcode sequence which corresponds to the sequence of interest. In some embodiments, a pre-determined first seeding batch barcode sequence can be linked to a given sequence of interest in the first plurality of linear library molecules (or can be linked to different sequences of interest in a first plurality of linear library molecules), thus the pre-determined first seeding batch barcode sequence corresponds to a given sequence of interest in the first plurality of linear library molecules.

In some embodiments, in step (b), individual linear library molecules in the first plurality comprise a sequence of interest, the same seeding batch sequencing primer binding site sequence which corresponds to the sequence of interest, and individual linear library molecules further comprise a splint capture primer binding site, and a first seeding batch barcode sequence which corresponds to the sequence of interest.

In some embodiments, in step (b), the sequences of interest in the first plurality of linear library molecules comprise the same sequence or a mixture of different sequences. In some embodiments, the sequences of interest in the first plurality of linear library molecules are about 50-250 bases in length, or about 250-500 bases in length, or about 500-800 bases in length, or about 800-1200 bases in length, or up to 2000 bases in length.

In some embodiments, in step (b), the concentration of the first plurality of linear library molecules that are distributed onto the support can be about 0.1-1 pM, or about 1-5 pM, or about 5-10 pM, or about 10-50 pM.

In some embodiments, in the methods for re-seeding a support of step (b), the first plurality of linear library molecules comprise a plurality of sub-populations of linear library molecules including at least a first and second sub-population of linear library molecules.

In some embodiments, in step (b), individual linear library molecules in the first sub-population comprise the same first sub-population seeding batch sequencing primer binding site sequence and have the same sequence of interest or different sequences of interest. In some embodiments, the first sub-population seeding batch sequencing primer binding site sequence corresponds to the first sequence of interest, or the first sub-population seeding batch sequencing primer binding site sequence corresponds to one of the sequences of interest in the first sub-population. In some embodiments, a pre-determined first sub-population seeding batch sequencing primer binding site sequence can be linked to a given sequence of interest in the first sub-population of linear library molecules (or can be linked to different sequences of interest in a first sub-population of linear library molecules), thus the pre-determined first sub-population seeding batch sequencing primer binding site sequence corresponds to a given sequence of interest in the first sub-population of linear library molecules.

In some embodiments, in step (b), individual linear library molecules in the first sub-population further comprise a first sub-population seeding batch barcode sequence which corresponds to the first sequence of interest, or the first sub-population seeding batch barcode sequence corresponds to one of the sequences of interest in the first sub-population. In some embodiments, a pre-determined first sub-population seeding batch barcode sequence can be linked to a given sequence of interest in the first sub-population of linear library molecules (or can be linked to different sequences of interest in a first sub-population of linear library molecules), thus the pre-determined first sub-population seeding batch barcode sequence corresponds to a given sequence of interest in the first sub-population of linear library molecules.

In some embodiments, in step (b), individual linear library molecules in the first sub-population further comprise a sample index sequence that can be used in a multiplex assay to distinguish sequences of interest obtained from different sample sources. In some embodiments, individual linear library molecules in the first sub-population further comprise a splint capture primer binding site. In some embodiments, individual linear library molecules in the first sub-population further comprise a pinning primer binding site. In some embodiments, individual linear library molecules in the first sub-population further comprise a compaction oligonucleotide binding site.

In some embodiments, in step (b), the sequences of interest in the first sub-population of linear nucleic acid library molecules are about 50-250 bases in length, or about 250-500 bases in length, or about 500-800 bases in length, or about 800-1200 bases in length, or up to 2000 bases in length.

In some embodiments, in the methods for re-seeding a support of step (b), the method comprises conducting a rolling circle amplification reaction, in a template-dependent manner using individual covalently closed circular library molecules in the first sub-population, thereby generating a plurality of first sub-population concatemer template molecules immobilized to the support, wherein a subset of the splint capture primers hybridize to individual covalently closed circular library molecules to generate the plurality of first sub-population concatemer template molecules.

In some embodiments, in step (b), the plurality of first sub-population concatemer template molecules can be immobilized to the support at random and non-predetermined positions on the support, or at pre-determined positions on the support (e.g., patterned support).

In some embodiments, in the methods for re-seeding a support of step (b), individual linear library molecules in the second sub-population comprise the same second sub-population seeding batch sequencing primer binding site sequence and have the same sequence of interest or different sequences of interest. In some embodiments, the second sub-population seeding batch sequencing primer binding site sequence corresponds to the second sequence of interest, or the second sub-population seeding batch sequencing primer binding site sequence corresponds to one of the sequences of interest in the second sub-population. In some embodiments, a pre-determined second sub-population seeding batch sequencing primer binding site sequence can be linked to a given sequence of interest in the second sub-population of linear library molecules (or can be linked to different sequences of interest in a second sub-population of linear library molecules), thus the pre-determined second sub-population seeding batch sequencing primer binding site sequence corresponds to a given sequence of interest in the second sub-population of linear library molecules.

In some embodiments, in step (b), individual linear library molecules in the second sub-population further comprise a second sub-population seeding batch barcode sequence which corresponds to the second sequence of interest, or the second sub-population seeding batch barcode sequence corresponds to one of the sequences of interest in the second sub-population. In some embodiments, a pre-determined second sub-population seeding batch barcode sequence can be linked to a given sequence of interest in the second sub-population of linear library molecules (or can be linked to different sequences of interest in a second sub-population of linear library molecules), thus the pre-determined second subs-population seeding batch barcode sequence corresponds to a given sequence of interest in the second sub-population of linear library molecules.

In some embodiments, in step (b), individual linear library molecules in the second sub-population further comprise a sample index sequence that can be used in a multiplex assay to distinguish sequences of interest obtained from different sample sources. In some embodiments, individual linear library molecules in the second sub-population further comprise a splint capture primer binding site. In some embodiments, individual linear library molecules in the second sub-population further comprise a pinning primer binding site. In some embodiments, individual linear library molecules in the second sub-population further comprise a compaction oligonucleotide binding site.

In some embodiments, in step (b), the sequences of interest in the second sub-population of linear library molecules are about 50-250 bases in length, or about 250-500 bases in length, or about 500-800 bases in length, or about 800-1200 bases in length, or up to 2000 bases in length.

In some embodiments, in step (b), the first sub-population seeding batch sequencing primer binding site sequence and second sub-population seeding batch sequencing primer binding site sequence have different sequences.

In some embodiments, in the methods for re-seeding a support of step (b), the method comprises conducting a rolling circle amplification reaction, in a template-dependent manner using individual linear library molecules in the second sub-population, thereby generating a plurality of second sub-population concatemer template molecules immobilized to the support, wherein a subset of the splint capture primers hybridize to individual linear library molecules to generate the plurality of second sub-population concatemer template molecules.

In some embodiments, in step (b), the plurality of second sub-population concatemer template molecules can be immobilized to the support at random and non-predetermined positions on the support, or at pre-determined positions on the support (e.g., patterned support).

In some embodiments, in the methods for re-seeding a support of step (b), the rolling circle amplification reaction comprises contacting the covalently closed circular library molecules, which are hybridized to immobilized splint capture primers (200), with a plurality of a strand displacing polymerase, and a plurality of nucleotides which include dATP, dCTP, dGTP, dTTP.

In some embodiments, in step (b), the plurality of nucleotide further comprises a plurality of a nucleotide having a scissile moiety (e.g., uracil).

In some embodiments, in step (b), the rolling circle amplification reaction mixture lacks or includes a strand displacing polymerase. In some embodiments, the rolling circle amplification reaction mixture lacks a strand displacing polymerase. In some embodiments, when the rolling circle amplification reaction mixture lacks strand displacing polymerases, then the rolling circle amplification reaction is catalyzed by the strand displacing polymerase present in the ligation reaction mixture of step (d).

In some embodiments, in step (b), the rolling circle amplification reaction is conducted within a pH range at which a strand displacing polymerase is active. For example, the pH buffering agent in the rolling circle amplification reaction mixture can be a pH range of about 7-9, a pH range of about 7.5-9, a pH range of about 8-9, a pH range of about 8.1-8.9, a pH range of about 8.2-8.8, a pH range of about 8.3-8.7, or a pH range of about 8.4-8.6.

In some embodiments, the rolling circle amplification reaction of step (b) can be conducted in the presence, or in the absence, of a plurality of compaction oligonucleotides.

In some embodiments, the methods for re-seeding a support further comprise step (c): sequencing at least a subset of the first plurality of immobilized concatemer template molecules thereby generating a first plurality of sequencing read products. In some embodiments, the sequencing of step (c) comprises imaging a region of the support to detect the sequencing reactions of the first plurality of template molecules.

In some embodiments, in step (c), the immobilized concatemer template molecules in the first plurality are sequenced. For example, at least 30-50%, or at least 50-70%, or at least 70-90% of the immobilized concatemer template molecules in the first plurality are sequenced.

In some embodiments, in step (c), the full length of the immobilized concatemer template molecules in the first plurality are sequenced. In some embodiments, only a partial length of the immobilized concatemer template molecules in the first plurality are sequenced.

In some embodiments, in step (c), the immobilized concatemer template molecules in the first plurality are subjected to no more than 200 sequencing cycles.

In some embodiments, in step (c), a partial length of the immobilized concatemer template molecules in the first plurality are reiteratively sequenced.

In some embodiments, in the methods for re-seeding a support of step (c), a first sub-population of the immobilized concatemer template molecules in the first plurality are sequenced using the first batch sequencing primer binding sites in the first sub-population of immobilized concatemer template molecules.

In some embodiments, in step (c), the full length of the immobilized concatemer template molecules in the first sub-population are sequenced. In some embodiments, only a partial length of the immobilized concatemer template molecules in the first sub-population are sequenced.

In some embodiments, in step (c), the immobilized concatemer template molecules in the first sub-population are subjected to no more than 200 sequencing cycles.

In some embodiments, in step (c), a partial length of the immobilized concatemer template molecules in the first sub-population are reiteratively sequenced.

In some embodiments, in the methods for re-seeding a support of step (c), a second sub-population of the immobilized concatemer template molecules in the second plurality are sequenced using the second batch sequencing primer binding sites in the second sub-population of immobilized concatemer template molecules.

In some embodiments, in step (c), the full length of the immobilized concatemer template molecules in the second sub-population are sequenced. In some embodiments, only a partial length of the immobilized concatemer template molecules in the second sub-population are sequenced.

In some embodiments, in step (c), the immobilized concatemer template molecules in the second sub-population are subjected to no more than 200 sequencing cycles.

In some embodiments, in step (c), a partial length of the immobilized concatemer template molecules in the second sub-population are reiteratively sequenced.

In some embodiments, the methods for re-seeding a support further comprise step (d): distributing on the support a second plurality of linear nucleic acid library molecules under a condition suitable for hybridizing individual linear library molecules of the second plurality to individual splint capture primers to generate a second plurality of open circle library molecules each having a nick or gap, or having a 5′ overhang flap structure. In some embodiments, the first plurality of open circle library molecules having a nick or gap are reacted with one or more enzymatic reagents to close the nick or gap to generate a first plurality of covalently closed circular library molecules. In some embodiments, the first plurality of open circle library molecules having a 5′ overhang flap structure are reacted with a flap cleaving reagent to cleave the 5′ flap and ligate the newly cleaved 5′ end to the 3′ end to generate a first plurality of covalently closed circular library molecules.

In some embodiments, in step (d), the first plurality of covalently closed circular library molecules are subjected to a second rolling circle amplification reaction, in a template-dependent manner using individual covalently closed circular library molecules library molecules in the second plurality, thereby generating a second plurality of nucleic acid concatemer template molecules immobilized to the support. In some embodiments, individual concatemer template molecules in the second plurality comprise a plurality of tandem copies of a polynucleotide unit, where each polynucleotide unit comprises a sequence of interest and a batch seeding sequencing primer binding site sequence. In some embodiments, the first plurality of concatemer template molecules of step (c) can be completely sequenced or the sequencing can be interrupted at any time prior to distributing the second plurality of linear nucleic acid library molecules onto the support of step (d).

In some embodiments, in the methods for re-seeding a support of step (d), the open circle library molecules comprise a gap, and the gap in the open circle library molecules can be filled-in using a non-strand displacing polymerase and a plurality of nucleotides to form open circle library molecules having a nick. In some embodiments, open circle library molecules having a nick can be reacted with a ligation reaction mixture comprising one or more DNA ligase(s), a pH buffering agent, and ATP. In some embodiments, the ligation reaction mixture comprises one or more DNA ligase(s), a pH buffering agent, ATP, a plurality of nucleotides, and the ligation reaction mixture lacks or includes a strand displacing polymerase. In some embodiments, the ligation reaction can be conducted using a ligation reaction mixture comprising at least one DNA ligase and a strand displacing polymerase. In some embodiments, the pH buffering agent in the ligation reaction mixture is within a pH range at which a strand displacing polymerase is inactive. For example, the pH buffering agent in the ligation reaction mixture can be a pH range of about 4-9, a pH range of about 5-8.5, a pH range of about 5.5-8, a pH range of about 6-7.9, a pH range of about 6.5-7.8, a pH range of about 7-7.9, or a pH range of about 7-7.5.

In some embodiments, in the methods for re-seeding a support of step (d), the 5′ overhang flap in the open circle library molecules can be cleaved with a flap cleaving reagent comprising at least one 5′ flap endonuclease. In some embodiments, the at least one 5′ flap endonuclease originates from a thermophilic organism, a eukaryote organism or an archaeal organism. In some embodiments, the 5′ flap endonuclease comprises a thermostable enzyme. In some embodiments, the 5′ flap endonuclease comprises FEN1.

In some embodiments, in the methods for re-seeding the support of step (d), individual linear library molecules in the second plurality comprise a sequence of interest and a seeding batch sequencing primer binding site sequence which corresponds to the sequence of interest. In some embodiments, individual linear library molecules in the second plurality further comprise any one or any combination of two or adaptor sequences arranged in any order: (i) a first universal binding site (120) for a first portion of a splint capture primer (or a complementary sequence thereof); (ii) a universal binding site for a first non-splint capture primer (123) (or a complementary sequence thereof); (iii) at least one sample index sequence (e.g., (160) and/or (170) which can be used to distinguish sequences of interest obtained from different sample sources in a multiplex assay; (iv) at least one universal binding site for a forward sequencing primer (140) (or a complementary sequence thereof); (v) at least one universal binding site for a reverse sequencing primer (150) (or a complementary sequence thereof); (vi) at least one universal binding site for a compaction oligonucleotide (or a complementary sequence thereof); (vii) at least one unique molecular index sequence (UMI) (e.g., (180) and/or (190)) which can be used to uniquely identify a nucleic acid molecule (e.g., having a sequence of interest) to which the unique molecular index sequence is appended; (viii) at least one universal binding site for a pinning primer (or a complementary sequence thereof); (ix) at least one batch-specific barcode sequence; (x) a universal binding site for a second non-splint capture primer (133) (or a complementary sequence thereof); (xi) at least one short random sequence (e.g., NNNN) (132) which provides nucleotide sequence diversity and is about 3-20 nucleotides in length; and/or (xii) a second universal binding site (130) for a second portion of the immobilized splint capture (or a complementary sequence thereof). In some embodiments, the universal binding site for a forward sequencing primer (140) comprises a seeding batch forward sequencing primer binding site which can be employed for re-seeding batch sequencing. In some embodiments, the universal binding site for a reverse sequencing primer (150) comprises a seeding batch reverse sequencing primer binding site which can be employed for re-seeding batch sequencing. In some embodiments, the at least one sample index sequence (e.g., (160) and/or (170) comprises a sample index sequence joined to a short random sequence (e.g., NNN), where the short random sequence provides nucleotide sequence diversity and is about 3-20 nucleotides in length.

Exemplary linear library molecules are shown in FIGS. 20-24. The skilled artisan will recognize that linear library molecules having adaptor sequences constructed with other arrangements are possible.

In some embodiments, in step (d), a pre-determined second seeding batch sequencing primer binding site sequence can be linked to a given sequence of interest in the second plurality of linear library molecules (or can be linked to different sequences of interest in a second plurality of linear library molecules), thus the pre-determined second seeding batch sequencing primer binding site sequence corresponds to a given sequence of interest in the second plurality of linear library molecules.

In some embodiments, in step (d), individual linear library molecules in the second plurality further comprise a seeding batch barcode sequence which corresponds to the sequence of interest.

In some embodiments, in step (d), a pre-determined second seeding batch barcode sequence can be linked to a given sequence of interest in the second plurality of linear library molecules (or can be linked to different sequences of interest in a second plurality of linear library molecules), thus the pre-determined second seeding batch barcode sequence corresponds to a given sequence of interest in the second plurality of linear library molecules.

In some embodiments, in step (d), individual linear library molecules in the second plurality comprise a sequence of interest, the same seeding batch sequencing primer binding site sequence which corresponds to the sequence of interest, and individual linear library molecules further comprise a splint capture primer binding site, and a second seeding batch barcode sequence which corresponds to the sequence of interest.

In some embodiments, in step (d), the sequences of interest in the second plurality of linear nucleic acid library molecules comprise the same sequence or a mixture of different sequences. In some embodiments, the sequences of interest in the second plurality of linear nucleic acid library molecules are about 50-250 bases in length, or about 250-500 bases in length, or about 500-800 bases in length, or about 800-1200 bases in length, or up to 2000 bases in length.

In some embodiments, in step (d), the concentration of the second plurality of linear nucleic acid library molecules that are distributed onto the support can be about 0.1-1 pM, or about 1-5 pM, or about 5-10 pM, or about 10-50 pM.

In some embodiments, in the methods for re-seeding a support of step (d), the second plurality of linear nucleic acid library molecules comprise a plurality of sub-populations of linear library molecules including at least a third and fourth sub-population of linear library molecules.

In some embodiments, in step (d), individual linear library molecules in the third sub-population comprise the same third sub-population seeding batch sequencing primer binding site sequence and have the same sequence of interest or different sequences of interest. In some embodiments, the third sub-population seeding batch sequencing primer binding site sequence corresponds to the third sequence of interest, or the third sub-population seeding batch sequencing primer binding site sequence corresponds to one of the sequences of interest in the third sub-population. In some embodiments, a pre-determined third sub-population seeding batch sequencing primer binding site sequence can be linked to a given sequence of interest in the third sub-population of linear library molecules (or can be linked to different sequences of interest in a third sub-population of linear library molecules), thus the pre-determined third sub-population seeding batch sequencing primer binding site sequence corresponds to a given sequence of interest in the third sub-population of linear library molecules.

In some embodiments, in step (d), individual linear library molecules in the third sub-population further comprise a third sub-population seeding batch barcode sequence which corresponds to the third sequence of interest, or the third sub-population seeding batch barcode sequence corresponds to one of the sequences of interest in the third sub-population. In some embodiments, a pre-determined third sub-population seeding batch barcode sequence can be linked to a given sequence of interest in the third sub-population of linear library molecules (or can be linked to different sequences of interest in a third sub-population of linear library molecules), thus the pre-determined third sub-population seeding batch barcode sequence corresponds to a given sequence of interest in the third sub-population of linear library molecules.

In some embodiments, in step (d), individual linear library molecules in the third sub-population further comprise a sample index sequence that can be used in a multiplex assay to distinguish sequences of interest obtained from different sample sources. In some embodiments, individual linear library molecules in the third sub-population further comprise a splint capture primer binding site. In some embodiments, individual linear library molecules in the third sub-population further comprise a pinning primer binding site. In some embodiments, individual linear library molecules in the third sub-population further comprise a compaction oligonucleotide binding site.

In some embodiments, in step (d), the sequences of interest in the third sub-population of linear nucleic acid library molecules are about 50-250 bases in length, or about 250-500 bases in length, or about 500-800 bases in length, or about 800-1200 bases in length, or up to 2000 bases in length.

In some embodiments, in the methods for re-seeding a support of step (d), the method comprises conducting a rolling circle amplification reaction, in a template-dependent manner using individual covalently closed circular library molecules in the third sub-population, thereby generating a plurality of third sub-population concatemer template molecules immobilized to the support, wherein a subset of the splint capture primers hybridize to individual covalently closed circular library molecules to generate the plurality of third sub-population concatemer template molecules.

In some embodiments, in step (d), the plurality of third sub-population concatemer template molecules can be immobilized to the support at random and non-predetermined positions, or at pre-determined positions (e.g., patterned support).

In some embodiments, in the methods for re-seeding a support of step (d), individual linear library molecules in the fourth sub-population comprise the same fourth sub-population seeding batch sequencing primer binding site sequence and have the same sequence of interest or different sequences of interest. In some embodiments, the fourth sub-population seeding batch sequencing primer binding site sequence corresponds to the fourth sequence of interest, or the fourth sub-population seeding batch sequencing primer binding site sequence corresponds to one of the sequences of interest in the fourth sub-population. In some embodiments, a pre-determined fourth sub-population seeding batch sequencing primer binding site sequence can be linked to a given sequence of interest in the fourth sub-population of linear library molecules (or can be linked to different sequences of interest in a fourth sub-population of linear library molecules), thus the pre-determined fourth sub-population seeding batch sequencing primer binding site sequence corresponds to a given sequence of interest in the fourth sub-population of linear library molecules.

In some embodiments, in step (d), individual linear library molecules in the fourth sub-population further comprise a fourth sub-population seeding batch barcode sequence which corresponds to the fourth sequence of interest, or the fourth sub-population seeding batch barcode sequence corresponds to one of the sequences of interest in the fourth sub-population. In some embodiments, a pre-determined fourth sub-population seeding batch barcode sequence can be linked to a given sequence of interest in the fourth sub-population of linear library molecules (or can be linked to different sequences of interest in a fourth sub-population of linear library molecules), thus the pre-determined fourth subs-population seeding batch barcode sequence corresponds to a given sequence of interest in the fourth sub-population of linear library molecules.

In some embodiments, in step (d), individual linear library molecules in the fourth sub-population further comprise a sample index sequence that can be used in a multiplex assay to distinguish sequences of interest obtained from different sample sources. In some embodiments, individual linear library molecules in the fourth sub-population further comprise a splint capture primer binding site. In some embodiments, individual linear library molecules in the fourth sub-population further comprise a pinning primer binding site. In some embodiments, individual linear library molecules in the fourth sub-population further comprise a compaction oligonucleotide binding site.

In some embodiments, in step (d), the sequences of interest in the fourth sub-population of linear nucleic acid library molecules are about 50-250 bases in length, or about 250-500 bases in length, or about 500-800 bases in length, or about 800-1200 bases in length, or up to 2000 bases in length.

In some embodiments, in step (d), the third sub-population seeding batch sequencing primer binding site sequence and fourth sub-population seeding batch sequencing primer binding site sequence have different sequences.

In some embodiments, in the methods for re-seeding a support of step (d), the method comprises conducting a rolling circle amplification reaction, in a template-dependent manner using individual covalently closed circular library molecules in the fourth sub-population, thereby generating a plurality of fourth sub-population concatemer template molecules immobilized to the support, wherein a subset of the splint capture primers hybridize to individual covalently closed circular library molecules to generate the plurality of fourth sub-population concatemer template molecules.

In some embodiments, in step (d), the plurality of fourth sub-population concatemer template molecules can be immobilized to the support at random and non-predetermined positions, or at pre-determined positions (e.g., patterned support).

In some embodiments, in the methods for re-seeding a support of step (d), the rolling circle amplification reaction comprises contacting the covalently closed circular library molecules, which are hybridized to immobilized splint capture primers, with a plurality of a strand displacing polymerase, and a plurality of nucleotides which include dATP, dCTP, dGTP, dTTP. In some embodiments, the plurality of nucleotide further comprises a plurality of a nucleotide having a scissile moiety (e.g., uracil).

In some embodiments, in step (d), the rolling circle amplification reaction is conducted within a pH range at which a strand displacing polymerase is active. For example, the pH buffering agent in the rolling circle amplification reaction mixture can be a pH range of about 7-9, a pH range of about 7.5-9, a pH range of about 8-9, a pH range of about 8.1-8.9, a pH range of about 8.2-8.8, a pH range of about 8.3-8.7, or a pH range of about 8.4-8.6.

In some embodiments, in step (d), the rolling circle amplification reaction of step (d) can be conducted in the presence, or in the absence, of a plurality of compaction oligonucleotides.

In some embodiments, the methods for re-seeding a support further comprise step (e): sequencing at least a subset of the second plurality of immobilized concatemer template molecules thereby generating a second plurality of sequencing read products. In some embodiments, the sequencing of step (e) comprises imaging a region of the support to detect the sequencing reactions of the second plurality of template molecules. In some embodiments, the same region of the support is sequenced in steps (c) and (e). In some embodiments, different regions of the support are sequenced in steps (c) and (e).

In some embodiments, in step (e), the immobilized concatemer template molecules in the second plurality are sequenced. For example, at least 30-50%, or at least 50-70%, or at least 70-90% of the immobilized concatemer template molecules in the second plurality are sequenced.

In some embodiments, in step (e), the full length of the immobilized concatemer template molecules in the second plurality are sequenced. In some embodiments, only a partial length of the immobilized concatemer template molecules in the second plurality are sequenced.

In some embodiments, in step (e), the immobilized concatemer template molecules in the second plurality are subjected to no more than 200 sequencing cycles.

In some embodiments, in step (e), a partial length of the immobilized concatemer template molecules in the second plurality are reiteratively sequenced.

In some embodiments, in the methods for re-seeding a support of step (e), the third sub-population of the immobilized concatemer template molecules in the second plurality are sequenced using the third batch sequencing primer binding sites in the third sub-population of immobilized concatemer template molecules.

In some embodiments, in step (e), the full length of the immobilized concatemer template molecules in the third sub-population are sequenced. In some embodiments, only a partial length of the immobilized concatemer template molecules in the third sub-population are sequenced.

In some embodiments, in step (e), the immobilized concatemer template molecules in the third sub-population are subjected to no more than 200 sequencing cycles.

In some embodiments, in step (e), a partial length of the immobilized concatemer template molecules in the third sub-population are reiteratively sequenced.

In some embodiments, in the methods for re-seeding a support of step (e), the fourth sub-population of the immobilized concatemer template molecules in the second plurality are sequenced using the fourth batch sequencing primer binding sites in the fourth sub-population of immobilized concatemer template molecules.

In some embodiments, in step (e), the full length of the immobilized concatemer template molecules in the fourth sub-population are sequenced. In some embodiments, only a partial length of the immobilized concatemer template molecules in the fourth sub-population are sequenced.

In some embodiments, in step (e), the immobilized concatemer template molecules in the fourth sub-population are subjected to no more than 200 sequencing cycles.

In some embodiments, in step (e), a partial length of the immobilized concatemer template molecules in the fourth sub-population are reiteratively sequenced.

In some embodiments, the methods for re-seeding a support further comprise reiteratively sequencing the first sub-population of concatemer template molecules, which comprises step (c1): conducting short read sequencing by performing no more than 200 sequencing cycles of the first sub-population of concatemer template molecules to generate a plurality of first sub-population batch sequencing read products that comprise no more than 200 bases in length.

In some embodiments, in step (c1), the first sub-population batch sequencing read products comprise at least a portion of the first sequence of interest. In some embodiments, the first sub-population batch sequencing read products comprise the first sub-population seeding batch barcode sequence. In some embodiments, the first sub-population batch sequencing read products comprise the first sub-population seeding batch barcode sequence and the sample index sequence. In some embodiments, the first sub-population batch sequencing read products comprise the first sub-population seeding batch barcode sequence and at least a portion of the first sequence of interest. In some embodiments, the first sub-population batch sequencing read products comprise the first sub-population seeding batch barcode sequence, the sample index sequence, and at least a portion of the first sequence of interest.

In some embodiments, the methods for re-seeding a support further comprise step (c2): stopping/blocking the short read sequencing of step (c1). In some embodiments, the stopping/blocking comprises incorporating a chain terminating nucleotide to the 3′ terminal end of the first sub-population batch sequencing read products to inhibit further sequencing reactions. Exemplary chain terminating nucleotides include dideoxynucleotide or a nucleotide having a 2′ or 3′ chain terminating moiety.

In some embodiments, the methods for re-seeding a support further comprise step (c3): removing the plurality of first sub-population batch sequencing read products from the concatemer template molecules of the first sub-population, and retaining the concatemer template molecules of the first sub-population. In some embodiments, step (c3) is optional.

In some embodiments, the methods for re-seeding a support further comprise step (c4): reiteratively sequencing the concatemer template molecules of the first sub-population by repeating steps (c1)-(c3) at least once. In some embodiments, the reiterative sequencing can be conducted 1-10 times, or 10-25 times, or 25-50 times or more.

In some embodiments, the sequences of the first sub-population batch sequencing read products can be determined and aligned with a first reference sequence to confirm the presence of the first sequence of interest. The first reference sequence can be the first sub-population seeding batch barcode and/or the first sequence of interest.

In some embodiments, the methods for re-seeding a support further comprise reiteratively sequencing the third sub-population of concatemer template molecules in a manner similar to steps (c1)-(c4) as described above for the first sub-population of concatemer template molecules.

In some embodiments, the methods for re-seeding a support further comprise reiteratively sequencing the second sub-population of concatemer template molecules, which comprises step (c1): conducting short read sequencing by performing no more than 200 sequencing cycles of the second sub-population of concatemer template molecules to generate a plurality of second sub-population batch sequencing read products that comprise no more than 200 bases in length.

In some embodiments, in step (c1), the second sub-population batch sequencing read products comprise at least a portion of the second sequence of interest. In some embodiments, the second sub-population batch sequencing read products comprise the second sub-population seeding batch barcode sequence. In some embodiments, the second sub-population batch sequencing read products comprise the second sub-population seeding batch barcode sequence and the sample index sequence. In some embodiments, the second sub-population batch sequencing read products comprise the second sub-population seeding batch barcode sequence and at least a portion of the second sequence of interest. In some embodiments, the second sub-population batch sequencing read products comprise the second sub-population seeding batch barcode sequence, the sample index sequence, and at least a portion of the second sequence of interest.

In some embodiments, the methods for re-seeding a support further comprise step (c2): stopping or blocking the short read sequencing of step (c1). In some embodiments, the stopping or blocking comprises incorporating a chain terminating nucleotide to the 3′ terminal end of the second sub-population batch sequencing read products to inhibit further sequencing reactions. Exemplary chain terminating nucleotides include dideoxynucleotide or a nucleotide having a 2′ or 3′ chain terminating moiety.

In some embodiments, the methods for re-seeding a support further comprise step (c3): removing the plurality of second sub-population batch sequencing read products from the concatemer template molecules of the second sub-population, and retaining the concatemer template molecules of the second sub-population. In some embodiments, step (c3) is optional.

In some embodiments, the methods for re-seeding a support further comprise step (c4): reiteratively sequencing the concatemer template molecules of the second sub-population by repeating steps (c1)-(c3) at least once. In some embodiments, the reiterative sequencing can be conducted 1-10 times, or 10-25 times, or 25-50 times or more.

In some embodiments, the sequences of the second sub-population batch sequencing read products can be determined and aligned with a second reference sequence to confirm the presence of the second sequence of interest. The second reference sequence can be the second sub-population seeding batch barcode and/or the second sequence of interest.

In some embodiments, the methods for re-seeding a support further comprise reiteratively sequencing the fourth sub-population of concatemer template molecules in a manner similar to steps (c1)-(c4) as described above for the second sub-population of concatemer template molecules.

### Compaction Oligonucleotides

The present disclosure provides compaction oligonucleotides which can be employed in any of the methods for generating a plurality of nucleic acid concatemers immobilized to a support described herein. In some embodiments, the compaction oligonucleotides comprise single stranded oligonucleotides comprising DNA, RNA, or a combination of DNA and RNA. The compaction oligonucleotides can be any length, including 20-150 nucleotides, or 30-100 nucleotides, or 40-80 nucleotides in length. In some embodiments, the compaction oligonucleotides include a 5′ region, an optional internal region (intervening region), and a 3′ region. The 5′ and 3′ regions of the compaction oligonucleotide can hybridize to binding sites in the concatemer to pull together distal portions of the concatemer causing compaction of the concatemer to form a DNA nanoball.

In some embodiments, the 5′ region of the compaction oligonucleotides can be wholly complementary or partially complementary along its length to a first portion of a concatemer template molecule. In some embodiments, the 3′ region of the compaction oligonucleotides can be wholly complementary or partially complementary along its length to a second portion of a concatemer template molecule. In some embodiments, the 5′ region of the compaction oligonucleotides can hybridize to a first universal sequence portion of a concatemer template molecule. In some embodiments, the 3′ region of the compaction oligonucleotides can hybridize to a second universal sequence portion of the same concatemer template molecule.

In some embodiments, the 5′ region of the compaction oligonucleotide can have the same sequence as the 3′ region. In some embodiments, the 5′ region of the compaction oligonucleotide can have a sequence that is different from the 3′ region. In some embodiments, the 3′ region of the compaction oligonucleotide can have a sequence that is a reverse sequence of the 5′ region. In some embodiments, the 5′ region of the compaction oligonucleotide can have a sequence that is a reverse sequence of the 3′ region.

In some embodiments, the 3′ region of any of the compaction oligonucleotides can include an additional three bases at the terminal 3′ end which comprises 2′-O-methyl RNA bases (e.g., designated mUmUmU) or the terminal 3′ end lacks additional 2′-O-methyl RNA bases.

In some embodiments, the compaction oligonucleotides comprise one or more modified bases or linkages at their 5′ or 3′ ends to confer certain functionalities. In some embodiments, the compaction oligonucleotides comprise at least one phosphorothioate linkages at their 5′ and/or 3′ ends to confer exonuclease resistance. In some embodiments, at least one nucleotide at or near the 3′ end comprises a 2′ fluoro base which confers exonuclease resistance. In some embodiments, the terminal 3′ ends of the compaction oligonucleotides are non-extendible. In some embodiments, the 3′ end of the compaction oligonucleotides comprise at least one 2′-O-methyl RNA base which blocks polymerase-catalyzed extension. For example, the 3′ end of the compaction oligonucleotide comprises three bases comprising 2′-O-methyl RNA base (e.g., designated mUmUmU). In some embodiments, the compaction oligonucleotides comprise a 3′ inverted dT at their 3′ ends which blocks polymerase-catalyzed extension. In some embodiments, the compaction oligonucleotides comprise 3′ phosphorylation which blocks polymerase-catalyzed extension. In some embodiments, the internal region of the compaction oligonucleotides comprise at least one locked nucleic acid (LNA) which increases the thermal stability of duplexes formed by hybridizing a compaction oligonucleotide to a concatemer template molecule. In some embodiments, the compaction oligonucleotides comprise a phosphorylated 5′ end (e.g., using a polynucleotide kinase).

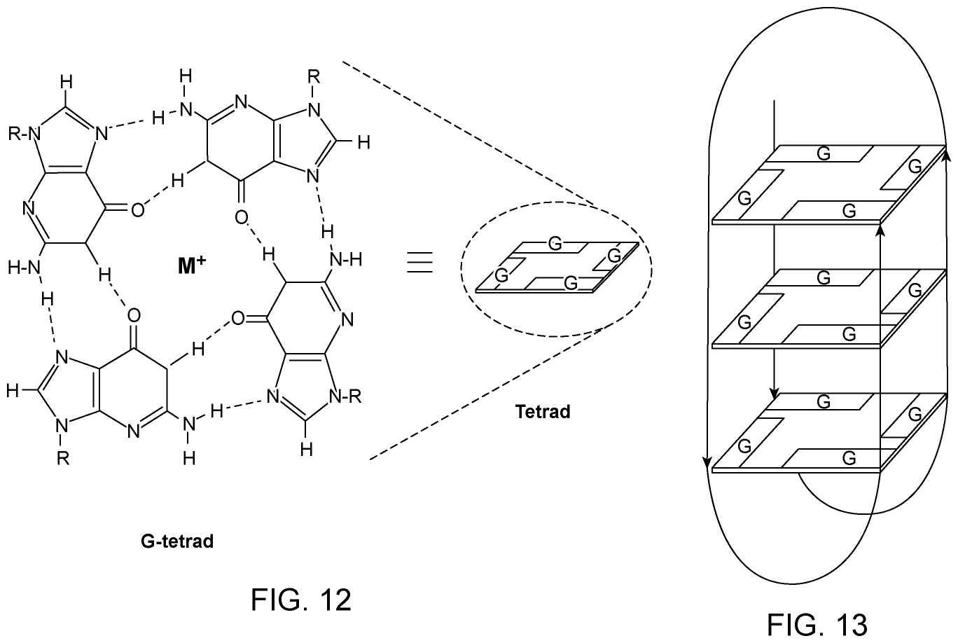

In some embodiments, the compaction oligonucleotides can include at least one region having consecutive guanines. For example, the compaction oligonucleotides can include at least one region having 2, 3, 4, 5, 6 or more consecutive guanines. In some embodiments, the compaction oligonucleotides comprise four consecutive guanines which can form a guanine tetrad structure (e.g., FIG. 12). The guanine tetrad structure can be stabilized via Hoogsteen hydrogen bonding. The guanine tetrad structure can be stabilized by a central cation including potassium, sodium, lithium, rubidium or cesium.

At least one compaction oligonucleotide can form a guanine tetrad (e.g., FIG. 12) and hybridize to the universal binding sequences in a concatemer which can cause the concatemer to fold to form an intramolecular G-quadruplex structure (e.g., FIG. 13). The concatemers can self-collapse to form compact nanoballs. Formation of the guanine tetrads and G-quadruplexes in the nanoballs may increase the stability of the nanoballs to retain their compact size and shape which can withstand changes in pH, temperature and/or repeated flows of reagents during multiple sequencing cycles.

In some embodiments, the plurality of compaction oligonucleotides in the rolling circle amplification reaction have the same sequence. Alternatively, the plurality of compaction oligonucleotides in the rolling circle amplification reaction comprise a mixture of two or more different populations of compaction oligonucleotides having different sequences.

In some embodiment, the immobilized concatemer template molecule can self-collapse into a compact nucleic acid nanoball. The nanoballs can be imaged and a FWHM measurement can be obtained to give the shape/size of the nanoballs.

In some embodiments, inclusion of compaction oligonucleotides in the rolling circle amplification reaction can promote collapsing of a concatemer into a DNA nanoball. Conducting RCA with compaction oligonucleotides helps retain the compact size and shape of a DNA nanoball during multiple sequencing cycles which can improve FWHM (full width half maximum) of a spot image of the DNA nanoball. In some embodiments, the DNA nanoball does not unravel during multiple sequencing cycles. In some embodiments, the spot image of the DNA nanoball does not enlarge during multiple sequencing cycles. In some embodiments, the spot image of the DNA nanoball remains a discrete spot during multiple sequencing cycles. The spot image can be represented as a Gaussian spot and the size can be measured as a FWHM. A smaller spot size as indicated by a smaller FWHM typically correlates with an improved image of the spot. In some embodiments, the FWHM of a nanoball spot can be about 10 um or smaller.

### Nucleic Acid Library Preparation

The present disclosure provides methods for preparing a nucleic acid library which can be employed in any of the methods for generating a plurality of nucleic acid concatemers immobilized to a support described herein. In some embodiments, a nucleic acid library typically comprises a plurality of nucleic acid library molecules where individual nucleic acid library molecules comprise a sequence of interest (e.g., insert region) covalently joined to at least one adaptor sequence. Individual library molecules can have an insert sequence that is the same or different insert sequence as other library molecules. Individual library molecules in the population can have an adaptor sequence that is the same (e.g., a universal adaptor sequence) or different (e.g., unique molecular identifier) adaptor sequence as other library molecules in the population.

In some embodiments, the nucleic acid library molecules comprise DNA, RNA, cDNA or chimeric DNA/RNA. The nucleic acid library molecules can be single-stranded or double-stranded, or can include single-stranded or double-stranded portions. The nucleic acid library molecules can be linear, covalently closed circular, dumbbell, hairpin or other forms.

The insert region of a nucleic acid library molecule comprises a sequence of interest extracted from any source including a biological sample (e.g., fresh or live sample) such as a single cell, a plurality of cells or tissue. The insert region can be isolated from healthy or diseases cells or tissues. The insert region can be obtained from an archived sample such as a fresh frozen paraffin embedded (FFPE) sample, or from needle biopsies, circulating tumor cells, cell free circulating DNA (e.g., from tumor cells or a fetus). Cells or tissues are typically treated with a lysis buffer to release their DNA and RNA, and the desired nucleic acid is separated from non-desired macromolecules such as proteins.

The insert region (also referred to herein as “sequence of interest” or “insert”) of a nucleic acid library molecule can be isolated in any form, including chromosomal, genomic (e.g., whole genomic), organellar (e.g., mitochondrial, chloroplast or ribosomal), recombinant molecules, cloned or amplified. The insert region of a nucleic acid library molecule can be methylated or non-methylated.

The insert region can be isolated from any organism including viruses, fungi, prokaryotes or eukaryotes. The insert region can be isolated from any organism including human, simian, ape, canine, feline, bovine, equine, murine, porcine, caprine, lupine, ranine, piscine, plant, insect or bacteria. The insert region can be isolated from organisms borne in air, water, soil or food.

The insert region can be isolated from any biological fluid, including blood, urine, serum, lymph, tumor, saliva, anal secretions, vaginal secretions, amniotic samples, perspiration, semen, environmental samples or culture samples. The insert region can be isolated from any organ, including head, neck, brain, breast, ovary, cervix, colon, rectum, endometrium, gallbladder, intestines, bladder, prostate, testicles, liver, lung, kidney, esophagus, pancreas, thyroid, pituitary, thymus, skin, heart, larynx, or other organs.

The insert region can be prepared using recombinant nucleic acid technology including but not limited to any combination of vector cloning, transgenic host cell preparation, host cell culturing and/or PCR amplification.

The insert region can be prepared using chemical synthesis procedures using native nucleotides with or without nucleotide analogs or modified nucleotide linkages that confer certain properties, including resistance to enzymatic digestion, or increased thermal stability. Examples of nucleotide analogs and modified nucleotide linkages that inhibit nuclease digestion include phosphorothioate, 2′-O-methyl RNA, inverted dT, and 2′ 3′ dideoxy-dT. Insert regions that include locked nucleic acids (LNA) have increased thermal stability.

The insert region of a library molecule can be in fragmented or un-fragmented form. Fragmented insert regions can be obtained by subjecting input nucleic acids to mechanical force, enzymatic and/or chemical fragmentation methods. The fragmented insert regions can be generated using procedures that yield a population of fragments having overlapping sequences or non-overlapping sequences.

Mechanical fragmentation typically generates randomly fragmented nucleic acid molecules. Mechanical fragmentation methods include mechanical shearing such as fluid shear, constant shear and pulsatile shear. Mechanical fragmentation methods also include mechanical stress including sonication, nebulization and acoustic cavitation. In some embodiments focused acoustic energy can be used to randomly fragment nucleic acid molecules. A commercially-available apparatus (e.g., Covaris) can be used to fragment nucleic acid molecules using focused acoustic energy.

Enzymatic fragmentation procedures can be conducted under conditions suitable to generate randomly or non-randomly fragmented nucleic acid molecules. For example, restriction endonuclease enzyme digestion can be conducted to completion to generate non-randomly fragmented nucleic acid molecule. Alternatively, partial or incomplete restriction enzyme digestion can be conducted to generate randomly-fragmented nucleic acid molecules. Enzymatic fragmentation using restriction endonuclease enzymes includes any one or any combination of two or more restriction enzymes selected from a group consisting of type I, type II, type IIs, type IIB, type III, or type IV restriction enzymes. Enzymatic fragmentation includes digestion of the nucleic acid with a rare-cutting restriction enzyme, comprising Not I, Asc I, Bae I, AspC I, Pac I, Fse I, Sap I, Sfi I or Psr I. Enzymatic fragmentation includes use of any combination of a nicking restriction endonuclease, endonuclease and/or exonuclease. Enzymatic fragmentation can be achieved by conducting a nick translation reaction.

In some embodiments, enzymatic fragmentation can be achieved by reacting nucleic acids with an enzyme mixture, for example an enzyme that generates single-stranded nicks and another enzyme that catalyzes double-stranded cleavage. An exemplary enzyme mixture is FRAGMENTASE® (e.g., from New England Biolabs).

The fragments can be generated by employing PCR using sequence-specific primers that hybridize to target regions in the input nucleic acids (e.g., genomic DNA) to generate insert regions having known fragment lengths and sequences.

Targeted genome fragmentation methods using CRISPR/Cas9 can be used to generate the fragments.

The fragments can also be generated using a transposase-based tagmentation method using NEXTERA® (from Epicentre).

The insert region can be single-stranded or double-stranded. The ends of the double-stranded insert region can be blunt-ended, or have a 5′ overhang or a 3′ overhang end, or any combination thereof. One or both ends of the insert region can be subjected to an enzymatic tailing reaction to generate a non-template poly-A tail by employing a terminal transferase reaction. The ends of the insert region can be compatible for joining to at least one adaptor sequence.

The insert region can be any length, for example the insert region can be about 50-250, or about 250-500, or about 500-750, or about 750-1000 bases or base pairs in length. In some embodiments, the insert region can be 1000-5000 bases or base pairs in length.

The fragments containing the insert region can be subjected to a size selection process, or the fragments are not size selected. For example, the fragments can be size selected by gel electrophoresis and gel slice extraction. The fragments can be size selected using a solid phase adherence/immobilization method which typically employs micro paramagnetic beads coated with a chemical functional group that interacts with nucleic acids under certain ionic strength conditions with or without polyethylene glycol or polyalkylene glycol. Commercially-available solid phase adherence beads include SPRI (Solid Phase Reversible Immobilization) beads from Beckman Coulter (AMPUR XP paramagnetic beads, catalog No. B23318), MAGNA PURE magnetic glass particles (Roche Diagnostics, catalog No. 03003990001), MAGNASIL paramagnetic beads from Promega (catalog No. MD1360), MAGTRATION paramagnetic beads and system from Precision System Science (catalog Nos. A1120 and A1060), MAG-BIND from Omega Bio-Tek (catalog No. M1378-01), MAGPREP silica from Millipore (catalog No. 101193), SNARE DNA purification systems from Bangs Laboratories (catalog Nos. BP691, BP692 and BP693), and CHEMAGEN M-PVA beads from Perkin Elmer (catalog No. CMG-200).

In some embodiments, the fragmented nucleic acids can be subjected to enzymatic reactions for end-repair and/or A-tailing. The fragmented nucleic acids can be contacted with a plurality of enzymes under a condition suitable to generate nucleic acid fragments having blunt-ended 5′ phosphorylated ends. In some embodiments, the plurality of enzymes generates blunt-ended fragment having a non-template A-tail at their 3′ ends. The plurality of enzymes comprise two or more enzymes that can catalyze nucleic acid end-repair, phosphorylation and/or A-tailing. In some embodiments, the end-repair enzymes include a DNA polymerase (e.g., T4 DNA polymerase) and/or Klenow fragment. In some embodiments, the 5′ end phosphorylation enzyme comprises T4 polynucleotide kinase. In some embodiments, the A-tailing enzyme includes a Taq polymerase (e.g., non-proof-reading polymerase) and dATP. In some embodiments, the fragmenting, end-repair, phosphorylation and A-tailing can be conducted in a one-pot reaction using a mixture of enzymes or can be conducted in separate reactions.

### Enrichment Workflows

The present disclosure provides methods for preparing a nucleic acid library. In some embodiments, the nucleic acids (e.g., sequence of interest) can be subjected to an enrichment workflow before and/or after appending the nucleic acids to at least one adaptor sequence.

In some embodiments, prior to appending the nucleic acids to at least one adaptor, the nucleic acids can be subjected to an enrichment workflow. For example, the mixture of nucleic acids comprises target and non-target sequences. In some embodiments, the mixture of nucleic acids can be hybridized to a plurality of enrichment oligonucleotides which are immobilized to a support as part of an enrichment array. In some embodiments, the enrichment oligonucleotides comprise the same target sequence or a mixture of different target sequences that can selectively hybridize to the nucleic acids having a target sequence. In some embodiments, the enrichment oligonucleotides of the enrichment array may or may not include a cleavable moiety (e.g., a restriction endonuclease recognition sequence or at least one uracil). The conditions for hybridization to the enrichment array can be suitable for selectively hybridizing the target nucleic acids to the enrichment array. In some embodiments, after the hybridization step the non-hybridized nucleic acids can be washed away under a condition suitable to retain the target nucleic acids which are hybridized to the enrichment array. In some embodiments, the target nucleic acids can be eluted from the enrichment array thereby generating enriched target nucleic acids. In some embodiments, the enrichment oligonucleotides can be cleaved to release the target nucleic acids from the enrichment array thereby generating enriched target nucleic acids. In some embodiments the enriched nucleic acids can be appended to at least one adaptor sequence.

In some embodiments, after appending the nucleic acids to at least one adaptor to generate nucleic acid library molecules, the library molecules can be subjected to an enrichment workflow. In some embodiments, individual library molecules comprise an insert sequence appended with at least one adaptor sequence (e.g., universal adaptor sequence). In some embodiments, the library molecules comprise a mixture of target and non-target insert sequences. In some embodiments, the library molecules can be subjected to the step of hybridizing to a plurality of blocking oligonucleotides that selectively hybridize to an adaptor sequence (e.g., a universal adaptor sequence) of the library molecules. In some embodiments, at least one blocking oligonucleotide may or may not include a cleavable moiety (e.g., a restriction endonuclease recognition sequence or at least one uracil). In some embodiments, the library molecules can be hybridized to first blocker oligonucleotides which selectively hybridize to a first universal adaptor sequence in a library molecules. In some embodiments, in an optional step, the library molecules can be hybridized to second blocker oligonucleotides which selectively hybridize to a second universal adaptor sequence in a library molecules. In some embodiments, the first and/or second blocker oligonucleotides comprise biotinylated blocker oligonucleotides which are capable of binding streptavidin on paramagnetic beads. In some embodiments, the first and/or second blocking oligonucleotides comprise at least one uracil base. In some embodiments, the library molecules can be hybridized to a plurality of enrichment oligonucleotides which are immobilized to a support as part of an enrichment array. In some embodiments, the enrichment oligonucleotides comprise the same target sequence or a mixture of different target sequences that can selectively hybridize to the target insert regions of the library molecules. In some embodiments, the enrichment oligonucleotides of the enrichment array may or may not include a cleavable moiety (e.g., a restriction endonuclease recognition sequence or at least one uracil). The conditions for hybridization to the enrichment array can be suitable for selectively hybridizing the target insert regions of the library molecules to the enrichment array. In some embodiments, after the hybridization step the non-hybridized library molecules can be washed away under a condition suitable to retain the target library molecules which are hybridized to the enrichment array. In some embodiments, the target library molecules can be eluted from the enrichment array thereby generating a plurality of library molecules enriched for target insert regions. In some embodiments, the enrichment oligonucleotides can be cleaved to release the target library molecules from the enrichment array thereby generating a plurality of library molecules enriched for target insert regions. In some embodiments, the enriched library molecules can optionally be appended to at least one additional adaptor sequence. In some embodiments, the blocking oligonucleotides that are hybridized to the eluted or released library molecules can be reacted with a restriction enzyme to cleave the cleavable moiety in the blocking oligonucleotide thereby removing the blocking oligonucleotide from the library molecule. In some embodiments, the blocking oligonucleotides that are hybridized to the eluted or released library molecules can be treated with an enzyme or enzyme cocktail to convert the uracil in the blocking oligo to an abasic site and the abasic site can be removed to generate a gap-containing blocking oligonucleotide. In some embodiments, the gap-containing blocking oligonucleotide can be removed from the enriched library molecules. In some embodiments, the enzymes that generated the abasic site and gap site comprise uracil DNA glycosylase (UDG) and DNA glycosylase-lyase endonuclease VIII, respectively.

In some embodiments, prior to appending the nucleic acids to at least one adaptor, the nucleic acids can be subjected to an enrichment workflow. For example, the mixture of nucleic acids comprises target and non-target sequences. In some embodiments, the mixture of nucleic acids can be hybridized to a plurality of soluble enrichment oligonucleotides which are biotinylated and capable of binding streptavidin on paramagnetic beads. In some embodiments, the enrichment oligonucleotides comprise the same target sequence or a mixture of different target sequences that can selectively hybridize to the nucleic acids having a target sequence. In some embodiments, the enrichment oligonucleotides may or may not include a cleavable moiety (e.g., a restriction endonuclease recognition sequence or at least one uracil). The conditions for hybridization can be suitable for selectively hybridizing the target nucleic acids to the soluble enrichment oligonucleotides. In some embodiments, after the hybridization step, the biotinylated enrichment oligonucleotides can be reacted with a plurality of streptavidin-coated paramagnetic beads to form enrichment oligonucleotide-bead complexes which include target nucleic acids hybridized to biotinylated enrichment oligonucleotides. In some embodiments, a magnet can be used to separate the enrichment oligonucleotide-bead complexes from the non-hybridized nucleic acids. The non-hybridized nucleic acids can be washed away under a condition suitable to retain the target nucleic acids which are hybridized to the biotinylated enrichment oligonucleotides. In some embodiments, the target nucleic acids can be eluted from the enrichment oligonucleotides and appended to at least one adaptor sequence. In some embodiments, the enrichment oligonucleotides can be cleaved to release the target nucleic acids, and the released target nucleic acids can be appended to at least one adaptor sequence.

In some embodiments, after appending the nucleic acids to at least one adaptor to generate nucleic acid library molecules, the library molecules can be subjected to an enrichment workflow. In some embodiments, individual library molecules comprise an insert sequence appended with at least one adaptor sequence (e.g., universal adaptor sequence). In some embodiments, the library molecules comprise a mixture of target and non-target insert sequences. In some embodiments, the library molecules can be subjected to the step of hybridizing to a plurality of blocking oligonucleotides that selectively hybridize to an adaptor sequence (e.g., a universal adaptor sequence) of the library molecules. In some embodiments, at least one blocking oligonucleotide may or may not include a cleavable moiety (e.g., a restriction endonuclease recognition sequence or at least one uracil). In some embodiments, the library molecules can be hybridized to first blocker oligonucleotides which selectively hybridize to a first universal adaptor sequence in a library molecules. In some embodiments, in an optional step, the library molecules can be hybridized to second blocker oligonucleotides which selectively hybridize to a second universal adaptor sequence in a library molecules. In some embodiments, the first and/or second blocker oligonucleotides comprise biotinylated blocker oligonucleotides which are capable of binding streptavidin on paramagnetic beads. In some embodiments, the first and/or second blocking oligonucleotides comprise at least one uracil base. In some embodiments, the enrichment method further comprises hybridizing the library molecules to a plurality of soluble enrichment oligonucleotides which are biotinylated and capable of binding streptavidin on a paramagnetic bead. In some embodiments, the enrichment oligonucleotides comprise the same target sequence or a mixture of different target sequences that can hybridize to the library molecules having an insert region with a target sequence. In some embodiments, the enrichment oligonucleotides may or may not include a cleavable moiety (e.g., a restriction endonuclease recognition sequence or at least one uracil). The conditions for hybridization can be suitable for selectively hybridizing the target insert regions of the library molecules to the soluble enrichment oligonucleotides. In some embodiments, after the hybridization step, the biotinylated enrichment oligonucleotides can be reacted with a plurality of streptavidin-coated paramagnetic beads to form enrichment oligonucleotide-bead complexes which include target library molecules hybridized to biotinylated enrichment oligonucleotides. In some embodiments, a magnet can be used to separate the enrichment oligonucleotide-bead complexes from the non-hybridized library molecules. The non-hybridized library molecules can be washed away under a condition suitable to retain the target library molecules which are hybridized to the biotinylated enrichment oligonucleotides. In some embodiments, the target library molecules can be eluted from the enrichment oligonucleotides thereby generating a plurality of library molecules enriched for target insert regions. In some embodiments, the enrichment oligonucleotides can be cleaved to release the target library molecules thereby generating a plurality of library molecules enriched for target insert regions. In some embodiments, the enriched library molecules can optionally be appended to at least one additional adaptor sequence. In some embodiments, the blocking oligonucleotides that are hybridized to the eluted or released library molecules can be reacted with a restriction enzyme to cleave the cleavable moiety in the blocking oligonucleotide thereby removing the blocking oligonucleotide from the library molecule. In some embodiments, the blocking oligonucleotides that are hybridized to the eluted or released library molecules can be treated with an enzyme or enzyme cocktail to convert the uracil in the blocking oligo to an abasic site and the abasic site can be removed to generate a gap-containing blocking oligonucleotide. In some embodiments, the gap-containing blocking oligonucleotide can be removed from the enriched library molecules. In some embodiments, the enzymes that generated the abasic site and gap site comprise uracil DNA glycosylase (UDG) and DNA glycosylase-lyase endonuclease VIII, respectively.

### Appending Adaptors to Nucleic Acids

The present disclosure provides methods for preparing a nucleic acid library. In some embodiments, the nucleic acids can be appended to one or more adaptors. In some embodiments, an adaptor comprises an oligonucleotide that can be operably linked (appended) to a nucleic acid, where the adaptor comprises a sequence that confers a function to the co-joined adaptor-nucleic acid molecule. In some embodiments, individual nucleic acids can be covalently joined to at least one universal adaptor sequence for library preparation. In general, a nucleic acid can be covalently joined at both ends to one or more universal adaptors to generate a linear library molecule having the arrangement left adaptor-insert-right adaptor. In some embodiments, at least one nucleic acid in the population of nucleic acids comprises a sequence of interest. Individual library molecules in the population of library molecules can have an insert region that is the same or different as other library molecules in the population. In some embodiments, about 1-10 ng, or about 10-50 ng, or about 50-100 ng of input nucleic acids (e.g., fragmented nucleic acids) can be appended to one or more universal adaptors to generate a linear library.

Individual nucleic acids can be appended on one or both ends to at least one universal adaptor sequence to form a recombinant nucleic acid linear library molecule having the general arrangement left adaptor-insert-right adaptor.

In some embodiments, the nucleic acids (e.g., sequence of interest or insert region) can be appended with any one or any combination of two or more adaptor sequences in any order, where the adaptor sequences comprise: (i) a first universal binding site (120) for a first portion of a splint capture primer (or a complementary sequence thereof); (ii) a universal binding site for a first non-splint capture primer (123) (or a complementary sequence thereof); (iii) at least one sample index sequence (e.g., (160) and/or (170) which can be used to distinguish sequences of interest obtained from different sample sources in a multiplex assay; (iv) at least one universal binding site for a forward sequencing primer (140) (or a complementary sequence thereof); (v) at least one universal binding site for a reverse sequencing primer (150) (or a complementary sequence thereof); (vi) at least one universal binding site for a compaction oligonucleotide (or a complementary sequence thereof); (vii) at least one unique molecular index sequence (UMI) (e.g., (180) and/or (190)) which can be used to uniquely identify a nucleic acid molecule (e.g., having a sequence of interest) to which the unique molecular index sequence is appended; (viii) at least one universal binding site for a pinning primer (or a complementary sequence thereof); (ix) at least one universal binding site for a soluble amplification primer; (x) at least one batch-specific barcode sequence; (xi) a universal binding site for a second non-splint capture primer (133) (or a complementary sequence thereof); (xii) at least one short random sequence (e.g., NNNN) (132) which provides nucleotide sequence diversity and is about 3-20 nucleotides in length; and/or (xiii) a second universal binding site (130) for a second portion of the immobilized splint capture (or a complementary sequence thereof).

In some embodiments, the universal binding site for a forward sequencing primer (140) comprises a batch-specific forward sequencing primer binding site which can be employed for batch sequencing.

In some embodiments, the universal binding site for a reverse sequencing primer (150) comprises a batch-specific reverse sequencing primer binding site which can be employed for batch sequencing.

In some embodiments, the at least one sample index sequence (e.g., (160) and/or (170) comprises a sample index sequence joined to a short random sequence (e.g., NNN), where the short random sequence provides nucleotide sequence diversity and is about 3-20 nucleotides in length.

The universal adaptors can be prepared using chemical synthesis procedures using native nucleotides with or without nucleotide analogs or modified nucleotide linkages that confer certain properties, including resistance to enzymatic digestion, or increased thermal stability. Examples of nucleotide analogs and modified nucleotide linkages that inhibit nuclease digestion include phosphorothioate, 2′-O-methyl RNA, inverted dT, and 2′ 3′ dideoxy-dT. Insert regions that include locked nucleic acids (LNA) have increased thermal stability.

In some embodiments, the insert region can be joined at one or both ends to at least one universal adaptor sequence using a ligase enzyme and/or primer extension reaction to generate a linear library molecule. Covalent linkage between an insert region and the universal adaptor(s) can be achieved with a DNA or RNA ligase. Exemplary DNA ligases that can ligate double-stranded DNA molecules include T4 DNA ligase and T7 DNA ligase. A universal adaptor sequence can be appended to an insert sequence by PCR using a tailed primer having 5′ region carrying a universal adaptor sequence and a 3′ region that is complementary to a portion of the insert sequence. A universal adaptor sequence can be appended to an insert sequence which is flanked on one side or both sides with first and second universal adaptor sequences by PCR using a tailed primer having 5′ region carrying a third universal adaptor sequence and a 3′ region that is complementary to a portion of the first or second adaptor sequence.

In some embodiments, a linear library molecule comprises a sequence of interest and any one or any combination of two or more adaptor sequences in any order, where the adaptor sequences comprise: (i) a first universal binding site (120) for a first portion of a splint capture primer (or a complementary sequence thereof); (ii) a universal binding site=for a first non-splint capture primer (123) (or a complementary sequence thereof); (iii) at least one sample index sequence (e.g., (160) and/or (170) which can be used to distinguish sequences of interest obtained from different sample sources in a multiplex assay; (iv) at least one universal binding site for a forward sequencing primer (140) (or a complementary sequence thereof); (v) at least one universal binding site for a reverse sequencing primer (150) (or a complementary sequence thereof); (vi) at least one universal binding site for a compaction oligonucleotide (or a complementary sequence thereof); (vii) at least one unique molecular index sequence (UMI) (e.g., (180) and/or (190)) which can be used to uniquely identify a nucleic acid molecule (e.g., having a sequence of interest) to which the unique molecular index sequence is appended; (viii) at least one universal binding site for a pinning primer (or a complementary sequence thereof); (ix) at least one universal binding site for a soluble amplification primer; (x) at least one batch-specific barcode sequence; (xi) a universal binding site for a second non-splint capture primer (133) (or a complementary sequence thereof); (xii) at least one short random sequence (e.g., NNNN) (132) which provides nucleotide sequence diversity and is about 3-20 nucleotides in length; and/or (xiii) a second universal binding site (130) for a second portion of the immobilized splint capture (or a complementary sequence thereof).

In some embodiments, the universal binding site for a forward sequencing primer (140) comprises a batch-specific forward sequencing primer binding site which can be employed for batch sequencing.

In some embodiments, the universal binding site for a reverse sequencing primer (150) comprises a batch-specific reverse sequencing primer binding site which can be employed for batch sequencing.

In some embodiments, the at least one sample index sequence (e.g., (160) and/or (170) comprises a sample index sequence joined to a short random sequence (e.g., NNN), where the short random sequence provides nucleotide sequence diversity and is about 3-20 nucleotides in length.

In some embodiments, the sequence of interest can be joined to at least one adaptor sequence by appending one or more double stranded linear adaptor(s) and/or Y-shaped adaptors which carry any one or any combination of two or more of the adaptor sequences listed above.

Exemplary linear library molecules are shown in FIGS. 20-24. The skilled artisan will recognize that linear library molecules having adaptor sequences constructed with other arrangements are possible.

In some embodiments, the linear library molecule (100) comprises a left unique molecular index sequence (180) and/or a right unique molecular index sequence (190). In some embodiments, the left unique molecular index sequence (180) and the right unique molecular index sequence (190) are unique molecular tags each comprising a sequence that can be used to uniquely identify an individual sequence of interest (e.g., insert sequence) to which the unique molecular index is/are appended in a population of other sequence of interest molecules. In some embodiments, the left unique molecular index sequence (180) and/or the right unique molecular index sequence (190) can be used for molecular tagging. In some embodiments, the unique molecular index sequence (180) and/or (190) comprises 2-12 or more nucleotides having a known sequence. For example, the unique molecular index sequence comprises a known random sequence where a nucleotide at each position is randomly selected from nucleotides having a base A, G, C, T or U. The unique molecular index sequences (180) and/or (190) can be used for molecular tagging procedures.

Multiplex workflows are enabled by preparing sample-indexed linear libraries using one or both sample index sequences (e.g., left and/or right sample index sequences). The left sample index sequences (160) and/or right sample index sequences (170) can be employed to prepare separate sample-indexed libraries using input nucleic acids isolated from different sources. The sample-indexed libraries can be pooled together to generate a multiplex library mixture, and the pooled libraries can be circularized, amplified and/or sequenced. The sequences of the insert region (110) along with the left sample index sequence (160) and/or right sample index sequence (170) can be used to identify the source of the input nucleic acids. The sample index sequences can be used to distinguish sequences of interest obtained from different sample sources in a multiplex assay. In some embodiments, any number of sample-indexed libraries can be pooled together, for example 2-10, or 10-50, or 50-100, or 100-200, or more than 200 sample-indexed libraries can be pooled together. Exemplary nucleic acid sources include naturally-occurring, recombinant, or chemically-synthesized sources. Exemplary nucleic acid sources include single cells, a plurality of cells, tissue, biological fluid, environmental sample or whole organism. Exemplary nucleic acid sources include fresh, frozen, fresh-frozen or archived sources (e.g., formalin-fixed paraffin-embedded; FFPE). The skilled artisan will recognize that the nucleic acids can be isolated from many other sources. The sample-indexed linear library molecules can be prepared in single-stranded or double-stranded form.

In some embodiments, the left sample index sequence (160) can be 3-20 nucleotides in length. In some embodiments, the right sample index sequence (170) can be 3-20 nucleotides in length. The sequences of the left and right sample index sequences (e.g., (160) and (170)) can be the same or different from each other. In some embodiments, one or both of the sample index sequences (160) and/or (170) includes a short random sequence (e.g., NNN). The short random sequence can be about 3-20 nucleotides in length.

In some embodiments, the linear library molecule (100) comprises a universal binding site for a first non-splint capture primer (123) (or a complementary sequence thereof) and a universal binding site for a second non-splint primer (133) (or a complementary sequence thereof). In some embodiments, the first and second non-splint capture primers comprises capture primer that are employed for conducting bridge amplification. In some embodiments, the first non-splint capture primer comprises a P5 capture primer. In some embodiments, the second non-splint capture primer comprises a P7 capture primer.

In some embodiments, the linear library molecule (100) comprises a universal binding site for a first non-splint capture primer (123) which comprises the sequence 5′-AATGATACGGCGACCACCGA-3′ (SEQ ID NO:161) where the universal binding site (123) or the complementary sequence can bind a P5 capture primer.

In some embodiments, the linear library molecule (100) comprises a universal binding site for a second non-splint capture primer (133) which comprises the sequence 5′-TCGTATGCCGTCTTCTGCTTG-3′ (SEQ ID NO:162) where the universal binding site (133) or the complementary sequence can bind a P7 capture primer.

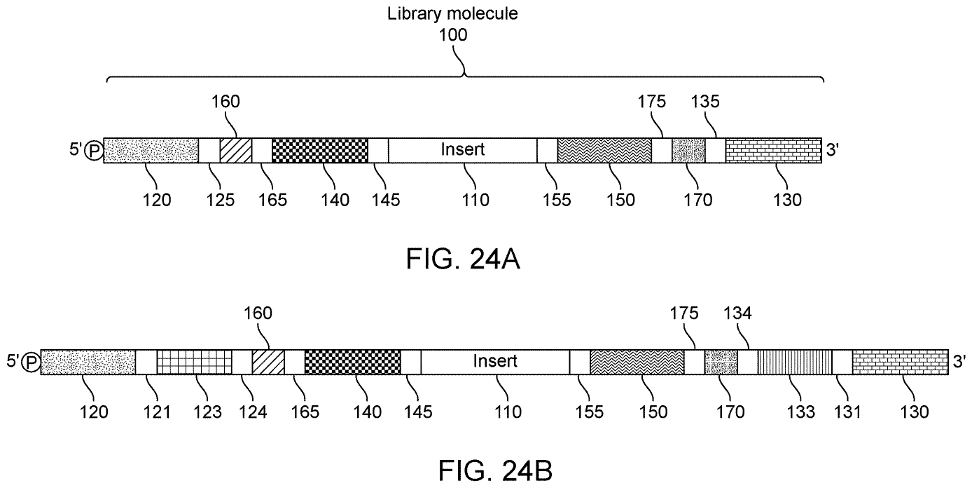

In some embodiments, the linear library molecule (100) further comprises at least one junction adaptor sequence located between any of the universal adaptor sequences described herein (e.g., see FIG. 24, top schematic).

For example, a junction adaptor sequence (125) can be located between a first universal binding site (120) for a first portion of a splint capture primer and a left sample index sequence (160). A junction adaptor sequence (165) can be located between a left sample index sequence (160) and a universal binding site for a forward sequencing primer (140). A junction adaptor sequence (145) can be located between a universal binding site for a forward sequencing primer (140) and the sequence of interest (110). A junction adaptor sequence (135) can be located between a second universal binding site (130) for a second portion of the immobilized splint capture and a right sample index sequence (170). A junction adaptor sequence (175) can be located between a right sample index sequence (170) and universal binding site for a reverse sequencing primer (150). A junction adaptor sequence (155) can be located between universal binding site for a reverse sequencing primer (150) and the sequence of interest (110).

In some embodiments, the linear library molecule (100) further comprises at least one junction adaptor sequence located between any of the universal adaptor sequences described herein (e.g., FIG. 24, bottom schematic).

For example, a junction adaptor sequence (121) can be located between a first universal binding site (120) for a first portion of a splint capture primer (200) and a universal binding site for a first non-splint capture primer (123). A junction adaptor sequence (124) can be located between a universal binding site for a first non-splint capture primer (123) and a left sample index sequence (160). A junction adaptor sequence (165) can be located between a left sample index sequence (160) and a universal binding site for a forward sequencing primer (140). A junction adaptor sequence (145) can be located between a universal binding site for a forward sequencing primer (140) and the sequence of interest (110). A junction adaptor sequence (131) can be located between a second universal binding site (130) for a second portion of the immobilized splint capture and a universal binding site for a second non-splint capture primer (133). A junction adaptor sequence (134) can be located between a universal binding site for a second non-splint capture primer (133) and a right sample index sequence (170). A junction adaptor sequence (175) can be located between a right sample index sequence (170) and a universal binding site for a reverse sequencing primer (150). A junction adaptor sequence (155) can be located between universal binding site for a reverse sequencing primer (150) and the sequence of interest (110).

Any of the junction adaptor sequences can comprise any sequence and can be 3-60 nucleotides in length. Any of the junction adaptor sequences can comprise a universal sequence or a unique sequence. Any of the junction adaptor sequences comprise a random sequence (e.g., NNN) having 3-20 nucleotides. Any of the junction adaptor sequences can comprise a binding sequence for an amplification primer, a sequencing primer or a compaction oligonucleotide. Any of the junction adaptor sequences can comprise a binding sequence for an splint capture primer (200) which is immobilized on the support. Any of the junction adaptor sequences can comprise a sample index sequence. Any of the junction adaptor sequences can comprise a unique molecular index sequence. Any of the junction adaptor sequences, particularly junction adaptor sequence (145) can comprise a Tn5 transposon-end sequence 5′-AGATGTGTATAAGAGACAG-3′ (SEQ ID NO: 1). Any of the junction adaptor sequences, particularly junction adaptor sequence (155) can comprise a Tn5 transposon-end sequence 5′-CTGTCTCTTATACACATCT-3′ (SEQ ID NO: 2). The Tn5 transposon-end sequences can be introduced into the linear library molecule (100) via a transposase-mediated reaction which includes contacting double-stranded input DNA (e.g., genomic DNA) with a Tn-5 type transposase enzyme, and a double-stranded oligonucleotide comprising the Tn transposon-end sequence (SEQ ID NO: 1) linked to a universal adaptor sequence or a sample index sequence under a condition that is suitable to form a transposon synaptic complex. In the double-stranded oligonucleotide, the Tn transposon-end sequence (SEQ ID NO:1) can be located 5′ or 3′ relative to the universal adaptor sequence or a sample index sequence.

In some embodiments, a linear library molecule (100) can be generated by employing a ligation reaction and an optional primer extension reaction. The library molecule can be generated by joining at least one end of a double-stranded insert region (110) to a double-stranded adaptor which comprises two nucleic acid strands that are fully complementary along their lengths. In some embodiments, the library molecule can be generated by joining the first end of a double-stranded insert region (110) to a first double-stranded adaptor, and joining the second end of the double-stranded insert region (110) to a second double-stranded adaptor.

In some embodiments, the library molecule can be generated by joining the first end of a double-stranded insert region (110) to a first double-stranded adaptor having a universal binding site for a forward sequencing primer (140), and joining the second end of the double-stranded insert region (110) to a second double-stranded adaptor having universal binding site for a reverse sequencing primer (150), wherein the joining is conducted using a DNA ligase enzyme to generate a double-stranded recombinant molecule. In some embodiments, the first double-stranded adaptor further comprises a left sample index sequence (160). In some embodiments, the second double-stranded adaptor further comprises a right sample index sequence (170). In some embodiments, the ligating end of the first and/or the second double-stranded adaptors comprise a blunt end, or an overhang end (e.g., 5′ or 3′ overhang end).

In some embodiments the first and second double-stranded adaptors carry the same adaptor sequences. In some embodiments the first and second double-stranded adaptors carry different adaptor sequences.

In some embodiments, the first double-stranded adaptor comprises any one or any combination of two or more adaptor sequences and in any order: at least a portion of universal binding site for a forward sequencing primer (140) (or a complementary sequence thereof); a left sample index sequence (160); and/or a first universal binding site (120) for a first portion of a splint capture primer (or a complementary sequence thereof). In some embodiments, the left sample index sequence (160) comprises a short random sequence (e.g., NNN) that is about 3-20 nucleotides in length.

In some embodiments, the second double-stranded adaptor comprises any one or any combination of two or more adaptor sequences and in any order: at least a portion of a universal binding site for a reverse sequencing primer (150) (or a complementary sequence thereof); a right sample index sequence (170); and/or a second universal binding site (130) for a second portion of the immobilized splint capture (or a complementary sequence thereof). In some embodiments, the right sample index sequence (170) comprises a short random sequence (e.g., NNN) that is about 3-20 nucleotides in length.

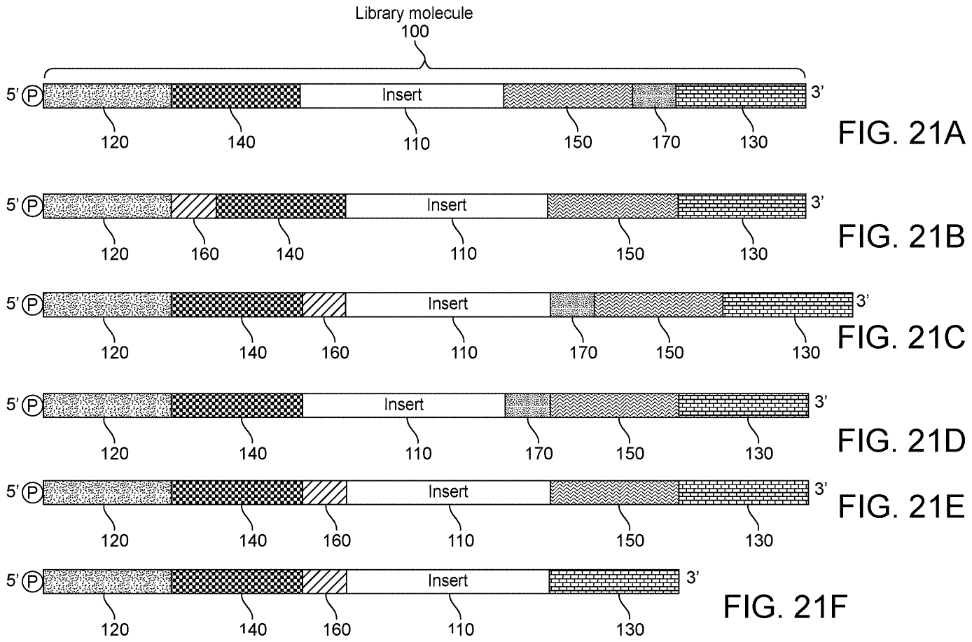

In some embodiments, the double-stranded insert region (110) can be joined to the first and second double-stranded adaptors using a DNA ligase enzyme to generate a double-stranded recombinant molecule comprising an insert region (110) flanked on one side with adaptor sequences comprising (i) a universal binding site for a forward sequencing primer (140) (or a complementary sequence thereof), (ii) a left sample index sequence (160), and (iii) a first universal binding site (120) for a first portion of a splint capture primer (or a complementary sequence thereof); and the insert region (110) can be flanked on the other side with adaptor sequences comprising (i) a universal binding site for a reverse sequencing primer (150) (or a complementary sequence thereof); (ii) a right sample index sequence (170); and (iii) a second universal binding site (130) for a second portion of the immobilized splint capture (or a complementary sequence thereof) (e.g., see FIGS. 20A-D and FIGS. 21A-F).

In some embodiments, the first double-stranded adaptor comprises any one or any combination of two or more adaptor sequences and in any order: at least a portion of universal binding site for a forward sequencing primer (140) (or a complementary sequence thereof); a left sample index sequence (160); a universal binding site for a first non-splint capture primer (123) (or a complementary sequence thereof); and/or a first universal binding site (120) for a first portion of a splint capture primer (or a complementary sequence thereof). In some embodiments, the left sample index sequence (160) comprises a short random sequence (e.g., NNN) that is about 3-20 nucleotides in length.

In some embodiments, the second double-stranded adaptor comprises any one or any combination of two or more adaptor sequences and in any order: at least a portion of a universal binding site for a reverse sequencing primer (150), a right sample index sequence (170), a universal binding site for a second non-splint capture primer (133) (or a complementary sequence thereof), and a second universal binding site (130) for a second portion of the immobilized splint capture (or a complementary sequence thereof). In some embodiments, the right sample index sequence (170) comprises a short random sequence (e.g., NNN) that is about 3-20 nucleotides in length.

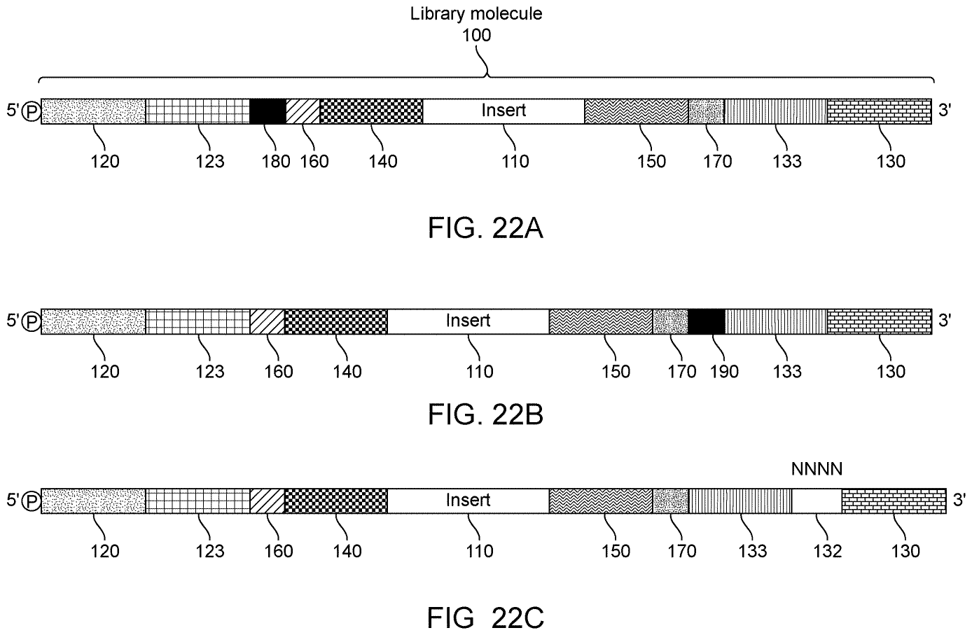

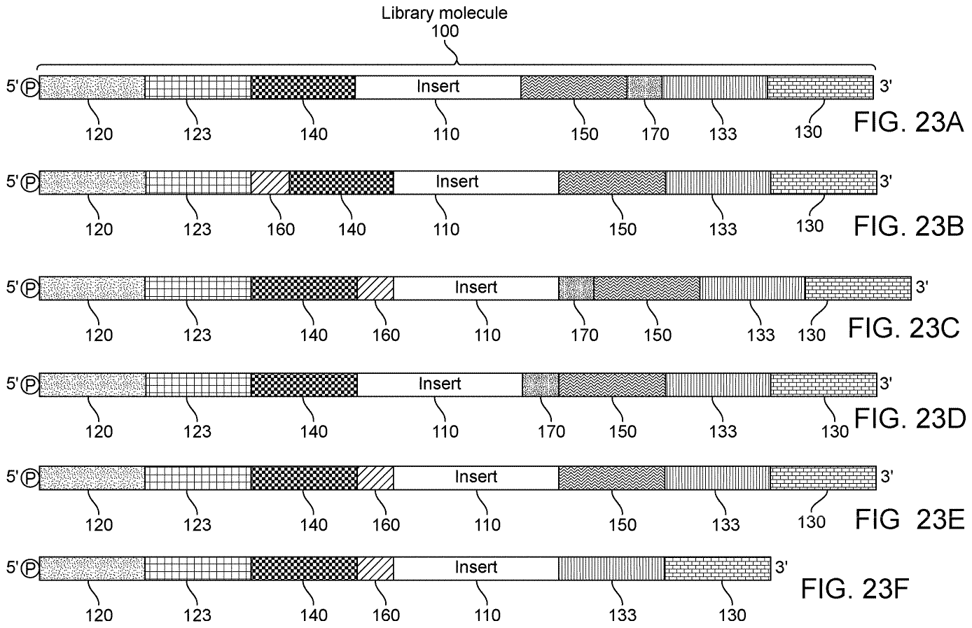

In some embodiments, the double-stranded insert region (110) can be joined to the first and second double-stranded adaptors using a DNA ligase enzyme to generate a double-stranded recombinant molecule comprising an insert region (110) flanked on one side with adaptor sequences comprising (i) a universal binding site for a forward sequencing primer (140) (or a complementary sequence thereof), (ii) a left sample index sequence (160), (iii) a universal binding site for a first non-splint capture primer (123) (or a complementary sequence thereof), and (iv) a first universal binding site (120) for a first portion of a splint capture primer (or a complementary sequence thereof); and the insert region (110) can be flanked on the other side with adaptor sequences comprising (i) a universal binding site for a reverse sequencing primer (150) (or a complementary sequence thereof); (ii) a right sample index sequence (170); (iii) a universal binding site for a second non-splint capture primer (133) (or a complementary sequence thereof), and (iv) a second universal binding site (130) for a second portion of the immobilized splint capture (or a complementary sequence thereof) (e.g., see FIGS. 22A-22C and FIGS. 23A-23F).

In some embodiments, a linear library molecule (100) can be generated by employing a ligation reaction and optionally a primer extension reaction. The library molecule can be generated by joining at least one end of a double-stranded insert region (110) to a double-stranded Y-shaped adaptor which comprises two nucleic acid strands, where a portion of the two strands are fully complementary to each other and are annealed together and another portion of the two strands are not complementary to each other and are mismatched. In some embodiments, the library molecule can be generated by joining the first end of a double-stranded insert region (110) to a first double-stranded Y-shaped adaptor (e.g., a first forked adaptor), and joining the second end of a double-stranded insert region (110) to a second double-stranded Y-shaped adaptor (e.g., a second forked adaptor). In some embodiments, the ligating end of the first and second Y-shaped adaptors comprise an annealed portion that forms a blunt end or an overhang end (e.g., 5′ or 3′ overhang end). In some embodiments the first and second Y-shaped adaptors carry the same adaptor sequences. In some embodiments the first and second Y-shaped adaptors carry different adaptor sequences. In some embodiments, the first Y-shaped adaptors comprise partial length “stubby” Y-shaped adaptors or full length Y-shaped adaptors. In some embodiments, the second Y-shaped adaptors comprise partial length “stubby” Y-shaped adaptors or full length Y-shaped adaptors.

In some embodiments, the first strand of the annealed portion and/or the mismatched portion of the Y-shaped adaptor comprises any one or any combination of two or more adaptor sequences and in any order: at least a portion of a universal binding site for a forward sequencing primer (140) (or a complementary sequence thereof); a left sample index sequence (160); and/or a first universal binding site (120) for a first portion of a splint capture primer (or a complementary sequence thereof). In some embodiments, the left sample index sequence (160) comprises a short random sequence (e.g., NNN) that is about 3-20 nucleotides in length.

In some embodiments, the second strand of the annealed portion and/or the mismatched portion of the Y-shaped adaptor comprises any one or any combination of two or more adaptor sequences and in any order: at least a portion of universal binding site for a reverse sequencing primer (150) (or a complementary sequence thereof); a right sample index sequence (170); and/or a second universal binding site (130) for a second portion of the immobilized splint capture (or a complementary sequence thereof). In some embodiments, the right sample index sequence (170) comprises a short random sequence (e.g., NNN) that is about 3-20 nucleotides in length.

In some embodiments, the double-stranded insert region (110) can be joined to the first and second Y-shaped adaptors using a DNA ligase enzyme to generate a double-stranded recombinant molecule comprising an insert region (110) flanked on one side with adaptor sequences comprising (i) a universal binding site for a forward sequencing primer (140) (or a complementary sequence thereof), (ii) a left sample index sequence (160), and (iii) a first universal binding site (120) for a first portion of a splint capture primer (or a complementary sequence thereof); and the insert region (110) can be flanked on the other side with adaptor sequences comprising (i) a universal binding site for a reverse sequencing primer (150) (or a complementary sequence thereof); (ii) a right sample index sequence (170); and (iii) a second universal binding site (130) for a second portion of the immobilized splint capture (or a complementary sequence thereof) (e.g., see FIGS. 20A-D and FIGS. 21A-F).

In some embodiments, the first strand of the annealed portion and/or the mismatched portion of the Y-shaped adaptor comprises any one or any combination of two or more adaptor sequences and in any order: at least a portion of a universal binding site for a forward sequencing primer (140) (or a complementary sequence thereof); a left sample index sequence (160); a universal binding site for a first non-splint capture primer (123) (or a complementary sequence thereof); and/or a first universal binding site (120) for a first portion of a splint capture primer (or a complementary sequence thereof). In some embodiments, the left sample index sequence (160) comprises a short random sequence (e.g., NNN) that is about 3-20 nucleotides in length.

In some embodiments, the second strand of the annealed portion and/or the mismatched portion of the Y-shaped adaptor comprises any one or any combination of two or more adaptor sequences and in any order: at least a portion of universal binding site for a reverse sequencing primer (150) (or a complementary sequence thereof); a right sample index sequence (170); a universal binding site for a second non-splint capture primer (133) (or a complementary sequence thereof); a short random sequence (e.g., NNNN) (132) which provides nucleotide sequence diversity; and/or a second universal binding site (130) for a second portion of the immobilized splint capture (or a complementary sequence thereof). In some embodiments, the right sample index sequence (170) comprises a short random sequence (e.g., NNN) that is about 3-20 nucleotides in length. In some embodiments, the short random sequence (e.g., NNNN) (132) is about 3-20 nucleotides in length.

In some embodiments, the double-stranded insert region (110) can be joined to the first and second Y-shaped adaptors using a DNA ligase enzyme to generate a double-stranded recombinant molecule comprising an insert region (110) flanked on one side with adaptor sequences comprising (i) a universal binding site for a forward sequencing primer (140) (or a complementary sequence thereof), (ii) a left sample index sequence (160), (iii) a universal binding site for a first non-splint capture primer (123) (or a complementary sequence thereof), and (iv) a first universal binding site (120) for a first portion of a splint capture primer (or a complementary sequence thereof); and the insert region (110) can be flanked on the other side with adaptor sequences comprising (i) a universal binding site for a reverse sequencing primer (150) (or a complementary sequence thereof); (ii) a right sample index sequence (170); (iii) a universal binding site for a second non-splint capture primer (133) (or a complementary sequence thereof), and (iv) a second universal binding site (130) for a second portion of the immobilized splint capture (or a complementary sequence thereof) (e.g., see FIGS. 22A-22C and FIGS. 23A-23F).

In some embodiments, the double-stranded recombinant molecules which are generated by ligating the insert region (110) to double-stranded adaptors or Y-shaped adaptors can be subjected to a denaturing condition to generate single-stranded recombinant molecules which can be subjected to a primer extension reaction to append additional adaptor sequences. At least one additional adaptor sequence can be appended to the recombinant molecules by conducting a primer extension reaction using tailed primers (e.g., tailed PCR primers), by contacting/hybridizing the single-stranded recombinant molecules with a plurality of first tailed primers and conducting at least one primer extension reaction. In some embodiments, at least one additional adaptor sequence can be appended to the recombinant molecules by conducting a primer extension reaction using tailed primers (e.g., tailed PCR primers), by contacting/hybridizing the single-stranded recombinant molecules with a plurality of second tailed primers and conducting at least one primer extension reaction.

In some embodiments, the plurality of first tailed primers each comprise a 5′ region carrying a second universal binding site (130) for a second portion of the immobilized splint capture (or a complementary sequence thereof), and a 3′ region that is complementary to at least a portion of a universal binding site for a reverse sequencing primer (150) (or a complementary sequence thereof) of the single-stranded recombinant molecules.

In some embodiments, the plurality of first tailed primers each comprise a 5′ region carrying a second universal binding site (130) for a second portion of the immobilized splint capture (or a complementary sequence thereof) and a sample index sequence (170), and a 3′ region that is complementary to at least a portion of a universal binding site for a reverse sequencing primer (150) (or a complementary sequence thereof) of the single-stranded recombinant molecules. In some embodiments, the sample index sequence (170) comprises a short random sequence (e.g., NNN) that is about 3-20 nucleotides in length.

The method further comprises conducting a second primer extension reaction using a second tailed primer. In some embodiments, the plurality of second tailed primers comprise a 5′ region carrying a first universal binding site (120) for a first portion of a splint capture primer (or a complementary sequence thereof), and a 3′ region that is complementary to at least a portion of universal binding site for a forward sequencing primer (140) (or a complementary sequence thereof) of the first double-stranded tailed molecule.

In some embodiments, the plurality of second tailed primers comprise a 5′ region carrying a first universal binding site (120) for a first portion of a splint capture primer (or a complementary sequence thereof) and a sample index sequence (160), and a 3′ region that is complementary to at least a portion of universal binding site for a forward sequencing primer (140) (or a complementary sequence thereof) of the first double-stranded tailed molecule. In some embodiments, the left sample index sequence (160) comprises a short random sequence (e.g., NNN) that is about 3-20 nucleotides in length.

In some embodiments, the first and second tailed primers can be employed in at least two primer extension reactions to append additional adaptor sequences to the insert region (110) to generate a double-stranded recombinant molecule comprising an insert region (110) flanked on one side with adaptor sequences comprising (i) a universal binding site for a forward sequencing primer (140) (or a complementary sequence thereof), (ii) a left sample index sequence (160), and (iii) a first universal binding site (120) for a first portion of a splint capture primer (200) (or a complementary sequence thereof); and the insert region (110) can be flanked on the other side with adaptor sequences comprising (i) a universal binding site for a reverse sequencing primer (150) (or a complementary sequence thereof); (ii) a right sample index sequence (170); and (iii) a second universal binding site (130) for a second portion of the immobilized splint capture (or a complementary sequence thereof) (e.g., see FIGS. 20A-20D and FIGS. 21A-21F).

In some embodiments, individual first tailed primers in the plurality comprise a 5′ region carrying a second universal binding site (130) for a second portion of the immobilized splint capture (or a complementary sequence thereof), and a 3′ region that is complementary to at least a portion of a universal binding site for a second non-splint capture primer (133) (or a complementary sequence thereof).

In some embodiments, individual first tailed primers in the plurality comprise a 5′ region carrying a second universal binding site (130) for a second portion of the immobilized splint capture (or a complementary sequence thereof), a universal binding site for a second non-splint capture primer (133) (or a complementary sequence thereof), and a sample index sequence (170). In some embodiments, the plurality of first tailed primers comprise a 3′ region that is complementary to at least a portion of a universal binding site for a reverse sequencing primer (150) (or a complementary sequence thereof) of the single-stranded recombinant molecules. In some embodiments, the sample index sequence (170) comprises a short random sequence (e.g., NNN) that is about 3-20 nucleotides in length.

In some embodiments, individual first tailed primers in the plurality comprise a 5′ region carrying a second universal binding site (130) for a second portion of the immobilized splint capture (or a complementary sequence thereof), short random sequence (e.g., NNNN) (132) which provides nucleotide sequence diversity, a universal binding site for a second non-splint capture primer (133) (or a complementary sequence thereof), and a sample index sequence (170). In some embodiments, the plurality of first tailed primers comprise a 3′ region that is complementary to at least a portion of a universal binding site for a reverse sequencing primer (150) (or a complementary sequence thereof) of the single-stranded recombinant molecules. In some embodiments, the sample index sequence (170) comprises a short random sequence (e.g., NNN) that is about 3-20 nucleotides in length. In some embodiments, the short random sequence (e.g., NNNN) (132) is about 3-20 nucleotides in length.

The method further comprises conducting a second primer extension reaction using a second tailed primer. In some embodiments, the plurality of second tailed primers comprise a 5′ region carrying a first universal binding site (120) for a first portion of a splint capture primer (or a complementary sequence thereof), and a 3′ region that is complementary to at least a portion of a universal binding site for a first non-splint capture primer (123) (or a complementary sequence thereof).

In some embodiments, individual second tailed primers in the plurality comprise a 5′ region carrying a first universal binding site (120) for a first portion of a splint capture primer (or a complementary sequence thereof), a universal binding site for a first non-splint capture primer (123) (or a complementary sequence thereof), and a left sample index sequence (160). In some embodiments, the plurality of second tailed primers comprise a 3′ region that is complementary to at least a portion of universal binding site for a forward sequencing primer (140) (or a complementary sequence thereof) of the first double-stranded tailed molecule. In some embodiments, the left sample index sequence (160) comprises a short random sequence (e.g., NNN) that is about 3-20 nucleotides in length.

In some embodiments, the 5′ region of the first tailed primer comprises the sequence: 5′-GATCAGGTGAGGCTGCGACGACT-3′ (SEQ ID NO:3) which provides a second universal binding site (130) for a second portion of the immobilized splint capture primer. In some embodiments, the terminal 5′ nucleotides of the 5′ region of the first tailed primer can be truncated by 1, 2, 3, 4, 5 or 6 nucleotides. In some embodiments, the terminal 3′ nucleotides of the 5′ region of the first tailed primer can be truncated by 1, 2, 3, 4, 5 or 6 nucleotides.

In some embodiments, the 5′ region of the first tailed primer comprises the sequence: 5′-ATCAGGTGAGGCTGCGACGACT-3′ (SEQ ID NO:4) which provides a second universal binding site (130) for a second portion of the immobilized splint capture primer. In some embodiments, the terminal 5′ nucleotides of the 5′ region of the first tailed primer can be truncated by 1, 2, 3, 4, 5 or 6 nucleotides. In some embodiments, the terminal 3′ nucleotides of the 5′ region of the first tailed primer can be truncated by 1, 2, 3, 4, 5 or 6 nucleotides.

In some embodiments, the 5′ region of the first tailed primer comprises the sequence: 5′-GATCAGGTGAGGCT-3′ (SEQ ID NO:5) which provides a second universal binding site (130) for a second portion of the immobilized splint capture primer. In some embodiments, the terminal 5′ nucleotides of the 5′ region of the first tailed primer can be truncated by 1, 2, 3, 4, 5 or 6 nucleotides. In some embodiments, the terminal 3′ nucleotides of the 5′ region of the first tailed primer can be truncated by 1, 2, 3, 4, 5 or 6 nucleotides.

In some embodiments, the 5′ region of the first tailed primer comprises the sequence: 5′-GATCAGGTGAGGCTGCG-3′ (SEQ ID NO:6) which provides a second universal binding site (130) for a second portion of the immobilized splint capture primer. In some embodiments, the terminal 5′ nucleotides of the 5′ region of the first tailed primer can be truncated by 1, 2, 3, 4, 5 or 6 nucleotides. In some embodiments, the terminal 3′ nucleotides of the 5′ region of the first tailed primer can be truncated by 1, 2, 3, 4, 5 or 6 nucleotides.

In some embodiments, the 5′ region of the first tailed primer comprises the sequence: 5′-GTTGAGGAACCGAGTGT-3′ (SEQ ID NO:7) which provides a second universal binding site (130) for a second portion of the immobilized splint capture primer. In some embodiments, the terminal 5′ nucleotides of the 5′ region of the first tailed primer can be truncated by 1, 2, 3, 4, 5 or 6 nucleotides. In some embodiments, the terminal 3′ nucleotides of the 5′ region of the first tailed primer can be truncated by 1, 2, 3, 4, 5 or 6 nucleotides.

In some embodiments, the 5′ region of the first tailed primer comprises the sequence: 5′-GACTATTCCAGCGGTACG-3′ (SEQ ID NO:8) which provides a second universal binding site (130) for a second portion of the immobilized splint capture primer. In some embodiments, the terminal 5′ nucleotides of the 5′ region of the first tailed primer can be truncated by 1, 2, 3, 4, 5 or 6 nucleotides. In some embodiments, the terminal 3′ nucleotides of the 5′ region of the first tailed primer can be truncated by 1, 2, 3, 4, 5 or 6 nucleotides.

In some embodiments, the 3′ region of the first tailed primer comprises the sequence: 5′-CAAGCAGAAGACGGCATACGA-3′ (SEQ ID NO:9) which provides a universal binding site for a second non-splint capture primer (133). In some embodiments, the terminal 5′ nucleotides of the 3′ region of the first tailed primer can be truncated by 1, 2, 3, 4, 5 or 6 nucleotides. In some embodiments, the terminal 3′ nucleotides of the 5′ region of the first tailed primer can be truncated by 1, 2, 3, 4, 5 or 6 nucleotides.

In some embodiments, the 3′ region of the first tailed primer comprises the sequence: 5′-CAAGCAGAAGACGGCATACGAGAT-3′ (SEQ ID NO:10) which provides a universal binding site for a second non-splint capture primer (133). In some embodiments, the terminal 5′ nucleotides of the 3′ region of the first tailed primer can be truncated by 1, 2, 3, 4, 5 or 6 nucleotides. In some embodiments, the terminal 3′ nucleotides of the 5′ region of the first tailed primer can be truncated by 1, 2, 3, 4, 5 or 6 nucleotides.

In some embodiments, the 3′ region of the first tailed primer comprises the sequence: 5′-AAGCAGAAGACGGCATACGA-3′ (SEQ ID NO:11) which provides a universal binding site for a second non-splint capture primer (133). In some embodiments, the terminal 5′ nucleotides of the 3′ region of the first tailed primer can be truncated by 1, 2, 3, 4, 5 or 6 nucleotides. In some embodiments, the terminal 3′ nucleotides of the 5′ region of the first tailed primer can be truncated by 1, 2, 3, 4, 5 or 6 nucleotides.

In some embodiments, the 3′ region of the first tailed primer comprises the sequence: 5′-AGCAGAAGACGGCATACGA-3′ (SEQ ID NO:12) which provides a universal binding site for a second non-splint capture primer (133). In some embodiments, the terminal 5′ nucleotides of the 3′ region of the first tailed primer can be truncated by 1, 2, 3, 4, 5 or 6 nucleotides. In some embodiments, the terminal 3′ nucleotides of the 5′ region of the first tailed primer can be truncated by 1, 2, 3, 4, 5 or 6 nucleotides.

In some embodiments, the 3′ region of the first tailed primer comprises the sequence: 5′-GCAGAAGACGGCATACGA-3′ (SEQ ID NO:13) which provides a universal binding site for a second non-splint capture primer (133). In some embodiments, the terminal 5′ nucleotides of the 3′ region of the first tailed primer can be truncated by 1, 2, 3, 4, 5 or 6 nucleotides. In some embodiments, the terminal 3′ nucleotides of the 5′ region of the first tailed primer can be truncated by 1, 2, 3, 4, 5 or 6 nucleotides.

In some embodiments, the 3′ region of the first tailed primer comprises the sequence: 5′-CAGAAGACGGCATACGA-3′ (SEQ ID NO:14) which provides a universal binding site for a second non-splint capture primer (133). In some embodiments, the terminal 5′ nucleotides of the 3′ region of the first tailed primer can be truncated by 1, 2, 3, 4, 5 or 6 nucleotides. In some embodiments, the terminal 3′ nucleotides of the 5′ region of the first tailed primer can be truncated by 1, 2, 3, 4, 5 or 6 nucleotides.

In some embodiments, the 3′ region of the first tailed primer comprises the sequence: 5′-AGAAGACGGCATACGA-3′ (SEQ ID NO:15) which provides a universal binding site for a second non-splint capture primer (133). In some embodiments, the terminal 5′ nucleotides of the 3′ region of the first tailed primer can be truncated by 1, 2, 3, 4, 5 or 6 nucleotides. In some embodiments, the terminal 3′ nucleotides of the 5′ region of the first tailed primer can be truncated by 1, 2, 3, 4, 5 or 6 nucleotides.

In some embodiments, the 3′ region of the first tailed primer comprises the sequence: 5′-GAAGACGGCATACGA-3′ (SEQ ID NO:16) which provides a universal binding site for a second non-splint capture primer (133). In some embodiments, the terminal 5′ nucleotides of the 3′ region of the first tailed primer can be truncated by 1, 2, 3, 4, 5 or 6 nucleotides. In some embodiments, the terminal 3′ nucleotides of the 5′ region of the first tailed primer can be truncated by 1, 2, 3, 4, 5 or 6 nucleotides.

In some embodiments, the 3′ region of the first tailed primer comprises the sequence: 5′-AAGCAGAAGACGGCATACGAGAT-3′ (SEQ ID NO: 17) which provides a universal binding site for a second non-splint capture primer (133). In some embodiments, the terminal 5′ nucleotides of the 3′ region of the first tailed primer can be truncated by 1, 2, 3, 4, 5 or 6 nucleotides. In some embodiments, the terminal 3′ nucleotides of the 5′ region of the first tailed primer can be truncated by 1, 2, 3, 4, 5 or 6 nucleotides.

In some embodiments, the 3′ region of the first tailed primer comprises the sequence: 5′-GAAGACGGCATACGAGAT-3′ (SEQ ID NO:18) which provides a universal binding site for a second non-splint capture primer (133). In some embodiments, the terminal 5′ nucleotides of the 3′ region of the first tailed primer can be truncated by 1, 2, 3, 4, 5 or 6 nucleotides. In some embodiments, the terminal 3′ nucleotides of the 5′ region of the first tailed primer can be truncated by 1, 2, 3, 4, 5 or 6 nucleotides.

In some embodiments, the 3′ region of the first tailed primer comprises the sequence: 5′-TCAGCAGCGTCGGAGTGGACTAG-3′ (SEQ ID NO:19) which provides a universal binding site for a second non-splint capture primer (133). In some embodiments, the terminal 5′ nucleotides of the 3′ region of the first tailed primer can be truncated by 1, 2, 3, 4, 5 or 6 nucleotides. In some embodiments, the terminal 3′ nucleotides of the 5′ region of the first tailed primer can be truncated by 1, 2, 3, 4, 5 or 6 nucleotides.

In some embodiments, the first tailed primer comprises the sequence: 5′-GATCAGGTGAGGCTGCGACGACTCAAGCAGAAGACGGCATACGAGAT-3′ (SEQ ID NO:20) or a complementary sequence thereof.

In some embodiments, the first tailed primer comprises the sequence: 5′-GATCAGGTGAGGCTGCGACGACTAAGCAGAAGACGGCATA CGAGAT-3′ (SEQ ID NO:21) or a complementary sequence thereof.

In some embodiments, the first tailed primer comprises the sequence: 5′-GATCAGGTGAGGCTGCGACGACTGAAGACGGCATACGAGAT-3′ (SEQ ID NO:22) or a complementary sequence thereof.

In some embodiments, the first tailed primer comprises the sequence: 5′-ATCAGGTGAGGCTGCGACGACTCAAGCAGAAGACGGCATACGAGAT-3′ (SEQ ID NO:23) or a complementary sequence thereof.

In some embodiments, the first tailed primer comprises the sequence: 5′-GATCAGGTGAGGCTGCGACGACTCAAGCAGAAGACGGCATACGA-3′ (SEQ ID NO:24) or a complementary sequence thereof.

In some embodiments, the first tailed primer comprises the sequence: 5′-GATCAGGTGAGGCTAAGCAGAAGACGGCATACGAGAT-3′ (SEQ ID NO:25) or a complementary sequence thereof.

In some embodiments, the first tailed primer comprises the sequence: 5′-GATCAGGTGAGGCTGCGAAGCAGAAGACGGCATACGAGAT-3′ (SEQ ID NO:26) or a complementary sequence thereof.

In some embodiments, the first tailed primer comprises the sequence: 5′-GTTGAGGAACCGAGTGTTCAGCAGCGTCGGAGTGGACTAG-3′ (SEQ ID NO:27) or a complementary sequence thereof.

In some embodiments, the first tailed primer comprises the sequence: 5′-GACTATTCCAGCGGTACGTCAGCAGCGTCGGAGTGGACTAG-3′ (SEQ ID NO:28) or a complementary sequence thereof.

In some embodiments, the 5′ region of the second tailed primer comprises the sequence: 5′-AATGCACGTACTTTCAGGGT-3′ (SEQ ID NO:29) which provides a first universal binding site (120) for a first portion of a splint capture primer.

In some embodiments, the 5′ region of the second tailed primer comprises the sequence: 5′-CATGTAATGCACGTACTTTCAGGGT-3′ (SEQ ID NO:30) which provides a first universal binding site (120) for a first portion of a splint capture primer.

In some embodiments, the 5′ region of the second tailed primer comprises the sequence: 5′-CCATGTAATGCACGTACTTTCAGGGT-3′ (SEQ ID NO:31) which provides a first universal binding site (120) for a first portion of a splint capture primer.

In some embodiments, the 3′ region of the second tailed primer comprises the sequence: 5′-AATGATACGGCGACCACCGA-3′ (SEQ ID NO:32) which provides a universal binding site for a first non-splint capture primer (123). In some embodiments, the terminal 5′ nucleotides of the 3′ region of the second tailed primer can be truncated by 1, 2, 3, 4, 5, 6, 7, 8, 9, 10 or 11 nucleotides. In some embodiments, the terminal 3′ nucleotides of the 3′ region of the second tailed primer can be truncated by 1, 2, 3, 4, 5, 6, 7, 8, 9, 10 or 11 nucleotides.

In some embodiments, the 3′ region of the second tailed primer comprises the sequence: 5′-ATGATACGGCGACCACCGA-3′ (SEQ ID NO:33) which provides a universal binding site for a first non-splint capture primer (123). In some embodiments, the terminal 5′ nucleotides of the 3′ region of the second tailed primer can be truncated by 1, 2, 3, 4, 5, 6, 7, 8, 9, 10 or 11 nucleotides. In some embodiments, the terminal 3′ nucleotides of the 3′ region of the second tailed primer can be truncated by 1, 2, 3, 4, 5, 6, 7, 8, 9, 10 or 11 nucleotides.

In some embodiments, the 3′ region of the second tailed primer comprises the sequence: 5′-TGATACGGCGACCACCGA-3′ (SEQ ID NO:34) which provides a universal binding site for a first non-splint capture primer (123). In some embodiments, the terminal 5′ nucleotides of the 3′ region of the second tailed primer can be truncated by 1, 2, 3, 4, 5, 6, 7, 8, 9, 10 or 11 nucleotides. In some embodiments, the terminal 3′ nucleotides of the 3′ region of the second tailed primer can be truncated by 1, 2, 3, 4, 5, 6, 7, 8, 9, 10 or 11 nucleotides.

In some embodiments, the 3′ region of the second tailed primer comprises the sequence: 5′-GATACGGCGACCACCGA-3′ (SEQ ID NO:35) which provides a universal binding site for a first non-splint capture primer (123). In some embodiments, the terminal 5′ nucleotides of the 3′ region of the second tailed primer can be truncated by 1, 2, 3, 4, 5, 6, 7, 8, 9, 10 or 11 nucleotides. In some embodiments, the terminal 3′ nucleotides of the 3′ region of the second tailed primer can be truncated by 1, 2, 3, 4, 5, 6, 7, 8, 9, 10 or 11 nucleotides.

In some embodiments, the 3′ region of the second tailed primer comprises the sequence: 5′-ATACGGCGACCACCGA-3′ (SEQ ID NO:36) which provides a universal binding site for a first non-splint capture primer (123). In some embodiments, the terminal 5′ nucleotides of the 3′ region of the second tailed primer can be truncated by 1, 2, 3, 4, 5, 6, 7, 8, 9, 10 or 11 nucleotides. In some embodiments, the terminal 3′ nucleotides of the 3′ region of the second tailed primer can be truncated by 1, 2, 3, 4, 5, 6, 7, 8, 9, 10 or 11 nucleotides.

In some embodiments, the 3′ region of the second tailed primer comprises the sequence: 5′-TACGGCGACCACCGA-3′ (SEQ ID NO:37) which provides a universal binding site for a first non-splint capture primer (123). In some embodiments, the terminal 5′ nucleotides of the 3′ region of the second tailed primer can be truncated by 1, 2, 3, 4, 5, 6, 7, 8, 9, 10 or 11 nucleotides. In some embodiments, the terminal 3′ nucleotides of the 3′ region of the second tailed primer can be truncated by 1, 2, 3, 4, 5, 6, 7, 8, 9, 10 or 11 nucleotides.

In some embodiments, the 3′ region of the second tailed primer comprises the sequence: 5′-ACGGCGACCACCGA-3′ (SEQ ID NO:38) which provides a universal binding site for a first non-splint capture primer (123). In some embodiments, the terminal 5′ nucleotides of the 3′ region of the second tailed primer can be truncated by 1, 2, 3, 4, 5, 6, 7, 8, 9, 10 or 11 nucleotides. In some embodiments, the terminal 3′ nucleotides of the 3′ region of the second tailed primer can be truncated by 1, 2, 3, 4, 5, 6, 7, 8, 9, 10 or 11 nucleotides.

In some embodiments, the 3′ region of the second tailed primer comprises the sequence: 5′-GACCACCGA-3′ which provides a universal binding site for a first non-splint capture primer (123). In some embodiments, the terminal 5′ nucleotides of the 3′ region of the second tailed primer can be truncated by 1, 2, 3, 4, 5, 6, 7, 8, 9, 10 or 11 nucleotides. In some embodiments, the terminal 3′ nucleotides of the 3′ region of the second tailed primer can be truncated by 1, 2, 3, 4, 5, 6, 7, 8, 9, 10 or 11 nucleotides.

In some embodiments, the 3′ region of the second tailed primer comprises the sequence: 5′-AATGATACGGCGACCACCGAG-3′ (SEQ ID NO:40) which provides a universal binding site for a first non-splint capture primer (123). In some embodiments, the terminal 5′ nucleotides of the 3′ region of the second tailed primer can be truncated by 1, 2, 3, 4, 5, 6, 7, 8, 9, 10 or 11 nucleotides. In some embodiments, the terminal 3′ nucleotides of the 3′ region of the second tailed primer can be truncated by 1, 2, 3, 4, 5, 6, 7, 8, 9, 10 or 11 nucleotides.

In some embodiments, the 3′ region of the second tailed primer comprises the sequence: 5′-ATGATACGGCGACCACCGAG-3′ (SEQ ID NO:41) which provides a universal binding site for a first non-splint capture primer (123). In some embodiments, the terminal 5′ nucleotides of the 3′ region of the second tailed primer can be truncated by 1, 2, 3, 4, 5, 6, 7, 8, 9, 10 or 11 nucleotides. In some embodiments, the terminal 3′ nucleotides of the 3′ region of the second tailed primer can be truncated by 1, 2, 3, 4, 5, 6, 7, 8, 9, 10 or 11 nucleotides.

In some embodiments, the 3′ region of the second tailed primer comprises the sequence: 5′-GACCACCGAGATCTACAC-3′ (SEQ ID NO:42) which provides a universal binding site for a first non-splint capture primer (123). In some embodiments, the terminal 5′ nucleotides of the 3′ region of the second tailed primer can be truncated by 1, 2, 3, 4, 5, 6, 7, 8, 9, 10 or 11 nucleotides. In some embodiments, the terminal 3′ nucleotides of the 3′ region of the second tailed primer can be truncated by 1, 2, 3, 4, 5, 6, 7, 8, 9, 10 or 11 nucleotides.

In some embodiments, the 3′ region of the second tailed primer comprises the sequence: 5′-GAACGACATGGCTACGATCC-3′ (SEQ ID NO:43) which provides a universal binding site for a first non-splint capture primer (123). In some embodiments, the terminal 5′ nucleotides of the 3′ region of the second tailed primer can be truncated by 1, 2, 3, 4, 5, 6, 7, 8, 9, 10 or 11 nucleotides. In some embodiments, the terminal 3′ nucleotides of the 3′ region of the second tailed primer can be truncated by 1, 2, 3, 4, 5, 6, 7, 8, 9, 10 or 11 nucleotides.

In some embodiments, the 3′ region of the second tailed primer comprises the sequence: 5′-CTCTCAGTACGTCAGCAGTT-3′ (SEQ ID NO:44) which provides a universal binding site for a first non-splint capture primer (123). In some embodiments, the terminal 5′ nucleotides of the 3′ region of the second tailed primer can be truncated by 1, 2, 3, 4, 5, 6, 7, 8, 9, 10 or 11 nucleotides. In some embodiments, the terminal 3′ nucleotides of the 3′ region of the second tailed primer can be truncated by 1, 2, 3, 4, 5, 6, 7, 8, 9, 10 or 11 nucleotides.

In some embodiments, the second tailed primer comprises the sequence: 5′-AATGCACGTACTTTCAGGGTAATGATACGGCGACCACCGA-3′ (SEQ ID NO:45) or a complementary sequence thereof.

In some embodiments, the second tailed primer comprises the sequence: 5′-AATGCACGTACTTTCAGGGTATGATACGGCGACCACCGA-3′ (SEQ ID NO:46) or a complementary sequence thereof.

In some embodiments, the second tailed primer comprises the sequence: 5′-AATGCACGTACTTTCAGGGTAATGATACGGCGACCACCGAG-3′ (SEQ ID NO:47) or a complementary sequence thereof.

In some embodiments, the second tailed primer comprises the sequence: 5′-AATGCACGTACTTTCAGGGTATGATACGGCGACCACCGAG-3′ (SEQ ID NO:48) or a complementary sequence thereof.

In some embodiments, the second tailed primer comprises the sequence: 5′-AATGCACGTACTTTCAGGGTGACCACCGAGATCTACAC-3′ (SEQ ID NO:49) or a complementary sequence thereof.

In some embodiments, the second tailed primer comprises the sequence: 5′-CATGTAATGCACGTACTTTCAGGGTAATGATACGGCGACCACCGA-3′ (SEQ ID NO:50) or a complementary sequence thereof.

In some embodiments, the second tailed primer comprises the sequence: 5′-CATGTAATGCACGTACTTTCAGGGTATGATACGGCGACCACCGA-3′ (SEQ ID NO:51) or a complementary sequence thereof.

In some embodiments, the second tailed primer comprises the sequence: 5′-CATGTAATGCACGTACTTTCAGGGTAATGATACGGCGACCACCGAG-3′ (SEQ ID NO:52) or a complementary sequence thereof.

In some embodiments, the second tailed primer comprises the sequence: 5′-CATGTAATGCACGTACTTTCAGGGTATGATACGGCGACCACCGAG-3′ (SEQ ID NO:53) or a complementary sequence thereof.

In some embodiments, the second tailed primer comprises the sequence: 5′-CATGTAATGCACGTACTTTCAGGGTGACCACCGAGATCTACAC-3′ (SEQ ID NO:54) or a complementary sequence thereof.

In some embodiments, the second tailed primer comprises the sequence: 5′-CCATGTAATGCACGTACTTTCAGGGTAATGATACGGCGACCACCGA-3′ (SEQ ID NO:55) or a complementary sequence thereof.

In some embodiments, the second tailed primer comprises the sequence: 5′-CCATGTAATGCACGTACTTTCAGGGTATGATACGGCGACCACCGA-3′ (SEQ ID NO:56) or a complementary sequence thereof.

In some embodiments, the second tailed primer comprises the sequence: 5′-CCATGTAATGCACGTACTTTCAGGGTAATGATACGGCGACCACCGAG-3′ (SEQ ID NO:57) or a complementary sequence thereof.

In some embodiments, the second tailed primer comprises the sequence: 5′-CCATGTAATGCACGTACTTTCAGGGTATGATACGGCGACCACCGAG-3′ (SEQ ID NO:58) or a complementary sequence thereof.

In some embodiments, the second tailed primer comprises the sequence: 5′-CCATGTAATGCACGTACTTTCAGGGTGACCACCGAGATCTACAC-3′ (SEQ ID NO:59) or a complementary sequence thereof.

In some embodiments, the second tailed primer comprises the sequence: 5′-CATGTAATGCACGTACTTTCAGGGTGAACGACATGGCTACGATCC-3′ (SEQ ID NO:60) or a complementary sequence thereof.

In some embodiments, the second tailed primer comprises the sequence: 5′-CATGTAATGCACGTACTTTCAGGGTCTCTCAGTACGTCAGCAGTT-3′ (SEQ ID NO:61) or a complementary sequence thereof.

In some embodiments, the first and second tailed primers can be employed in at least two primer extension reactions to append additional adaptor sequences to the insert region (110) to generate a double-stranded recombinant molecule comprising an insert region (110) flanked on one side with adaptor sequences comprising (i) a universal binding site for a forward sequencing primer (140) (or a complementary sequence thereof), (ii) a left sample index sequence (160), (iii) a universal binding site for a first non-splint capture primer (123) (or a complementary sequence thereof), and (iv) a first universal binding site (120) for a first portion of a splint capture primer (or a complementary sequence thereof); and the insert region (110) can be flanked on the other side with adaptor sequences comprising (i) a universal binding site for a reverse sequencing primer (150) (or a complementary sequence thereof); (ii) a right sample index sequence (170); (iii) a universal binding site for a second non-splint capture primer (133) (or a complementary sequence thereof), and (iv) a second universal binding site (130) for a second portion of the immobilized splint capture (or a complementary sequence thereof) (e.g., see FIGS. 22A-C and FIGS. 23A-F).

In some embodiments, the first tailed primers can be used to conduct a first primer extension reaction and the second tailed primers can be used conduct a second primer extension to generate library molecules comprising an insert region appended on both sides with at least one adaptor sequence. In some embodiments, the first and second tailed primers can be used to conduct multiple PCR cycles (e.g., about 2-20 PCR cycles) to generate library molecules comprising an insert region appended on both sides with at least one adaptor sequence.

In some embodiments, in the library molecules, the first universal binding site (120) for binding the first portion of the splint capture primer comprises the sequence:

(SEQ ID NO: 62)
5′-CATGTAATGCACGTACTTTCAGGGT-3′.

In some embodiments, in the library molecules, the first universal binding site (120) for binding the first portion of the splint capture primer comprises the sequence:

(SEQ ID NO: 63)
5′-CCATGTAATGCACGTACTTTCAGGGT-3′.

In some embodiments, in the library molecules, the universal binding site for a first non-splint capture primer (123) comprises the sequence:

(SEQ ID NO: 64)
5′-AATGATACGGCGACCACCGA-3′.

In some embodiments, in the library molecules, the universal binding site for a first non-splint capture primer (123) comprises the sequence:

(SEQ ID NO: 65)
5′-CTCTCAGTACGTCAGCAGTT-3′.

In some embodiments, in the library molecules, the universal binding site for a forward sequencing primer (140) comprises the sequence:

(SEQ ID NO: 66)
5′-CGTGCTGGATTGGCTCACCAGACACCTTCCGACAT-3′.

In some embodiments, in the library molecules, the universal binding site for a forward sequencing rimer 140 comprises the sequence:

(SEQ ID NO: 67)
5′-ACACTCTTTCCCTACACGACGCTCTTCCGATCT-3′.

In some embodiments, in the library molecules, the universal binding site for a forward sequencing primer (140) comprises the sequence:

(SEQ ID NO: 68)
5′-TCGTCGGCAGCGTCAGATGTGTATAAGAGACAG-3′.

In some embodiments, in the library molecules, the universal binding site for a forward sequencing primer (140) comprises the sequence:

(SEQ ID NO: 69)
5′-GCTCACAGAACGACATGGCTACGATCCGACTT-3′.

In some embodiments, in the library molecules, the universal binding site for a forward sequencing primer (140) comprises the sequence:

(SEQ ID NO: 160)
5′-GAACGACATGGCTACGATCCGACTT-3′.

In some embodiments, in the library molecules, universal binding site for a reverse sequencing primer (150) comprises the sequence:

(SEQ ID NO: 70)
5′-ATGTCGGAAGGTGTGCAGGCTACCGCTTGTCAACT-3′.

In some embodiments, in the library molecules, universal binding site for a reverse sequencing primer (150) comprises the sequence:

(SEQ ID NO: 71)
5′-AGATCGGAAGAGCACACGTCTGAACTCCAGTCAC-3′.

In some embodiments, in the library molecules, universal binding site for a reverse sequencing primer (150) comprises the sequence:

(SEQ ID NO: 72)
5′-CTGTCTCTTATACACATCTCCGAGCCCACGAGAC-3′.

In some embodiments, in the library molecules, universal binding site for a reverse sequencing primer (150) comprises the sequence:

(SEQ ID NO: 73)
5′-TTGTCTTCCTAAGACCGCTTGGCCTCCGACTT-3′.

In some embodiments, in the library molecules, the universal binding site for a second non-splint capture primer (133) comprises the sequence:

(SEQ ID NO: 74)
5′-TCGTATGCCGTCTTCTGCTTG-3′.

In some embodiments, in the library molecules, the universal binding site for a second non-splint capture primer (133) comprises the sequence:

(SEQ ID NO: 75)
5′-CTGATAAGGTCGCCATGC-3′.

In some embodiments, in the library molecules, the second universal binding sequence (130) for binding the second portion of the splint capture primer comprises the sequence: 5′-AGTCGTCGCAGCCTCACCTGATC-3′ (SEQ ID NO:76).

In some embodiments, in the library molecules, the second universal binding sequence (130) for binding the second portion of the splint capture primer comprises the sequence: 5′-AGTCGTCGCAGCCTCACCTGAT-3′ (SEQ ID NO:77).

In some embodiments, the universal adaptor sequence in the linear library molecules (100) comprise a sequence that can bind a compaction oligonucleotide, or can bind a sequence that is complementary to the compaction oligonucleotide, where the compaction oligonucleotide comprises the sequence 5′-CATGTAATGCACGTACTTTCAGGGTAAACATGTAA TGCACGTACTTTCAGGGTUUU-3 (SEQ ID NO:78) or the compaction oligonucleotide comprises the sequence 5′-GATCAGGTGAGGCTGCGACGACTAAAGATCA GGTGAGGCTGCGACGACTUUU-3′ (SEQ ID NO:79).

In some embodiments, the splint capture primer (200) comprises the sequence:

(SEQ ID NO: 80)
5′-ATTACATGGATCAGGTGAGGCT-3′.

In some embodiments, the splint capture primer (200) comprises the sequence:

(SEQ ID NO: 81)
5′-GAGGCTGCGACGACT-3′.

In some embodiments, the splint capture primer (200) comprises the sequence:

(SEQ ID NO: 82)
5′-GATCAGGTGAGGCTGCGACGACT-3′.

In some embodiments, the splint capture primer (200) comprises the sequence:

(SEQ ID NO: 83)
5′-ATTACATGGATCAGGTGAGGCTGCG-3′.

In some embodiments, the splint capture primer (200) comprises the sequence:

(SEQ ID NO: 84)
5′-CATTACATGGATCAGGTGAGGCTGCG-3′.

In some embodiments, the splint capture primer (200) comprises the sequence:

(SEQ ID NO: 85)
5′-GCATTACATGGATCAGGTGAGGCTGCG-3′.

In some embodiments, the splint capture primer (200) comprises the sequence:

(SEQ ID NO: 86)
5′-GCATTACATGGATCAGGTGAGGCTGCGACGAC-3′.

In some embodiments, the splint capture primer (200) comprises the sequence:

(SEQ ID NO: 87)
5′-GTATCATTCAAGCAGAAGACGG-3′.

In some embodiments, the splint capture primer (200) comprises the sequence:

(SEQ ID NO: 88)
5′-CCGTATCATTCAAGCAGAAGACGGCAT-3′.

In some embodiments, the splint capture primer (200) comprises the sequence:

(SEQ ID NO: 89)
5′-GTATCATTCAAGCAGAAGACGGCATACG-3′.

In some embodiments, the splint capture primer (200) comprises the sequence:

(SEQ ID NO: 90)
5′-CCGTATCATTCAAGCAGAAGACGGCATACG-3′.

In some embodiments, the splint capture primer (200) comprises the sequence:

(SEQ ID NO: 91)
5′-GTATCATTCAAGCAGAAGACGGCATACGAG-3′.

In some embodiments, the splint capture primer (200) comprises the sequence:

(SEQ ID NO: 92)
5′-CGTATCATTCAAGCAGAAGACGGCATACGAG-3′.

In some embodiments, the splint capture primer (200) comprises the sequence:

(SEQ ID NO: 93)
5′-CCGTATCATTCAAGCAGAAGACGGCATACGAG-3′.

In some embodiments, the splint capture primer (200) comprises the sequence:

(SEQ ID NO: 94)
5′-TCGGTGGTCGCCGTATCATTCAAGCAGAAG-3′.

In some embodiments, the splint capture primer (200) comprises the sequence:

(SEQ ID NO: 95)
5′-TCGGTGGTCGCCGTATCATTCAAGCAGAA-3′.

In some embodiments, the splint capture primer (200) comprises the sequence:

(SEQ ID NO: 96)
5′-TCGGTGGTCGCCGTATCATTTCAAGCAGA-3′.

In some embodiments, the splint capture primer (200) comprises the sequence:

(SEQ ID NO: 97)
5′-TCGGTGGTCGCCGTATCATTTCAAGCAGAA-3′.

In some embodiments, the splint capture primer (200) comprises the sequence:

(SEQ ID NO: 98)
5′-GTATCATTTCAAGCAGAAGACGGCATACG-3′.

In some embodiments, the splint capture primer (200) comprises the sequence:

(SEQ ID NO: 99)
5′-GTATCATTTCAAGCAGAAGACGGCATACGAG-3′.

In some embodiments, the splint capture primer (200) comprises the sequence:

(SEQ ID NO: 100)
5′-CGTATCATTTCAAGCAGAAGACGGCATACGAG-3′.

In some embodiments, the splint capture primer (200) comprises the sequence:

(SEQ ID NO: 101)
5′-CGTATCATTCCAAGCAGAAGACGGCATACGAG-3′.

In some embodiments, the splint capture primer (200) comprises the sequence:

(SEQ ID NO: 102)
5′-GTCGCCGTATCAAGCAGAAGACGGCATACGAG-3′.

In some embodiments, the splint capture primer (200) comprises the sequence:

(SEQ ID NO: 103)
5′-GTCGCCGTATCAAGCAGAAGACGGCATACG-3′.

In some embodiments, the splint capture primer (200) comprises the sequence:

(SEQ ID NO: 104)
5′-GTCGCCGTATCAAGCAGAAGACGICATACGAG-3′.

In some embodiments, the splint capture primer (200) comprises the sequence:

(SEQ ID NO: 105)
5′-GTCGCCGTATCAAGCAGAAGACGGCAUACGAG-3′.

In some embodiments, the splint capture primer (200) comprises the sequence:

(SEQ ID NO: 106)
5′-CGCCGTATCAAGCAGAAGACGGCATACGAG-3′.

In some embodiments, the splint capture primer (200) comprises the sequence:

(SEQ ID NO: 107)
5′-CGTATCATTAAGCAGAAGACGGCATACGAGA-3′.

In some embodiments, the splint capture primer (200) comprises the sequence:

(SEQ ID NO: 108)
5′-CGTATCATTGCAGAAGACGGCATACGAGAT-3′.

In some embodiments, the splint capture primer (200) comprises the sequence:

(SEQ ID NO: 109)
5′-GTCGCCGTCAAGCAGAAGACGGCATACGAG-3′.

In some embodiments, the splint capture primer (200) comprises the sequence:

(SEQ ID NO: 110)
5′-CGCCGTATCAAGCAGAAGACGGCATACGAG-3′.

In some embodiments, the splint capture primer (200) comprises the sequence:

(SEQ ID NO: 111)
5′-TCGCCGTATCAAGCAGAAGACGGCATACGAG-3′.

In some embodiments, the splint capture primer (200) comprises the sequence:

(SEQ ID NO: 112)
5′-TTCGCCGTATCAAGCAGAAGACGGCATACGAG-3′.

In some embodiments, the splint capture primer (200) comprises the sequence:

(SEQ ID NO: 113)
5′-GTTCGCCGTATCAAGCAGAAGACGGCATACGAG-3′.

In some embodiments, the splint capture primer (200) comprises the sequence:

(SEQ ID NO: 114)
5′-CGTTCGCCGTATCAAGCAGAAGACGGCATACGAG-3′.

In some embodiments, the splint capture primer (200) comprises the sequence:

(SEQ ID NO: 115)
5′-AGATCTCGGTGGTCGCCGTATCAAGCAGAA-3′.

In some embodiments, the splint capture primer (200) comprises the sequence:

(SEQ ID NO: 116)
5′-AGATCTCGGTGGTCGCCGTATCAAGCAGAAG-3′.

In some embodiments, the splint capture primer (200) comprises the sequence:

(SEQ ID NO: 117)
5′-AGATCTCGGTGGTCGCCGTATCAAGCAGAAGA-3′.

In some embodiments, the splint capture primer (200) comprises the sequence:

(SEQ ID NO: 118)
5′-AGATCTCGGTGGTCGCCGTATCAAGCAGAAGAC-3′.

In some embodiments, the splint capture primer (200) comprises a sequence selected from the group consisting of SEQ ID NOS: 80-118.

In some embodiments, the pinning primer (500) comprises the sequence: 5′-AATGCACGTACTTTCAGGGT-3′ (SEQ ID NO:119) wherein the pinning primer comprises a non-extendible terminal 3′ end.

In some embodiments, the pinning primer (500) comprises the sequence: 5′-CATGTAATGCACGTACTTTCAGGGT-3′ (SEQ ID NO:120) wherein the pinning primer comprises a non-extendible terminal 3′ end.

In some embodiments, the pinning primer (500) comprises the sequence: 5′-GCGACCACCGAGATCTACAC-3′ (SEQ ID NO:121) wherein the pinning primer comprises a non-extendible terminal 3′ end.

In some embodiments, the pinning primer (500) comprises the sequence:

(SEQ ID NO: 122)
5′-AATGCACGTACTTTCAGGGTGCGACCACCGAGATCTACAC-3′.

wherein the pinning primer comprises a non-extendible terminal 3′ end.

In some embodiments, the pinning primer (500) comprises the sequence:

(SEQ ID NO: 123)
5′-GCGACCACCGAGATCTACACAATGCACGTACTTTCAGGGT-3′.

wherein the pinning primer comprises a non-extendible terminal 3′ end.

In some embodiments, the pinning primer (500) comprises the sequence: 5′-AGTCGTCGCAGCCTCACC-3′ (SEQ ID NO:124) wherein the pinning primer comprises a non-extendible terminal 3′ end.

In some embodiments, the pinning primer (500) comprises the sequence: 5′-ATCTCGTATGCCGTCTTCTG-3′ (SEQ ID NO:125) wherein the pinning primer comprises a non-extendible terminal 3′ end.

In some embodiments, the pinning primer (500) comprises a sequence selected from the group consisting of SEQ ID NOS:119-125.

In some embodiments, a first non-splint capture primer (e.g., P7) comprises the sequence: 5′-CAAGCAGAAGACGGCATACGA-3′ (SEQ ID NO:126) wherein the first non-splint primer comprises an extendible terminal 3′ end.

In some embodiments, a second non-splint capture primer (e.g., P5) comprises the sequence: 5′-AATGATACGGCGACCACCGA-3′ (SEQ ID NO:127) wherein the second non-splint primer comprises an extendible terminal 3′ end.

### Methods for Sequencing

The present disclosure provides methods for sequencing a plurality of concatemer template molecules which are immobilized to a support, for example any of the immobilized concatemer template molecules described herein. In some embodiments, the sequencing reactions employ a plurality of sequencing primers, a plurality of sequencing polymerases, and nucleotide reagents comprising any one or any combination of nucleotides and/or multivalent molecules. In some embodiments, the nucleotide reagents comprise canonical nucleotides. In some embodiments, the nucleotide reagents comprise nucleotide analogs. In some embodiments, the nucleotide analogs comprise detectably labeled nucleotides. In some embodiments, the nucleotide reagents comprise nucleotides carrying a removable or non-removable chain terminating moiety. In some embodiments, the nucleotide reagents comprise multivalent molecules each comprising a central core attached to multiple polymer arms each having a nucleotide moiety at the end of the arms. In some embodiments, the sequencing reactions employ binding non-labeled nucleotides without incorporation. In some embodiments, the sequencing reactions employ incorporating non-labeled nucleotide analogs. In some embodiments, the sequencing reactions employ incorporating detectably labeled nucleotides having removable chain terminating moiety. In some embodiments, the sequencing reactions employ a two-stage sequencing reaction comprising binding detectably labeled multivalent molecules without incorporation, and incorporating nucleotide analogs. In some embodiments, the sequence reactions employ incorporating a nucleotide moiety from an arm of a multivalent molecule. In some embodiments, the sequencing reactions employ phosphate chain-labeled nucleotides.

### Methods for Sequencing Using Chain Terminating Nucleotide Analogs

The present disclosure provides methods for sequencing concatemer template molecules using reversible chain terminating nucleotides, comprising step (a): contacting (i) a plurality of sequencing polymerases, (ii) a plurality of concatemer template molecules immobilized to a support and (iii) a plurality of nucleic acid sequencing primers, where the contacting is conducted under a condition suitable to form a plurality of sequencing polymerase complexes comprising a sequencing polymerase bound to a nucleic acid duplex, wherein the nucleic acid duplex comprises a portion of a concatemer template molecule hybridized to a nucleic acid sequencing primer. In some embodiments, the sequencing polymerases comprise a recombinant mutant sequencing polymerase that can bind and incorporate nucleotide analogs. In some embodiments, the nucleic acid sequencing primers comprise 3′ extendible ends or 3′ blocked end that can be converted into a 3′ extendible end.

In some embodiments, the methods for sequencing concatemer template molecules further comprise step (b): contacting the plurality of sequencing polymerase complexes with a plurality of nucleotides under a condition suitable for binding at least one nucleotide to one of the sequencing polymerase complexes, and the condition is suitable for promoting polymerase-catalyzed nucleotide incorporation. In some embodiments, the sequencing polymerase complex is contacted with the plurality of nucleotides in the presence of at least one catalytic cation comprising magnesium and/or manganese. In some embodiments, the plurality of nucleotides comprises at least one nucleotide analog having a chain terminating moiety at the sugar 2′ or 3′ position. In some embodiments, the chain terminating moiety is removable from the sugar 2′ or 3′ position to convert the chain terminating moiety to an OH or H group. In some embodiments, the plurality of nucleotides comprises at least one nucleotide that lacks a chain terminating moiety. In some embodiments, at least one nucleotide is labeled with a detectable reporter moiety (e.g., fluorophore). In some embodiments, the plurality of nucleotides comprise one type of nucleotides selected from a group consisting of dATP, dGTP, dCTP, dTTP or dUTP. In some embodiments, the plurality of nucleotides comprise a mixture of any two or more types of nucleotides comprising dATP, dGTP, dCTP, dTTP and/or dUTP.

In some embodiments, the methods for sequencing concatemer template molecules further comprise step (c): incorporating at least one nucleotide into the 3′ end of an extendible sequencing primer of at least one sequencing polymerase complex. In some embodiments, the nucleotide incorporation reaction of step (c) comprises a primer extension reaction.

In some embodiments, the methods for sequencing concatemer template molecules further comprise step (d): repeating steps (b) and (c) at least once.

In some embodiments, in step (b), the fluorophore is attached to the nucleotide base. In some embodiments, the fluorophore is attached to the nucleotide base with a linker which is cleavable/removable from the base. In some embodiments, at least one of the nucleotides in the plurality is not labeled with a detectable reporter moiety. In some embodiments, a particular detectable reporter moiety (e.g., fluorophore) that is attached to the nucleotide can correspond to the nucleotide base (e.g., dATP, dGTP, dCTP, dTTP or dUTP) to permit detection and identification of the nucleotide base. In some embodiments, the nucleotide analog comprises a fluorophore attached to the nucleotide base with a linker which is cleavable/removable from the base, and the nucleotide analog further comprises a chain terminating moiety attached to the 2′ or 3′ sugar position by a linker which is cleavable/removable using the same condition (e.g., chemical cleaving condition) that cleaves the fluorophore from the base.

In some embodiments, the method further comprises detecting the at least one incorporated nucleotide at step (c) and/or (d). In some embodiments, the method further comprises identifying the at least one incorporated nucleotide at step (c) and/or (d). In some embodiments, the sequence of the template molecule can be determined by detecting and identifying the nucleotide that binds the sequencing polymerase, thereby determining the sequence of the template molecule. In some embodiments, the sequence of the concatemer template molecule can be determined by detecting and identifying the nucleotide that incorporates into the 3′ end of the primer, thereby determining the sequence of the template molecule.

### Two-Stage Methods for Nucleic Acid Sequencing

The present disclosure provides a two-stage method for sequencing any of the immobilized concatemer template molecules described herein. In some embodiments, the first stage generally comprises binding multivalent molecules to polymerase complexes to form multivalent-binding polymerase complexes, and detecting the multivalent-binding polymerase complexes. In some embodiments, the second stage comprises nucleotide incorporation and extension of the sequencing primer. In some embodiments, one sequencing cycle comprises completion of a first and second stage. In some embodiments, any of the workflows that employ a two-stage sequencing method comprises conducting 5-25 sequencing cycles, or 25-50 sequencing cycles, or 50-75 sequencing cycles, or 75-100 sequencing cycles, or 100-200 sequencing cycles, or 200-500 sequencing cycles, or 500-750 sequencing cycles, or 750-1000 sequencing cycles.

In some embodiments, the first stage comprises step (a): contacting (i) a first plurality of sequencing polymerases, (ii) a plurality of concatemer template molecules immobilized to a support and (iii) a plurality of nucleic acid sequencing primers, where the contacting is conducted under a condition suitable to form a first plurality of sequencing polymerase complexes each complex comprising a first sequencing polymerase bound to a nucleic acid duplex wherein the nucleic acid duplex comprises a portion of a concatemer template molecule hybridized to a nucleic acid sequencing primer. In some embodiments, the nucleic acid sequencing primers comprise 3′ extendible ends. In some embodiments, the nucleic acid sequencing primers comprise 3′ non-extendible ends.

In some embodiments, the methods for sequencing concatemer template molecules further comprise step (b): contacting the first plurality of polymerase complexes with a plurality of multivalent molecules to form a plurality of multivalent-binding polymerase complexes (e.g., binding complexes). In some embodiments, individual multivalent molecules in the plurality of multivalent molecules comprise a core attached to multiple nucleotide arms and each nucleotide arm is attached to a nucleotide moiety (e.g., FIGS. 1-4). In some embodiments, the contacting of step (b) is conducted under a condition suitable for binding complementary nucleotide moieties of the multivalent molecules to at least two of the polymerase complexes in the first plurality thereby forming a plurality of multivalent-binding polymerase complexes. In some embodiments, the condition is suitable for inhibiting polymerase-catalyzed incorporation of the complementary nucleotide moieties into the primers of the plurality of multivalent-binding polymerase complexes. In some embodiments, the contacting of step (b) is conducted in the presence of at least one non-catalytic cation which inhibits polymerase-catalyzed nucleotide incorporation. In some embodiments, the at least one non-catalytic cation comprises strontium, barium and/or calcium.

In some embodiments, in the method of step (b), at least one of the multivalent molecules in the plurality of multivalent molecules is labeled with a detectable reporter moiety. In some embodiments, the detectable reporter moiety comprises a fluorophore.

In some embodiments, in the method of step (b), individual nucleotide arms of a multivalent molecule comprise (i) a core attachment moiety, (ii) a spacer comprising a PEG moiety, (iii) a linker, and (iv) a nucleotide moiety, wherein the core is attached to the plurality of nucleotide arms, wherein the spacer is attached to the linker, wherein the linker is attached to the nucleotide moiety. In some embodiments, individual linker arms of a multivalent molecule lack a linker, wherein the spacer is attached to the nucleotide moiety. In some embodiments, the labeled multivalent molecules comprise a fluorophore attached to the core, spacer, linker and/or nucleotide moiety of the multivalent molecules.

In some embodiments, in the method of step (b), the plurality of multivalent molecules comprise at least one multivalent molecule having multiple nucleotide arms (e.g., FIGS. 1-4) each attached with a nucleotide analog (e.g., nucleotide analog moiety), where the nucleotide analog includes a chain terminating moiety at the sugar 2′ and/or 3′ position. In some embodiments, the plurality of multivalent molecules comprises at least one multivalent molecule comprising multiple nucleotide arms each attached with a nucleotide moiety that lacks a chain terminating moiety.

In some embodiments, the methods for sequencing further comprise step (c): detecting the plurality of multivalent-binding polymerase complexes. In some embodiments, the detecting includes detecting the multivalent molecules that are bound to the polymerase complexes in the first plurality, where the complementary nucleotide moieties of the multivalent molecules are bound to the primers but incorporation of the complementary nucleotide moieties is inhibited. In some embodiments, the multivalent molecules are labeled with a detectable reporter moiety to permit detection.

In some embodiments, the methods for sequencing further comprise step (d): identifying the nucleo-base of the complementary nucleotide moieties that are bound to the first plurality of polymerase complexes, thereby determining the sequence of the concatemer template molecule. In some embodiments, the multivalent molecules are labeled with a detectable reporter moiety that corresponds to the particular nucleotide moieties attached to the nucleotide arms to permit identification of the complementary nucleotide moieties (e.g., nucleotide base adenine, guanine, cytosine, thymine or uracil) that are bound to the first plurality of polymerase complexes.

In some embodiments, the second stage of the two-stage sequencing method generally comprises nucleotide incorporation. In some embodiments, the methods for sequencing further comprise step (e): dissociating the plurality of multivalent-binding polymerase complexes and removing the first plurality of sequencing polymerases and their bound multivalent molecules, and retaining the plurality of nucleic acid duplexes.

In some embodiments, the methods for sequencing further comprises step (f): contacting the plurality of the retained nucleic acid duplexes of step (e) with a second plurality of sequencing polymerases, wherein the contacting is conducted under a condition suitable for binding the second plurality of sequencing polymerases to the plurality of the retained nucleic acid duplexes, thereby forming a second plurality of polymerase complexes each complex comprising a second sequencing polymerase bound to a nucleic acid duplex. In some embodiments, the second sequencing polymerase comprises a recombinant mutant sequencing polymerase.

In some embodiments, the plurality of first sequencing polymerases of step (a) have an amino acid sequence that is 100% identical to the amino acid sequence as the plurality of the second sequencing polymerases of step (f). In some embodiments, the plurality of first sequencing polymerases of step (a) have an amino acid sequence that differs from the amino acid sequence of the plurality of the second sequencing polymerases of step (f).

In some embodiments, the methods for sequencing further comprise step (g): contacting the second plurality of polymerase complexes with a plurality of nucleotides, wherein the contacting is conducted under a condition suitable for binding complementary nucleotides from the plurality of nucleotides to at least two of the second polymerase complexes thereby forming a plurality of nucleotide-polymerase complexes. In some embodiments, the contacting of step (g) is conducted under a condition that is suitable for promoting polymerase-catalyzed incorporation of the bound complementary nucleotides into the primers of the nucleotide-polymerase complexes. In some embodiments, the incorporating the nucleotide into the 3′ end of the primer in step (g) comprises a primer extension reaction. In some embodiments, the contacting of step (g) is conducted in the presence of at least one catalytic cation which promotes nucleotide incorporation. In some embodiments, the at least one catalytic cation comprises magnesium and/or manganese. In some embodiments, the plurality of nucleotides comprise native nucleotides (e.g., non-analog nucleotides) or nucleotide analogs. In some embodiments, the plurality of nucleotides comprise a 2′ and/or 3′ chain terminating moiety which is removable or is not removable. In some embodiments, the plurality of nucleotides comprises a plurality of nucleotides labeled with detectable reporter moiety. The detectable reporter moiety comprises a fluorophore. In some embodiments, the fluorophore is attached to the nucleotide base. In some embodiments, the fluorophore is attached to the nucleotide base with a linker which is cleavable/removable from the base or is not removable from the base. In some embodiments, at least one of the nucleotides in the plurality is not labeled with a detectable reporter moiety. In some embodiments, the plurality of nucleotides are non-labeled nucleotides. In some embodiments, a particular detectable reporter moiety (e.g., fluorophore) that is attached to the nucleotide can correspond to the nucleotide base (e.g., dATP, dGTP, dCTP, dTTP or dUTP) to permit detection and identification of the nucleotide base.

In some embodiments, when the plurality of nucleotides in steps (g) comprises labeled nucleotides, the methods for sequencing further comprise step (h): detecting the complementary nucleotides which are incorporated into the primers of the nucleotide-polymerase complexes. In some embodiments, the plurality of nucleotides are labeled with a detectable reporter moiety to permit detection. In some embodiments, when the plurality of nucleotides in steps (g) comprises non-labeled nucleotides, the detecting of step (h) is omitted.

In some embodiments, when the plurality of nucleotides in steps (g) comprises labeled nucleotides, the methods for sequencing further comprise step (i): identifying the bases of the complementary nucleotides which are incorporated into the primers of the nucleotide-polymerase complexes. In some embodiments, the identification of the incorporated complementary nucleotides in step (i) can be used to confirm the identity of the complementary nucleotides of the multivalent molecules that are bound to the first plurality of polymerase complexes in step (d). In some embodiments, the identifying of step (i) can be used to determine the sequence of the concatemer template molecules. In some embodiments, when the plurality of nucleotides in steps (g) comprises non-labeled nucleotides, the identifying of step (i) is omitted.

In some embodiments, when the plurality of nucleotides in step (g) comprise 2′ and/or 3′ chain terminating nucleotides, the methods for sequencing further comprise step (j): removing the chain terminating moiety from the incorporated nucleotides.

In some embodiments, the methods for sequencing further comprise step (k): repeating steps (a)-(j) at least once. In some embodiments, the sequence of the concatemer template molecules can be determined by detecting and identifying the multivalent molecules that bind the sequencing polymerases but do not incorporate into the 3′ end of the primer at steps (c) and (d). In some embodiments, the sequence of the concatemer template molecule can be determined (or confirmed) by detecting and identifying the nucleotide that incorporates into the 3′ end of the primer at steps (h) and (i).

### Forming Avidity Complexes

In some embodiments, in any of the two-stage sequencing methods, the binding of the first plurality of polymerase complexes with the plurality of multivalent molecules forms at least one avidity complex, the method comprising the steps: (1) binding a first sequencing primer, a first sequencing polymerase, and a first multivalent molecule to a first portion of a concatemer template molecule thereby forming a first binding complex, wherein a first nucleotide moiety of the first multivalent molecule binds to the first sequencing polymerase; and (2) binding a second sequencing primer, a second sequencing polymerase, and the first multivalent molecule to a second portion of the same concatemer template molecule thereby forming a second binding complex, wherein a second nucleotide moiety of the first multivalent molecule binds to the second sequencing polymerase, wherein the first and second binding complexes which include the same multivalent molecule forms an avidity complex. The concatemer template molecule comprises tandem repeat sequences of a sequence of interest and at least one universal site for binding a sequencing primer. The first and second sequencing primers can bind to a sequencing primer binding site along the concatemer template molecule. Exemplary multivalent molecules are shown in FIGS. 1-4.

Forming Avidity Complexes with Detecting and Identifying

In some embodiments, in any of the two-stage sequencing methods, wherein the method includes binding the first plurality of polymerase complexes with the plurality of multivalent molecules to form at least one avidity complex, the method comprising the steps: (1) contacting the plurality of sequencing polymerases and the plurality of sequencing primers with different portions of a concatemer template molecule to form at least first and second polymerase complexes on the same concatemer template molecule; (2) contacting a plurality of multivalent molecules to the at least first and second polymerase complexes on the same concatemer template molecule, under conditions suitable to bind a single multivalent molecule from the plurality to the first and second polymerase complexes, wherein at least a first nucleotide moiety of the single multivalent molecule is bound to the first polymerase complex which includes a first sequencing primer hybridized to a first portion of the concatemer template molecule thereby forming a first binding complex (e.g., first ternary complex), and wherein at least a second nucleotide moiety of the single multivalent molecule is bound to the second polymerase complex which includes a second sequencing primer hybridized to a second portion of the concatemer template molecule thereby forming a second binding complex (e.g., second ternary complex), wherein the contacting is conducted under a condition suitable to inhibit polymerase-catalyzed incorporation of the bound first and second nucleotide moieties in the first and second binding complexes, and wherein the first and second binding complexes which are bound to the same multivalent molecule forms an avidity complex; and (3) detecting the first and second binding complexes on the same concatemer template molecule, and (4) identifying the first nucleotide moiety in the first binding complex thereby determining the sequence of the first portion of the concatemer template molecule, and identifying the second nucleotide moiety in the second binding complex thereby determining the sequence of the second portion of the concatemer template molecule.

The concatemer template molecule comprises tandem repeat sequences of a sequence of interest and at least one universal site for binding a sequencing primer. The plurality of nucleic acid primers can bind to a sequencing primer binding site along the concatemer template molecule. Exemplary multivalent molecules are shown in FIGS. 1-4.

### Sequencing-by-Binding

The present disclosure provides methods for sequencing concatemer template molecules comprising a sequencing-by-binding (SBB) procedure which employs non-labeled chain-terminating nucleotides. In some embodiments, the sequencing-by-binding (SBB) method comprises the steps of (a) sequentially contacting a primed template nucleic acid (e.g., a an immobilized concatemer template molecule hybridized to a sequencing primer) with at least two separate mixtures under ternary complex stabilizing conditions, wherein the at least two separate mixtures each include a polymerase and a nucleotide, whereby the sequentially contacting results in the primed template nucleic acid being contacted, under the ternary complex stabilizing conditions, with nucleotide cognates for first, second and third base type base types in the template; (b) examining the at least two separate mixtures to determine whether a ternary complex formed; and (c) identifying the next correct nucleotide for the primed template nucleic acid molecule, wherein the next correct nucleotide is identified as a cognate of the first, second or third base type if ternary complex is detected in step (b), and wherein the next correct nucleotide is imputed to be a nucleotide cognate of a fourth base type based on the absence of a ternary complex in step (b); (d) adding a next correct nucleotide to the primer of the primed template nucleic acid after step (b), thereby producing an extended primer; and (e) repeating steps (a) through (d) at least once on the primed template nucleic acid that comprises the extended primer. Exemplary sequencing-by-binding methods are described in U.S. Pat. Nos. 10,246,744 and 10,731,141 (where the contents of both patents are hereby incorporated by reference in their entireties).

In some embodiments, in step (a) of all of the sequencing methods described herein, the plurality of concatemer template molecules are immobilized to a support at a density of about 10²-10¹⁵ per mm².

In some embodiments, in step (a) of all of the sequencing methods described herein, the plurality of concatemer template molecules are immobilized to a support at pre-determined positions on the support, or immobilized to random positions on the support.

In some embodiments, in step (a) of all of the sequencing methods described herein, the support is passivated/coated with at least one polymer layer. In some embodiments, at least one of the polymer layers comprises a plurality of capture primers tethered to the polymer layer. In some embodiments, individual capture primers serve to attach a template molecule to the polymer layer. In some embodiments, the plurality of concatemer template molecules are immobilized to the at least one polymer layer at pre-determined positions on the polymer layer, or immobilized to random positions on the polymer layer.

### Methods for Sequencing Using Phosphate Chain-Labeled Nucleotide Analogs

The present disclosure provides methods for sequencing concatemer template molecules using a plurality of phosphate chain-labeled nucleotides, comprising step (a): contacting (i) a plurality of sequencing polymerases, (ii) a plurality of concatemer template molecules immobilized to a support and (iii) a plurality of nucleic acid sequencing primers, where the contacting is conducted under a condition suitable to form a plurality of sequencing polymerase complexes each complex comprising a sequencing polymerase bound to a nucleic acid duplex wherein the nucleic acid duplex comprises a portion of a concatemer template molecule hybridized to a nucleic acid sequencing primer. In some embodiments, the sequencing polymerases comprise a recombinant mutant sequencing polymerase that can bind and incorporate nucleotide analogs. In some embodiments, the sequencing primers comprise 3′ extendible ends or 3′ blocked end that can be converted into a 3′ extendible end.

In some embodiments, the methods for sequencing concatemer template molecules further comprise step (b): contacting the plurality of sequencing polymerase complexes with a plurality of phosphate chain-labeled nucleotides under a condition suitable for binding at least one phosphate chain-labeled nucleotide to one of the sequencing polymerase complexes, and the condition is suitable for promoting polymerase-catalyzed nucleotide incorporation. In some embodiments, the sequencing polymerase complex is contacted with the plurality of nucleotides in the presence of at least one catalytic cation comprising magnesium and/or manganese. In some embodiments, individual phosphate chain-labeled nucleotides in the plurality comprise an aromatic base, a five carbon sugar (e.g., ribose or deoxyribose), and phosphate chain comprising 3-20 phosphate groups, where the terminal phosphate group is linked to a detectable reporter moiety (e.g., a fluorophore). The first, second and third phosphate groups can be referred to as alpha, beta and gamma phosphate groups. In some embodiments, a particular detectable reporter moiety which is attached to the terminal phosphate group corresponds to the nucleotide base (e.g., dATP, dGTP, dCTP, dTTP or dUTP) to permit detection and identification of the nucleo-base. In some embodiments, the sequencing polymerases are capable of binding a complementary phosphate chain labeled nucleotide and incorporating the complementary nucleotide opposite a nucleotide in a template molecule. In some embodiment, the polymerase-catalyzed nucleotide incorporation reaction cleaves between the alpha and beta phosphate groups thereby releasing a multi-phosphate chain linked to the detectable reporter moiety. In some embodiments, the plurality of phosphate chain-labeled nucleotides comprises one type or a mixture of any two or more types of nucleotides comprising dATP, dGTP, dCTP, dTTP and/or dUTP.

In some embodiments, the sequencing method further comprises step (c): detecting the fluorescent signal emitted by the phosphate chain labeled nucleotide that is bound by the sequencing polymerase, and incorporated into the terminal end of the sequencing primer. In some embodiments, step (c) further comprises identifying the phosphate chain labeled nucleotide that is bound by the sequencing polymerase, and incorporated into the terminal end of the sequencing primer.

In some embodiments, the sequencing method further comprises step (d): repeating steps (b)-(c) at least once. In some embodiments, sequencing methods that employ phosphate chain labeled nucleotides can be conducted according to the methods described in U.S. Pat. Nos. 7,170,050; 7,302,146; and/or 7,405,281.

In some embodiments, in step (a), the plurality of concatemer template molecules are immobilized to a support which comprises a plurality of separate compartments. In some embodiments, the plurality of sequencing polymerases are in solution in a compartment. In some embodiments, at least one sequencing polymerase is immobilized to the bottom of individual compartments. In some embodiments, the separate compartments comprise a silica bottom through which light can penetrate. In some embodiments, the separate compartments comprise a silica bottom configured with a nanophotonic confinement structure comprising a hole in a metal cladding film (e.g., aluminum cladding film). In some embodiments, the hole in the metal cladding has a small aperture, for example, approximately 70 nm. In some embodiments, the height of the nanophotonic confinement structure is approximately 100 nm. In some embodiments, the nanophotonic confinement structure comprises a zero mode waveguide (ZMW). In some embodiments, the nanophotonic confinement structure contains a liquid.

### Sequencing Polymerases

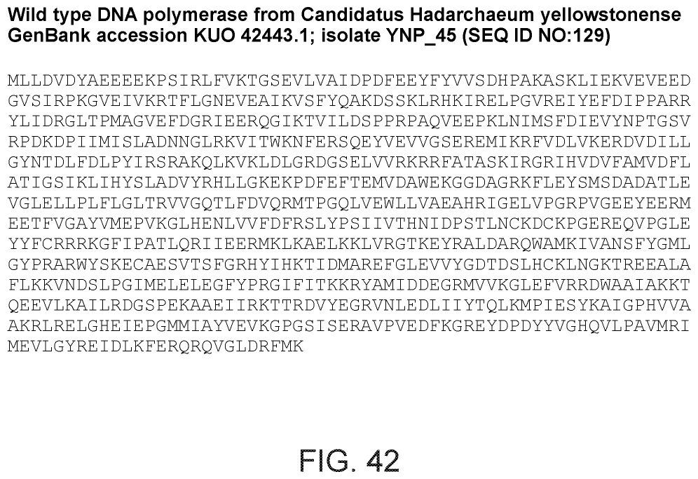

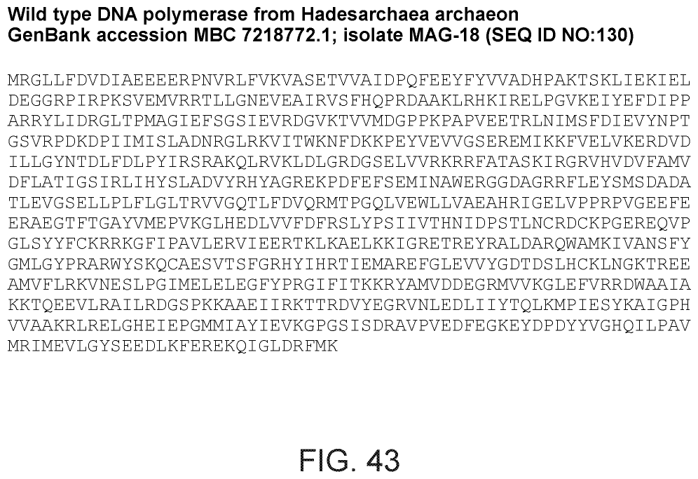

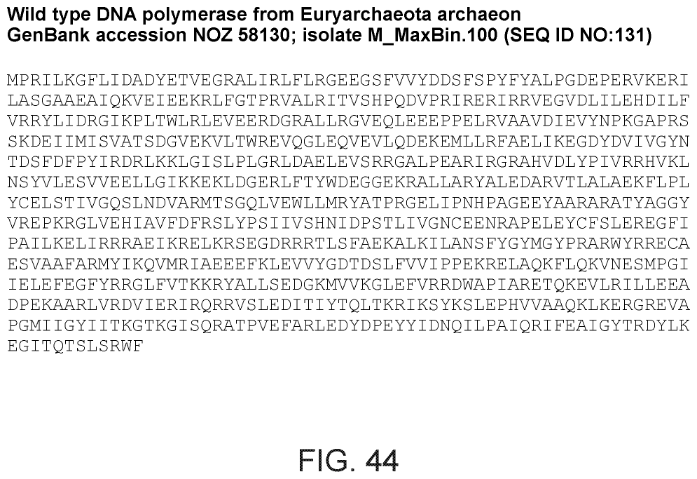

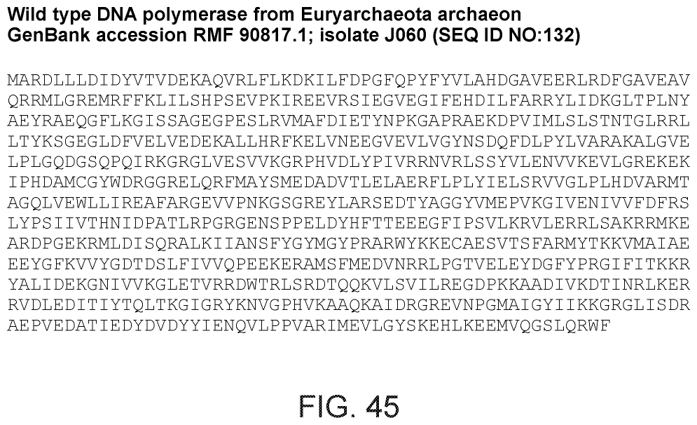

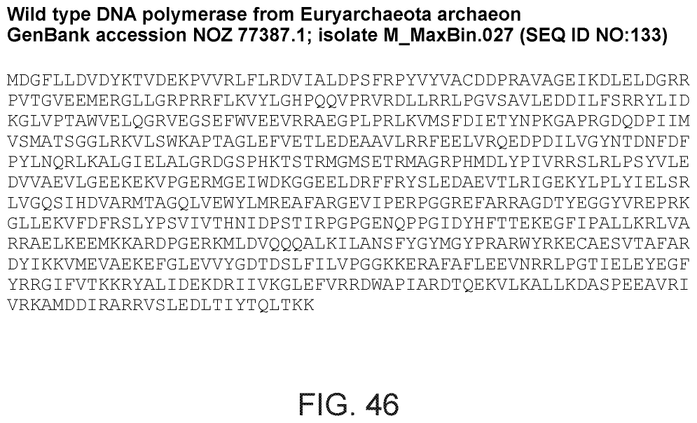

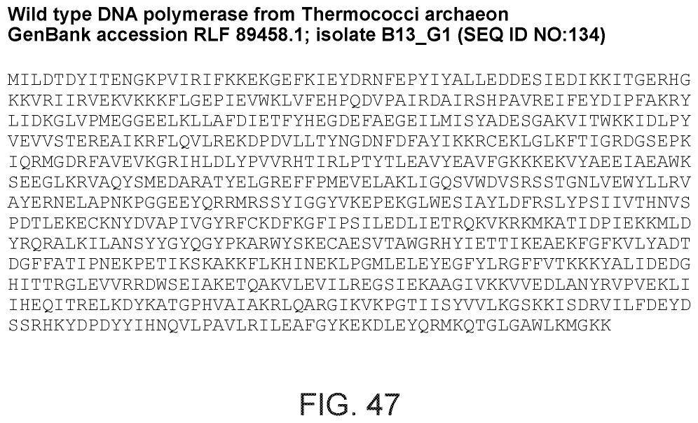

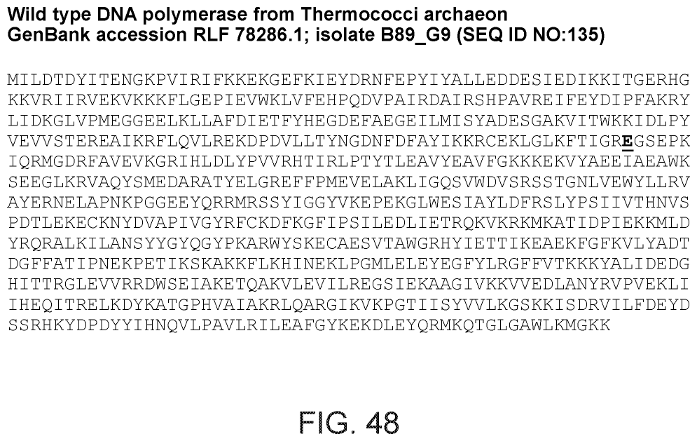

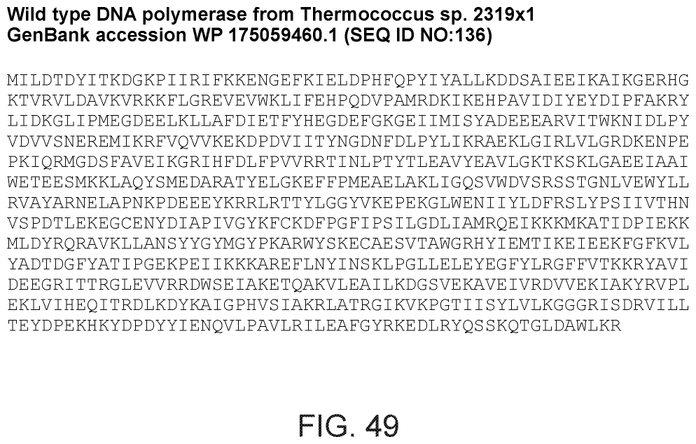

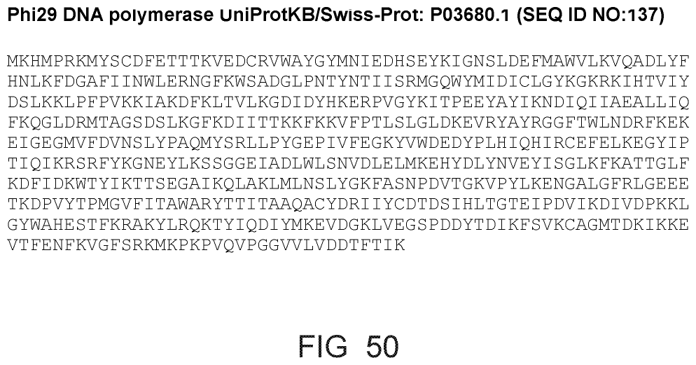

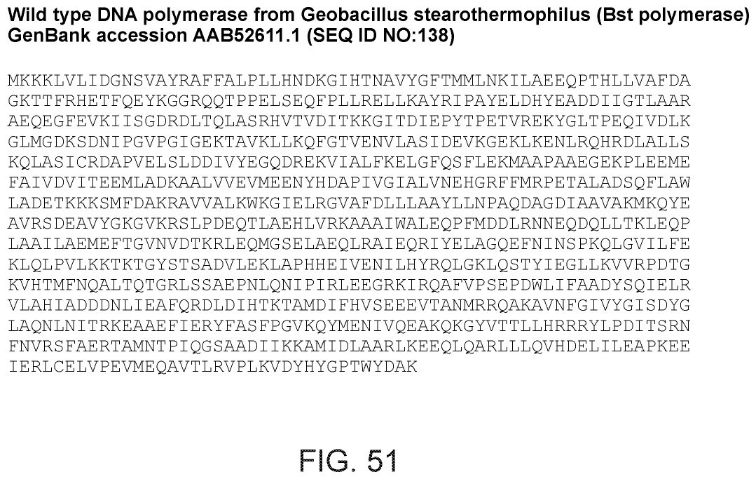

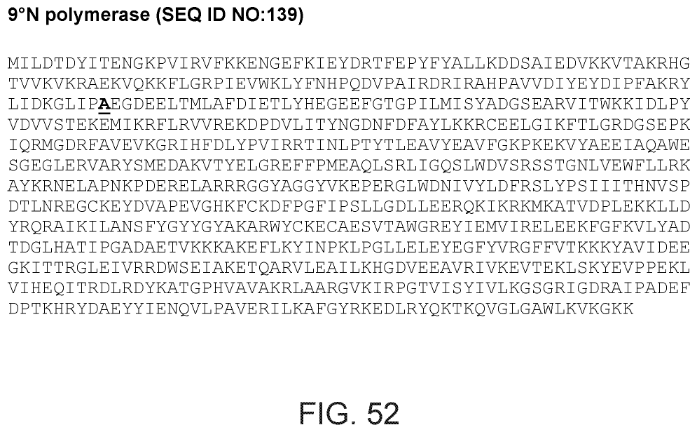

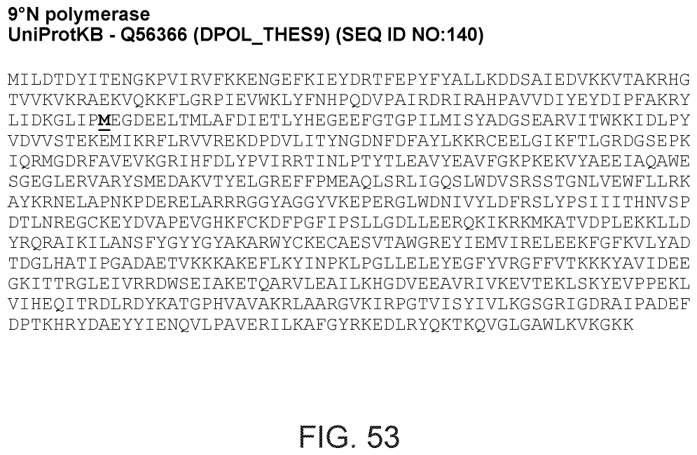

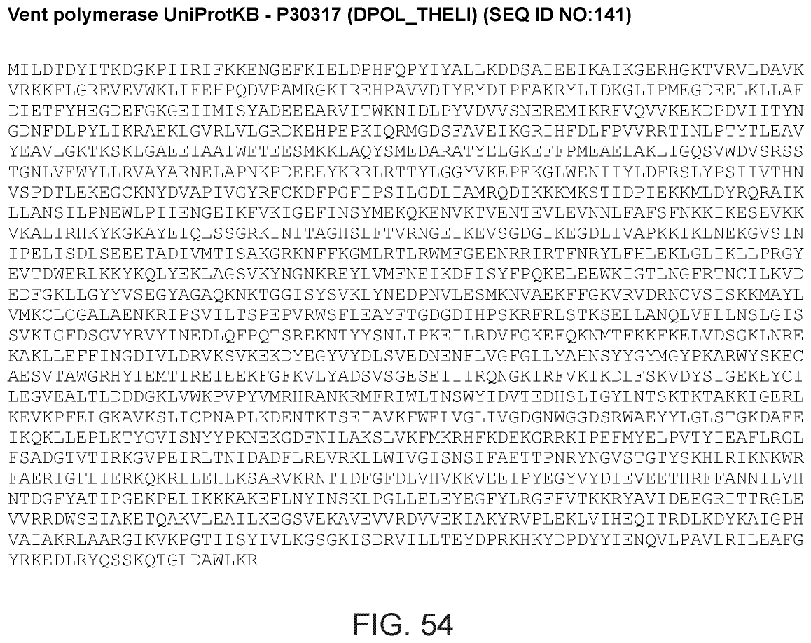

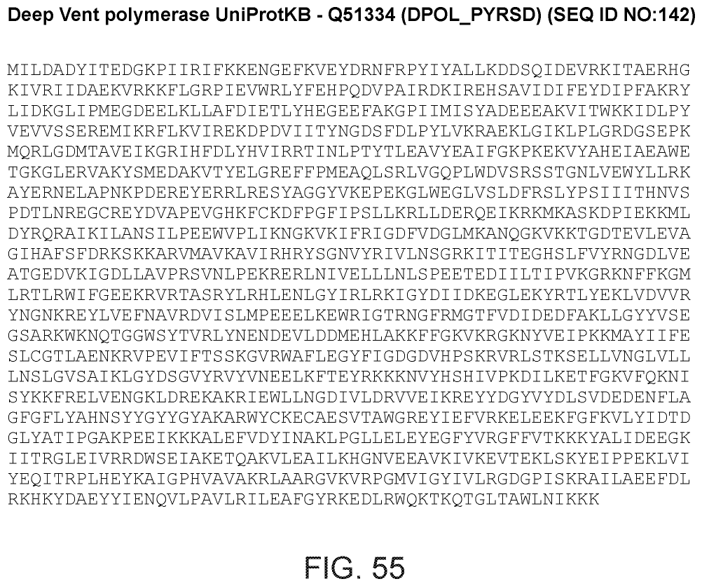

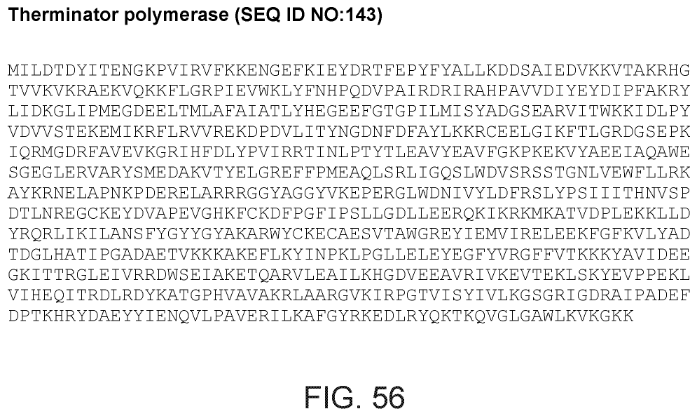

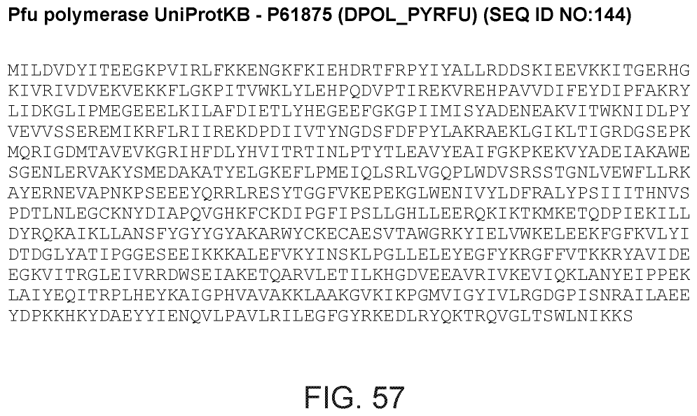

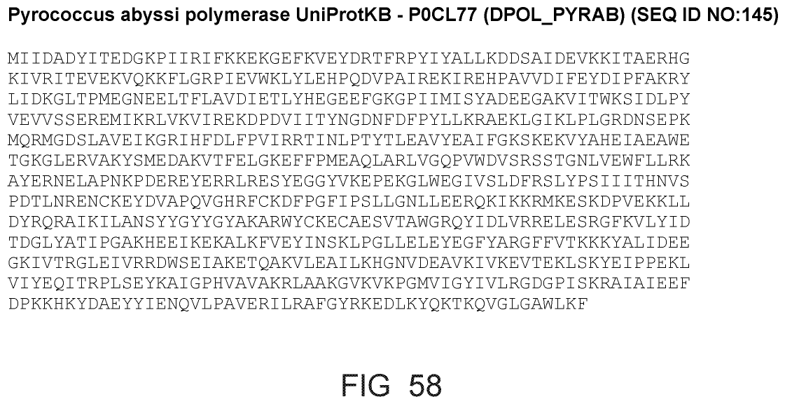

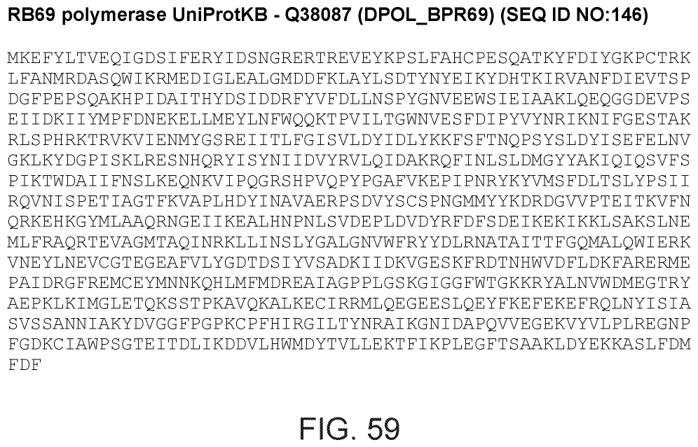

The present disclosure provides methods for sequencing concatemer template molecules, wherein the sequencing polymerase(s) is/are capable of binding a complementary nucleotide opposite a nucleotide in a concatemer template molecule. The present disclosure provides methods for sequencing concatemer template molecules, wherein the sequencing polymerase(s) is/are capable of incorporating a complementary nucleotide opposite a nucleotide in a concatemer template molecule. The present disclosure provides methods for sequencing concatemer template molecules, wherein, the sequencing polymerase(s) is/are capable of binding a complementary nucleotide moiety of a multivalent molecule opposite a nucleotide in a concatemer template molecule. In some embodiments, the plurality of sequencing polymerases comprise recombinant mutant polymerases. Examples of suitable polymerases for use in sequencing with nucleotides and/or multivalent molecules include but are not limited to: Klenow DNA polymerase; Thermus aquaticus DNA polymerase I (Taq polymerase); KlenTaq polymerase; Candidatus altiarchaeales archaeon (e.g., RLI89578.1, FIG. 41, SEQ ID NO:128); Candidatus Hadarchaeum Yellowstonense (e.g., KUO 58130, FIG. 42, SEQ ID NO:129); Hadesarchaea archaeon (e.g., MBC 7218772, FIG. 43, SEQ ID NO:130); Euryarchaeota archaeon (e.g., NOZ 58130, FIG. 44, SEQ ID NO:131), (e.g., RMF 90817, FIG. 45, SEQ ID NO:132), (e.g., NOZ 77387, FIG. 46, SEQ ID NO:133); Thermococcus archaeon (e.g., RLF 89458, FIG. 47, SEQ ID NO:134), (e.g., RLF 78286, FIG. 48, SEQ ID NO: 135), (e.g., WP 175059460, FIG. 49, SEQ ID NO:136); Thermoplasmata archaeon; Thermococcus polymerases such as Thermococcus litoralis, bacteriophage T7 DNA polymerase; human alpha, delta and epsilon DNA polymerases; bacteriophage polymerases such as T4, RB69 and phi29 bacteriophage DNA polymerases (e.g., FIG. 50, SEQ ID NO:137); Pyrococcus furiosus DNA polymerase (Pfu polymerase); Bacillus subtilis DNA polymerase III; E. coli DNA polymerase III alpha and epsilon; 9 degree N polymerase; reverse transcriptases such as HIV type M or O reverse transcriptases; avian myeloblastosis virus reverse transcriptase; Moloney Murine Leukemia Virus (MMLV) reverse transcriptase; or telomerase. Further non-limiting examples of DNA polymerases include those from various Archaea genera, such as, Aeropyrum, Archaeglobus, Desulfurococcus, Pyrobaculum, Pyrococcus, Pyrolobus, Pyrodictium, Staphylothermus, Stetteria, Sulfolobus, Thermococcus, and Vulcanisaeta and the like or variants thereof, including such polymerases as are known in the art such as Geobacillus stearothermophilus polymerase (e.g., FIG. 51, SEQ ID NO:138), 9 degrees N polymerase (e.g., FIGS. 52-53, SEQ ID NOS:139-140), VENT polymerase (e.g., FIG. 54, SEQ ID NO:141), DEEP VENT polymerase (e.g., FIG. 55, SEQ ID NO:142), THERMINATOR polymerase (e.g., FIG. 56, SEQ ID NO:143), Pfu polymerase (e.g., FIG. 57, SEQ ID NO:144), Pyrococcus abyssi polymerase (e.g., FIG. 58, SEQ ID NO:145), RB69 polymerase (e.g., FIG. 59, SEQ ID NO:146), KOD polymerase, Pfx polymerase, and Tgo polymerase.

In some embodiments, any of the sequencing methods can be conducted using a plurality of engineered polymerases comprising at least 70%, 75%, 80%, 85%, 90%, 91%, 92%, 93%, 94%, 95%, 96%, 97%, 98%, 99%, or greater than 99% sequence identity to any of SEQ ID NOS:128-146 (e.g., FIGS. 41-59 respectively).

In some embodiments, the engineered polymerases comprise a backbone sequence of RLI 89578.1 (SEQ ID NO:128, FIG. 41) and comprise at least one amino acid substitution mutation that reduces 3′ to 5′ exonuclease activity compared to a polymerase that lacks an exo-minus mutation. For example, the mutant polymerases comprise at least one amino acid substitution at positions D141 and/or E143. In some embodiments, the mutant polymerases comprise a mutation of D141A, D141V, D141L, D141I, D141F, D141Y, D141N, D141T and/or D141S. In some embodiments, the mutant polymerases comprise a mutation E143A, E143V, E143L, E1431, E143F, E143Y, E143N, E143T or E143S. In some embodiments, the mutant polymerases comprise any combination of mutations at the D141 and the E143 sites.

In some embodiments, the engineered polymerases comprise a backbone sequence of RLI 89578.1 (SEQ ID NO:128, FIG. 41) and comprise at least one amino acid substitution mutations of an LYP motif, for example at positions L416, Y417 and P418. In some embodiments, at least one mutation in the LYP motif can increase the incorporation rate of nucleotide analogs. In some embodiments, any one or any combination of the first, second and/or third positions of the LYP motif can be mutated. For example, mutations of the LYP motif can include YAG, FAG, YGP, YAP, FGP, SAP, AAA, YGA, YAA, FGA, FTA, AAG, AAP, AAV, AAI, AGA, AGG, AGI, AGP, AGV, FAA, FAI, FAP, FAV, FGG, FGV, LAG, LAI, LAP, LGG, LGI, LGV, SAA, SAG, SAI, SAV, SGA, SGG, SGI, YAI, YGG, YGI, LAA, LAV, LGP, LGA, FGI, SGV, YAV, YGV, SYP, SGP, LFP, IFP, VFP, LMP, VMP, IMP, LLP, VLP, ILP, LDP, VDP, IDP, LTP, VTP, ITP, LIP, TIP, NNP, NDP, NAP or SYG.

In some embodiments, the engineered polymerases comprise a backbone sequence of KUO 42443.1 (SEQ ID NO:129, FIG. 42) and comprise at least one amino acid substitution mutation that reduces 3′ to 5′ exonuclease activity compared to a polymerase that lacks an exo-minus mutation. For example, the mutant polymerases comprise at least one amino acid substitution at positions D170 and/or E172. In some embodiments, the mutant polymerases comprise a mutation of D170A, D170V, D170L, D1701, D170F, D170Y, D170N, D170T or D170S. In some embodiments, the mutant polymerases comprise a mutation of E172A, E172V, E172L, E1721, E172F, E172Y, E172N, E172T and/or E172S. In some embodiments, the mutant polymerases comprise any combination of mutations at the D170 and the E172 sites.

In some embodiments, the engineered polymerases comprise a backbone sequence of KUO 42443.1 (SEQ ID NO:129, FIG. 42) and comprise at least one amino acid substitution mutations of an LYP motif, for example at positions L448, Y449 and P450. In some embodiments, at least one mutation in the LYP motif can increase the incorporation rate of nucleotide analogs. In some embodiments, any one or any combination of the first, second and/or third positions of the LYP motif can be mutated. For example, mutations of the LYP motif can include AAA, SAG, SGP, SAP, LAP, AAG, AGA, AGP, FAA, FAG, FGA, FGP, SAA, SGA, YAG, YGA, YGP, FAP, YAP, AAP, YAA, AGV, AGG, AGI, AAV, AAI, FAI, FTA, FAV, FGG, FGV, LAG, LAI, LGG, LGI, LGV, SAI, SAV, SGG, SGI, YAI, YGG, YGI, LAA, LAV, LGP, LGA, FGI, SGV, YAV, YGV, SYP, LFP, IFP, VFP, LMP, VMP, IMP, LLP, VLP, ILP, LDP, VDP, IDP, LTP, VTP, ITP, LIP, TIP, NNP, NDP, NAP or SYG.

In some embodiments, the engineered polymerases comprise a backbone sequence of MBC 7218772.1 (SEQ ID NO:130, FIG. 43) and comprise at least one amino acid substitution mutation that reduces 3′ to 5′ exonuclease activity compared to a polymerase that lacks an exo-minus mutation. For example, the mutant polymerases comprise at least one amino acid substitution at positions D173 and/or E175. In some embodiments, the mutant polymerases comprise a mutation of D173A, D173V, D173L, D1731, D173F, D173Y, D173N, D173T or D173S. In some embodiments, the mutant polymerases comprise a mutation of E175A, E175V, E175L, E1751, E175F, E175Y, E175N, E175T or E175S. In some embodiments, the mutant polymerases comprise any combination of mutations at the D173 and the E175 sites.

In some embodiments, the engineered polymerases comprise a backbone sequence of MBC 7218772.1 (SEQ ID NO:130, FIG. 43) and comprise at least one amino acid substitution mutations of an LYP motif, for example at positions L451, Y452 and P453. In some embodiments, at least one mutation in the LYP motif can increase the incorporation rate of nucleotide analogs. In some embodiments, any one or any combination of the first, second and/or third positions of the LYP motif can be mutated. For example, mutations of the LYP motif can include AAA, AAG, AGA, AGP, FAA, FAG, FGA, FGP, SAA, SAG, SGA, SGP, YAA, YAG, YGA, YGP, FAP, SAP, YAP, AAP, LAP, AGV, AGG, AGI, AAV, AAI, FAI, FTA, FAV, FGG, FGV, LAG, LAI, LGG, LGI, LGV, SAI, SAV, SGG, SGI, YAI, YGG, YGI, LAA, LAV, LGP, LGA, FGI, SGV, YAV, YGV, SYP, LFP, IFP, VFP, LMP, VMP, IMP, LLP, VLP, ILP, LDP, VDP, IDP, LTP, VTP, ITP, LIP, TIP, NNP, NDP, NAP or SYG.

In some embodiments, the engineered polymerases comprise a backbone sequence of NOZ 58130.1 (SEQ ID NO:131, FIG. 44) and comprise at least one amino acid substitution mutation that reduces 3′ to 5′ exonuclease activity compared to a polymerase that lacks an exo-minus mutation. For example, the mutant polymerases comprise at least one amino acid substitution at positions D168 and/or E170. In some embodiments, the mutant polymerases comprise a mutation of D168A, D168V, D168L, D1681, D168F, D168Y, D168N, D168T and/or D168S. In some embodiments, the mutant polymerases comprise a mutation of E170A, E170V, E170L, E1701, E170F, E170Y, E170N, E170T and/or E170S. In some embodiments, the mutant polymerases comprise any combination of mutations at the D168 and the E170 sites.

In some embodiments, the engineered polymerases comprise a backbone sequence of NOZ 58130.1 (SEQ ID NO:131, FIG. 44) and comprise at least one amino acid substitution mutations of an LYP motif, for example at positions L440, Y441 and P442. In some embodiments, at least one mutation in the LYP motif can increase the incorporation rate of nucleotide analogs. In some embodiments, any one or any combination of the first, second and/or third positions of the LYP motif can be mutated. For example, mutations of the LYP motif can include YAG, FAG, YGP, YAP, FGP, SAP, AAA, YGA, YAA, FGA, FTA, AAG, AAP, AAV, AAI, AGA, AGG, AGI, AGP, AGV, FAA, FAI, FAP, FAV, FGG, FGV, LAG, LAI, LAP, LGG, LGI, LGV, SAA, SAG, SAI, SAV, SGA, SGG, SGI, YAI, YGG, YGI, LAA, LAV, LGP, LGA, FGI, SGV, YAV, YGV, SYP, SGP, LFP, IFP, VFP, LMP, VMP, IMP, LLP, VLP, ILP, LDP, VDP, IDP, LTP, VTP, ITP, LIP, TIP, NNP, NDP, NAP or SYG.

In some embodiments, the engineered polymerases comprise a backbone sequence of RMF 90817.1 (SEQ ID NO:132, FIG. 45) and comprise at least one amino acid substitution mutation that reduces 3′ to 5′ exonuclease activity compared to a polymerase that lacks an exo-minus mutation. For example, the mutant polymerases comprise at least one amino acid substitution at positions D149 and/or E151. In some embodiments, the mutant polymerases comprise a mutation of D149A, D149V, D149L, D1491, D149F, D149Y, D149N, D149T and/or D149S. In some embodiments, the mutant polymerases comprise a mutation of E151A, E151V, E151L, E151I, E151F, E151Y, E151N, E151T and/or E151S. In some embodiments, the mutant polymerases comprise any combination of mutations at the D149 and the E151 sites.

In some embodiments, the engineered polymerases comprise a backbone sequence of RMF 90817.1 (SEQ ID NO:132, FIG. 45) and comprise at least one amino acid substitution mutations of an LYP motif, for example at positions L421, Y422 and P423. In some embodiments, at least one mutation in the LYP motif can increase the incorporation rate of nucleotide analogs. In some embodiments, any one or any combination of the first, second and/or third positions of the LYP motif can be mutated. For example, mutations of the LYP motif can include AAA, AAG, AGA, AGP, FAA, FAG, FGA, FGP, SAA, SAG, SGA, SGP, YAA, YAG, YGA, YGP, FAP, SAP, YAP, AAP, AGV, AGG, AGI, AAV, AAI, FAI, FTA, FAV, FGG, FGV, LAG, LAI, LAP, LGG, LGI, LGV, SAI, SAV, SGG, SGI, YAI, YGG, YGI, LAA, LAV, LGP, LGA, FGI, SGV, YAV, YGV, SYP, LFP, IFP, VFP, LMP, VMP, IMP, LLP, VLP, ILP, LDP, VDP, IDP, LTP, VTP, ITP, LIP, TIP, NNP, NDP, NAP or SYG.

In some embodiments, the engineered polymerases comprise a backbone sequence of NOZ 77387.1 (SEQ ID NO:133, FIG. 46) and comprise at least one amino acid substitution mutation that reduces 3′ to 5′ exonuclease activity compared to a polymerase that lacks an exo-minus mutation. For example, the mutant polymerases comprise at least one amino acid substitution at positions D161 and/or E163. In some embodiments, the mutant polymerases comprise a mutation of D161A, D161V, D161L, D161I, D161F, D161Y, D161N, D161T and/or D161S. In some embodiments, the mutant polymerases comprise a mutation of E163A, E163V, E163L, E1631, E163F, E163Y, E163N, E163T and/or E163S. In some embodiments, the mutant polymerases comprise any combination of mutations at the D161 and the E163 sites.

In some embodiments, the engineered polymerases comprise a backbone sequence of NOZ 77387.1 (SEQ ID NO:133, FIG. 46) and comprise at least one amino acid substitution mutations of an LYP motif, for example at positions L448, Y449 and P450. In some embodiments, at least one mutation in the LYP motif can increase the incorporation rate of nucleotide analogs. In some embodiments, any one or any combination of the first, second and/or third positions of the LYP motif can be mutated. For example, mutations of the LYP motif can include AAA, SAP, FAG, FGP, YAG, YGP, FAP, YAP, SAG, SGP, LAP, AAG, AGA, AGP, FAA, FGA, SAA, SGA, YGA, AAP, YAA, AGV, AGG, AGI, AAV, AAI, FAI, FTA, FAV, FGG, FGV, LAG, LAI, LGG, LGI, LGV, SAI, SAV, SGG, SGI, YAI, YGG, YGI, LAA, LAV, LGP, LGA, FGI, SGV, YAV, YGV, SYP, LFP, IFP, VFP, LMP, VMP, IMP, LLP, VLP, ILP, LDP, VDP, IDP, LTP, VTP, ITP, LIP, TIP, NNP, NDP, NAP or SYG.

In some embodiments, the engineered polymerases comprise a backbone sequence of RLF 89458.1 (SEQ ID NO:134, FIG. 47) or RLF 78286.1 (SEQ ID NO:135, FIG. 48) and comprise at least one amino acid substitution mutation that reduces 3′ to 5′ exonuclease activity compared to a polymerase that lacks an exo-minus mutation. For example, the mutant polymerases comprise at least one amino acid substitution at positions D141 and/or E143. In some embodiments, the mutant polymerases comprise a mutation of D141A, D141V, D141L, D141I, D141F, D141Y, D141N, D141T and/or D141S. In some embodiments, the mutant polymerases comprise a mutation of E143A, E143V, E143L, E1431, E143F, E143Y, E143N, E143T and/or E143S. In some embodiments, the mutant polymerases comprise any combination of mutations at the D141 and the E143 sites.

In some embodiments, the engineered polymerases comprise a backbone sequence of RLF 89458.1 RLF 89458.1 (SEQ ID NO:134, FIG. 47) or RLF 78286.1 (SEQ ID NO:135, FIG. 48) and comprise at least one amino acid substitution mutations of an LYP motif, for example at positions L409, Y410 and P411. In some embodiments, at least one mutation in the LYP motif can increase the incorporation rate of nucleotide analogs. In some embodiments, any one or any combination of the first, second and/or third positions of the LYP motif can be mutated. For example, mutations of the LYP motif can include AAG, AAP, AAV, AAI, AGA, AGG, AGI, AGP, AGV, FAA, FAG, FAI, FAP, FAV, FGA, FGG, FGP, FGV, LAG, LAI, LAP, LGG, LGI, LGV, SAA, SAG, SAI, SAV, SGA, SGG, SGI, YAA, YAG, YAI, YAP, YGA, YGG, YGI, YGP, LAA, LAV, LGP, LGA, FGI, SGV, YAV, YGV, SYP, SAP, AAA, SGP, LFP, IFP, VFP, LMP, VMP, IMP, LLP, VLP, ILP, LDP, VDP, IDP, LTP, VTP, ITP, LIP, TIP, NNP, NDP, NAP, SYG or FTA.

In some embodiments, the engineered polymerases comprise a backbone sequence of WP 175059460.1 (SEQ ID NO:136, FIG. 49) and comprise at least one amino acid substitution mutation that reduces 3′ to 5′ exonuclease activity compared to a polymerase that lacks an exo-minus mutation. For example, the mutant polymerases comprise at least one amino acid substitution at positions D141 and/or E143. In some embodiments, the mutant polymerases comprise a mutation of D141A, D141V, D141L, D141I, D141F, D141Y, D141N, D141T and/or D141S. In some embodiments, the mutant polymerases comprise a mutation of E143A, E143V, E143L, E1431, E143F, E143Y, E143N, E143T and/or E143S. In some embodiments, the mutant polymerases comprise any combination of mutations at the D141 and the E143 sites.

In some embodiments, the engineered polymerases comprise a backbone sequence of WP 175059460.1 (SEQ ID NO:136, FIG. 49) and comprise at least one amino acid substitution mutations of an LYP motif, for example at positions L451, Y452 and P453. In some embodiments, at least one mutation in the LYP motif can increase the incorporation rate of nucleotide analogs. In some embodiments, any one or any combination of the first, second and/or third positions of the LYP motif can be mutated. For example, mutations of the LYP motif can include AAA, AAG, AGA, AGP, FAA, FAG, FGA, FGP, SAA, SAG, SGA, SGP, YAA, YAG, YGA, YGP, FAP, SAP, YAP, AAP, LAP, AGV, AGG, AGI, AAV, AAI, FAI, FTA, FAV, FGG, FGV, LAG, LAI, LGG, LGI, LGV, SAI, SAV, SGG, SGI, YAI, YGG, YGI, LAA, LAV, LGP, LGA, FGI, SGV, YAV, YGV, SYP, LFP, IFP, VFP, LMP, VMP, IMP, LLP, VLP, ILP, LDP, VDP, IDP, LTP, VTP, ITP, LIP, TIP, NNP, NDP, NAP or SYG.

Exemplary polymerase suitable for nucleic acid sequences are described in U.S. patent application Ser. Nos. 17/705,011, 17/705,020, 17/705,043 (all 3 filed Mar. 25, 2022), all patent applications entitled ‘Engineered Polymerases’, are hereby expressly incorporated by reference in their entireties.

Exemplary polymerases suitable for nucleic acid sequences are described in published U.S. patent application Nos. 2023/0265400, 2023/0265401, and 2023/0265402, all patent applications entitled ‘Engineered Polymerases with Reduced Sequence-Specific Errors’, are hereby expressly incorporated by reference in their entireties.

### Nucleotides

The present disclosure provides methods for sequencing concatemer template molecules, where any of the sequencing methods described herein employ a nucleotide reagent comprising at least one nucleotide. The nucleotides comprise a base, sugar and at least one phosphate group. In some embodiments, at least one nucleotide in the plurality comprises an aromatic base, a five carbon sugar (e.g., ribose or deoxyribose), and one or more phosphate groups (e.g., 1-10 phosphate groups). The plurality of nucleotides can comprise at least one type of nucleotide selected from a group consisting of dATP, dGTP, dCTP, dTTP and dUTP. The plurality of nucleotides can comprise at a mixture of any combination of two or more types of nucleotides selected from a group consisting of dATP, dGTP, dCTP, dTTP and/or dUTP. In some embodiments, at least one nucleotide in the plurality is not a nucleotide analog. In some embodiments, at least one nucleotide in the plurality comprises a nucleotide analog.

In some embodiments, in any of the methods for sequencing concatemer template molecules described herein, at least one nucleotide in the plurality of nucleotides comprise a chain of one, two or three phosphorus atoms where the chain is typically attached to the 5′ carbon of the sugar moiety via an ester or phosphoramide linkage. In some embodiments, at least one nucleotide in the plurality is an analog having a phosphorus chain in which the phosphorus atoms are linked together with intervening O, S, NH, methylene or ethylene. In some embodiments, the phosphorus atoms in the chain include substituted side groups including 0, S or BH₃. In some embodiments, the chain includes phosphate groups substituted with analogs including phosphoramidate, phosphorothioate, phosphorodithioate, and O-methyl-phosphoroamidite groups.

In some embodiments, in any of the methods for sequencing concatemer template molecules described herein, at least one nucleotide in the plurality of nucleotides comprises a terminator nucleotide analog having a chain terminating moiety (e.g., blocking moiety) at the sugar 2′ position, at the sugar 3′ position, or at the sugar 2′ and 3′ position. In some embodiments, the chain terminating moiety can inhibit polymerase-catalyzed incorporation of a subsequent nucleotide moiety or free nucleotide in a nascent strand during a primer extension reaction. In some embodiments, the chain terminating moiety is attached to the 3′ sugar hydroxyl position where the sugar comprises a ribose or deoxyribose sugar moiety. In some embodiments, the chain terminating moiety is removable/cleavable from the 3′ sugar hydroxyl position to generate a nucleotide having a 3′OH sugar group which is extendible with a subsequent nucleotide in a polymerase-catalyzed nucleotide incorporation reaction. In some embodiments, the chain terminating moiety comprises an alkyl group, alkenyl group, alkynyl group, allyl group, aryl group, benzyl group, azide group, amine group, amide group, keto group, isocyanate group, phosphate group, thio group, disulfide group, carbonate group, urea group, acetal group or silyl group. In some embodiments, the chain terminating moiety is cleavable/removable from the nucleotide, for example by reacting the chain terminating moiety with a chemical agent, pH change, light or heat. In some embodiments, the chain terminating moieties alkyl, alkenyl, alkynyl and allyl are cleavable with tetrakis(triphenylphosphine)palladium(0) (Pd(PPh₃)₄) with piperidine, or with 2,3-Dichloro-5,6-dicyano-1,4-benzo-quinone (DDQ). In some embodiments, the chain terminating moieties aryl and benzyl are cleavable with H2 Pd/C. In some embodiments, the chain terminating moieties amine, amide, keto, isocyanate, phosphate, thio, disulfide are cleavable with phosphine or with a thiol group including beta-mercaptoethanol or dithiothritol (DTT). In some embodiments, the chain terminating moiety carbonate is cleavable with potassium carbonate (K₂CO₃) in MeOH, with triethylamine in pyridine, or with Zn in acetic acid (AcOH). In some embodiments, the chain terminating moieties urea and silyl are cleavable with tetrabutylammonium fluoride, pyridine-HF, with ammonium fluoride, or with triethylamine trihydrofluoride.

In some embodiments, in any of the methods for sequencing concatemer template molecules described herein, at least one nucleotide in the plurality of nucleotides comprises a terminator nucleotide analog having a chain terminating moiety (e.g., blocking moiety) at the sugar 2′ position, at the sugar 3′ position, or at the sugar 2′ and 3′ position. In some embodiments, the chain terminating moiety comprises an azide, azido or azidomethyl group. In some embodiments, the chain terminating moiety comprises a 3′-O-azido or 3′-O-azidomethyl group. In some embodiments, the chain terminating moieties azide, azido and azidomethyl group are cleavable/removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (BS-TPP) or Tri(hydroxyproyl)phosphine (THPP). In some embodiments, the cleaving agent comprises 4-dimethylaminopyridine (4-DMAP).

In some embodiments, in any of the methods for sequencing concatemer template molecules described herein, the nucleotide comprises a chain terminating moiety which is selected from a group consisting of 3′-deoxy nucleotides, 2′,3′-dideoxynucleotides, 3′-methyl, 3′-azido, 3′-azidomethyl, 3′-O-azidoalkyl, 3′-O-ethynyl, 3′-O-aminoalkyl, 3′-O-fluoroalkyl, 3′-fluoromethyl, 3′-difluoromethyl, 3′-trifluoromethyl, 3′-sulfonyl, 3′-malonyl, 3′-amino, 3′-O-amino, 3′-sulfhydral, 3′-aminomethyl, 3′-ethyl, 3′ butyl, 3′-tert butyl, 3′-Fluorenylmethyloxycarbonyl, 3′ tert-Butyloxycarbonyl, 3′-O-alkyl hydroxylamino group, 3′-phosphorothioate, and 3-O-benzyl, or derivatives thereof.

In some embodiments, in any of the methods for sequencing concatemer template molecules described herein, the plurality of nucleotides comprises a plurality of nucleotides labeled with detectable reporter moiety. The detectable reporter moiety comprises a fluorophore. In some embodiments, the fluorophore is attached to the nucleotide base. In some embodiments, the fluorophore is attached to the nucleotide base with a linker which is cleavable/removable from the base. In some embodiments, at least one of the nucleotides in the plurality is not labeled with a detectable reporter moiety. In some embodiments, a particular detectable reporter moiety (e.g., fluorophore) that is attached to the nucleotide can correspond to the nucleotide base (e.g., dATP, dGTP, dCTP, dTTP or dUTP) to permit detection and identification of the nucleotide base.

In some embodiments, in any of the methods for sequencing concatemer template molecules described herein, the cleavable linker on the nucleotide base comprises a cleavable moiety comprising an alkyl group, alkenyl group, alkynyl group, allyl group, aryl group, benzyl group, azide group, amine group, amide group, keto group, isocyanate group, phosphate group, thio group, disulfide group, carbonate group, urea group, acetal group or silyl group. In some embodiments, the cleavable linker on the base is cleavable/removable from the base by reacting the cleavable moiety with a chemical agent, pH change, light or heat. In some embodiments, the cleavable moieties alkyl, alkenyl, alkynyl and allyl are cleavable with tetrakis(triphenylphosphine)palladium(0) (Pd(PPh₃)₄) with piperidine, or with 2,3-Dichloro-5,6-dicyano-1,4-benzo-quinone (DDQ). In some embodiments, the cleavable moieties aryl and benzyl are cleavable with H2 Pd/C. In some embodiments, the cleavable moieties amine, amide, keto, isocyanate, phosphate, thio, disulfide are cleavable with phosphine or with a thiol group including beta-mercaptoethanol or dithiothritol (DTT). In some embodiments, the cleavable moiety carbonate is cleavable with potassium carbonate (K₂CO₃) in MeOH, with triethylamine in pyridine, or with Zn in acetic acid (AcOH). In some embodiments, the cleavable moieties urea and silyl are cleavable with tetrabutylammonium fluoride, pyridine-HF, with ammonium fluoride, or with triethylamine trihydrofluoride.

In some embodiments, in any of the methods for sequencing concatemer template molecules described herein, the cleavable linker on the nucleotide base comprises cleavable moiety including an azide, azido or azidomethyl group. In some embodiments, the cleavable moieties azide, azido and azidomethyl group are cleavable/removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (BS-TPP) or Tri(hydroxyproyl)phosphine (THPP). In some embodiments, the cleaving agent comprises 4-dimethylaminopyridine (4-DMAP).

In some embodiments, in any of the methods for sequencing concatemer template molecules described herein, the chain terminating moiety (e.g., at the sugar 2′ and/or sugar 3′ position) and the cleavable linker on the nucleotide base have the same or different cleavable moieties. In some embodiments, the chain terminating moiety (e.g., at the sugar 2′ and/or sugar 3′ position) and the detectable reporter moiety linked to the base are chemically cleavable/removable with the same chemical agent. In some embodiments, the chain terminating moiety (e.g., at the sugar 2′ and/or sugar 3′ position) and the detectable reporter moiety linked to the base are chemically cleavable/removable with different chemical agents.

### Multivalent Molecules

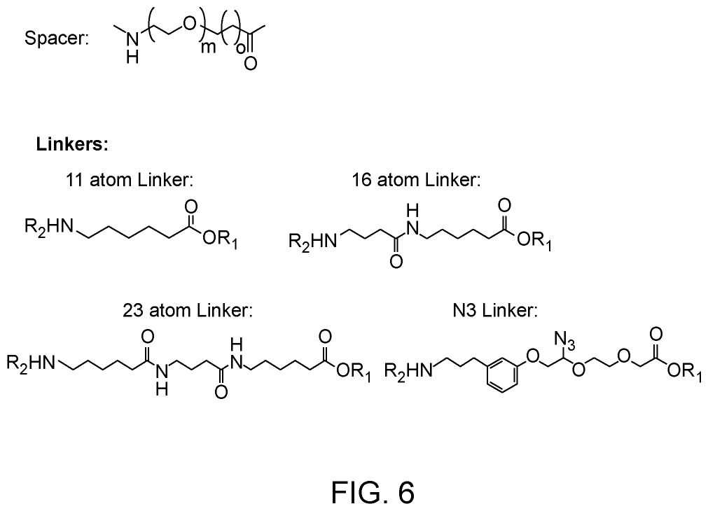

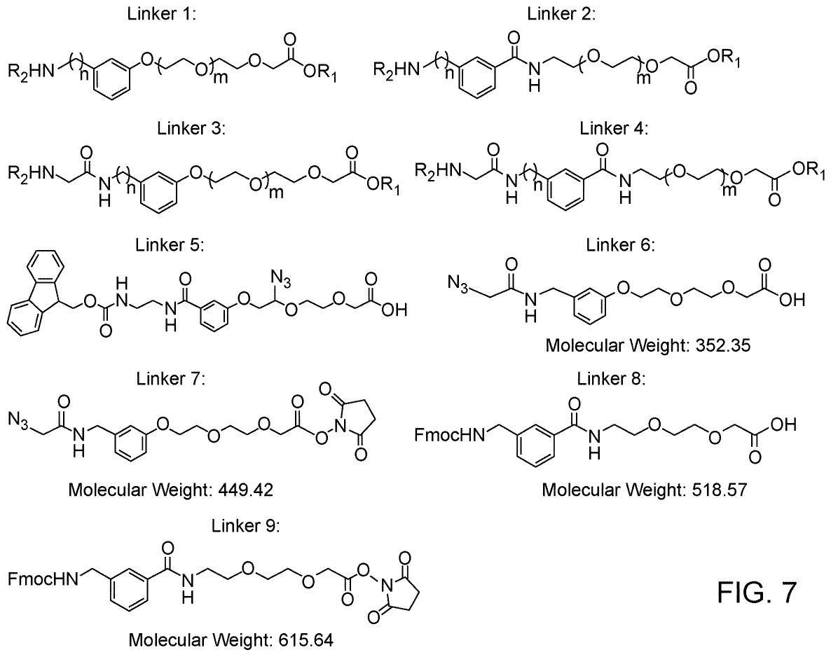

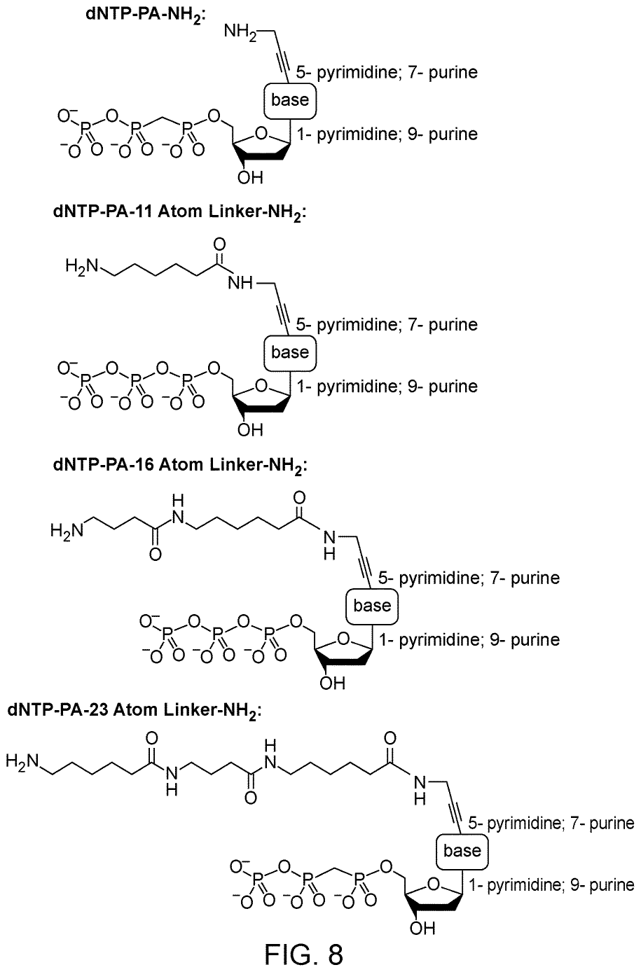

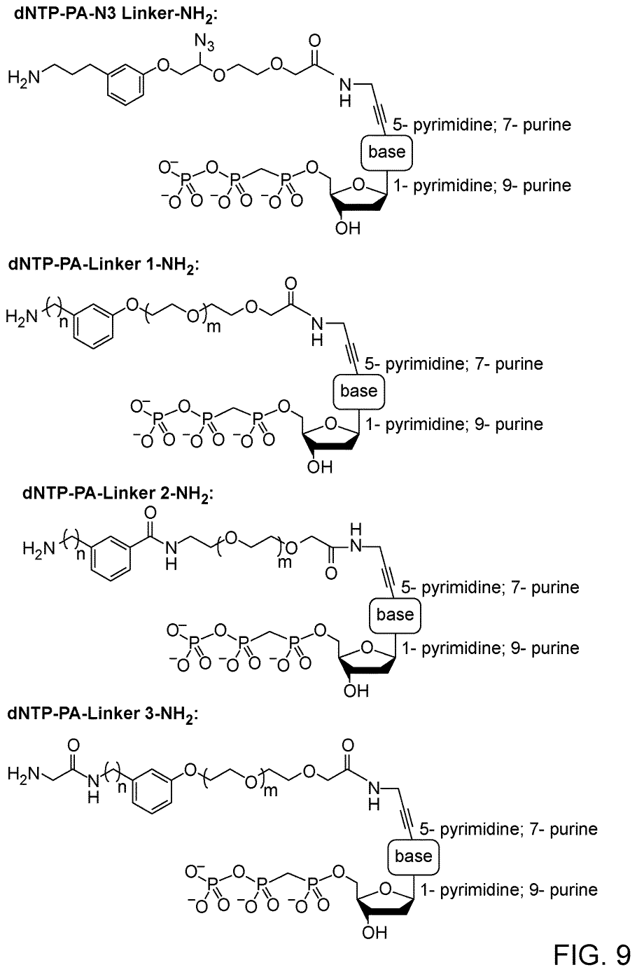

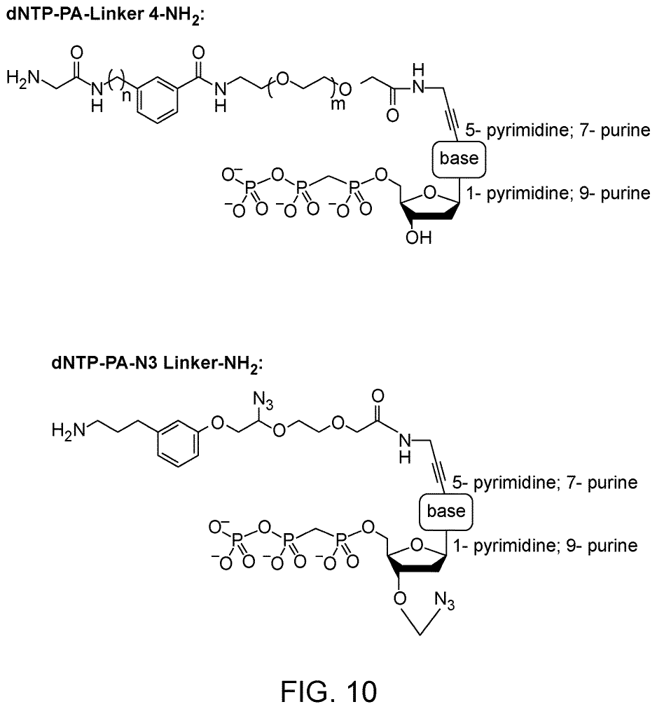

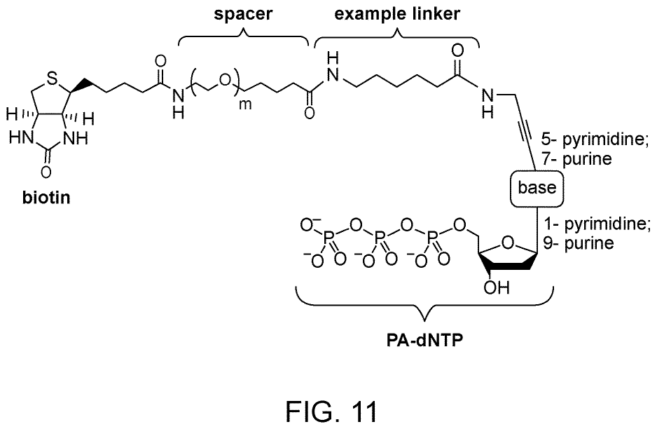

The present disclosure provides methods for sequencing concatemer template molecules, where any of the sequencing methods described herein employ a nucleotide reagent comprising at least one multivalent molecule. In some embodiments, the multivalent molecule comprises a plurality of nucleotide arms attached to a core and having any configuration including a starburst, helter skelter, or bottle brush configuration (e.g., FIG. 1). In some embodiments, multivalent molecule comprises: (1) a core; and (2) a plurality of nucleotide arms which comprise (i) a core attachment moiety, (ii) a spacer comprising a PEG moiety, (iii) a linker, and (iv) a nucleotide moiety, wherein the core is attached to the plurality of nucleotide arms, wherein the spacer is attached to the linker, wherein the linker is attached to the nucleotide moiety. In some embodiments, the multivalent molecule lacks a linker. In some embodiments, the nucleotide moiety comprises a base, sugar and at least one phosphate group, and the linker is attached to the nucleotide moiety through the base. In some embodiments, the linker comprises an aliphatic chain or an oligo ethylene glycol chain where both linker chains having 2-6 subunits. In some embodiments, the linker also includes an aromatic moiety. An exemplary nucleotide arm is shown in FIG. 5. Exemplary multivalent molecules are shown in FIGS. 1-4. An exemplary spacer is shown in FIG. 6 (top) and exemplary linkers are shown in FIG. 6 (bottom) and FIG. 7. Exemplary nucleotides attached to a linker are shown in FIGS. 8-10. An exemplary biotinylated nucleotide arm is shown in FIG. 11.

In some embodiments, a multivalent molecule comprises a core attached to multiple nucleotide arms, and wherein the multiple nucleotide arms have the same type of nucleotide moiety which is selected from a group consisting of dATP, dGTP, dCTP, dTTP and dUTP.

In some embodiments, a multivalent molecule comprises a core attached to multiple nucleotide arms, where each arm includes a nucleotide moiety. The nucleotide moiety comprises an aromatic base, a five carbon sugar (e.g., ribose or deoxyribose), and one or more phosphate groups (e.g., 1-10 phosphate groups). The plurality of multivalent molecules can comprise one type multivalent molecule having one type of nucleotide moiety selected from a group consisting of dATP, dGTP, dCTP, dTTP and dUTP. The plurality of multivalent molecules can comprise at a mixture of any combination of two or more types of multivalent molecules, where individual multivalent molecules in the mixture comprise nucleotide moieties selected from a group consisting of dATP, dGTP, dCTP, dTTP and/or dUTP.

In some embodiments, the nucleotide moiety comprises a chain of one, two or three phosphorus atoms where the chain is typically attached to the 5′ carbon of the sugar moiety via an ester or phosphoramide linkage. In some embodiments, at least one nucleotide moiety is a nucleotide analog having a phosphorus chain in which the phosphorus atoms are linked together with intervening O, S, NH, methylene or ethylene. In some embodiments, the phosphorus atoms in the chain include substituted side groups including 0, S or B3. In some embodiments, the chain includes phosphate groups substituted with analogs including phosphoramidate, phosphorothioate, phosphorodithioate, and O-methylphosphoroamidite groups.

In some embodiments, the multivalent molecule comprises a core attached to multiple nucleotide arms, and wherein individual nucleotide arms comprise a nucleotide moiety which is a nucleotide analog having a chain terminating moiety (e.g., blocking moiety) at the sugar 2′ position, at the sugar 3′ position, or at the sugar 2′ and 3′ position. In some embodiments, the nucleotide moiety comprises a chain terminating moiety (e.g., blocking moiety) at the sugar 2′ position, at the sugar 3′ position, or at the sugar 2′ and 3′ position. In some embodiments, the chain terminating moiety can inhibit polymerase-catalyzed incorporation of a subsequent nucleotide moiety or free nucleotide in a nascent strand during a primer extension reaction. In some embodiments, the chain terminating moiety is attached to the 3′ sugar hydroxyl position where the sugar comprises a ribose or deoxyribose sugar moiety. In some embodiments, the chain terminating moiety is removable/cleavable from the 3′ sugar hydroxyl position to generate a nucleotide having a 3′OH sugar group which is extendible with a subsequent nucleotide in a polymerase-catalyzed nucleotide incorporation reaction. In some embodiments, the chain terminating moiety comprises an alkyl group, alkenyl group, alkynyl group, allyl group, aryl group, benzyl group, azide group, amine group, amide group, keto group, isocyanate group, phosphate group, thio group, disulfide group, carbonate group, urea group, acetal group or silyl group. In some embodiments, the chain terminating moiety is cleavable/removable from the nucleotide moiety, for example by reacting the chain terminating moiety with a chemical agent, pH change, light or heat. In some embodiments, the chain terminating moieties alkyl, alkenyl, alkynyl and allyl are cleavable with tetrakis(triphenylphosphine)palladium(0) (Pd(PPh₃)₄) with piperidine, or with 2,3-Dichloro-5,6-dicyano-1,4-benzo-quinone (DDQ). In some embodiments, the chain terminating moieties aryl and benzyl are cleavable with H2 Pd/C. In some embodiments, the chain terminating moieties amine, amide, keto, isocyanate, phosphate, thio, disulfide are cleavable with phosphine or with a thiol group including beta-mercaptoethanol or dithiothritol (DTT). In some embodiments, the chain terminating moiety carbonate is cleavable with potassium carbonate (K₂CO₃) in MeOH, with triethylamine in pyridine, or with Zn in acetic acid (AcOH). In some embodiments, the chain terminating moieties urea and silyl are cleavable with tetrabutylammonium fluoride, pyridine-HF, with ammonium fluoride, or with triethylamine trihydrofluoride.

In some embodiments, the nucleotide moiety of a nucleotide arm comprises a chain terminating moiety (e.g., blocking moiety) at the sugar 2′ position, at the sugar 3′ position, or at the sugar 2′ and 3′ position. In some embodiments, the chain terminating moiety comprises an azide, azido or azidomethyl group. In some embodiments, the chain terminating moiety comprises a 3′-O-azido or 3′-O-azidomethyl group. In some embodiments, the chain terminating moieties azide, azido and azidomethyl group are cleavable/removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (BS-TPP) or Tri(hydroxyproyl)phosphine (THPP). In some embodiments, the cleaving agent comprises 4-dimethylaminopyridine (4-DMAP).

In some embodiments, the nucleotide moiety of a nucleotide arm comprises a chain terminating moiety which is selected from a group consisting of 3′-deoxy nucleotides, 2′,3′-dideoxynucleotides, 3′-methyl, 3′-azido, 3′-azidomethyl, 3′-O-azidoalkyl, 3′-O-ethynyl, 3′-O-aminoalkyl, 3′-O-fluoroalkyl, 3′-fluoromethyl, 3′-difluoromethyl, 3′-trifluoromethyl, 3′-sulfonyl, 3′-malonyl, 3′-amino, 3′-O-amino, 3′-sulfhydral, 3′-aminomethyl, 3′-ethyl, 3′ butyl, 3′-tert butyl, 3′-Fluorenylmethyloxycarbonyl, 3′ tert-Butyloxycarbonyl, 3′-O-alkyl hydroxylamino group, 3′-phosphorothioate, and 3-O-benzyl, or derivatives thereof.

In some embodiments, the multivalent molecule comprises a core attached to multiple nucleotide arms, wherein the nucleotide arms comprise a spacer, linker and nucleotide moiety, and wherein the core, linker and/or nucleotide moiety is labeled with detectable reporter moiety. In some embodiments, the detectable reporter moiety comprises a fluorophore. In some embodiments, a particular detectable reporter moiety (e.g., fluorophore) that is attached to the multivalent molecule can correspond to the base (e.g., dATP, dGTP, dCTP, dTTP or dUTP) of the nucleotide moiety to permit detection and identification of the nucleotide base.

In some embodiments, at least one nucleotide arm of a multivalent molecule has a nucleotide moiety that is attached to a detectable reporter moiety. In some embodiments, the detectable reporter moiety is attached to the nucleotide base. In some embodiments, the detectable reporter moiety is attached to a phosphate moiety of the phosphate-chain. In some embodiments, the detectable reporter moiety comprises a fluorophore. In some embodiments, a particular detectable reporter moiety (e.g., fluorophore) that is attached to the multivalent molecule can correspond to the base (e.g., dATP, dGTP, dCTP, dTTP or dUTP) of the nucleotide moiety to permit detection and identification of the nucleotide base.

In some embodiments, the core of a multivalent molecule comprises an avidin-like or streptavidin-like moiety and the core attachment moiety comprises biotin. In some embodiments, the core comprises an streptavidin-type or avidin-type moiety which includes an avidin protein, as well as any derivatives, analogs and other non-native forms of avidin that can bind to at least one biotin moiety. Other forms of avidin moieties include native and recombinant avidin and streptavidin as well as derivatized molecules, e.g., non-glycosylated avidin and truncated streptavidins. For example, avidin moiety includes de-glycosylated forms of avidin, bacterial streptavidin produced by Streptomyces (e.g., Streptomyces avidinii), as well as derivatized forms, for example, N-acyl avidins, e.g., N-acetyl, N-phthalyl and N-succinyl avidin, and the commercially-available products EXTRAVIDIN, CAPTAVIDIN, NEUTRAVIDIN and NEUTRALITE AVIDIN.

In some embodiments, any of the methods for sequencing concatemer template molecules described herein can include forming a binding complex, where the binding complex comprises (i) a polymerase, a nucleic acid concatemer template molecule hybridized with a primer, and a nucleotide, or the binding complex comprises (ii) a polymerase, a nucleic acid concatemer template molecule hybridized with a primer, and a nucleotide moiety of a multivalent molecule. In some embodiments, the binding complex has a persistence time of greater than about 0.1, 0.2, 0.3, 0.4, 0.5, 0.6, 0.7, 0.8, 0.9, 1 second or 30 seconds or more than 30 seconds. The binding complex has a persistence time of greater than about 0.1-0.25 seconds, or about 0.25-0.5 seconds, or about 0.5-0.75 seconds, or about 0.75-1 second, or about 1-2 seconds, or about 2-3 seconds, or about 3-4 second, or about 4-5 seconds, or about 5-30 seconds, or more than 30 seconds and/or wherein the method is or may be carried out at a temperature of at or above 15° C., at or above 20° C., at or above 25° C., at or above 35° C., at or above 37° C., at or above 42° C. at or above 55° C. at or above 60° C., or at or above 72° C., or at or above 80° C., or within a range defined by any of the foregoing. In some embodiments, the binding complexes may have a persistence time of less than Is, greater than Is, greater than 2 s, greater than 3 s, greater than 5 s, greater than 10 s, greater than 15 s, greater than 20 s, greater than 30 s, greater than 60 s, greater than 120 s, greater than 360 s, greater than 3600 s, or more, or for a time lying within a range defined by any two or more of these values. The binding complex (e.g., ternary complex) remains stable until subjected to a condition that causes dissociation of interactions between any of the polymerase, template molecule, primer and/or the nucleotide moiety or the nucleotide. For example, a dissociating condition comprises contacting the binding complex with any one or any combination of a detergent, EDTA and/or water. In some embodiments, the present disclosure provides said method wherein the binding complex is deposited on, attached to, or hybridized to, a surface showing a contrast to noise ratio in the detecting step of greater than 20. In some embodiments, the present disclosure provides said method wherein the contacting is performed under a condition that stabilizes the binding complex when the nucleotide or nucleotide moiety is complementary to a next base of the template nucleic acid, and destabilizes the binding complex when the nucleotide or nucleotide moiety is not complementary to the next base of the template nucleic acid.

### Sample Indexes for Improved Base Calling

Generally, it is desirable to prepare nucleic acid linear libraries that will be distributed onto a support (e.g., coated flow cell), where the library molecules are circularized and amplified to generate concatemer template molecules that are immobilized at a high density to the support for massively parallel sequencing. For concatemer template molecules that are immobilized at high densities at random locations on the support, the ability to resolve high density fluorescent images for accurate base calling during sequencing runs becomes challenging.

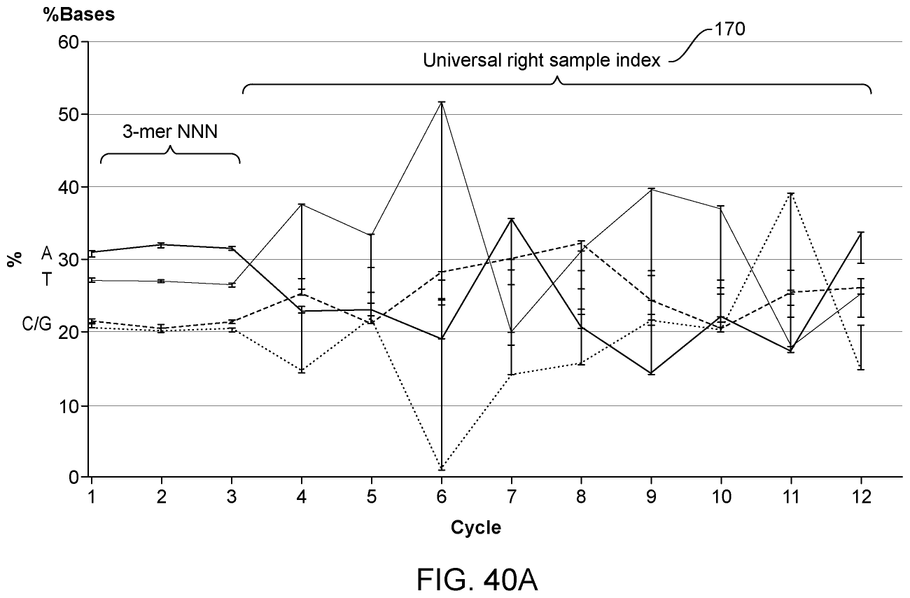

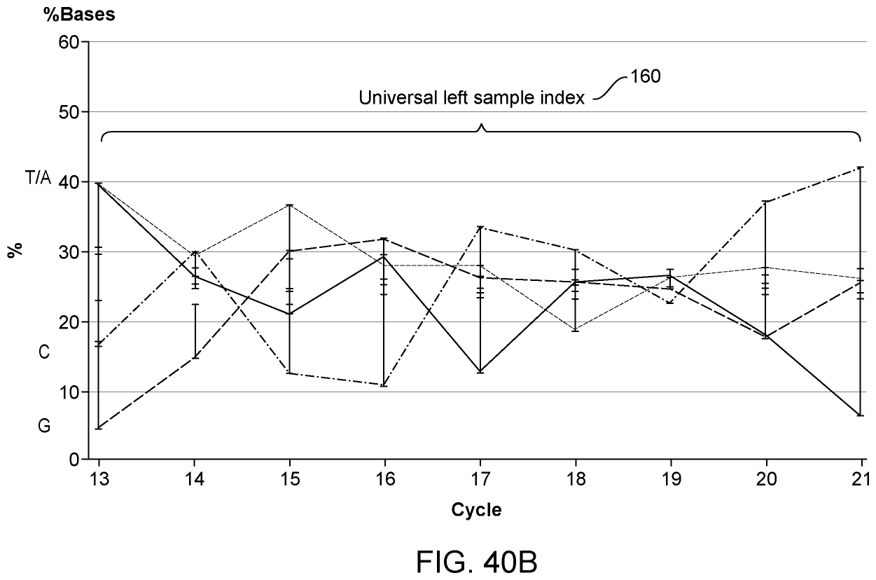

The nucleotide diversity of a population of immobilized concatemer template molecules refers to the relative proportion of nucleotides A, G, C and T that are present in each sequencing cycle. An optimal high diversity library will generally include sequence of interest (insert) regions having approximately equal proportions of all four nucleotides bases (e.g., A, G, C and T/U) represented in each cycle of a sequencing run. A low diversity library will generally include sequence of interest (insert) regions having a high proportion of certain nucleotides and low proportion of other nucleotides. To overcome the problem of low diversity libraries, a small amount of a high diversity library prepared from PhiX bacteriophage is typically mixed with the library-of-interest (e.g., PhiX spike-in library) and sequenced together on the same flow cell. While the PhiX library spike-in library provides nucleotide diversity it also occupies space on the flow cell thereby replacing the target libraries carrying the sequence of interest and reduces the amount of sequencing data obtainable from the target libraries (e.g., reduces sequencing throughput). Another method to overcome the problem of low diversity libraries is to prepare library molecules having at least one sample index sequence that is designed to be color-balanced. However it may be desirable to design a large number of sample index sets, for example a set of single index sample sequences or paired index sample sequences for 16-plex, 24-plex, 96-plex or larger plexy levels. It is challenging to design sample index sequences, as a single or paired sample indexes, for large sample index sets where all of the sample index sequences are color-balanced (e.g., see FIG. 40B).

An alternative method to overcome the challenges of sequencing low diversity library molecules (e.g., at high density on the support) is to prepare libraries having at least one sample index sequence comprising a short random sequence (e.g., 3-mer random sequence, NNN) linked directly to a universal sample index sequence, where the short random sequence provides nucleotide diversity and color balance. For example, any of the linear library molecules described herein can include at least one sample index sequence comprising a short random sequence, and the linear library molecules can be used to generate concatemers immobilized to a support. In a population of sample-indexed library molecules, the short random sequence of the sample index (e.g., (170)) provides high nucleotide diversity which includes approximately equal proportions of all four nucleotides (e.g., A, G, C, T and/or U) that will be represented in each cycle of a sequencing run (e.g., FIG. 40A). The high nucleotide diversity of the short random sequence also provide color balance during each cycle of the sequencing run. The advantage of designing sample index sequences (e.g., (160) and/or (170)) to include a short random sequence (e.g., NNN) is that, in a low-plexy population of library molecules (e.g., 2-plex or 4-plex), the universal sample index sequences that identify the two or four different samples need not exhibit nucleotide diversity (e.g., FIG. 40B). Additionally, the nucleotide diversity of the short random sequence (e.g., NNN) can obviate the need to include a PhiX spike-in library, or permits use of a reduced amount of PhiX spike-in library to be distributed onto the flow cell and sequenced.

The target library molecule can include a single sample index sequence (e.g., right sample index sequence (170)) which includes a short random sequence (e.g., NNN) and a universal sample index sequence. In some embodiments, the sequencing data from only the single sample index sequence (e.g., (170)) is used for polony mapping and template registration because the short random sequence (e.g., NNN) provides sufficient nucleotide diversity and color balance. The sequencing data from the universal sample index sequence can be used to distinguish sequences of interest obtained from different sample sources in a multiplex assay.

The target library molecule can further include a second sample index sequence (e.g., dual sample index) comprising a left sample index sequence (e.g., (160)). In some embodiments, the sequencing data from only the single sample index sequence (e.g., (170)) is used for polony mapping and/or template registration because the short random sequence provides sufficient nucleotide diversity and color balance. The sequencing data from the first universal sample index sequence (170) and the second universal sample index sequence (160) can be used as dual sample indexes to distinguish sequences of interest obtained from different sample sources in a multiplex assay. In some embodiments, the second sample index sequence (e.g., (160)) may or may not include a second short random sequence (e.g., NNN).

The order of sequencing the sequence of interest region and the sample index region(s) can also be used to improve the challenges of sequencing low diversity library molecules. For example, the sample index region can be sequenced first before sequencing the sequence of interest region, and the sample index sequence can be associated with the sequence of interest region. For example, sample index region can be sequenced first including sequencing the short random sequence (e.g., NNN) and optionally sequencing at least a portion of the universal sample index), and then sequencing the sequence of interest region. In a population of sample indexed library molecules, the short random sequence (e.g., NNN) provides nucleotide diversity which may not be provided by the sequence of interest regions of the library molecules. The sequence of the sample index provides improved nucleotide diversity and color balance for polony mapping and template registration.

Additionally, when sequencing the sample index region first, the length of the sequenced sample index region is relatively short (e.g., less than 30 nucleotides in length) so that de-hybridization of the product of the sequenced sample index region is more complete. Gentler de-hybridization conditions can be used to remove most or all of the product of the sequenced sample index region which reduces the level of residual signals from any sequencing products remaining hybridized to the concatemer template molecules. By contrast, the sequence of interest region is typically much longer than the sample index region (e.g., more than 100 nucleotides in length). When the sequence of interest region is sequenced before the sample index region, the product of the sequenced sequence of interest region must be subjected to harsher de-hybridization conditions to remove any products remaining hybridized to the concatemer template molecules which may damage the concatemer template molecules.

The present disclosure provides linear library molecules (100) each comprising at least one sample index sequence that can be used to distinguish sequences of interest obtained from different sample sources in a multiplex assay, where the at least one sample index sequence comprises a short random sequence (e.g., NNN) linked to a universal sample index sequence. In some embodiments, the left sample index (160) comprises a short random sequence (e.g., NNN) linked to a universal left sample index sequence and/or the right sample index sequence (170) comprises a short random sequence (e.g., NNN) linked to a right universal sample index sequence. The at least one sample index sequence can include sequence diversity for improved base calling. The at least one sample index sequence can be used to improve base calling accuracy.

In some embodiments, the short random sequence (e.g., NNN) is positioned upstream of the universal sample index sequence (e.g., (170) and/or (160)) so that during a sequencing run the random sequence portion is sequenced before the universal sample index sequence. In some embodiments, the short random sequence is positioned downstream of the universal sample index sequence so that during a sequencing run the random portion is sequenced after the universal sample index sequence.

In some embodiments, in the random sequence each base “N” at a given position is independently selected from A, G, C, T or U. In some embodiments, the random sequence lacks consecutive repeat sequences having 2 or 3 of the same nucleo-base, for example AA, TT, CC, GG, UU, AAA, TTT, CCC, GGG or UUU. In some embodiments, in a population of library molecules the universal sample index sequences (e.g., (170) and/or (160)) include a short random sequence having a high diversity sequence which includes approximately equal proportions of all four nucleotides (e.g., A, G, C, T and/or U) that will be represented in each cycle of a sequencing run.

In some embodiments, the short random sequence (e.g., NNN) comprises 3-20 nucleotides, or 3-10 nucleotides, or 3-8 nucleotides, or 3-6 nucleotides, or 3-5 nucleotides, or 3-4 nucleotides.

In some embodiments, the short random sequence (e.g., NNN) includes, but is not limited to, AGC, AGT, GAC, GAT, CAT, CAG, TAG, TAC. The skilled artisan will recognize that many more random sequences can be prepared (e.g., 64 possible combinations) where each base “N” at a given position in the random sequence is independently selected from A, G, C, T or U.

In some embodiments, the universal sample index sequence comprises 5-20 nucleotides, or 7-18 nucleotides, or 9-16 nucleotides.

In some embodiments, individual right sample index sequences (e.g., (170)) in a population of right sample index sequences comprise a universal sample index sequence and a short random sequence (e.g., NNN). In some embodiments, the short random sequences in the population of right sample index sequences have an overall base composition of about 25% or about 20-30% of all four nucleotide bases (e.g., A, G, C and T/U) to provide nucleotide diversity at each sequencing cycle during sequencing the short random sequence (e.g., NNN).

In some embodiments, in the population of right sample index sequences the proportion of adenine (A) at any given position in the short random sequence is about 20-30% or about 15-35% or about 10-40%. In some embodiments, in the population of right sample index sequences the proportion of guanine (G) at any given position in the short random sequence is about 20-30% or about 15-3⁵% or about 10-40%. In some embodiments, in the population of right sample index sequences the proportion of cytosine (C) at any given position in the short random sequence is about 20-30% or about 15-35% or about 10-40%. In some embodiments, in the population of right sample index sequences the proportion of thymine (T) or uracil (U) at any given position in the short random sequence is about 20-30% or about 15-35% or about 10-40%.

In some embodiments, in the population of right sample index sequences the proportion of adenine (A) and thymine (T), or the proportion of adenine (A) and uracil (U), at any given position in the short random sequence is about 10-65%. In some embodiments, in the population of right sample index sequences the proportion of guanine (G) and cytosine (C) at any given position in the short random sequence is about 10-65%.

In some embodiments, in the population of right sample index sequences the sequence diversity of the short random sequences ensures that no sequencing cycle is presented with fewer than four different nucleotide bases during sequencing at least the short random sequence (e.g., NNN).

Exemplary sample index sequence that include a short random sequence NNN linked directly to a universal sample index sequence include but are not limited to: NNNGTAGGAGCC; NNNCCGCTGCTA; NNNAACAACAAG; NNNGGTGGTCTA; NNNTTGGCCAAC; NNNCAGGAGTGC; and NNNATCACACTA. The skilled artisan will recognize that the universal sample index can be any length and have any sequence that can be used to distinguish sequences of interest obtained from different sample sources in a multiplex assay. In a population of a given sample index, for example NNNGTAGGAGCC, the population contains a mixture of individual sample index molecules each carrying the same universal sample index sequence (e.g., GTAGGAGCC) and a different short random sequence (e.g., NNN) where up to 64 different short random sequences may be present in the population of the given sample index.

In some embodiments, the random sequence (e.g., NNN) provides a balanced ratio of nucleo-bases adenine, cytosine, guanine, thymine and/or uracil (see FIG. 40A). In some embodiments, in a population of sample-indexed library molecules, the random sequence (e.g., NNN) together with at least a portion of the universal sample index sequence provide a balanced ratio of nucleo-bases adenine, cytosine, guanine, thymine and/or uracil represented in each cycle of a sequencing run.

In some embodiments, a sequencing reaction includes use of polymerases and nucleotide (e.g., nucleotide analogs) that are labeled with a different fluorophore that corresponds to the nucleo-base. In some embodiments, sequencing the random sequence (e.g., NNN) using labeled nucleotides provides a balanced ratio of fluorescent colors that correspond to the nucleo-bases adenine, cytosine, guanine, thymine and/or uracil in each cycle of a sequencing run. In some embodiments, sequencing the random sequence (e.g., NNN) and at least a portion of the universal sample index sequence using labeled nucleotides provides a balanced ratio of fluorescent colors that correspond to nucleo-bases adenine, cytosine, guanine, thymine and/or uracil (e.g., see FIG. 40A). The labeled nucleotides emit fluorescent signals during the sequencing reactions. In some embodiments, the sequencing reaction is conducted on a sequencing apparatus having a detector that captures fluorescent images from sequencing reactions on the immobilized concatemer template molecules. The sequencing apparatus can be configured to relay the fluorescent imaging data captured by the detector to a computer system that is programmed to determine the location (e.g., mapping) of the immobilized concatemer template molecules on the flow cell. The computer system can generate a map of the locations of the immobilized concatemer template molecules based on the fluorescent imaging data of only the random sequence (e.g., NNN), or based on the random sequence (e.g., NNN) and at least a portion the universal sample index sequence. Thus the few numbers of sequencing cycles used to sequence the random sequence (e.g., NNN) and optionally a portion of the universal sample index sequence can be used to generate a map of the location of the immobilized concatemer template molecules. The computer system can be configured to extract the fluorescent color and intensity of only the random sequence (e.g., NNN), or the random sequence (e.g., NNN) and at least a portion of the universal sample index sequence. The computer system can be configured to use the location of a given immobilized template molecule and the fluorescent color and intensity associated with the given template molecule (which were established while sequencing the random sequence) for base calling while sequencing the insert region (110). The computer system can be configured to detect phasing and pre-phasing while sequencing the random sequence (e.g., NNN) and the universal sample index sequence, and the insert region (110). In some embodiments, the balanced ratio of fluorescent colors provided by the random sequence (e.g., NNN) at each sequencing cycle can improve the quality of the data which is processed from the fluorescent images captured by the detector, and can in turn improve the capability by the computer system to determine the location of the immobilized concatemer template molecules on the flow cell, and the color and intensity, all of which can improve base calling accuracy and quality scores of the sequenced insert region (110).

In some embodiments, a sequencing reaction includes use of polymerases and multivalent molecules that are labeled with a different fluorophore that corresponds to the nucleo-base (e.g., adenine, guanine, cytosine, thymine or uracil) of the nucleotide moieties that are attached to the nucleotide arms in a given multivalent molecule. In some embodiments, the core of individual multivalent molecules is attached to a fluorophore which corresponds to the nucleotide moieties (e.g., adenine, guanine, cytosine, thymine or uracil) that are attached to the nucleotide arms in a given multivalent molecule (e.g., see FIGS. 1-4). In some embodiments, at least one of the nucleotide arms of the multivalent molecule comprises a linker and/or nucleotide base that is attached to a fluorophore, and wherein the fluorophore which is attached to a given linker or nucleotide base corresponds to the nucleotide base (e.g., adenine, guanine, cytosine, thymine or uracil) of the nucleotide arm. In some embodiments, sequencing the random sequence (e.g., NNN) using labeled multivalent molecules provides a balanced ratio of fluorescent colors that correspond to the nucleo-bases adenine, cytosine, guanine, thymine and/or uracil in each cycle of a sequencing run. In some embodiments, sequencing the random sequence (e.g., NNN) and at least a portion of the universal sample index sequence using labeled multivalent molecules provides a balanced ratio of fluorescent colors that correspond to nucleo-bases adenine, cytosine, guanine, thymine and/or uracil (e.g., see FIG. 40A). The labeled multivalent molecules emit fluorescent signals during the sequencing reactions. In some embodiments, the sequencing reaction is conducted on a sequencing apparatus having a detector that captures fluorescent images from sequencing reactions on the immobilized concatemer template molecules. The sequencing apparatus can be configured to relay the fluorescent imaging data captured by the detector to a computer system that is programmed to determine the location (e.g., mapping) of the immobilized concatemer template molecules (polonies) on the flow cell. The computer system can generate a map of the locations of the immobilized concatemer template molecules based on the fluorescent imaging data of only the random sequence (e.g., NNN), or based on the random sequence (e.g., NNN) and at least a portion of the universal sample index sequence. Thus the few numbers of sequencing cycles used to sequence the random sequence (e.g., NNN) and optionally a portion of the universal sample index sequence can be used to generate a map of the location of the immobilized concatemer template molecules. The computer system can be configured to extract the fluorescent color and intensity of only the random sequence (e.g., NNN) or the random sequence (e.g., NNN) and the universal sample index sequence. The computer system can be configured to use the location of a given immobilized template molecule and the fluorescent color and intensity associated with the given template molecule (which were established while sequencing the random sequence) for base calling while sequencing the insert region (110). The computer system can be configured to detect phasing and pre-phasing while sequencing the random sequence (e.g., NNN) and the universal sample index sequence, and the insert region (110). In some embodiments, the balanced ratio of fluorescent colors provided by the random sequence (e.g., NNN) at each sequencing cycle can improve the quality of the data which is processed from the fluorescent images captured by the detector, and can in turn improve the capability by the computer system to determine the location of the immobilized concatemer template molecules on the flow cell, and the color and intensity, all of which can improve base calling accuracy and quality scores of the sequenced insert region (110).

### A First Embodiment: Order of Sequencing Sample Index Sequences

In some embodiments, the order of sequencing comprises: (1) sequencing the right sample index sequence (170) where the right sample index comprises a first random sequence (e.g., NNN) and a right universal sample index sequence; (2) sequencing the left sample index (160); and (3) sequencing the insert region (110). In some embodiments, the left sample index (160) comprises a left universal sample index sequence. In some embodiments, the left sample index (160) comprises a second random sequence (e.g., NNN) and a left universal sample index sequence. In some embodiments, sequencing the right sample index sequence (170) region, including the first random sequence (e.g., NNN) and right universal sample index sequence, may provide enough nucleotide diversity so that sequencing the left sample index sequence (160) can be omitted.

In some embodiments, the method for sequencing comprises step (a): hybridizing the concatemer template molecules with a first plurality of soluble sequencing primers that hybridize to the universal binding sequence for a reverse sequencing primer (150) and sequencing the right sample index sequence (170) including sequencing the short random sequence (e.g., NNN) and the right universal sample index sequence thereby generating a first plurality of sample index extension products that are hybridized to the immobilized concatemer template molecules, wherein the first plurality of sample index extension products are complementary to the right sample index sequence having a short random sequence (e.g., NNN) linked directly to a right universal sample index sequence (170).

In some embodiments, the methods for sequencing further comprise step (b): removing the first plurality of sample index extension products and retaining the immobilized concatemer template molecules.

In some embodiments, the methods for sequencing further comprise step (c): hybridizing the retained immobilized concatemer template molecules with a second plurality of soluble sequencing primers that hybridize to the first universal binding site (120) for the first portion of the splint capture primer and sequencing the left sample index sequence (160) thereby generating a second plurality of sample index extension products that are hybridized to the immobilized concatemer template molecules, wherein the second plurality of sample index extension products are complementary to the left sample index sequence (160) having a left universal sample index sequence.

In some embodiments, the methods for sequencing further comprise step (d): removing the second plurality of sample index extension products and retaining the immobilized concatemer template molecules.

In some embodiments, the methods for sequencing further comprise step (e): hybridizing the retained immobilized concatemer template molecules with a third plurality of soluble sequencing primers that hybridize to the universal binding sequence for the forward sequencing primer (140) and sequencing the insert region (110), also referred to as sequence of interest, thereby generating a plurality of insert extension products that are hybridized to the immobilized concatemer template molecules, wherein the plurality of insert extension products are complementary to the sequence of interest (110).

In some embodiments, the methods for sequencing further comprise step (f1): assigning the sequence of (i) the insert region (110) to (ii) the right sample index sequence having a short random sequence (e.g., NNN) linked directly to a right universal sample index sequence (170), thereby identifying the insert region as being obtained from a first source.

In some embodiments, the methods for sequencing further comprise step (f2): assigning the sequence of (i) the insert region (110) to (ii) the right sample index sequence having a short random sequence (e.g., NNN) linked directly to a right universal sample index sequence (170), and (iii) the left sample index sequence (160), thereby identifying the insert region as being obtained from a first source.

In some embodiments, the removing of the plurality of sequencing extension products of steps (b) and (d) can be conducted using a denaturation reagent comprising SSC (e.g., saline-sodium citrate) buffer with or without formamide, at a temperature that promotes nucleic acid denaturation such as for example 50-90° C.

In some embodiments, the sequencing of steps (a), (c) and (e) include conducting any of the sequencing methods described herein that employ sequencing polymerases and detectably labeled nucleotide analogs. In some embodiments, the sequencing of steps (a), (c) and (e) include conducting any of the two-stage sequencing methods described herein that employ sequencing polymerases, detectably labeled multivalent molecules, and nucleotide analogs. In some embodiments, the sequencing of steps (a), (c) and (e) include conducting any of the sequencing-by-binding methods described herein. In some embodiments, the sequencing of steps (a), (c) and (e) include conducting any of the sequencing methods described herein that employ sequencing polymerases and a nucleotide reagent which comprises labeled chain terminating nucleotides, phosphate chain-labeled nucleotides or labeled multivalent molecules.

In some embodiments, the density of the plurality of concatemer template molecules immobilized to the support is about 10²-10¹⁵ per mm². In some embodiments, the plurality of concatemer template molecules are immobilized at random locations on the support. In some embodiments, the plurality of concatemer template molecules are immobilized on the support in a predetermined pattern.

### A Second Embodiment: Order of Sequencing Sample Index Sequences

In some embodiments, the order of sequencing comprises: (1) sequencing the right sample index sequence (170) where the right index comprises a random sequence (e.g., NNN) and a universal sample index sequence; (2) sequencing the insert region (110); and (3) sequencing the left sample index (160). In some embodiments, the left sample index (160) comprises a left universal sample index sequence. In some embodiments, the left sample index (160) comprises a second random sequence (e.g., NNN) and a left universal sample index sequence. In some embodiments, sequencing the right sample index sequence (170) region, including the first random sequence (e.g., NNN) and right universal sample index sequence, may provide enough nucleotide diversity so that sequencing the left sample index (160) can be omitted.

In some embodiments, the method for sequencing comprises step (a): hybridizing the concatemer template molecules with a first plurality of soluble sequencing primers that hybridize to the universal binding sequence for a reverse sequencing primer (150) and sequencing the right sample index sequence (170) including sequencing the short random sequence (e.g., NNN) and the right universal sample index sequence thereby generating a first plurality of sample index extension products that are hybridized to the immobilized concatemer template molecules, wherein the first plurality of sample index extension products are complementary to the right sample index sequence (170) having a short random sequence (e.g., NNN) linked directly to a right universal sample index sequence.

In some embodiments, the methods for sequencing further comprise step (b): removing the first plurality of sample index extension products and retaining the immobilized concatemer template molecules.

In some embodiments, the methods for sequencing further comprise step (c): hybridizing the retained immobilized concatemer template molecules with a second plurality of soluble sequencing primers that hybridize to the universal binding sequence for a forward sequencing primer (140) and sequencing the insert region (110) thereby generating a plurality of insert extension products that are hybridized to the immobilized concatemer template molecules, wherein the plurality of insert extension products are complementary to the sequence of interest (110).

In some embodiments, the methods for sequencing further comprise step (d): removing the plurality of insert extension products and retaining the immobilized concatemer template molecules.

In some embodiments, the methods for sequencing further comprise step (e): hybridizing the retained immobilized concatemer template molecules with a third plurality of soluble sequencing primers that hybridize to the first universal binding site (120) for the first portion of the splint capture primer and sequencing the left sample index sequence (160) thereby generating a second plurality of sample index extension products that are hybridized to the immobilized concatemer template molecules, wherein the second plurality of sample index extension products are complementary to the left sample index sequence (160) having a left universal sample index sequence.

In some embodiments, the methods for sequencing further comprise step (f1): assigning the sequence of (i) the insert region (110) to (ii) the right sample index sequence (170) having a short random sequence (e.g., NNN) linked directly to a right universal sample index sequence, thereby identifying the insert region as being obtained from a first source.

In some embodiments, the methods for sequencing further comprise step (f2): assigning the sequence of (i) the insert region (110) to (ii) the right sample index sequence (170) having a short random sequence (e.g., NNN) linked directly to a right universal sample index sequence, and (iii) the left sample index sequence (160), thereby identifying the insert region as being obtained from a first source.

In some embodiments, the removing of the plurality of sequencing extension products of steps (b) and (d) can be conducted using a denaturation reagent comprising SSC (e.g., saline-sodium citrate) buffer with or without formamide, at a temperature that promotes nucleic acid denaturation such as for example 50-90° C.

In some embodiments, the sequencing of steps (a), (c) and (e) include conducting any of the sequencing methods described herein that employ sequencing polymerases and detectably labeled nucleotide analogs. In some embodiments, the sequencing of steps (a), (c) and (e) include conducting any of the two-stage sequencing methods described herein that employ sequencing polymerases, detectably labeled multivalent molecules, and nucleotide analogs. In some embodiments, the sequencing of steps (a), (c) and (e) include conducting any of the sequencing-by-binding methods described herein. In some embodiments, the sequencing of steps (a), (c) and (e) include conducting any of the sequencing methods described herein that employ sequencing polymerases and a nucleotide reagent which comprises labeled chain terminating nucleotides, phosphate chain-labeled nucleotides or labeled multivalent molecules.

In some embodiments, the density of the plurality of concatemer template molecules immobilized to the support is about 10²-10¹⁵ per mm². In some embodiments, the plurality of concatemer template molecules are immobilized at random locations on the support. In some embodiments, the plurality of concatemer template molecules are immobilized on the support in a predetermined pattern.

### A Third Embodiment: Order of Sequencing Sample Index Sequences

In some embodiments, the order of sequencing comprises: (1) sequencing the insert region (110); (2) sequencing the right sample index sequence (170) where the right index comprises a random sequence (e.g., NNN) and a universal sample index sequence; and (3) sequencing the left sample index (160). In some embodiments, the left sample index (160) comprises a left universal sample index sequence. In some embodiments, the left sample index (160) comprises a second random sequence (e.g., NNN) and a left universal sample index sequence. In some embodiments, sequencing the right sample index sequence (170) region, including the first random sequence (e.g., NNN) and right universal sample index sequence, may provide enough nucleotide diversity so that sequencing the left sample index (160) can be omitted.

In some embodiments, the method for sequencing comprises step (a): hybridizing the concatemer template molecules with a first plurality of soluble sequencing primers that hybridize to the universal binding sequence for a forward sequencing primer (140) and sequencing the insert region (110) thereby generating a plurality of insert extension products that are hybridized to the immobilized concatemer template molecules, wherein the plurality of insert extension products are complementary to the sequence of interest (110).

In some embodiments, the methods for sequencing further comprise step (b): removing the plurality of insert extension products and retaining the immobilized concatemer template molecules.

In some embodiments, the methods for sequencing further comprise step (c): hybridizing the concatemer template molecules with a second plurality of soluble sequencing primers that hybridize to the universal binding sequence for a reverse sequencing primer (150) and sequencing the right sample index sequence (170) including sequencing the short random sequence (e.g., NNN) and the right universal sample index sequence thereby generating a first plurality of sample index extension products that are hybridized to the immobilized concatemer template molecules, wherein the first plurality of sample index extension products are complementary to the right sample index sequence having a short random sequence (e.g., NNN) linked directly to a right sample index sequence (170), which is a universal sample index sequence.

In some embodiments, the methods for sequencing further comprise step (d): removing the first plurality of sample index extension products and retaining the immobilized concatemer template molecules.

In some embodiments, the methods for sequencing further comprise step (e): hybridizing the retained immobilized concatemer template molecules with a third plurality of soluble sequencing primers that hybridize to the first universal binding site (120) for the first portion of the splint capture primer and sequencing the left sample index sequence (160) thereby generating a second plurality of sample index extension products that are hybridized to the immobilized concatemer template molecules, wherein the second plurality of sample index extension products are complementary to the left sample index sequence (160) having a left universal sample index sequence.

In some embodiments, the methods for sequencing further comprise step (f1): assigning the sequence of (i) the insert region (110) to (ii) the right sample index sequence having a short random sequence (e.g., NNN) linked directly to a right sample index sequence (170), which is a universal sample index sequence, thereby identifying the insert region as being obtained from a first source.

In some embodiments, the methods for sequencing further comprise step (f2): assigning the sequence of (i) the insert region (110) to (ii) the right sample index sequence having a short random sequence (e.g., NNN) linked directly to a right sample index sequence (170), which is a universal sample index sequence, and (iii) the left sample index sequence (160), thereby identifying the insert region as being obtained from a first source.

In some embodiments, the removing of the plurality of sequencing extension products of steps (b) and (d) can be conducted using a denaturation reagent comprising SSC (e.g., saline-sodium citrate) buffer with or without formamide, at a temperature that promotes nucleic acid denaturation such as for example 50-90° C.

In some embodiments, the sequencing of steps (a), (c) and (e) include conducting any of the sequencing methods described herein that employ sequencing polymerases and detectably labeled nucleotide analogs. In some embodiments, the sequencing of steps (a), (c) and (e) include conducting any of the two-stage sequencing methods described herein that employ sequencing polymerases, detectably labeled multivalent molecules, and nucleotide analogs. In some embodiments, the sequencing of steps (a), (c) and (e) include conducting any of the sequencing-by-binding methods described herein. In some embodiments, the sequencing of steps (a), (c) and (e) include conducting any of the sequencing methods described herein that employ sequencing polymerases and a nucleotide reagent which comprises labeled chain terminating nucleotides, phosphate chain-labeled nucleotides or labeled multivalent molecules.

In some embodiments, the density of the plurality of concatemer template molecules immobilized to the support is about 10²-10¹⁵ per mm². In some embodiments, the plurality of concatemer template molecules are immobilized at random locations on the support. In some embodiments, the plurality of concatemer template molecules are immobilized on the support in a predetermined pattern.

### A Fourth Embodiment: Order of Sequencing

In some embodiments, the order of sequencing comprises: (1) sequencing the first 3-5 bases of the insert region (110); (2) sequencing the right sample index sequence (170) where the right index comprises an optional random sequence (e.g., NNN) and a universal sample index sequence; (3) sequencing the left sample index sequence (160); and (4) sequencing the insert region (110). In some embodiments, the left sample index is not sequenced. In some embodiments, the left sample index sequence (160) comprises a left universal sample index sequence. In some embodiments, the left sample index sequence (160) comprises a second random sequence (e.g., NNN) and a left universal sample index sequence. In some embodiments, sequencing the first 3-5 bases of the insert region (110) may provide enough sequence diversity so that the right sample index sequence (170) and the left sample index sequence (160) need not include a short random sequence (e.g., NNN). In some embodiments, sequencing the right sample index sequence (170) region, including the first random sequence (e.g., NNN) and right universal sample index sequence, may provide enough nucleotide diversity so that sequencing the left sample index (160) can be omitted.

In some embodiments, the method for sequencing comprises step (a): hybridizing the concatemer template molecules with a first plurality of soluble sequencing primers that hybridize to the universal binding sequence for a forward sequencing primer (140) and sequencing the first 3-5 bases of the insert region (110) thereby generating a plurality of insert extension products that are hybridized to the immobilized concatemer template molecules, wherein the plurality of insert extension products are complementary to the sequence of interest (110). The sequence of the first 3-5 bases of the insert region (110) may provide sufficient sequence diversity and color balance for polony mapping and template registration.

In some embodiments, the methods for sequencing further comprise step (b): removing the plurality of insert extension products and retaining the immobilized concatemer template molecules.

In some embodiments, the methods for sequencing further comprise step (c): hybridizing the concatemer template molecules with a second plurality of soluble sequencing primers that hybridize to the universal binding sequence for a reverse sequencing primer (150) and sequencing the right sample index sequence (170) including sequencing the short random sequence (e.g., NNN) if present and the right universal sample index sequence thereby generating a first plurality of sample index extension products that are hybridized to the immobilized concatemer template molecules, wherein the first plurality of sample index extension products are complementary to the right sample index sequence (170).

In some embodiments, the methods for sequencing further comprise step (d): removing the first plurality of sample index extension products and retaining the immobilized concatemer template molecules.

In some embodiments, the methods for sequencing further comprise step (e): hybridizing the retained immobilized concatemer template molecules with a third plurality of soluble sequencing primers that hybridize to the first universal binding site (120) for the first portion of the splint capture primer and sequencing the left sample index sequence (160) thereby generating a second plurality of sample index extension products that are hybridized to the immobilized concatemer template molecules, wherein the second plurality of sample index extension products are complementary to the left sample index sequence (160) having a left universal sample index sequence.

In some embodiments, the methods for sequencing further comprise step (f): removing the second plurality of sample index extension products and retaining the immobilized concatemer template molecules.

In some embodiments, the methods for sequencing further comprise step (g): hybridizing the concatemer template molecules with a fourth plurality of soluble sequencing primers that hybridize to the universal binding sequence for a forward sequencing primer (140) and sequencing the full length of the insert region (110) thereby generating a plurality of full length insert extension products that are hybridized to the immobilized concatemer template molecules, wherein the plurality of full length insert extension products are complementary to the sequence of interest (110). In some embodiments, in step (g), the full length of the insert region (110) can be sequenced using detectably labeled nucleotide reagents. In some embodiments, in step (g), the first 3-5 bases of the insert region (110) can be subjected to “dark” sequencing using unlabeled nucleotide reagents and the remainder of the insert region (110) can be sequenced using detectably labeled nucleotide reagents.

In some embodiments, the methods for sequencing further comprise step (h1): assigning the full length sequence of (i) the insert region (110) to (ii) the right sample index sequence (170), thereby identifying the insert region as being obtained from a first source.

In some embodiments, the methods for sequencing further comprise step (h2): assigning the full length sequence of (i) the insert region (110) to (ii) the right sample index sequence (170), and (iii) the left sample index sequence (160), thereby identifying the insert region as being obtained from a first source.

In some embodiments, the removing of the plurality of sequencing extension products of steps (b), (d) and (f) can be conducted using a denaturation reagent comprising SSC (e.g., saline-sodium citrate) buffer with or without formamide, at a temperature that promotes nucleic acid denaturation such as for example 50-90° C.

In some embodiments, the sequencing of steps (a), (c), (e) and (g) include conducting any of the sequencing methods described herein that employ sequencing polymerases and detectably labeled nucleotide analogs. In some embodiments, the sequencing of steps (a), (c), (e) and (g) include conducting any of the two-stage sequencing methods described herein that employ sequencing polymerases, detectably labeled multivalent molecules, and nucleotide analogs. In some embodiments, the sequencing of steps (a), (c), (e) and (g) include conducting any of the sequencing-by-binding methods described herein. In some embodiments, the sequencing of steps (a), (c), (e) and (g) include conducting any of the sequencing methods described herein that employ sequencing polymerases and a nucleotide reagent which comprises labeled chain terminating nucleotides, phosphate chain-labeled nucleotides or labeled multivalent molecules.

In some embodiments, the density of the plurality of concatemer template molecules immobilized to the support is about 10²-10¹⁵ per mm². In some embodiments, the plurality of concatemer template molecules are immobilized at random locations on the support. In some embodiments, the plurality of concatemer template molecules are immobilized on the support in a predetermined pattern.

### A Fifth Embodiment: Order of Sequencing

In some embodiments, the order of sequencing comprises: (1) sequencing the first 3-5 bases of the insert region (110) of the immobilized template molecule (e.g., sequencing in a forward direction); (2) sequencing the right sample index sequence (170) where the right index comprises a first random sequence (e.g., NNN) and a right universal sample index sequence; (3) sequencing the left sample index (160); (4) conducting a pairwise turn reaction so that the immobilized template molecule is replaced with an immobilized strand that is complementary to the template molecule; and (5) sequencing the full-length of the insert region (110) of the immobilized complementary strand (e.g., sequencing in the reverse direction). In some embodiments, the sequences of the first 3-5 bases of the insert region (110) of a population of library molecules may provide enough sequence diversity for improved base-calling accuracy and/or for polony mapping and template registration. In some embodiments, the left sample index sequence (160) comprises a left universal sample index sequence. In some embodiments, the left sample index sequence (160) comprises a second random sequence (e.g., NNN) and a left universal sample index sequence. In some embodiments, sequencing the right sample index sequence (170) region, including the first random sequence (e.g., NNN) and right universal sample index sequence, may provide enough nucleotide diversity so that sequencing the left sample index (160) can be omitted.

In some embodiments, the method for sequencing comprises step (a): hybridizing the concatemer template molecules with a first plurality of soluble sequencing primers (e.g., forward sequencing primers) that hybridize to the universal binding sequence for a forward sequencing primer (140) and sequencing the first 3-5 bases of the insert region (110) thereby generating a plurality of insert extension products that are hybridized to the immobilized concatemer template molecules, wherein the plurality of insert extension products are complementary to the sequence of interest (110).

In some embodiments, the methods for sequencing further comprise step (b): removing the plurality of insert extension products and retaining the immobilized concatemer template molecules.

In some embodiments, the methods for sequencing further comprise step (c): hybridizing the concatemer template molecules with a second plurality of soluble sequencing primers that hybridize to the universal binding sequence for a reverse sequencing primer (150) and sequencing the right sample index sequence (170) including sequencing the short random sequence (e.g., NNN) and the right universal sample index sequence thereby generating a first plurality of sample index extension products that are hybridized to the immobilized concatemer template molecules, wherein the first plurality of sample index extension products are complementary to the right sample index sequence having a short random sequence (e.g., NNN) linked directly to a right sample index sequence (170), which is a universal sample index sequence.

In some embodiments, the methods for sequencing further comprise step (d): removing the first plurality of sample index extension products and retaining the immobilized concatemer template molecules.

In some embodiments, the methods for sequencing further comprise step (e): hybridizing the retained immobilized concatemer template molecules with a third plurality of soluble sequencing primers that hybridize to the first universal binding site (120) for the first portion of the splint capture primer and sequencing the left sample index sequence (160) thereby generating a second plurality of sample index extension products that are hybridized to the immobilized concatemer template molecules, wherein the second plurality of sample index extension products are complementary to the left sample index sequence (160) having a left universal sample index sequence.

In some embodiments, the methods for sequencing further comprise step (f): replacing the second plurality of sample index extension products that are hybridized to the immobilized concatemer template molecules by conducting a primer extension reaction using strand-displacing polymerases and a plurality of nucleotides to generate an extension product that is hybridized to the immobilized concatemer template molecules including the immobilized capture primer.

In some embodiments, the methods for sequencing further comprise step (g): removing the immobilized concatemer template molecules by generating abasic sites in the immobilized concatemer template molecules at the uracil sites and generating gaps at the abasic sites thereby generating gap-containing concatemer template molecules while retaining the extension products that was generated in step (f) where individual extension products are retained by being hybridized to an immobilized capture primer. In some embodiments, pairwise turn is achieved by conducting steps (g) and (h).

In some embodiments, the methods for sequencing further comprise step (h): hybridizing the retained extension products with a fourth plurality of soluble sequencing primers (e.g., reverse sequencing primers) that hybridize to universal binding sequence for a reverse sequencing primer (150) and sequencing the insert region (110) (e.g., sequencing at least a portion or the full length of the insert region (110)).

In some embodiments, the methods for sequencing further comprise step (i1): assigning the sequence of (i) the insert region (110) to (ii) the right sample index sequence having a short random sequence (e.g., NNN) linked directly to a right sample index sequence (170), which is a universal sample index sequence, thereby identifying the insert region as being obtained from a first source.

In some embodiments, the methods for sequencing further comprise step (i2): assigning the sequence of (i) the insert region (110) to (ii) the right sample index sequence having a short random sequence (e.g., NNN) linked directly to a right sample index sequence (170), which is a universal sample index sequence, and (iii) the left sample index sequence (160), thereby identifying the insert region as being obtained from a first source.

In some embodiments, the removing of the plurality of sequencing extension products of steps (b) and (d) can be conducted using a denaturation reagent comprising SSC (e.g., saline-sodium citrate) buffer with or without formamide, at a temperature that promotes nucleic acid denaturation such as for example 50-90° C.

In some embodiments, the sequencing of steps (a), (c), (e) and (h) include conducting any of the sequencing methods described herein that employ sequencing polymerases and detectably labeled nucleotide analogs. In some embodiments, the sequencing of steps (a), (c), (e) and (h) include conducting any of the two-stage sequencing methods described herein that employ sequencing polymerases, detectably labeled multivalent molecules, and nucleotide analogs. In some embodiments, the sequencing of steps (a), (c), (e) and (h) include conducting any of the sequencing-by-binding methods described herein. In some embodiments, the sequencing of steps (a), (c), (e) and (h) include conducting any of the sequencing methods described herein that employ sequencing polymerases and a nucleotide reagent which comprises labeled chain terminating nucleotides, phosphate chain-labeled nucleotides or labeled multivalent molecules.

In some embodiments, the density of the plurality of concatemer template molecules immobilized to the support is about 10²-10¹⁵ per mm². In some embodiments, the plurality of concatemer template molecules are immobilized at random locations on the support. In some embodiments, the plurality of concatemer template molecules are immobilized on the support in a predetermined pattern.

In some embodiments, any of the order of sequencing methods described above can be conducted on concatemer template molecules immobilized to a support, wherein individual concatemer template molecules comprise two or more tandem repeat units and wherein individual units comprise: (i) a first universal binding site (120) for a first portion of a splint capture primer (or a complementary sequence thereof); (ii) a universal binding site for a first non-splint capture primer (123) (or a complementary sequence thereof); (iii) at least one sample index sequence (e.g., (160) and/or (170) which can be used to distinguish sequences of interest obtained from different sample sources in a multiplex assay; (iv) at least one universal binding site for a forward sequencing primer (140) (or a complementary sequence thereof); (v) a sequence of interest; (vi) at least one universal binding site for a reverse sequencing primer (150) (or a complementary sequence thereof); (vii) at least one universal binding site for a compaction oligonucleotide (or a complementary sequence thereof); (viii) at least one unique molecular index sequence (UMI) (e.g., (180) and/or (190)) which can be used to uniquely identify a nucleic acid molecule (e.g., having a sequence of interest) to which the unique molecular index sequence is appended; (ix) at least one universal binding site for a pinning primer (or a complementary sequence thereof); (x) at least one batch-specific barcode sequence; (xi) a universal binding site for a second non-splint capture primer (133) (or a complementary sequence thereof); (xii) at least one short random sequence (e.g., NNNN) (132) which provides nucleotide sequence diversity and is about 3-20 nucleotides in length; (xiii) a second universal binding site (130) for a second portion of the immobilized splint capture (or a complementary sequence thereof); and/or (xiv) a junction adaptor sequence located between any of the adaptor sequences listed above (e.g., any of the linear library molecules shown in FIGS. 20-24).

### Air Bubble Fluidics

The present disclosure provides systems and methods for handling fluidics that can be employed to conduct any of the methods for generating a plurality of nucleic acid concatemers immobilized to a support which are described herein, including any of the on-support ligation and rolling circle amplification reactions and/or any of the sequencing reactions. On-support amplification and sequencing workflows that are conducted on a flow cell in a massively parallel manner require numerous reagent changes to conduct different biochemical reactions throughout the workflow. A fluidic dispensing system can be used to deliver the reagents to the flow cell. The fluidic dispensing system needs to handle very small fluid volumes and reduce mixing of different reagents that are sequentially flowed onto the flow cell. In some embodiments, air bubbles can be introduced into the fluidics system to separate different reagents flowing onto the flow cell and reduce mixing.

The system comprises a flow cell fluidically connected to a reagent reservoir and a syringe pump. The flow cell includes an inlet on the upstream side and an outlet on the downstream side. The reagent reservoir is connected to the inlet of the flow cell and the syringe pump is connected to the outlet of the flow cell, in a manner that forms a fluid flow path from the reagent reservoir, through the flow cell, and towards the syringe pump. The system further includes an air bubble injector fluidically connected to the fluid flow path and configured to inject air bubbles between different reagents flowing through the fluid path. Fluidic lines can be used to fluidically connect the reagent reservoir, bubble injector, flow cell and syringe pump.

The system can be used to flow very small volumes of different reagents in the fluid flow path and reduce mixing of the different reagents. For example, a first fluid from the reagent reservoir enters the flow path via a fluidic line that connects the reagent reservoir and the inlet of the flow cell. An air bubble is injected into the flow path behind the first reagent in the fluidic line. A second fluid from the reagent reservoir enters the flow path via the fluidic line that connects the reagent reservoir and the inlet of the flow cell and the second reagent is behind the air bubble. The air bubble prevents mixing of the first and second reagents in the fluidic line before the reagents reach the flow cell. Subsequent air bubbles and reagents can be flowed through the flow cell. The system further comprises an air bubble aspirator that removes the air bubble between the first and second reagents so that the first and second reagents are flowed sequentially onto the flow cell with no air bubble. The air bubble aspirator is configured to enable introduction of air bubbles between different reagents in the fluidic line before entry onto the flow cell so that air bubbles do not flow onto the flow cell. The syringe pump is configured to pull the first and second fluids and the air bubble along the flow path from the reagent reservoir and towards the outlet of the flow cell. In this manner, different reagents can be flowed sequentially onto the flow cell with no air bubbles.

The system includes at least one reagent reservoir each reservoir having a plurality of separate compartments, and each compartment holding a reagent for conducting a biochemical or biological reaction. The reservoir(s) contain reagents for conducting nucleic acid amplification and/or nucleic acid sequencing reactions on the flow cell. Each compartment can hold a different reagent in fluid form for conducting a nucleic acid amplification reaction, a nucleic acid sequencing reaction, or washing reagent.

For example, a first reservoir compartment can hold a plurality of linear library molecules. A second reservoir compartment can hold a plurality of amplification polymerases (e.g., strand displacing polymerases), a mixture of four different nucleotide triphosphates or analogs thereof (e.g., dATP, dGTP, dCTP or dTTP) and a nucleotide comprising a scissile moiety, and optionally a plurality of ligases. A third reservoir compartment can hold a plurality of sequencing polymerases, a plurality of soluble sequencing primers, and a mixture of nucleotide triphosphates or analogs thereof (e.g., dATP, dGTP, dCTP or dTTP). A fourth reservoir compartment can hold a plurality of sequencing polymerases, a plurality of soluble sequencing primers, and a mixture of multivalent molecules where individual multivalent molecules comprises nucleotide arms having nucleotide moieties of dATP, dGTP, dCTP or dTTP. A fifth reservoir compartment can hold a wash buffer. A sixth reservoir compartment can hold an imaging formulation. A seventh reservoir compartment can hold a formulation for replacing extended forward sequencing primer strands which includes a plurality of strand displacing polymerases and a plurality of nucleotides (e.g., dATP, dGTP, dCTP or dTTP), and optionally a plurality of soluble forward sequencing primer. An eighth reservoir compartment can hold a formulation for generating abasic sites and gaps on a concatemer template strand.

The system includes at least one flow cell having at least one channel. In some embodiments, the flow cell contains 1, 2, 3, 4, or more channels. In some embodiments, the system includes two or more flow cells. Each flow cell can be configured with a plurality of channels arranged in parallel to each other. Each channel has an inlet on the upstream side and an outlet on the downstream side. The flow cell(s) is/are mounted in the fluidics system so that the inlet side of each channel is/are fluidically connected to the reagent reservoir, and the outlet side of each channel is/are fluidically connected to the syringe pump.

The flow cell channels have a surface that can be coated with at least one hydrophilic polymer layer, or at least one functionalized polymer coating. The channel surface can include a plurality of splint capture primers (200) and/or a plurality of pining primers (500) immobilized on or embedded in the coating. The channel surface can include at least 1000 immobilized splint capture primers per mm². The channel surface can include at least 1000 immobilized pinning primers per mm². The immobilized splint capture and pinning primers on the surface of the channel can be in fluid communication with each other during fluid flow through the channel to permit essentially simultaneously reaction with the reagents in a massively parallel manner. The immobilized splint capture primers can hybridize to linear library molecules each having a sequence of interest and at least one universal adaptor for binding to at least a portion of a splint capture primer. The hybridized linear library molecules can be subjected to a ligation reaction and an amplification reaction (e.g., a rolling circle amplification reaction) in the channel using the fluidic system to generate a plurality of concatemers. The plurality of concatemers in the channel can be subjected to successive sequencing reactions using the fluidic system.

### The Support

The present disclosure provides a support for use in conducting any of the methods including: methods for generating a plurality of immobilized nucleic acid concatemer template molecules, methods for batch sequencing, methods for reiterative sequencing and/or methods for re-seeding and sequencing, described herein. In some embodiments, the support is solid, semi-solid, or a combination of both. In some embodiments, the support is porous, semi-porous, non-porous, or any combination of porosity. In some embodiments, the support can be substantially planar, concave, convex, or any combination thereof. In some embodiments, the support can be cylindrical, for example comprising a capillary or interior surface of a capillary.

The support comprises any material, including but not limited to glass, fused-silica, silicon, a polymer (e.g., polystyrene (PS), macroporous polystyrene (MPPS), polymethylmethacrylate (PMMA), polycarbonate (PC), polypropylene (PP), polyethylene (PE), high density polyethylene (HDPE), cyclic olefin polymers (COP), cyclic olefin copolymers (COC), polyethylene terephthalate (PET)), or any combination thereof. Various compositions of both glass and plastic substrates are contemplated.

In some embodiments, the surface of the support can be substantially smooth and lack contours and texture. In some embodiments, the support can be regularly or irregularly contoured or textured, including protrusions, bumps, wells, etchings, pores, three-dimensional scaffolds, or any combination thereof. In some embodiments, the support comprises contours arranged in a pre-determined pattern. In some embodiments, the support comprises contours arranged in a repeating pattern. In some embodiments, the support comprises interstitial regions between the contours, where the interstitial regions are arranged in a pre-determined. In some embodiments, the interstitial regions are arranged in a repeating pattern.

In some embodiments, the contours and interstitial regions can be fabricated using any combination of photo-chemical, photo-lithography, electron beam lithography, micro- or nano-imprint lithography, ink-jet printing, or micron-scale printing and/or nano-scale printing.

In some embodiments, the contours can be functionalized to promote tethering/immobilizing nucleic acid molecules (e.g., splint capture primers (200), pinning primers (500) and/or nucleic acid concatemer template molecules) and/or for tethering an enzyme (e.g., a polymerase). In some embodiments, the interstitial regions can be modified to inhibit tethering nucleic acid molecules (e.g., splint capture primers (200), pinning primers (500) and/or nucleic acid concatemer template molecules) and/or for inhibiting tethering an enzyme (e.g., a polymerase).

In some embodiments, the support comprises at least one region (e.g., a feature) which can be functionalized to tether/immobilize nucleic acid molecules and/or enzymes. In some embodiments, the features are arranged on the support in a non-predetermined manner (e.g., randomly positioned features; e.g., FIG. 15A). In some embodiments, the features are arranged on the support in a predetermined manner (e.g., patterned features; e.g., FIGS. 16A and 16B). In some embodiments, the features are arranged on the support in repeating pattern (e.g., FIGS. 16A and 16B).

In some embodiments, a support comprises a plurality of features located at random and non-predetermined positions on the support, where individual features can attach to a nucleic acid molecule (e.g., splint capture primer (200), pinning primer (500) or nucleic acid concatemer template molecule). Each of the features on the support can be functionalized with a chemical compound to attach to a nucleic acid molecule.

For example, the features on the support can attach to splint capture primers (200) as shown in e.g., FIG. 15A. In some embodiments, the splint capture primers can be attached to the support such that some of the nearest neighbor splint capture primers touch each other and/or overlap each other when viewed from any angle of the support including above, below or side views of the support. The dotted lines that surround the four splint capture primers represents nearest neighbor splint capture primers that touch each other (e.g., FIG. 15A).

In some embodiments, the splint capture primers (200) on the support can attach to nucleic acid concatemer template molecules having one of four different batch sequences (e.g., FIG. 15B). In some embodiments, the template molecules can attach to the support (via attachment to the splint capture primers) such that some of the nearest neighbor template molecules touch each other and/or overlap each other when viewed from any angle of the support including above, below or side views of the support. The dotted lines that surround the four template molecules represent nearest neighbor template molecules that touch each other (e.g., FIG. 15B).

In some embodiments, the support comprises a contour and at least one feature on or near the contour for tethering nucleic acid molecules. For example, one or more wells (e.g., a plurality of contours) can be fabricated on the support where the bottom of individual wells include a feature having a chemical modification for tethering one or more nucleic acid molecules. The skilled artisan will recognize that the support can be fabricated with any type of contour(s) and feature(s) that are on or near the contour(s), where the features are designed to tether at least one nucleic acid molecule.

In some embodiments, the support lacks contours. In some embodiments, the support lacks features arranged in a pre-determined pattern where the features have a chemical functionality for tethering nucleic acid molecules and/or enzymes to the support. In some embodiments, the support comprises features positioned at random non-predetermined locations on the support. In some embodiments, the support lacks interstitial regions arranged in a pre-determined pattern where the interstitial regions are sites designed to inhibit tethering nucleic acid molecules or enzymes.

In some embodiments, any of the features for tethering nucleic acids and/or enzymes can be positioned on the support using ink-jet printing, or micron-scale or nano-scale printing. In some embodiments, the features can be made in any shape including, for example circular, square, triangular or rectangular (e.g., FIGS. 15A and 16B).

In some embodiments, at least one surface of the support can be modified with a chemical compound that enables attachment of a polymer coating to the support. For example, the support can be modified with a silane compound. In some embodiments, the silane compound can bind a polymer coating. In some embodiments, at least one surface of the support is passivated with at least one polymer coating layer (e.g., FIG. 14). In some embodiments, the support is passivated with 1, 2, 3, 4, 5, 6, 7, 8, 9, 10 or more polymer coating layers. In some embodiments, the coating forms a continuous layer on the support wherein the coating forms no pre-determined pattern.

In some embodiments, the surface coating may be patterned, such that the chemical modification layers are confined to one or more discrete regions of the support. For example, the coating may be patterned using photolithographic techniques to create an ordered array or random pattern of chemically-modified regions on the support. Alternately or in combination, the coating may be patterned using, e.g., contact printing and/or ink-jet printing techniques. In some embodiments, the coating is distributed on the support in a pre-determined pattern, for example the pre-determined pattern comprises or spots arranged in rows and/or columns or other pre-determined patterns. In some embodiments, the coating having a pre-determined pattern comprises at least one interstitial region that lacks a polymer coating. In some embodiments, the passivated layer forms a porous or semi-porous layer.

In some embodiments, at least one of the polymer coating layers comprises a hydrophilic polymer layer. In some embodiments, at least one polymer coating layer comprises polymer molecules having a molecular weight of at least 1000 Daltons. The hydrophilic polymer coating layer can comprise polyethylene glycol (PEG). The hydrophilic polymer layer can comprise unbranched PEG. The hydrophilic polymer layer can comprise branched PEG having at least 4 branches, for example the branched PEG comprises 4-16 branches. In some embodiments, the hydrophilic polymer layer comprises cross-linking or lacks cross-linking. In some embodiments, the hydrophilic polymer layer comprises cross-linking to form a hydrogel.

In some embodiments, the hydrophilic polymer layer comprises a monolayer having unbranched polymers which can form a brush monolayer. In some embodiments, the brush monolayer can form an extended brush monolayer. In some embodiments, the brush monolayer comprises a plurality of unbranched polymers where one end of a given unbranched polymer is attached to the support and the other end of the same given unbranched polymer is attached to an oligonucleotide primer (e.g., splint capture primer (200) or pinning primer (500)). In some embodiments, the density of the plurality of oligonucleotide primers attached to the brush monolayer is about 10²-10¹⁵ per um².

In some embodiments, the coating layer has a degree of hydrophilicity which can be measured as a water contact angle, where the water contact angle is no more than 45 degrees.

In some embodiments, any layer of the polymer coating includes a plurality of oligonucleotide primers covalently tethered to the polymer layer. In some embodiments, the plurality of oligonucleotide primers are distributed at a plurality of depths throughout any of the polymer layers. In some embodiments, the density of the plurality of oligonucleotide primers in any of the polymer layers is about 10²-10¹⁵ per um². In some embodiments, individual oligonucleotide primers comprise nucleic acid molecules comprising DNA, RNA, DNA/RNA chimeric or analogs thereof. In some embodiments, the plurality of oligonucleotide primers are about 10-100 nucleotides in length. In some embodiments, individual oligonucleotide primers in the plurality comprise 3′ extendible ends or 3′ non-extendible ends. In some embodiments, the 3′ non-extendible ends comprise a 3′ chain terminating moiety. In some embodiments, individual oligonucleotide primers have their 5′ or 3′ ends or an internal region attached to the polymer layer. In some embodiments, the 5′ ends of the plurality of oligonucleotide primers are attached to the polymer layer. In some embodiments, the plurality of oligonucleotide primer are randomly distributed throughout and embedded within at least one of the polymer layers. In some embodiments, the plurality of oligonucleotide primers are distributed in or on at least one of the polymer layers in a random manner or a pre-determined pattern. In some embodiments, the plurality of oligonucleotide primers are distributed in or on at least one of the polymer layers in a non-random pre-determined pattern, for example the pre-determined pattern comprises stripes or spots arranged in rows and/or columns or other pre-determined patterns.

In some embodiments, the support comprises a first layer comprising a first monolayer having hydrophilic polymer molecules tethered to the support. In some embodiments, at least some of the polymer molecules in the first layer are covalently tethered to oligonucleotide primers. In some embodiments, the tethered oligonucleotide primers in the first monolayer are arranged in a random manner or in a pre-determined pattern. In some embodiments, the polymer molecules in the first layer are not tethered to oligonucleotide primers.

In some embodiments, the support further comprises a second layer comprising a second monolayer having hydrophilic polymer molecules tethered to the first monolayer. In some embodiments, at least some of the polymer molecules in the second layer are covalently tethered to oligonucleotide primers. In some embodiments, the tethered oligonucleotide primers in the second monolayer are arranged in a random manner or in a pre-determined pattern. In some embodiments, the polymer molecules in the second layer are not tethered to oligonucleotide primers.

In some embodiments, the support further comprises a third layer comprising a third monolayer having hydrophilic polymer molecules tethered to the second monolayer. In some embodiments, at least some of the polymer molecules in the third layer are covalently tethered to oligonucleotide primers. In some embodiments, the tethered oligonucleotide primers in the third monolayer are arranged in a random manner or in a pre-determined pattern. In some embodiments, the polymer molecules in the third layer are not tethered to oligonucleotide primers.

In some embodiments, the support comprises a functionalized polymer coating layer covalently bound at least to a portion of the support via a chemical group on the support, a primer grafted to the functionalized polymer coating, and a water-soluble protective coating on the primer and the functionalized polymer coating. In some embodiments, the functionalized polymer coating comprises a poly(N-(5-azidoacetamidylpentyl)acrylamide-co-acrylamide (PAZAM).

In some embodiments, at least one of the polymer layers comprise oligonucleotide primers including splint capture primers (200), pinning primers (500), or a mixture of splint capture primers and pinning primers. In some embodiments, the plurality of oligonucleotide primers comprise one type of splint capture primer having that same splint capture primer sequence or a mixture of 2-100 different types of splint capture primers having 2-100 different splint capture primer sequences. In some embodiments, the plurality of oligonucleotide primers comprise one type of pinning primer having that same pinning primer sequence or a mixture of 2-100 different types of pinning primers having 2-100 different pinning primer sequences.

In some embodiments, individual splint capture primers (200) (e.g., which are tethered to and/or embedded in a polymer layer) can be used for conducting on-support ligation and amplification reactions. For example, individual splint capture primers hybridize to first and second splint capture primer binding sites in a linear library molecule to generate an open circle library molecule having a nick or gap, enzymatically closing the nick or gap, and conducting rolling circle amplification to generate a concatemer template molecule which is tethered and/or embedded in the polymer layer.

In some embodiments, the density of the splint capture primers (200) in a polymer layer can be modulated (e.g., increased or decreased) to achieve a desired density of immobilized concatemer template molecules on a support. Generally, a polymer layer having a high density of splint capture primers will generate concatemer template molecules that are tightly packed and immobilized to the support at a density of about 10⁵-10¹⁵ per mm² which cannot be achieved using supports fabricated to include nano-scale features for attachment of template molecules.

In some embodiments, a single pinning primer (e.g., which is tethered to or embedded in a polymer layer) can hybridize to a pinning primer binding site in a concatemer template molecule to generate a concatemer template molecule which is tethered or embedded (e.g., pinned down) in the polymer layer.

In some embodiments, at least one of the polymer layers comprise a plurality of splint capture primers (200) and/or pinning primers (500) having a cleavable region that is cleavable with a restriction endonuclease enzyme. For example, the cleavable region comprises a recognition site for a type I, type II, type IIs, type IIB, type III, or type IV restriction enzyme. In some embodiments, the plurality of splint capture primers and/or pinning primers include a cleavable region that is cleavable with an enzyme that generates an abasic site. For example, the cleavable region comprises at least one nucleotide having a scissile moiety including uridine, 8-oxo-7,8-dihydrogunine or deoxyinosine. In some embodiments, the plurality of splint capture primers and/or pinning primers lack a cleavable region.

In some embodiments, the support comprises at least one partition/barrier that creates separate regions of the support. For example, the partition/barrier can prevent fluid flow on one portion of the support. The partition/barrier can inhibit nucleic acid and/or enzyme reactions on a portion of the support. In some embodiments, the partition/barrier can be placed on the support. In some embodiments, the partition/barrier is not placed on the support but is positioned to block fluid flow onto the support.

In some embodiments, the support lacks partitions/barriers that would create separate regions of the support. For example, the support is passivated with at least one polymer coating formed as a continuous layer, and at least one of the polymer layers comprise a plurality of splint capture primers (200) that are randomly distributed throughout and on the polymer layer. The splint capture primers can be used to generate immobilized concatemer template molecules. Thus, the immobilized template molecules are in fluid communication with each other in a massively parallel manner with no barriers to physically separate different batches of template molecules. In some embodiments, sub-populations of template molecules carrying different batch sequencing primer binding sites can be used to conduct batch sequencing. Asynchronous sequencing can be achieved by conducting batch sequencing on concatemer template molecules in fluid communication with each other on the same non-partitioned support.

Surface Coatings with High Contrast-to-Noise Ratio

The present disclosure provides a support which can be employed in any of the methods for generating a plurality of immobilized nucleic acid concatemers described herein. In some embodiments, the support comprises a planar or non-planar support. The support can be solid or semi-solid. In some embodiments, the support can be porous, semi-porous or non-porous. In some embodiments, the surface of the support can be coated with one or more compounds to produce a passivated layer on the support. In some embodiments, the passivated layer forms a porous or semi-porous layer. In some embodiments, the nucleic acid primer, template and/or polymerase, can be attached to the passivated layer to immobilize the primer, template and/or polymerase to the support. In some embodiments, the support comprises a low non-specific binding surface that enable improved nucleic acid hybridization and amplification performance on the support. In general, the support may comprise one or more layers of a covalently or non-covalently attached low-binding, chemical modification layers, e.g., silane layers, polymer films, and one or more covalently or non-covalently attached oligonucleotides (e.g., splint capture primers (200)) that can be used for immobilizing a plurality of nucleic acid concatemer template molecules to the support. In some embodiments, the support can comprise a functionalized polymer coating layer covalently bound at least to a portion of the support via a chemical group on the support, a primer grafted to the functionalized polymer coating, and a water-soluble protective coating on the primer and the functionalized polymer coating. In some embodiments, the functionalized polymer coating comprises a poly(N-(5-azidoacetamidylpentyl)acrylamide-co-acrylamide (PAZAM). In some embodiments, the support comprises a surface coating having at least one hydrophilic polymer coating layer and at least one layer of a plurality of oligonucleotides. The hydrophilic polymer coating layer can comprise polyethylene glycol (PEG). The hydrophilic polymer coating layer can comprise branched PEG having at least 4 branches. In some embodiments, the low non-specific binding coating has a degree of hydrophilicity which can be measured as a water contact angle, where the water contact angle is no more than 45 degrees. In some embodiments, the density of the plurality of first polymerase complexes immobilized to the support or immobilized to the coating on the support is about 10²-10⁶ per mm², or about 10⁶-10⁹ per mm², or about 10⁹-10¹² per mm². In some embodiments, the plurality of first polymerase complexes is immobilized to the support or immobilized to the coating on the support at pre-determined sites on the support (or the coating on the support), or immobilized to the coating on the support at random sites on the support (or the coating on the support).

In some embodiments, the support is passivated with a low non-specific binding coating. The surface coatings described herein exhibit very low non-specific binding to reagents typically used for nucleic acid capture, amplification and sequencing workflows, such as dyes, nucleotides, enzymes, and nucleic acid primers. The surface coatings exhibit low background fluorescence signals or high contrast-to-noise (CNR) ratios compared to conventional surface coatings.

The low non-specific binding coating comprises one layer or multiple layers. In some embodiments, a plurality of surface primers (e.g., including splint capture primers (200) and pinning primers (500)) are immobilized to the low non-specific binding coating. In some embodiments, at least one splint capture primer and/or pinning primer is embedded within the low non-specific binding coating. The low non-specific binding coating enables improved nucleic acid hybridization and amplification performance. In general, the supports comprise a substrate (or support structure), one or more layers of a covalently or non-covalently attached low-binding, chemical modification layers, e.g., silane layers, polymer films, and one or more covalently or non-covalently attached splint capture primers and/or pinning primers that can be used for tethering nucleic acid library molecules or concatemer template molecules to the support. In some embodiments, the formulation of the coating, e.g., the chemical composition of one or more layers, the coupling chemistry used to cross-link the one or more layers to the support and/or to each other, and the total number of layers, may be varied such that non-specific binding of proteins, nucleic acid molecules, and other hybridization and amplification reaction components to the coating is minimized or reduced relative to a comparable monolayer. The formulation of the coating described herein may be varied such that non-specific hybridization on the coating is minimized or reduced relative to a comparable monolayer. The formulation of the coating may be varied such that non-specific amplification on the coating is minimized or reduced relative to a comparable monolayer. The formulation of the coating may be varied such that specific amplification rates and/or yields on the coating are maximized. Amplification levels suitable for detection are achieved in no more than 2, 3, 4, 5, 6, 7, 8, 9, 10, 15, 20, 25, 30, or more than 30 amplification cycles in some cases disclosed herein.

The support structure that comprises the one or more chemically-modified layers, e.g., layers of a low non-specific binding polymer, may be independent or integrated into another structure or assembly. For example, in some embodiments, the support structure may comprise one or more surfaces within an integrated or assembled microfluidic flow cell. The support structure may comprise one or more surfaces within a microplate format, e.g., the bottom surface of the wells in a microplate. In some embodiments, the support structure comprises the interior surface (such as the lumen surface) of a capillary. In some embodiments, the support structure comprises the interior surface (such as the lumen surface) of a capillary etched into a planar chip.

The attachment chemistry used to graft a first chemically-modified layer to the surface of the support will generally be dependent on both the material from which the surface is fabricated and the chemical nature of the layer. In some embodiments, the first layer may be covalently attached to the surface. In some embodiments, the first layer may be non-covalently attached, e.g., adsorbed to the support through non-covalent interactions such as electrostatic interactions, hydrogen bonding, or van der Waals interactions between the support and the molecular components of the first layer. In either case, the support may be treated prior to attachment or deposition of the first layer. Any of a variety of surface preparation techniques known to those of skill in the art may be used to clean or treat the surface. For example, glass or silicon surfaces may be acid-washed using a Piranha solution (a mixture of sulfuric acid (H₂SO₄) and hydrogen peroxide (H₂O₂)), base treatment in KOH and NaOH, and/or cleaned using an oxygen plasma treatment method.

Silane chemistries constitute non-limiting approaches for covalently modifying the silanol groups on glass or silicon surfaces to attach more reactive functional groups (e.g., amines or carboxyl groups), which may then be used in coupling linker molecules (e.g., linear hydrocarbon molecules of various lengths, such as C6, C12, C18 hydrocarbons, or linear polyethylene glycol (PEG) molecules) or layer molecules (e.g., branched PEG molecules or other polymers) to the surface. Examples of suitable silanes that may be used in creating any of the disclosed low binding coatings include, but are not limited to, (3-Aminopropyl) trimethoxysilane (APTMS), (3-Aminopropyl) triethoxysilane (APTES), any of a variety of PEG-silanes (e.g., comprising molecular weights of 1K, 2K, 5K, 10K, 20K, etc.), amino-PEG silane (i.e., comprising a free amino functional group), maleimide-PEG silane, biotin-PEG silane, and the like.

Any of a variety of molecules known to those of skill in the art including, but not limited to, amino acids, peptides, nucleotides, oligonucleotides, other monomers or polymers, or combinations thereof may be used in creating the one or more chemically-modified layers on the support, where the choice of components used may be varied to alter one or more properties of the layers, e.g., the surface density of functional groups and/or tethered oligonucleotide primers, the hydrophilicity/hydrophobicity of the layers, or the three three-dimensional nature (i.e., “thickness”) of the layer. Examples of polymers that may be used to create one or more layers of low non-specific binding material in any of the disclosed coatings include, but are not limited to, polyethylene glycol (PEG) of various molecular weights and branching structures, streptavidin, polyacrylamide, polyester, dextran, poly-lysine, and poly-lysine copolymers, or any combination thereof. Examples of conjugation chemistries that may be used to graft one or more layers of material (e.g. polymer layers) to the surface and/or to cross-link the layers to each other include, but are not limited to, biotin-streptavidin interactions (or variations thereof), his tag—Ni/NTA conjugation chemistries, methoxy ether conjugation chemistries, carboxylate conjugation chemistries, amine conjugation chemistries, NHS esters, maleimides, thiol, epoxy, azide, hydrazide, alkyne, isocyanate, and silane.

The low non-specific binding surface coating may be applied uniformly across the support. Alternatively, the surface coating may be patterned, such that the chemical modification layers are confined to one or more discrete regions of the support. For example, the coating may be patterned using photolithographic techniques to create an ordered array or random pattern of chemically-modified regions on the support. Alternately or in combination, the coating may be patterned using, e.g., contact printing and/or ink-jet printing techniques. In some embodiments, an ordered array or random pattern of chemically-modified regions may comprise at least 1, 5, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or 10,000 or more discrete regions.

In some embodiments, the low nonspecific binding coatings comprise hydrophilic polymers that are non-specifically adsorbed or covalently grafted to the support. Typically, passivation is performed utilizing poly(ethylene glycol) (PEG, also known as polyethylene oxide (PEO) or polyoxyethylene) or other hydrophilic polymers with different molecular weights and end groups that are linked to a support using, for example, silane chemistry. The end groups distal from the surface can include, but are not limited to, biotin, methoxy ether, carboxylate, amine, NHS ester, maleimide, and bis-silane. In some embodiments, two or more layers of a hydrophilic polymer, e.g., a linear polymer, branched polymer, or multi-branched polymer, may be deposited on the surface. In some embodiments, two or more layers may be covalently coupled to each other or internally cross-linked to improve the stability of the resulting coating. In some embodiments, splint capture primers (200) and/or pinning primers (500) with different nucleotide sequences and/or base modifications (or other biomolecules, e.g., enzymes or antibodies) may be tethered to the resulting layer at various surface densities. In some embodiments, for example, both surface functional group density and surface primer concentration may be varied to attain a desired surface primer density range. Additionally, surface primer density can be controlled by diluting the surface primers with other molecules that carry the same functional group. For example, amine-labeled surface primers can be diluted with amine-labeled polyethylene glycol in a reaction with an NHS-ester coated surface to reduce the final primer density. Surface primers with different lengths of linker between the hybridization region and the surface attachment functional group can also be applied to control surface density. Example of suitable linkers include poly-T and poly-A strands at the 5′ end of the primer (e.g., 0 to 20 bases), PEG linkers (e.g., 3 to 20 monomer units), and carbon-chain (e.g., C6, C12, C18, etc.). To measure the primer density, fluorescently-labeled primers may be tethered to the surface and a fluorescence reading then compared with that for a dye solution of known concentration.

In some embodiments, the low nonspecific binding coatings comprise a functionalized polymer coating layer covalently bound at least to a portion of the support via a chemical group on the support, a primer grafted to the functionalized polymer coating, and a water-soluble protective coating on the primer and the functionalized polymer coating. In some embodiments, the functionalized polymer coating comprises a poly(N-(5-azidoacetamidylpentyl)acrylamide-co-acrylamide (PAZAM).

In order to scale primer surface density and add additional dimensionality to hydrophilic or amphoteric coatings, supports comprising multi-layer coatings of PEG and other hydrophilic polymers have been developed. By using hydrophilic and amphoteric surface layering approaches that include, but are not limited to, the polymer/co-polymer materials described below, it is possible to increase primer loading density on the support significantly. Traditional PEG coating approaches use monolayer primer deposition, which have been generally reported for single molecule applications, but do not yield high copy numbers for nucleic acid amplification applications. As described herein “layering” can be accomplished using traditional crosslinking approaches with any compatible polymer or monomer subunits such that a surface comprising two or more highly crosslinked layers can be built sequentially. Examples of suitable polymers include, but are not limited to, streptavidin, poly acrylamide, polyester, dextran, poly-lysine, and copolymers of poly-lysine and PEG. In some embodiments, the different layers may be attached to each other through any of a variety of conjugation reactions including, but not limited to, biotin-streptavidin binding, azide-alkyne click reaction, amine-NHS ester reaction, thiol-maleimide reaction, and ionic interactions between positively charged polymer and negatively charged polymer. In some embodiments, high primer density materials may be constructed in solution and subsequently layered onto the surface in multiple steps.

Examples of materials from which the support structure may be fabricated include, but are not limited to, glass, fused-silica, silicon, a polymer (e.g., polystyrene (PS), macroporous polystyrene (MPPS), polymethylmethacrylate (PMMA), polycarbonate (PC), polypropylene (PP), polyethylene (PE), high density polyethylene (HDPE), cyclic olefin polymers (COP), cyclic olefin copolymers (COC), polyethylene terephthalate (PET)), or any combination thereof. Various compositions of both glass and plastic support structures are contemplated.

The support structure may be rendered in any of a variety of geometries and dimensions known to those of skill in the art, and may comprise any of a variety of materials known to those of skill in the art. For example, the support structure may be locally planar (e.g., comprising a microscope slide or the surface of a microscope slide). Globally, the support structure may be cylindrical (e.g., comprising a capillary or the interior surface of a capillary), spherical (e.g., comprising the outer surface of a non-porous bead), or irregular (e.g., comprising the outer surface of an irregularly-shaped, non-porous bead or particle). In some embodiments, the surface of the support structure used for nucleic acid hybridization and amplification may be a solid, non-porous surface. In some embodiments, the surface of the support structure used for nucleic acid hybridization and amplification may be porous, such that the coatings described herein penetrate the porous surface, and nucleic acid hybridization and amplification reactions performed thereon may occur within the pores.

The support structure that comprises the one or more chemically-modified layers, e.g., layers of a low non-specific binding polymer, may be independent or integrated into another structure or assembly. For example, the support structure may comprise one or more surfaces within an integrated or assembled microfluidic flow cell. The support structure may comprise one or more surfaces within a microplate format, e.g., the bottom surface of the wells in a microplate. In some embodiments, the support structure comprises the interior surface (such as the lumen surface) of a capillary. In some embodiments the support structure comprises the interior surface (such as the lumen surface) of a capillary etched into a planar chip.

As noted, the low non-specific binding supports of the present disclosure exhibit reduced non-specific binding of proteins, nucleic acids, and other components of the hybridization and/or amplification formulation used for solid-phase nucleic acid amplification. The degree of non-specific binding exhibited by a given support surface may be assessed either qualitatively or quantitatively. For example, exposure of the surface to fluorescent dyes (e.g., cyanins such as Cy3, or Cy5, etc., fluoresceins, coumarins, rhodamines, etc. or other dyes disclosed herein), fluorescently-labeled nucleotides, fluorescently-labeled oligonucleotides, and/or fluorescently-labeled proteins (e.g. polymerases) under a standardized set of conditions, followed by a specified rinse protocol and fluorescence imaging may be used as a qualitative tool for comparison of non-specific binding on supports comprising different surface formulations. In some embodiments, exposure of the surface to fluorescent dyes, fluorescently-labeled nucleotides, fluorescently-labeled oligonucleotides, and/or fluorescently-labeled proteins (e.g. polymerases) under a standardized set of conditions, followed by a specified rinse protocol and fluorescence imaging may be used as a quantitative tool for comparison of non-specific binding on supports comprising different surface formulations-provided that care has been taken to ensure that the fluorescence imaging is performed under conditions where fluorescence signal is linearly related (or related in a predictable manner) to the number of fluorophores on the support surface (e.g., under conditions where signal saturation and/or self-quenching of the fluorophore is not an issue) and suitable calibration standards are used. In some embodiments, other techniques known to those of skill in the art, for example, radioisotope labeling and counting methods may be used for quantitative assessment of the degree to which non-specific binding is exhibited by the different support surface formulations of the present disclosure.

Some surfaces disclosed herein exhibit a ratio of specific to nonspecific binding of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein. Some surfaces disclosed herein exhibit a ratio of specific to nonspecific fluorescence of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein.

The degree of non-specific binding exhibited by the disclosed low-binding supports may be assessed using a standardized protocol for contacting the surface with a labeled protein (e.g., bovine serum albumin (BSA), streptavidin, a DNA polymerase, a reverse transcriptase, a helicase, a single-stranded binding protein (SSB), etc., or any combination thereof), a labeled nucleotide, a labeled oligonucleotide, etc., under a standardized set of incubation and rinse conditions, followed be detection of the amount of label remaining on the surface and comparison of the signal resulting therefrom to an appropriate calibration standard. In some embodiments, the label may comprise a fluorescent label. In some embodiments, the label may comprise a radioisotope. In some embodiments, the label may comprise any other detectable label known to one of skill in the art. In some embodiments, the degree of non-specific binding exhibited by a given support surface formulation may thus be assessed in terms of the number of non-specifically bound protein molecules (or nucleic acid molecules or other molecules) per unit area. In some embodiments, the low-binding supports of the present disclosure may exhibit non-specific protein binding (or non-specific binding of other specified molecules, (e.g., cyanins such as Cy3, or Cy5, etc., fluoresceins, coumarins, rhodamines, etc. or other dyes disclosed herein)) of less than 0.001 molecule per μm², less than 0.01 molecule per μm², less than 0.1 molecule per μm², less than 0.25 molecule per μm², less than 0.5 molecule per μm², less than 1 molecule per μm², less than 10 molecules per μm², less than 100 molecules per μm², or less than 1,000 molecules per μm². Those of skill in the art will realize that a given support surface of the present disclosure may exhibit non-specific binding falling anywhere within this range, for example, of less than 86 molecules per μm². For example, some modified surfaces disclosed herein exhibit nonspecific protein binding of less than 0.5 molecule/μm² following contact with a 1 μM solution of Cy3 labeled streptavidin (GE Amersham) in phosphate buffered saline (PBS) buffer for 15 minutes, followed by 3 rinses with deionized water. Some modified surfaces disclosed herein exhibit nonspecific binding of Cy3 dye molecules of less than 0.25 molecules per μm². In independent nonspecific binding assays, 1 μM labeled Cy3 SA (ThermoFisher), 1 μM Cy5 SA dye (ThermoFisher), 10 pM Aminoallyl-dUTP-ATTO-647N (Jena Biosciences), 10 M Aminoallyl-dUTP-ATTO-Rhol 1 (Jena Biosciences), 10 pM Aminoallyl-dUTP-ATTO-Rhol 1 (Jena Biosciences), 10 μM 7-Propargylamino-7-deaza-dGTP-Cy5 (Jena Biosciences, and 10 μM 7-Propargylamino-7-deaza-dGTP-Cy3 (Jena Biosciences) were incubated on the low binding coated supports at 37° C. for 15 minutes in a 384 well plate format. Each well was rinsed 2-3× with 50 uL deionized RNase/DNase Free water and 2-3× with 25 mM ACES buffer pH 7.4. The 384 well plates were imaged on a GE Typhoon instrument using the Cy3, AF555, or Cy5 filter sets (according to dye test performed) as specified by the manufacturer at a PMT gain setting of 800 and resolution of 50-100 μm. For higher resolution imaging, images were collected on an Olympus IX83 microscope (e.g., inverted fluorescence microscope) (Olympus Corp., Center Valley, Pa.) with a total internal reflectance fluorescence (TIRF) objective (100×, 1.5 NA, Olympus), a CCD camera (e.g., an Olympus EM-CCD monochrome camera, Olympus XM-10 monochrome camera, or an Olympus DP80 color and monochrome camera), an illumination source (e.g., an Olympus 100 W Hg lamp, an Olympus 75 W Xe lamp, or an Olympus U-HGLGPS fluorescence light source), and excitation wavelengths of 532 nm or 635 nm. Dichroic mirrors were purchased from Semrock (IDEX Health & Science, LLC, Rochester, N.Y.), e.g., 405, 488, 532, or 633 nm dichroic reflectors/beamsplitters, and band pass filters were chosen as 532 LP or 645 LP concordant with the appropriate excitation wavelength. Some modified surfaces disclosed herein exhibit nonspecific binding of dye molecules of less than 0.25 molecules per μm². In some embodiments, the coated support was immersed in a buffer (e.g., 25 mM ACES, pH 7.4) while the image was acquired.

In some embodiments, the surfaces disclosed herein exhibit a ratio of specific to nonspecific binding of a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein. In some embodiments, the surfaces disclosed herein exhibit a ratio of specific to nonspecific fluorescence signals for a fluorophore such as Cy3 of at least 2, 3, 4, 5, 6, 7, 8, 9, 10, 11, 12, 13, 14, 15, 16, 17, 18, 19, 20, 25, 30, 35, 40, 50, 75, 100, or greater than 100, or any intermediate value spanned by the range herein.

The low-background surfaces consistent with the disclosure herein may exhibit specific dye attachment (e.g., Cy3 attachment) to non-specific dye adsorption (e.g., Cy3 dye adsorption) ratios of at least 4:1, 5:1, 6:1, 7:1, 8:1, 9:1, 10:1, 15:1, 20:1, 30:1, 40:1, 50:1, or more than 50 specific dye molecules attached per molecule nonspecifically adsorbed. Similarly, when subjected to an excitation energy, low-background surfaces consistent with the disclosure herein to which fluorophores, e.g., Cy3, have been attached may exhibit ratios of specific fluorescence signal (e.g., arising from Cy3-labeled oligonucleotides attached to the surface) to non-specific adsorbed dye fluorescence signals of at least 4:1, 5:1, 6:1, 7:1, 8:1, 9:1, 10:1, 15:1, 20:1, 30:1, 40:1, 50:1, or more than 50:1.

In some embodiments, the degree of hydrophilicity (or “wettability” with aqueous solutions) of the disclosed support surfaces may be assessed, for example, through the measurement of water contact angles in which a small droplet of water is placed on the surface and its angle of contact with the surface is measured using, e.g., an optical tensiometer. In some embodiments, a static contact angle may be determined. In some embodiments, an advancing or receding contact angle may be determined. In some embodiments, the water contact angle for the hydrophilic, low-binding support surfaced disclosed herein may range from about 0 degrees to about 30 degrees. In some embodiments, the water contact angle for the hydrophilic, low-binding support surfaced disclosed herein may no more than 50 degrees, 40 degrees, 30 degrees, 25 degrees, 20 degrees, 18 degrees, 16 degrees, 14 degrees, 12 degrees, 10 degrees, 8 degrees, 6 degrees, 4 degrees, 2 degrees, or 1 degree. In many cases the contact angle is no more than 40 degrees. Those of skill in the art will realize that a given hydrophilic, low-binding support surface of the present disclosure may exhibit a water contact angle having a value of anywhere within this range.

In some embodiments, the hydrophilic surfaces disclosed herein facilitate reduced wash times for bioassays, often due to reduced nonspecific binding of biomolecules to the low-binding surfaces. In some embodiments, adequate wash steps may be performed in less than 60, 50, 40, 30, 20, 15, 10, or less than 10 seconds. For example, adequate wash steps may be performed in less than 30 seconds.

Some low-binding surfaces of the present disclosure exhibit significant improvement in stability or durability to prolonged exposure to solvents and elevated temperatures, or to repeated cycles of solvent exposure or changes in temperature. For example, the stability of the disclosed surfaces may be tested by fluorescently labeling a functional group on the surface, or a tethered biomolecule (e.g., an oligonucleotide primer) on the surface, and monitoring fluorescence signal before, during, and after prolonged exposure to solvents and elevated temperatures, or to repeated cycles of solvent exposure or changes in temperature. In some embodiments, the degree of change in the fluorescence used to assess the quality of the surface may be less than 1%, 2%, 3%, 4%, 5%, 10%, 15%, 20%, or 25% over a time period of 1 minute, 2 minutes, 3 minutes, 4 minutes, 5 minutes, 10 minutes, 20 minutes, 30 minutes, 40 minutes, 50 minutes, 60 minutes, 2 hours, 3 hours, 4 hours, 5 hours, 6 hours, 7 hours, 8 hours, 9 hours, 10 hours, 15 hours, 20 hours, 25 hours, 30 hours, 35 hours, 40 hours, 45 hours, 50 hours, or 100 hours of exposure to solvents and/or elevated temperatures (or any combination of these percentages as measured over these time periods). In some embodiments, the degree of change in the fluorescence used to assess the quality of the surface may be less than 1%, 2%, 3%, 4%, 5%, 10%, 15%, 20%, or 25% over 5 cycles, 10 cycles, 20 cycles, 30 cycles, 40 cycles, 50 cycles, 60 cycles, 70 cycles, 80 cycles, 90 cycles, 100 cycles, 200 cycles, 300 cycles, 400 cycles, 500 cycles, 600 cycles, 700 cycles, 800 cycles, 900 cycles, or 1,000 cycles of repeated exposure to solvent changes and/or changes in temperature (or any combination of these percentages as measured over this range of cycles).

In some embodiments, the surfaces disclosed herein may exhibit a high ratio of specific signal to nonspecific signal or other background. For example, when used for nucleic acid amplification, some surfaces may exhibit an amplification signal that is at least 4, 5, 6, 7, 8, 9, 10, 15, 20, 30, 40, 50, 75, 100, or greater than 100 fold greater than a signal of an adjacent unpopulated region of the surface. Similarly, some surfaces exhibit an amplification signal that is at least 4, 5, 6, 7, 8, 9, 10, 15, 20, 30, 40, 50, 75, 100, or greater than 100 fold greater than a signal of an adjacent amplified nucleic acid population region of the surface.

In some embodiments, fluorescence images of the disclosed low background surfaces when used in nucleic acid hybridization or amplification applications to create polonies of hybridized or clonally-amplified nucleic acid molecules (e.g., that have been directly or indirectly labeled with a fluorophore) exhibit contrast-to-noise ratios (CNRs) of at least 10, 20, 30, 40, 50, 60, 70, 80, 90, 100, 110, 120, 130, 140, 150, 160, 170, 180, 190, 20, 210, 220, 230, 240, 250, or greater than 250.

One or more types of primer may be attached or tethered to the support surface. In some embodiments, the one or more types of adapters or primers may comprise spacer sequences, adapter sequences for hybridization to adapter-ligated library insert sequences, forward amplification primers, reverse amplification primers, sequencing primers, sample index sequences and/or unique molecular tagging sequences, or any combination thereof. In some embodiments, one type of surface primer or adapter sequence may be tethered to at least one layer of the surface. In some embodiments, at least 2, 3, 4, 5, 6, 7, 8, 9, 10, or more than 10 different types of primer or adapter sequences may be tethered to at least one layer of the surface.

In some embodiments, the tethered adapter and/or primer sequences may range in length from about 10 nucleotides to about 100 nucleotides. In some embodiments, the tethered adapter and/or primer sequences may be at least 10, at least 20, at least 30, at least 40, at least 50, at least 60, at least 70, at least 80, at least 90, or at least 100 nucleotides in length. In some embodiments, the tethered adapter and/or primer sequences may be at most 100, at most 90, at most 80, at most 70, at most 60, at most 50, at most 40, at most 30, at most 20, or at most 10 nucleotides in length. Any of the lower and upper values described in this paragraph may be combined to form a range included within the present disclosure, for example, in some embodiments the length of the tethered adapter and/or primer sequences may range from about 20 nucleotides to about 80 nucleotides. Those of skill in the art will recognize that the length of the tethered adapter and/or primer sequences may have any value within this range, e.g., about 24 nucleotides.

In some embodiments, the resultant surface density of primers (e.g., capture primers) on the low binding support surfaces of the present disclosure may range from about 100 primer molecules per μm² to about 100,000 primer molecules per μm². In some embodiments, the resultant surface density of primers on the low binding support surfaces of the present disclosure may range from about 1,000 primer molecules per μm² to about 1,000,000 primer molecules per μm². In some embodiments, the surface density of primers may be at least 1,000, at least 10,000, at least 100,000, or at least 1,000,000 molecules per μm². In some embodiments, the surface density of primers may be at most 1,000,000, at most 100,000, at most 10,000, or at most 1,000 molecules per μm². Any of the lower and upper values described in this paragraph may be combined to form a range included within the present disclosure, for example, in some embodiments the surface density of primers may range from about 10,000 molecules per μm² to about 100,000 molecules per μm². Those of skill in the art will recognize that the surface density of primer molecules may have any value within this range, e.g., about 455,000 molecules per μm². In some embodiments, the surface density of library molecules initially hybridized to adapter or surface primer sequences on the support surface may be less than or equal to that indicated for the surface density of tethered primers. In some embodiments, the surface density of clonally-amplified library molecules hybridized to adapter or primer sequences on the support surface may span the same range as that indicated for the surface density of tethered primers.

Local densities as listed above do not preclude variation in density across a surface, such that a surface may comprise a region having an oligo density of, for example, 500,000/μm², while also comprising at least a second region having a substantially different local density.

In some embodiments, the performance of nucleic acid hybridization and/or amplification reactions using the disclosed reaction formulations and low-binding supports may be assessed using fluorescence imaging techniques, where the contrast-to-noise ratio (CNR) of the images provides a key metric in assessing amplification specificity and non-specific binding on the support. CNR is commonly defined as: CNR=(Signal-Background)/Noise. The background term is commonly taken to be the signal measured for the interstitial regions surrounding a particular feature (diffraction limited spot, DLS) in a specified region of interest (ROI). While signal-to-noise ratio (SNR) is often considered to be a benchmark of overall signal quality, it can be shown that improved CNR can provide a significant advantage over SNR as a benchmark for signal quality in applications that require rapid image capture (e.g., sequencing applications for which cycle times must be minimized), as shown in the example below. At high CNR the imaging time required to reach accurate discrimination (and thus accurate base-calling in the case of sequencing applications) can be drastically reduced even with moderate improvements in CNR. Improved CNR in imaging data on the imaging integration time provides a method for more accurately detecting features such as clonally-amplified nucleic acid colonies on the support surface.

In most ensemble-based sequencing approaches, the background term is typically measured as the signal associated with ‘interstitial’ regions. In addition to “interstitial” background (Bᵢₙₜₑᵣ), “intrastitial” background (Bᵢₙₜᵣₐ) exists within the region occupied by an amplified DNA colony. The combination of these two background signals dictates the achievable CNR, and subsequently directly impacts the optical instrument requirements, architecture costs, reagent costs, run-times, cost/genome, and ultimately the accuracy and data quality for cyclic array-based sequencing applications. The Bᵢₙₜₑᵣ background signal arises from a variety of sources; a few examples include auto-fluorescence from consumable flow cells, non-specific adsorption of detection molecules that yield spurious fluorescence signals that may obscure the signal from the ROI, the presence of non-specific DNA amplification products (e.g., those arising from primer dimers). In typical next generation sequencing (NGS) applications, this background signal in the current field-of-view (FOV) is averaged over time and subtracted. The signal arising from individual DNA colonies (i.e., (Signal)-B(interstial) in the FOV) yields a discernable feature that can be classified. In some embodiments, the intrastitial background (B(intrastitial)) can contribute a confounding fluorescence signal that is not specific to the target of interest, but is present in the same ROI thus making it far more difficult to average and subtract.

Nucleic acid amplification on the low-binding coated supports described herein may decrease the B(interstitial) background signal by reducing non-specific binding, may lead to improvements in specific nucleic acid amplification, and may lead to a decrease in non-specific amplification that can impact the background signal arising from both the interstitial and intrastitial regions. In some embodiments, the disclosed low-binding coated supports, optionally used in combination with the disclosed hybridization and/or amplification reaction formulations, may lead to improvements in CNR by a factor of 2, 5, 10, 100, 250, 500 or 1000-fold over those achieved using conventional supports and hybridization, amplification, and/or sequencing protocols. Although described here in the context of using fluorescence imaging as the read-out or detection mode, the same principles apply to the use of the disclosed low-binding coated supports and nucleic acid hybridization and amplification formulations for other detection modes as well, including both optical and non-optical detection modes.

The present disclosure provides methods for determining the sequence of a nucleic acid template molecule, where the multivalent molecules are labeled with fluorophores and the detecting and/or identifying steps comprise use of fluorescence imaging. In some embodiments, the fluorescence imaging comprises dual wavelength excitation/four wavelength emission fluorescence imaging. In some embodiments, four different types of multivalent molecules are employed, each comprising a different nucleotide moiety (or nucleotide moiety analog). For example, a first type of multivalent molecules comprise dATP nucleotide moieties, a second type of multivalent molecules comprise dGTP nucleotide moieties, a third type of multivalent molecules comprise dCTP nucleotide moieties, and a fourth type of multivalent molecules comprise dTTP nucleotide moieties. In some embodiments, the four different types of multivalent molecules are labeled with a different fluorophore that corresponds to the nucleotide moieties attached to a given multivalent molecule to permit identification of the nucleotide moieties. In some embodiments, the detecting step comprises simultaneous or single excitation at a wavelength sufficient to excite all four fluorophores and imaging of fluorescence emission at wavelengths sufficient to detect each respective fluorophore. In some embodiments, the four labeled multivalent molecules are used to determine the identity of a terminal nucleotide in the nucleic acid template molecule. In some embodiments, the four types of multivalent molecules are labeled with different fluorophores, including for example fluorophores that emit different visible colors such as blue, green, yellow, orange or red. In some embodiments, the four types of multivalent molecules are labeled with different fluorophores, including for example Cy2 or a dye or fluorophore similar in excitation or emission properties, Cy3 or a dye or fluorophore similar in excitation or emission properties, Cy3.5 or a dye or fluorophore similar in excitation or emission properties, Cy5 or a dye or fluorophore similar in excitation or emission properties, Cy5.5 or a dye or fluorophore similar in excitation or emission properties, and Cy7 or a dye or fluorophore similar in excitation or emission properties. In some embodiments, the detecting step comprises simultaneous excitation at any two of 532 nm, 568 nm and 633 nm, and imaging of fluorescence emission at about 570 nm, 592 nm, 670 nm, and 702 nm, respectively. In some embodiments, the fluorescence imaging comprises dual wavelength excitation/dual wavelength emission fluorescence imaging. In some embodiments, the four different types of multivalent molecules are labeled with distinguishable fluorophores (or a set of fluorophores), and the detecting step comprises simultaneous or single excitation at a wavelength sufficient to excite one, two, three, or four fluorophores or sets of fluorophores, and imaging of fluorescence emission at wavelengths is sufficient to detect each respective fluorophore.

In some embodiments, the sequencing methods can be conducted with three different types of labeled multivalent molecules and one type of unlabeled multivalent molecule (e.g., a “dark” multivalent molecule), where the labeled multivalent molecules are labeled with a different fluorophore that corresponds to the nucleotide moieties attached to a given multivalent molecule to permit identification of the nucleotide moieties. In some embodiments, the detecting step comprises simultaneous excitation at a wavelength sufficient to excite the three types of fluorophores and imaging of fluorescence emission at wavelengths is sufficient to detect each respective fluorophore, and detection of the fourth type of multivalent molecule is determined or determinable with reference to the location of “dark” or unlabeled spots.

In some embodiments, the fluorophores comprise a FRET donor and accepter pair, such that multiple detections and identifications can be performed under a single excitation and imaging step. In some embodiments, a sequencing cycle comprises forming a plurality of polymerase complexes, contacting the polymerase complexes with a plurality of different types of fluorescently-labeled multivalent molecules, and detecting the fluorescently-labeled multivalent molecules that are bound to the polymerase complexes. In some embodiments, a sequencing cycle can be conducted in less than 30 minutes, in less than 20 minutes, or in less than 10 minutes. In some embodiments, conducting sequencing reactions with labeled multivalent molecules gives an average Q-score for base calling accuracy over a sequencing run which is greater than or equal to 30, and/or greater than or equal to 40. In some embodiments, at least 50%, at least 60%, at least 70%, at least 80%, or at least 90% of the base calls have a Q-score of greater than 30 and/or greater than or equal to 40. In some embodiments, the present disclosure provides the method, herein at least 95% of the base calls have a Q-score of greater than 30.

### EXAMPLES

The following examples are meant to be illustrative and can be used to further understand embodiments of the present disclosure and should not be construed as limiting the scope of the present teachings in any way.

### Example 1: On-Support Ligation and Rolling Circle Amplification

Input DNA from various organisms was fragmented using either focused acoustic energy (e.g., Covaris) or an enzymatic fragmentation and A-tailing mix (e.g., Watchmaker Genomics). The input DNA included human, E. coli, Haemophilus influenza, Rhodopseudomonas palustris and Rhodopseudomonas sphaeroides. The enzymatic method was conducted according to manufacturer's instructions.

An adaptor ligation reaction was conducted by reacting the fragmented DNA with double stranded adaptors to generate linear library molecules wherein individual library molecules comprised a sequence of interest (e.g., insert region (110)) flanked on both sides with universal adaptor sequences (e.g., see FIG. 20A). One strand of the double stranded adaptors included: (i) a first universal binding site (120) for a first portion of a splint capture primer (or a complementary sequence thereof); (ii) a left sample index sequence (160) which can be used to distinguish sequences of interest obtained from different sample sources in a multiplex assay; and (iii) a universal binding site for a forward sequencing primer (140) (or a complementary sequence thereof). The other strand of the double stranded adaptors included: (i) second a universal binding site (130) for a second portion of the immobilized splint capture (or a complementary sequence thereof); (ii) a right sample index sequence (170) which can be used to distinguish sequences of interest obtained from different sample sources in a multiplex assay; and (iii) a universal binding site for a reverse sequencing primer (150) (or a complementary sequence thereof). The resulting linear library was not subjected to PCR amplification.

The linear library molecules were subjected to a double sided size selection using SPRISELECT paramagnetic beads (from Beckman Coulter, catalog #B23317). Different ranges of insert sizes were selected including approximately 200-400 bases, approximately 500-700 bases, and approximately 800-1000 bases.

The size selected linear library and compaction oligonucleotides were loaded onto a sequencing flow cell which was placed on a sequencing apparatus configured with automated fluidics distribution and temperature modulation systems. The sequencing apparatus was also configured with lasers to induce fluorescence, a fluorescent microscope to image the fluorescent concatemers (e.g., DNA nanoballs) during sequencing, detectors for detecting fluorescent signals emitted from the fluorescent concatemer, and a computer for receiving the imaging data. The computer was programmed to determine the location (e.g., mapping) of the immobilized concatemer template molecules (polonies) on the flow cell and generate a map of the locations of the immobilized concatemer template molecules based on the fluorescent imaging data.

The surface of the flow cell was passivated with at least one hydrophilic polymer coating layer which included covalently linked splint capture primers (200) and pinning primers (500). Different loading amounts of the linear library were tested, including 8 pM, 12 pM, 20 pM, 25 pM and 28 pM.

The linear library was denatured using NaOH prior to loading onto the flow cell. The denatured library was hybridized to the immobilized splint capture primers (200) to generate open circle library molecules hybridized to splint capture primers, the open circle library molecules were ligated to generate covalently closed circular library molecules hybridized to splint capture primers, and rolling circle amplification was conducted to generate a plurality of concatemers immobilized to the flow cell. The rolling circle amplification reaction included a strand displacing polymerase and mixture of dATP, dCTP, dGTP, dTTP and dUTP. The flow cell was washed to remove unreacted linear library molecules. The immobilized concatemers were subjected to two stage sequencing (see Example 4 below) and pairwise sequencing workflows which is described herein (e.g., FIGS. 29-37). During the first stage of sequencing, fluorescent images emitted from the concatemers and fluorescently labeled multivalent molecules were used to generate sequencing data which is listed in Table 1 below. In Table 1 below, the ‘polony PF density’ is the density of polony locations that pass filtering based on sequencing performance in the filtering cycles. The ‘polony PF rate’ is calculated by polony PF density/polony density.

TABLE 1
Human DNA library

| Library | Polony | Extrapolated | Polony | | | |
|---|---|---|---|---|---|---|
| loading | count | reads | density | Polony PF | Polony PF | |
| (pM) | (Millions) | (millions) | (per mm²) | density | rate | % Q30 |
| 8 pM | 1,168 | 1054.5 | 600,081 | 542,524 | 90.4% | 93.9% |
| 8 pM | 1,136 | 1028.4 | 583,875 | 530,026 | 90.8% | 93.6% |
| 8 pM | 1,146 | 1029.5 | 587,739 | 527,717 | 89.8% | 93.5% |
| 8 pM | 1,028 | 937.8 | 525,186 | 479,255 | 91.3% | 92.5% |
| 8 pM | 987 | 865.8 | 505,369 | 442,546 | 87.6% | 92.5% |
| 8 pM | 989 | 912.2 | 502,514 | 465,296 | 92.6% | 93.4% |
| 6 pM | 987 | 895.0 | 504,359 | 456,394 | 90.5% | 92.6% |
| 8 pM | 956 | 897.2 | 491,913 | 462,240 | 94.0% | 95.3% |
| 6 pM | 955 | 899.7 | 495,095 | 466,071 | 94.1% | 95.4% |

### Example 2: On-Support Ligation and Rolling Circle Amplification

100 ng of input DNA from E. coli was fragmented using an enzymatic fragmentation and A-tailing mix (e.g., Watchmaker Genomics). An adaptor ligation reaction was conducted by reacting the fragmented DNA with double stranded adaptors from a commercially-available library prep kit (e.g., Kapa HyperPlus kit) to generate sample indexed linear library molecules in which individual library molecules comprised a sequence of interest (e.g., insert region (110)) flanked on both sides with universal adaptor sequences (e.g., see FIG. 22A). The libraries included either 24-plex or 96-plex sample indexes.

The linear library molecules were subjected to size selection using SPRISELECT paramagnetic beads (from Beckman Coulter, catalog #B23317). The average insert size was estimated to be approximately 350 bases.

One strand of the double stranded adaptors included: (i) a universal binding site for a first non-splint capture primer (123) (or a complementary sequence thereof); (ii) a left sample index sequence (160) which can be used to distinguish sequences of interest obtained from different sample sources in a multiplex assay; and (iii) a universal binding site for a forward sequencing primer (140) (or a complementary sequence thereof).

The other strand of the double stranded adaptors included: (i) a universal binding site for a second non-splint capture primer (133) (or a complementary sequence thereof); (ii) a right sample index sequence (170) which can be used to distinguish sequences of interest obtained from different sample sources in a multiplex assay; and (iii) a universal binding site for a reverse sequencing primer (150) (or a complementary sequence thereof).

The resulting linear library was subjected to 5 cycles of PCR amplification using first tailed primers (e.g., forward tailed primers) and second tailed primers (e.g., reverse tailed primers). The first tailed primers included a 5′ region carrying a second universal binding site (130) for a second portion of the immobilized splint capture (or a complementary sequence thereof), and a 3′ region that is complementary to at least a portion of a universal binding site for a second non-splint capture primer (133) (or a complementary sequence thereof). The second tailed primers included a 5′ region carrying a 5′ region carrying a first universal binding site (120) for a first portion of a splint capture primer (or a complementary sequence thereof), and a 3′ region that is complementary to at least a portion of a universal binding site for a first non-splint capture primer (123) (or a complementary sequence thereof).

Two different first tailed primers were tested.

For example, the first tailed primers-A comprised the sequence:

(SEQ ID NO: 21)
5′-GATCAGGTGAGGCTGCGACGACTAAGCAGAAGACGGCATA
CGAGAT-3′.

For example, the first tailed primers-B comprised the sequence:

(SEQ ID NO: 22)
5′-GATCAGGTGAGGCTGCGACGACTGAAGACGGCATACGAGAT-3′.

Two different second tailed primers were tested.

For example, the second tailed primers-A comprised the sequence:

(SEQ ID NO: 53)
5′-CATGTAATGCACGTACTTTCAGGGTATGATACGGCGACC
ACCGAG-3′.

For example, the second tailed primers-B comprised the sequence:

(SEQ ID NO: 54)
5′-CATGTAATGCACGTACTTTCAGGGTGACCACCGAGATCTACAC-3′.

PCR amplification using 2 uM of the first and second tailed primers generated linear library molecules having the structure shown in FIG. 22A but lacking the left unique molecular index sequence (UMI) (180). For example, a PCR reaction included first and second primers of set A, and a separate PCR reaction included first and second primers of set B.

The amplified linear library was heat denatured. The denatured library (10 pM) and compaction oligonucleotides were loaded onto a sequencing flow cell which was placed on a sequencing apparatus configured with automated fluidics distribution and temperature modulation systems. The sequencing flow cell was passivated with a hydrophilic polymer coating which included covalently linked capture and pinning primers. The splint capture primers (200) comprised the sequence 5′-ATTACATGGATCAGGTGAGGCT-3′ (SEQ ID NO:80). The pinning primers comprised the sequence 5′-AATGCACGTACTTTCAGGGT-3 (SEQ ID NO:119). The pinning primers were non-extendible since they were phosphorylated at their 3′ ends. The sequencing apparatus was also configured with lasers to induce fluorescence, a fluorescent microscope to image the fluorescent concatemers (e.g., DNA nanoballs) during sequencing, detectors for detecting fluorescent signals emitted from the fluorescent concatemer, and a computer for receiving the imaging data. The computer was programmed to determine the location (e.g., mapping) of the immobilized concatemer template molecules (polonies) on the flow cell and generate a map of the locations of the immobilized concatemer template molecules based on the fluorescent imaging data.

10 pM of denatured library was loaded onto the flow cell for hybridization to the immobilized splint capture primers (200) to generate open circle library molecules hybridized to splint capture primers, the open circle library molecules were ligated to generate covalently closed circular library molecules hybridized to splint capture primers, and rolling circle amplification was conducted to generate a plurality of concatemers immobilized to the flow cell. The rolling circle amplification reaction included a strand displacing polymerase and mixture of dATP, dCTP, dGTP, dTTP and dUTP. The flow cell was washed to remove unreacted linear library molecules. The immobilized concatemers were subjected to two-stage sequencing (see Example 4 below) and pairwise sequencing workflows which is described herein (e.g., FIGS. 29-36). During the first stage of sequencing, fluorescent images emitted from the concatemers and fluorescently labeled multivalent molecules were used to generate sequencing data which is listed in Table 2 below.

TABLE 2
E. coli DNA library

| | Polony count | Polony | | | Index |
|---|---|---|---|---|---|
| Description | (millions) | PF rate | R1 Q30 | R2 Q30 | assignment |
| Primer set A | 1018.1 | 95.05% | 97.1% | 95.1% | 96.94% |
| 24-plex | | | | | |
| Primer set B | 867.5 | 95.47% | 97.3% | 94.6% | 96.95% |
| 24-lpex | | | | | |
| Primer set A | 1140.3 | 92.23% | 96.4% | 95.6% | 96.8% |
| 96-plex | | | | | |
| Primer set B | 900.5 | 95.6% | 97.3% | 97.3% | 97.4% |
| 96-plex | | | | | |

### Example 3: On-Support Ligation and Rolling Circle Amplification

The sequencing flow cells were passivated with a hydrophilic polymer coating which included covalently linked splint capture primers and pinning primers. The polymer coating on the sequencing flow cells included two types of splint capture primers (200) and two types of pinning primers (500).

The first type of splint capture primers (200-A) were designed to hybridize to a first type of linear library molecule (e.g., see FIGS. 20A and 27A left). The first type of splint capture primers comprised the sequence:

(SEQ ID NO: 106)
5′-CGCCGTATCAAGCAGAAGACGGCATACGAG-3′.

The second type of splint capture primers (200-B) were designed to hybridize to a second type of linear library molecule (e.g., see FIGS. 22A and 27A right). The second type of splint capture primers comprised the sequence:

(SEQ ID NO: 86)
5′-GCATTACATGGATCAGGTGAGGCTGCGACGAC-3′.

The pinning primers were non-extendible since they were phosphorylated at their 3′ ends.

### Preparing the First Type of Library:

A first type of linear library (100-A) was prepared as a PCR-free human DNA library in a manner similar to the workflow described in Example 1 above. Briefly, the fragmented human DNA was subjected to an adaptor ligation reaction by reacting the fragmented DNA with double stranded adaptors to generate linear library molecules wherein individual library molecules comprised a sequence of interest (e.g., insert region (110-A)) flanked on both sides with universal adaptor sequences (e.g., see FIG. 20A). One strand of the double stranded adaptors included: (i) a universal binding site (120-A) for a first portion of a splint capture primer (or a complementary sequence thereof); (ii) a sample index sequence (160-A) which can be used to distinguish sequences of interest obtained from different sample sources in a multiplex assay; and (iii) a universal binding site (140-A) for a forward sequencing primer (or a complementary sequence thereof). The other strand of the double stranded adaptors included: (i) a universal binding site (130-A) for a second portion of the immobilized splint capture (or a complementary sequence thereof); (ii) a sample index sequence (170-A) which can be used to distinguish sequences of interest obtained from different sample sources in a multiplex assay; and (iii) a universal binding site (150-A) for a reverse sequencing primer (or a complementary sequence thereof). The resulting linear library was not subjected to PCR amplification. The first type of linear library construct (100-A) lacked a left unique molecular index (180) and the construct is shown in FIG. 20A.

### Preparing the Second Type of Library:

A second type of linear library (100-B) was prepared as a PCR-plus human DNA library in a manner similar to the workflow described in Example 2 above. Briefly, the fragmented human DNA was subjected to an adaptor ligation reaction by reacting the fragmented DNA with double stranded adaptors from a commercially-available library prep kit (e.g., Kapa HyperPlus kit) to generate sample indexed linear library molecules wherein individual library molecules comprised a sequence of interest (e.g., insert region (110-B)) flanked on both sides with universal adaptor sequences (e.g., see FIG. 22A).

One strand of the double stranded adaptors included: (i) a universal binding site for a first non-splint capture primer (123) (or a complementary sequence thereof); (ii) a sample index sequence (160-B) which can be used to distinguish sequences of interest obtained from different sample sources in a multiplex assay; and (iii) a universal binding site (140-B) for a forward sequencing primer (or a complementary sequence thereof).

The other strand of the double stranded adaptors included: (i) a universal binding site for a second non-splint capture primer (133) (or a complementary sequence thereof); (ii) a sample index sequence (170-B) which can be used to distinguish sequences of interest obtained from different sample sources in a multiplex assay; and (iii) a universal binding site (150-B) for a reverse sequencing primer (or a complementary sequence thereof).

The resulting linear library was subjected to 5 cycles of PCR amplification using first tailed primers (e.g., forward tailed primers) and second tailed primers (e.g., reverse tailed primers). The first tailed primers included a 5′ region carrying a universal binding site (130-B) for a second portion of the immobilized splint capture (or a complementary sequence thereof), and a 3′ region that is complementary to at least a portion of a universal binding site for a second non-splint capture primer (133) (or a complementary sequence thereof). The second tailed primers included a 5′ region carrying a 5′ region carrying a universal binding site (120-B) for a first portion of a splint capture primer (or a complementary sequence thereof), and a 3′ region that is complementary to at least a portion of a universal binding site for a first non-splint capture primer (123) (or a complementary sequence thereof). The second type of linear library construct (100-B) lacked a left unique molecular index (180) and the construct is shown in FIG. 22A.

In the linear library molecules of the first and second type, the sequences of the universal binding site ((120-A) and (120-B)) for a first portion of a splint capture primer had different sequences and therefore bind to a different immobilized splint capture primer.

In the linear library molecules of the first and second type, the sequences of the universal binding site ((130-A) and (130-B)) for a second portion of a splint capture primer had different sequences and therefore bind to a different immobilized splint capture primer.

In the linear library molecules of the first and second type, the sequences of universal binding site ((140-A) and (140-B)) for a forward sequencing primer had different sequences and therefore bind to a different forward sequencing primers.

In the linear library molecules of the first and second type, the sequences of universal binding site ((150-A) and (150-B)) for a reverse sequencing primer had different sequences and therefore bind to a different reverse sequencing primers.

### Loading the Flow Cell:

The first and second types of linear library were separately heat denatured. The denatured libraries (9 pM) were loaded onto separate flow cells which were placed on a sequencing apparatus configured with automated fluidics distribution and temperature modulation systems. The first and second types of linear libraries were loaded onto their respective flow cells with compaction oligonucleotides The sequencing apparatus was also configured with lasers to induce fluorescence, a fluorescent microscope to image the fluorescent concatemers (e.g., DNA nanoballs) during sequencing, detectors for detecting fluorescent signals emitted from the fluorescent concatemer, and a computer for receiving the imaging data. The computer was programmed to determine the location (e.g., mapping) of the immobilized concatemer template molecules (polonies) on the flow cells and generate a map of the locations of the immobilized concatemer template molecules based on the fluorescent imaging data.

9 pM of the first type of denatured library (100-A) was loaded onto one sequencing flow cell for hybridization to their cognate immobilized splint capture primers to generate open circle library molecules (300-A) hybridized to splint capture primers (200-A), where the open circle library molecules included a nick. The nick in the first type of open circle library molecules were ligated to generate a first type of covalently closed circular library molecules (400-A) hybridized to splint capture primers.

9 pM of the second type of denatured library (100-B) was loaded onto another sequencing flow cell for hybridization to their cognate immobilized splint capture primers (200-B) to generate open circle library molecules hybridized to splint capture primers, where the open circle library molecules included a 5′ overhang flap (e.g., FIGS. 28A(i) and 28A(ii)). The second type of open circle library molecules (300-B) were treated with a 5′ flap cleaving reagent which included a FEN-1 endonuclease (New England Biolabs, catalog #M0645S) to cleave off the 5′ flap. The second type of open circle library molecules were treated with a recombinant thermal tolerant T4 DNA ligase (e.g., Hi-T4 DNA ligase from New England Biolabs, catalog #M2622S) to ligate nicks and generate a second type of covalently closed circular library molecules (400-B) hybridized to splint capture primers.

### Rolling Circle Amplification

The first and second type of covalently closed circular library molecules were subjected to rolling circle amplification reactions to generate first and second types of concatemers immobilized to their respective sequencing flow cells. The rolling circle amplification reactions included a strand displacing polymerase, a mixture of dATP, dCTP, dGTP, dTTP and dUTP, and a plurality of compaction oligonucleotides. The flow cells were washed to remove unreacted linear library molecules. The immobilized concatemers were subjected to two-stage sequencing (see Example 4 below) and pairwise sequencing workflows which is described herein (e.g., FIGS. 29-36). During the first stage of sequencing, fluorescent images emitted from the concatemers and fluorescently labeled multivalent molecules were used to generate sequencing data which is listed in Table 3 below.

TABLE 3
Human DNA library

| | Polony | Polony | | |
|---|---|---|---|---|
| | count | PF density | | |
| Description | (millions) | (millions) | R1 Q30 | R2 Q30 |
| 1^{st} type of linear | 1058 | 910 | 97.0% | 94.1% |
| library | | | | |
| 2^{nd} type of linear | 1113 | 1019 | 96.9% | 93.9% |
| library | | | | |

### Example 4: Two Stage Sequencing

In separate sequencing runs, the immobilized concatemers described in Examples 1, 2 and 3 above were subjected to recursive two-stage sequencing reactions using fluorescently-labeled multivalent molecules in the first stage and un-labeled nucleotide analogs (e.g., 3′ chain terminator blocking group) in the second stage.

The two-stage sequencing reaction was conducted on flow cells having a plurality of concatemer template molecules immobilized thereon (e.g., immobilized polonies).

The first-stage sequencing reaction was conducted by hybridizing a plurality of a soluble sequencing primers to concatemer template molecules that were immobilized to a flow cells to form immobilized primer-concatemer duplexes. A plurality of a first sequencing polymerase was flowed onto the flow cells (e.g., contacting the immobilized primer-concatemer duplexes) and incubated under a condition suitable to bind the sequencing polymerase to the duplexes to form polymerase complexes. A mixture of fluorescently labeled multivalent molecules (e.g., at different concentrations of about 20-100 nM) was flowed onto the flow cells in the presence of a buffer that included a non-catalytic cation (e.g., strontium, barium and/or calcium) and incubated under conditions suitable to bind complementary nucleotide moieties of the multivalent molecules to the polymerase complexes to form avidity complexes without polymerase-catalyzed incorporation of the nucleotide moieties. The fluorescently labeled multivalent molecules were labeled at their cores. The polymerase complexes were washed. An image was obtained of the fluorescently labeled multivalent molecules that remined bound to the polymerase complexes. The first sequencing polymerases and multivalent molecules were removed, while retaining the sequencing primers hybridized to the immobilized concatemers (retained duplexes), by washing with a buffer comprising a detergent.

The first stage sequencing reaction was suitable for forming a plurality of avidity complexes on the concatemer template molecules (e.g., polonies). For example, the first stage sequencing reaction comprised: (a) binding a first nucleic acid primer, a first polymerase, and a first multivalent molecule to a first portion of a concatemer template molecule thereby forming a first binding complex, wherein a first nucleotide moiety of the first multivalent molecule was bound to the first polymerase; and (b) binding a second nucleic acid primer, a second polymerase, and the first multivalent molecule to a second portion of the same concatemer template molecule thereby forming a second binding complex, wherein a second nucleotide moiety of the first multivalent molecule was bound to the second polymerase, wherein the first and second binding complexes which included the same multivalent molecule formed a first avidity complex.

The second-stage sequencing reaction was conducted by contacting the retained duplexes with a plurality of second sequencing polymerases to form polymerase complexes. A mixture of non-labeled nucleotide analogs (e.g., 3′O-methylazido nucleotides) (e.g., at different concentrations of about 1-5 uM) was added to the polymerase complexes in the presence of a buffer that included a catalytic cation (e.g., magnesium and/or manganese) and incubated under conditions suitable to bind complementary nucleotides to the polymerase complexes and promote polymerase-catalyzed incorporation of the nucleotides to generate a nascent extended sequencing primer. The polymerase complexes were washed. No image was obtained. The incorporated non-labeled nucleotide analogs were reacted with a cleaving reagent that removes the 3′ O-methylazido group and generates an extendible 3′OH group.

The second sequencing polymerases were removed, while retaining the nascent extended sequencing primers hybridized to the concatemers (retained duplexes), by washing with a buffer comprising a detergent. Recurring sequencing reactions were conducted by performing multiple cycles of first-stage and second-stage sequencing reactions to generate extended forward sequencing primer strands.

In an alternative second stage sequencing reaction, a mixture of fluorescently labeled nucleotide analogs (e.g., 3′O-methylazido nucleotides) (e.g., about 1-5 uM) is added to the polymerase complexes in the presence of a buffer that includes a catalytic cation (e.g., magnesium and/or manganese) and is incubated under conditions suitable to bind complementary nucleotides to the polymerase complexes and promote polymerase-catalyzed incorporation of the nucleotides to generate a nascent extended sequencing primer. The polymerase complexes are washed. An image is obtained of the incorporated fluorescently labeled nucleotide analogs as a part of the polymerase complexes. The incorporated fluorescently labeled nucleotide analogs is reacted with a cleaving reagent that removes the 3′ O-methylazido group and generates an extendible 3′OH group.

1. A method for sequencing a plurality of nucleic acid concatemer template molecules immobilized to a support, comprising:
a) providing a support having a plurality of splint capture primers (200) and a plurality of pinning primers (500) immobilized thereon,
wherein individual splint capture primers (200) in the plurality comprise a first portion (210) which binds a first universal binding site in a linear library molecule (100) and a second portion (220) which binds a second universal binding site in the same linear library molecule (100),
wherein a density of the splint capture primers (200) on the support is between 10⁵-10¹⁵ per mm²,
wherein individual pinning primers (500) bind at least a portion of individual nucleic acid concatemer template molecules, and wherein individual pinning primers (500) comprise a terminal 3′ non-extendible end;
b) providing a plurality of linear library molecules (100), wherein individual linear library molecules comprise a sequence of interest and any one or combination of:
(i) a first universal binding site (120) for a first portion of a splint capture primer (200) or a complementary sequence thereof;
(ii) a universal binding site for a first non-splint capture primer (123) or a complementary sequence thereof;
(iii) at least one sample index sequence comprising a left sample index sequence (160) and/or a right sample index sequence (170), wherein the left sample index sequence (160) and/or right sample index sequence (170) distinguishes sequences of interest obtained from different sample sources in a multiplex assay;
(iv) at least one universal binding site for a forward sequencing primer (140) or a complementary sequence thereof;
(v) at least one universal binding site for a reverse sequencing primer (150) or a complementary sequence thereof;
(vi) at least one universal binding site for a compaction oligonucleotide or a complementary sequence thereof;
(vii) at least one unique molecular index sequence (UMI) comprising a left unique molecular index sequence (180) and/or a right unique molecular index sequence (190) which can be used to uniquely identify a linear library molecule (100) to which the unique molecular index sequence is appended;
(viii) at least one universal binding site for a pinning primer (500) or a complementary sequence thereof;
(ix) at least one batch-specific barcode sequence;
(x) a universal binding site for a second non-splint capture primer (133) or a complementary sequence thereof;
(xi) at least one short random sequence (132) which is about 3-20 nucleotides in length and provides nucleotide sequence diversity; and/or
(xii) a second universal binding site (130) for a second portion of the splint capture primer (200 or a complementary sequence thereof;
c) contacting the plurality of splint capture primers (200) with the plurality of linear library molecules (100), wherein the contacting is conducted under a condition suitable for hybridizing individual linear library molecules to individual splint capture primers to form individual open circle library molecules (300) having a gap or nick between the terminal 5′ and 3′ ends of the open circle library molecule (300),
wherein individual linear library molecules comprise the first universal binding site (120) for the first portion of the splint capture primer (200) which is hybridized to the first portion (210) of a splint capture primer, and
wherein the same linear library molecule (100) comprises the second universal binding site (130) for the second portion of the splint capture primer (200) which is hybridized to the second portion (220) of the same splint capture primer (200), thereby generating a plurality of open circle library molecules (300) comprising nicks or gaps;
d) enzymatically closing the nicks or gaps in the plurality of open circle library molecules (300), thereby generating a plurality of covalently closed circular library molecules (400), wherein individual covalently closed circular library molecules (400) are hybridized to the splint capture primers (200);
e) contacting the plurality of covalently closed circular library molecules (400) with a rolling circle amplification reaction mixture and conducting a rolling circle amplification reaction, thereby generating the plurality of nucleic acid concatemer template molecules, wherein the plurality of nucleic acid concatemer template molecules are immobilized on the support, and wherein the density of the nucleic acid concatemer template molecules on the support is between 10⁵-10¹⁵ per mm²,
wherein the rolling circle amplification reaction mixture comprises a strand displacing polymerase and a mixture of nucleotides comprising dATP, dGTP, dCTP, dTTP and dUTP,
wherein the rolling circle amplification reaction mixture comprises a plurality of single-stranded nucleic acid compaction oligonucleotides,
wherein 5′ and 3′ regions of individual single-stranded nucleic acid compaction oligonucleotides hybridize to universal binding sites on an individual nucleic acid concatemer template molecule, thereby compacting the nucleic acid concatemer template molecule to form a DNA nanoball,
wherein terminal 3′ ends of individual single-stranded nucleic acid compaction oligonucleotides are non-extendible, and
wherein at least one portion of the individual nucleic acid concatemer template molecule is hybridized to an individual pinning primer (500); and
f) conducting at least one sequencing reaction to determine a sequence of at least a portion of the plurality of nucleic acid concatemer template molecules.

2. The method of claim 1, wherein step (c) comprises:
(i) contacting the plurality of splint capture primers (200) with the plurality of linear library molecules (100), wherein the contacting is conducted under a condition suitable for hybridizing individual linear library molecules (100) to individual splint capture primers (200) to form individual open circle library molecules (300) comprising a 5′ overhang flap structure, and
(ii) contacting the 5′ overhang flap structures with a flap cleaving reagent under a condition suitable to cleave the 5′ overhang flap structures to generate a plurality of open circle library molecules comprising a cleaved 5′ end and a non-cleaved 3′ end, and comprising a nick between the cleaved 5′ and non-cleaved 3′ ends of the open circle library molecule.

3. The method of claim 1, wherein the plurality of splint capture primers (200) of step (a) are located at random and non-predetermined positions on the support.

4. The method of claim 1, wherein the plurality of splint capture primers (200) of step (a) includes a plurality of nearest neighbor splint capture primers that contact each other and/or overlap with each other when the support is viewed from any angle including above, below or from the side.

5. The method of claim 1, wherein the plurality of splint capture primers (200) of step (a) comprises at least a first sub-population of splint capture primers having a first sequence and a second sub-population of splint capture primers having a second sequence which differs from the first sequence.

6. The method of claim 1, wherein the plurality of linear library molecules (100) in step (b) comprises at least a first sub-population of linear library molecules and a second sub-population of linear library molecules.

7. The method of claim 6, wherein the linear library molecules (100) in the first sub-population comprise a mixture of sequences of interest and the linear library molecules (100) in the second sub-population comprise a mixture of sequences of interest.

8. The method of claim 7, wherein
ii) the first sub-population of linear library molecules comprises:
a universal binding site for a first batch-specific forward sequencing primer (140-1) or a complementary sequence thereof,
a universal binding site for a first batch-specific reverse sequencing primer (150-1) or a complementary sequence thereof, and
a first batch-specific barcode sequence (142); and
(ii) the second sub-population of linear library molecules comprises:
a universal binding site for a second batch-specific forward sequencing primer (140-2) or a complementary sequence thereof,
a universal binding site for a second batch-specific reverse sequencing primer (150-2) or a complementary sequence thereof, and
a second batch-specific barcode sequence (152).

9. The method of claim 1, wherein the plurality of nucleic acid concatemer template molecules of step (e) comprises at least a first sub-population of nucleic acid concatemer template molecules and a second sub-population of nucleic acid concatemer template molecules.

10. The method of claim 9, wherein the first sub-population of nucleic acid concatemer template molecules and the second sub-population of concatemer template molecules are located at random and non-predetermined positions on the support, and wherein individual nucleic acid concatemer template molecules in the first and second sub-populations include nearest neighbor nucleic acid concatemer template molecules that contact each other or overlap with each other when the support is viewed from any angle including above, below or from the side.

11. The method of claim 10, wherein conducting the sequencing reaction of step (f) comprises conducting a first batch reiterative sequencing, comprising:
a) hybridizing the first sub-population of nucleic acid concatemer template molecules with a plurality of first batch-specific forward sequencing primers and conducting a plurality of sequencing reactions, thereby generating a plurality of first batch sequencing read products, wherein the first batch sequencing read products are no more than 50 bases in length;
b) inhibiting further sequencing reactions, thereby stopping the first batch reiterative sequencing;
c) removing the plurality of first batch sequencing read products from the first sub-population of nucleic acid concatemer template molecules and retaining the first sub-population of nucleic acid concatemer template molecules; and
d) reiteratively sequencing the first sub-population of nucleic acid concatemer template molecules by repeating steps (a)-(c) at least once.

12. The method of claim 11, wherein conducting the sequencing reaction of step (f) further comprises conducting a second batch reiterative sequencing which comprises:
a) hybridizing the second sub-population of nucleic acid concatemer template molecules with a plurality of second batch-specific forward sequencing primers and conducting a plurality of sequencing reactions, thereby generating a plurality of second batch sequencing read products, wherein the second batch sequencing read products are no more than 50 bases in length;
b) inhibiting further sequencing reactions, thereby stopping the second batch reiterative sequencing;
c) removing the plurality of second batch sequencing read products from the second sub-population of nucleic acid concatemer template molecules and retaining the second sub-population of nucleic acid concatemer template molecules; and
d) reiteratively sequencing the second sub-population of nucleic acid concatemer template molecules by repeating steps (a)-(c) at least once.

13. The method of claim 1, wherein:
(i) step (c) comprises distributing onto the support a first sub-population of linear library molecules under a condition suitable for hybridizing individual linear library molecules from the first sub-population to individual splint capture primers (200) to generate a first sub-population of open circle library molecules each having nick or gap, wherein the support comprises an excess of splint capture primers immobilized thereon compared to the first sub-population of linear library molecules;
(ii) step (d) comprises enzymatically closing the nick or gap to generate a first sub-population of covalently closed circular library molecules, wherein individual covalently closed circular library molecules are hybridized to a splint capture primer;
(iii) step (e) comprises conducting a rolling circle amplification reaction to generate a first sub-population of nucleic acid concatemer template molecules;
(iv) step (f) comprises sequencing at least a portion of the first sub-population of nucleic acid concatemer template molecules; and
(v) wherein the method further comprises:
halting the sequencing of the first sub-population of nucleic concatemer template molecules;
distributing onto the same support a second sub-population of linear library molecules under a condition suitable for hybridizing individual linear library molecules from the second sub-population to individual splint capture primers (200) to generate a second sub-population of open circle library molecules having a nick or gap, and enzymatically closing the nick or gap to generate a second sub-population of covalently closed circular library molecules, wherein individual covalently closed circular library molecules are hybridized to a splint capture primer, conducting a rolling circle amplification reaction to generate a second sub-population of concatemer template molecules; and
continuing the sequencing of at least a portion of the first sub-population of concatemer template molecules or sequencing at least a portion of the second sub-population of concatemer template molecules.

14. The method of claim 1, wherein conducting the sequencing reaction of step (f) comprises pairwise sequencing comprising:
a) generating a plurality of extended forward sequencing primer strands by contacting the plurality of nucleic acid concatemer template molecules with a plurality of forward sequencing primers under a condition suitable to hybridize at least one forward sequencing primer to at least one of the universal binding sites for a forward sequencing primer (140) of the nucleic acid concatemer template molecules, and conducting forward sequencing reactions using the hybridized first forward sequencing primers, a plurality of sequencing polymerases, and a plurality of nucleotide reagents;
b) retaining the plurality of nucleic acid concatemer template molecules and replacing the plurality of extended forward sequencing primer strands with a plurality of forward extension strands that are hybridized to the nucleic acid concatemer template molecules by conducting a primer extension reaction using the nucleic acid concatemer template molecules as template molecules;
c) removing the nucleic acid concatemer template molecules by generating abasic sites in the nucleic acid concatemer template molecules at uridine nucleotides in the nucleic acid concatemer template molecules and generating gaps at the abasic sites to generate a plurality of gap-containing nucleic acid concatemer template molecules while retaining the plurality of forward extension strands and retaining the pluralities of splint capture primers and pinning primers; and
d) sequencing the plurality of forward extension strands by contacting the plurality of forward extension strands with a plurality of soluble reverse sequencing primers, a plurality of sequencing polymerases, and a plurality of nucleotide reagents, and conducting reverse sequencing reactions thereby generating a plurality of extended reverse sequencing primer strands.

15. The method of claim 1, wherein the sequencing reaction of step (f) comprises chain terminator sequencing comprising:
a) contacting the plurality of nucleic acid concatemer template molecules with a plurality of sequencing polymerases and a plurality of forward sequencing primers, where the contacting is conducted under a condition suitable to form a plurality of sequencing polymerase complexes comprising a sequencing polymerase bound to a nucleic acid duplex, wherein the nucleic acid duplex comprises a portion of a nucleic acid concatemer template molecule hybridized to a sequencing primer;
b) contacting the plurality of sequencing polymerase complexes with a plurality of nucleotides comprising a detectable label and a blocking moiety at the 2′ or 3′ sugar position, where the contacting is conducted under a condition suitable for binding at least one nucleotide to one of the sequencing polymerase complexes, and the condition is suitable for promoting polymerase-catalyzed nucleotide incorporation;
c) incorporating a nucleotide into a 3′ end of the sequencing primer of at least one sequencing polymerase complex, thereby generating a sequencing polymerase complex comprising an incorporated nucleotide;
d) detecting the incorporated nucleotide;
e) removing the blocking moiety from the incorporated nucleotide; and
f) repeating steps (b)-(e) at least once.

16. The method of claim 1, wherein the sequencing reaction of step (f) comprises:
a) contacting the plurality of nucleic acid concatemer template molecules with a plurality of sequencing polymerases and a plurality of forward sequencing primers, wherein the contacting is conducted under a condition suitable to form a plurality of sequencing polymerase complexes comprising a sequencing polymerase bound to a nucleic acid duplex, wherein the nucleic acid duplex comprises a portion of a nucleic acid concatemer template molecule hybridized to a forward sequencing primer;
b) contacting the plurality of sequencing polymerase complexes with a plurality of nucleotides comprising detectable labels attached to a phosphate moiety of the phosphate chain, wherein the contacting is conducted under a condition suitable for binding at least one nucleotide to one of the sequencing polymerase complexes, and the condition is suitable for promoting polymerase-catalyzed nucleotide incorporation;
c) incorporating a nucleotide into the 3′ end of a sequencing primer of at least one sequencing polymerase complex, thereby generating a sequencing polymerase complex comprising an incorporated nucleotide;
d) detecting the incorporated nucleotide; and
e) repeating steps (b)-(d) at least once.

17. The method of claim 1, wherein the sequencing reaction of step (f) comprises:
a) contacting the plurality of nucleic acid concatemer template molecules with a plurality of a first sequencing polymerases and a plurality of forward sequencing primers, wherein the contacting is conducted under a condition suitable to bind the plurality of first polymerases to the plurality of nucleic acid concatemer template molecules and the plurality of sequencing primers, thereby forming a plurality of first polymerase complexes comprising a first sequencing polymerase bound to a nucleic acid duplex, wherein the nucleic acid duplex comprises a nucleic acid concatemer template molecule hybridized to a sequencing primer;
b) contacting the plurality of first polymerase complexes with a plurality of multivalent molecules, wherein the multivalent molecules are detectably labeled, and wherein individual multivalent molecules in the plurality comprise a core attached to multiple nucleotide arms, wherein individual nucleotide arms are attached to a nucleotide moiety, wherein the contacting is conducted under a condition suitable for binding complementary nucleotide moieties of the multivalent molecules to at least two of the plurality of first polymerase complexes, thereby forming a plurality of multivalent-binding polymerase complexes, and the condition is suitable for inhibiting incorporation of complementary nucleotide moieties into the sequencing primers of the plurality of multivalent-binding polymerase complexes;
c) detecting the plurality of multivalent-binding polymerase complexes; and
d) identifying the base of the nucleotide moieties in the plurality of multivalent-binding polymerase complexes, thereby determining the sequence of the nucleic acid concatemer template molecules.

18. The method of claim 17, further comprising:
e) dissociating the plurality of multivalent-binding polymerase complexes by removing the plurality of first sequencing polymerases and multivalent molecules, and retaining the nucleic acid duplexes, thereby generating a plurality of retained nucleic acid duplexes;
f) contacting the plurality of the retained nucleic acid duplexes of step (e) with a plurality of second sequencing polymerases under a condition suitable for binding the plurality of second polymerases to the plurality of the retained nucleic acid duplexes, thereby forming a plurality of second polymerase complexes comprising a second sequencing polymerase bound to the retained nucleic acid duplex; and
g) contacting the plurality of second polymerase complexes with a plurality of nucleotides, wherein the contacting is conducted under a condition suitable for binding complementary nucleotides from the plurality of nucleotides to at least two of the second polymerase complexes, thereby forming a plurality of nucleotide-polymerase complexes, and the condition is suitable for promoting nucleotide incorporation of the complementary nucleotides into the primers of the nucleotide-binding complexes.

19. The method of claim 18, wherein nucleotides in the plurality of nucleotides comprise detectable labels, and the method comprises: (h) detecting the complementary nucleotides which are incorporated into the nucleic acid sequencing primers of the nucleotide-polymerase complexes.

20. The method of claim 18, further comprising:
h) detecting the complementary nucleotides which are incorporated into the nucleic acid sequencing primers of the nucleotide-polymerase complexes; and
i) identifying the bases of the complementary nucleotides which are incorporated into the nucleic acid sequencing primers of the nucleotide-polymerase complexes.

21.-23. (canceled)

24. The method of claim 17, wherein individual multivalent molecules in the plurality of multivalent molecules comprise: (a) a core; and (b) a plurality of nucleotide arms which comprise (i) a core attachment moiety, (ii) a spacer, (iii) a linker, and (iv) a nucleotide moiety, wherein the core is attached to the plurality of nucleotide arms via a core attachment moiety, wherein the spacer is attached to the linker, and wherein the linker is attached to the nucleotide moiety.

25. (canceled)

26. The method of claim 20, wherein:
(i) the plurality of nucleotide arms attached to a given core have the same type of nucleotide moieties, and wherein the types of nucleotide moieties comprise dATP, dGTP, dCTP, dTTP or dUTP, (ii) the plurality of multivalent molecules comprises one type of multivalent molecule wherein each multivalent molecule in the plurality has the same type of nucleotide moiety selected from a group consisting of dATP, dGTP, dCTP, dTTP and dUTP, or
(iii) the plurality of multivalent molecules comprises a mixture of any combination of two or more types of multivalent molecules each type having nucleotide moieties selected from a group consisting of dATP, dGTP, dCTP, dTTP and/or dUTP.

27.-29. (canceled)

30. The method of claim 18, wherein the plurality of nucleotides of step (g) comprises one type of nucleotide selected from a group consisting of dATP, dGTP, dCTP, dTTP and dUTP, or comprise a mixture of any combination of two or more types of nucleotides selected from a group consisting of dATP, dGTP, dCTP, dTTP and/or dUTP.

31. The method of claim 18, wherein at least one of the nucleotides in the plurality of nucleotides in step (g) is labeled with a fluorophore, or wherein the plurality of nucleotides in step (g) lack a fluorophore label.

32.-33. (canceled)

34. The method of claim 20, comprising forming a plurality of binding complexes, comprising the steps:
a) binding a first sequencing primer, a first sequencing polymerase, and a first multivalent molecule to a first portion of a nucleic acid concatemer template molecule, thereby forming a first binding complex, wherein a first nucleotide moiety of the first multivalent molecule binds to the first polymerase; and
b) binding a second sequencing primer, a second sequencing polymerase, and the first multivalent molecule to a second portion of the same nucleic acid concatemer template molecule thereby forming a second binding complex, wherein a second nucleotide moiety of the first multivalent molecule binds to the second polymerase, and wherein the first and second binding complexes include the same multivalent molecule.

35. The method of claim 24, further comprising:
a) contacting the plurality of the first sequencing polymerases and the plurality of nucleic acid sequencing primers with different portions of an individual nucleic acid concatemer template molecule to form at least first polymerase complex and second polymerase complex on the same nucleic acid concatemer template molecule;
b) contacting a plurality of multivalent molecules comprising detectable labels with the at least first and second polymerase complexes, under conditions suitable to bind a single multivalent molecule from the plurality to the first and second polymerase complexes, wherein at least a first nucleotide moiety of the single multivalent molecule is bound to the first polymerase complex which includes a first nucleic acid sequencing primer hybridized to a first portion of the concatemer template molecule, thereby forming a first binding complex, and wherein at least a second nucleotide moiety of the single multivalent molecule is bound to the second polymerase complex which includes a second nucleic acid sequencing primer hybridized to a second portion of the concatemer template molecule thereby forming a second binding complex, and
wherein the contacting is conducted under a condition suitable to inhibit polymerase-catalyzed incorporation of the bound first and second nucleotide moieties in the first and second binding complexes, and
wherein the first and second binding complexes bound to the same multivalent molecule form an avidity complex;
c) detecting the first and second binding complexes on the same concatemer molecule; and
d) identifying the first nucleotide moiety in the first binding complex thereby determining the sequence of the first portion of the concatemer template molecule, and identifying the second nucleotide moiety in the second binding complex thereby determining the sequence of the second portion of the concatemer template molecule.

36. The method of claim 1, wherein conducting the at least one sequencing reaction at step (f) comprises a plurality of engineered sequencing polymerases, and wherein the plurality of engineered polymerases comprise at least 70%, 75%, 80%, 85%, 90%, 91%, 92%, 93%, 94%, 95%, 96%, 97%, 98%, 99%, or greater than 99% sequence identity to any of SEQ ID NOS:128-146.

37. The method of claim 15, wherein the plurality of sequencing polymerases of step (a) comprises a plurality of engineered polymerases comprising at least 70%, 75%, 80%, 85%, 90%, 91%, 92%, 93%, 94%, 95%, 96%, 97%, 98%, 99%, or greater than 99% sequence identity to any of SEQ ID NOS: 128-146.

38. The method of claim 16, wherein the plurality of sequencing polymerases comprises a plurality of engineered polymerases comprising at least 70%, 75%, 80%, 85%, 90%, 91%, 92%, 93%, 94%, 95%, 96%, 97%, 98%, 99%, or greater than 99% sequence identity to any of SEQ ID NOS: 128-146.

39. The method of claim 17, wherein the plurality of first sequencing polymerases comprises a plurality of engineered polymerases comprising at least 70%, 75%, 80%, 85%, 90%, 91%, 92%, 93%, 94%, 95%, 96%, 97%, 98%, 99%, or greater than 99% sequence identity to any of SEQ ID NOS: 128-146.

40. The method of claim 18, wherein the plurality of second sequencing polymerases comprises a plurality of engineered polymerases comprising at least 70%, 75%, 80%, 85%, 90%, 91%, 92%, 93%, 94%, 95%, 96%, 97%, 98%, 99%, or greater than 99% sequence identity to any of SEQ ID NOS: 128-146.

41. The method of claim 2, wherein the flap cleaving reagent comprises at least one 5′ flap endonuclease from a eukaryote or archaeal organism.

42. The method of claim 2, wherein the flap cleaving reagent comprises at least one archaeal 5′ flap endonuclease selected from the group consisting of
Archaeoglobus fulgidus (Afu FEN1),
Methanobacterium thermoautotrophicum (Mth FEN1),
Pyrococcus furiosus (Pfu FEN1),
Methanococcus jannaschii (Mja FEN1),
Pyrococcus woesei (Pwo FEN1),
Pyrococcus horikoshii (Pho FEN1),
Archaeoglobus veneficus (Ave FEN1),
Thermococcus kodakarensis (Tko FEN1),
Desulfurococcus amylolyticus (Dam FEN1),
Aeropyrum pernix (Ape FEN1),
Sulfolobus solfataricus (Sso FEN1), and
Thermococcus sp. 9 degrees North (9° N FEN1).

43.-44. (canceled)

45. The method of claim 1, wherein enzymatically closing the nicks comprises contacting the plurality of open circle library molecules with a DNA ligase comprising a T3 ligase, a T4 ligase, a T7 ligase, a Tfu ligase or a ligase from Thermococcus nautili.

46. The method of claim 2, wherein the flap cleaving reagent comprises a DNA ligase.

47. (canceled)

48. A method for generating a plurality of nucleic acid concatemer template molecules immobilized to a support, comprising:
a) providing a support comprising a plurality of splint capture primers (200) and a plurality of pinning primers (500) immobilized thereon,
wherein individual splint capture primers (200) in the plurality comprise a first portion (210) which binds a first universal binding site in a linear library molecule (100) and a second portion (220) which binds a second universal binding site in the same linear library molecule (100),
wherein density of the splint capture primers (200) on the support is between 10⁵-10⁵ per mm²,
wherein individual pinning primers (500) bind at least a portion of individual nucleic acid concatemer template molecules, and wherein individual pinning primers (500) comprise a terminal 3′ non-extendible end;
b) providing a plurality of linear library molecules (100), wherein individual linear library molecules comprise a sequence of interest and any one or any combination of:
(i) a first universal binding site (120) for a first portion of a splint capture primer (200) or a complementary sequence thereof;
(ii) a universal binding site for a first non-splint capture primer (123) or a complementary sequence thereof;
(iii) at least one sample index sequence comprising a left sample index sequence (160) and/or a right sample index sequence (170) distinguishes sequences of interest obtained from different sample sources in a multiplex assay;
(iv) at least one universal binding site for a forward sequencing primer (140) or a complementary sequence thereof;
(v) at least one universal binding site for a reverse sequencing primer (150) or a complementary sequence thereof;
(vi) at least one universal binding site for a compaction oligonucleotide or a complementary sequence thereof;
(vii) at least one unique molecular index sequence (UMI) comprising a left unique molecular index sequence (180) and/or a right unique molecular index sequence (190) which can be used to uniquely identify a linear library molecule (100 to which the unique molecular index sequence is appended;
(viii) at least one universal binding site for a pinning primer (500) or a complementary sequence thereof;
(ix) at least one batch-specific barcode sequence;
(x) a universal binding site for a second non-splint capture primer (133) or a complementary sequence thereof;
(xi) at least one short random sequence (132) which is about 3-20 nucleotides in length and provides nucleotide sequence diversity; and/or
(xii) a second universal binding site (130) for a second portion of the splint capture (200) or a complementary sequence thereof,
c) (i) contacting the plurality of splint capture primers (200) with the plurality of linear library molecules (100), wherein the contacting is conducted under a condition suitable for hybridizing individual linear library molecules (100) to individual splint capture primers (200) to form individual open circle library molecules (300) comprising a 5′ overhang flap structure, and
(ii) contacting the 5′ overhang flap structures with a flap cleaving reagent under a condition suitable to cleave the 5′ overhang flap structures to generate a plurality of open circle library molecules each having a newly cleaved 5′ end and a non-cleaved 3′ end and comprising a nick between the terminal 5′ and 3′ ends of the open circle library molecule,
wherein individual linear library molecules comprise at least a portion of the first universal binding site (120) for the first portion of a splint capture primer which hybridized to the first portion (210) of the splint capture primer (200),
wherein the same linear library molecule comprises at least a portion of the second universal binding site (130) for the second portion of the splint capture primer (200) which is hybridized to the second portion (220) of the same splint capture primer;
d) enzymatically closing the nicks in the plurality of open circle library molecules (300), thereby generating a plurality of covalently closed circular library molecules (400), wherein individual covalently closed circular library molecules are hybridized to splint capture primers (200);
e) contacting the plurality of covalently closed circular library molecules (400) with a rolling circle amplification reaction mixture and conducting a rolling circle amplification reaction, thereby generating a plurality of nucleic acid concatemer template molecules immobilized on the support, wherein the density of the concatemer template molecules on the support is 10⁵-10¹⁵ per mm²,
wherein the rolling circle amplification reaction mixture comprises a strand displacing polymerase and a mixture of nucleotides comprising dATP, dGTP, dCTP, dTTP and dUTP,
wherein the rolling circle amplification reaction mixture comprises a plurality of single-stranded nucleic acid compaction oligonucleotides,
wherein the 5′ and 3′ regions of individual single-stranded nucleic acid compaction oligonucleotides hybridize to universal binding sites on a nucleic acid concatemer template molecule to pull together distal portions of the concatemer template molecule thereby causing compaction of the concatemer template molecule to form a DNA nanoball,
wherein the terminal 3′ ends of the single-stranded nucleic acid compaction oligonucleotides are non-extendible, and
wherein at least one portion of the individual nucleic acid concatemer template molecule is hybridized to a pinning primer (500); and
f) conducting at least one sequencing reaction to determine the sequence of at least a portion of the plurality of nucleic acid concatemer template molecules.


SEQUENCE LISTING (162 sequences)

SEQ ID NO: 1 - DNA, 19 bp, synthetic construct
  source 1..19: mol_type = other DNA
agatgtgtataagagacag

SEQ ID NO: 2 - DNA, 19 bp, synthetic construct
  source 1..19: mol_type = other DNA
ctgtctcttatacacatct

SEQ ID NO: 3 - DNA, 23 bp, synthetic construct
  source 1..23: mol_type = other DNA
gatcaggtgaggctgcgacgact

SEQ ID NO: 4 - DNA, 22 bp, synthetic construct
  source 1..22: mol_type = other DNA
atcaggtgaggctgcgacgact

SEQ ID NO: 5 - DNA, 14 bp, synthetic construct
  source 1..14: mol_type = other DNA
gatcaggtgaggct

SEQ ID NO: 6 - DNA, 17 bp, synthetic construct
  source 1..17: mol_type = other DNA
gatcaggtgaggctgcg

SEQ ID NO: 7 - DNA, 17 bp, synthetic construct
  source 1..17: mol_type = other DNA
gttgaggaaccgagtgt

SEQ ID NO: 8 - DNA, 18 bp, synthetic construct
  source 1..18: mol_type = other DNA
gactattccagcggtacg

SEQ ID NO: 9 - DNA, 21 bp, synthetic construct
  source 1..21: mol_type = other DNA
caagcagaagacggcatacga

SEQ ID NO: 10 - DNA, 24 bp, synthetic construct
  source 1..24: mol_type = other DNA
caagcagaagacggcatacgagat

SEQ ID NO: 11 - DNA, 20 bp, synthetic construct
  source 1..20: mol_type = other DNA
aagcagaagacggcatacga

SEQ ID NO: 12 - DNA, 19 bp, synthetic construct
  source 1..19: mol_type = other DNA
agcagaagacggcatacga

SEQ ID NO: 13 - DNA, 18 bp, synthetic construct
  source 1..18: mol_type = other DNA
gcagaagacggcatacga

SEQ ID NO: 14 - DNA, 17 bp, synthetic construct
  source 1..17: mol_type = other DNA
cagaagacggcatacga

SEQ ID NO: 15 - DNA, 16 bp, synthetic construct
  source 1..16: mol_type = other DNA
agaagacggcatacga

SEQ ID NO: 16 - DNA, 15 bp, synthetic construct
  source 1..15: mol_type = other DNA
gaagacggcatacga

SEQ ID NO: 17 - DNA, 23 bp, synthetic construct
  source 1..23: mol_type = other DNA
aagcagaagacggcatacgagat

SEQ ID NO: 18 - DNA, 18 bp, synthetic construct
  source 1..18: mol_type = other DNA
gaagacggcatacgagat

SEQ ID NO: 19 - DNA, 23 bp, synthetic construct
  source 1..23: mol_type = other DNA
tcagcagcgtcggagtggactag

SEQ ID NO: 20 - DNA, 47 bp, synthetic construct
  source 1..47: mol_type = other DNA
gatcaggtgaggctgcgacgactcaagcagaagacggcatacgagat

SEQ ID NO: 21 - DNA, 46 bp, synthetic construct
  source 1..46: mol_type = other DNA
gatcaggtgaggctgcgacgactaagcagaagacggcatacgagat

SEQ ID NO: 22 - DNA, 41 bp, synthetic construct
  source 1..41: mol_type = other DNA
gatcaggtgaggctgcgacgactgaagacggcatacgagat

SEQ ID NO: 23 - DNA, 46 bp, synthetic construct
  source 1..46: mol_type = other DNA
atcaggtgaggctgcgacgactcaagcagaagacggcatacgagat

SEQ ID NO: 24 - DNA, 44 bp, synthetic construct
  source 1..44: mol_type = other DNA
gatcaggtgaggctgcgacgactcaagcagaagacggcatacga

SEQ ID NO: 25 - DNA, 37 bp, synthetic construct
  source 1..37: mol_type = other DNA
gatcaggtgaggctaagcagaagacggcatacgagat

SEQ ID NO: 26 - DNA, 40 bp, synthetic construct
  source 1..40: mol_type = other DNA
gatcaggtgaggctgcgaagcagaagacggcatacgagat

SEQ ID NO: 27 - DNA, 40 bp, synthetic construct
  source 1..40: mol_type = other DNA
gttgaggaaccgagtgttcagcagcgtcggagtggactag

SEQ ID NO: 28 - DNA, 41 bp, synthetic construct
  source 1..41: mol_type = other DNA
gactattccagcggtacgtcagcagcgtcggagtggactag

SEQ ID NO: 29 - DNA, 20 bp, synthetic construct
  source 1..20: mol_type = other DNA
aatgcacgtactttcagggt

SEQ ID NO: 30 - DNA, 24 bp, synthetic construct
  source 1..24: mol_type = other DNA
atgtaatgcacgtactttcagggt

SEQ ID NO: 31 - DNA, 26 bp, synthetic construct
  source 1..26: mol_type = other DNA
ccatgtaatgcacgtactttcagggt

SEQ ID NO: 32 - DNA, 20 bp, synthetic construct
  source 1..20: mol_type = other DNA
aatgatacggcgaccaccga

SEQ ID NO: 33 - DNA, 19 bp, synthetic construct
  source 1..19: mol_type = other DNA
atgatacggcgaccaccga

SEQ ID NO: 34 - DNA, 18 bp, synthetic construct
  source 1..18: mol_type = other DNA
tgatacggcgaccaccga

SEQ ID NO: 35 - DNA, 17 bp, synthetic construct
  source 1..17: mol_type = other DNA
gatacggcgaccaccga

SEQ ID NO: 36 - DNA, 16 bp, synthetic construct
  source 1..16: mol_type = other DNA
atacggcgaccaccga

SEQ ID NO: 37 - DNA, 15 bp, synthetic construct
  source 1..15: mol_type = other DNA
tacggcgaccaccga

SEQ ID NO: 38 - DNA, 14 bp, synthetic construct
  source 1..14: mol_type = other DNA
acggcgaccaccga

SEQ ID NO: 39 - skipped

SEQ ID NO: 40 - DNA, 21 bp, synthetic construct
  source 1..21: mol_type = other DNA
aatgatacggcgaccaccgag

SEQ ID NO: 41 - DNA, 20 bp, synthetic construct
  source 1..20: mol_type = other DNA
atgatacggcgaccaccgag

SEQ ID NO: 42 - DNA, 18 bp, synthetic construct
  source 1..18: mol_type = other DNA
gaccaccgagatctacac

SEQ ID NO: 43 - DNA, 20 bp, synthetic construct
  source 1..20: mol_type = other DNA
gaacgacatggctacgatcc

SEQ ID NO: 44 - DNA, 20 bp, synthetic construct
  source 1..20: mol_type = other DNA
ctctcagtacgtcagcagtt

SEQ ID NO: 45 - DNA, 40 bp, synthetic construct
  source 1..40: mol_type = other DNA
aatgcacgtactttcagggtaatgatacggcgaccaccga

SEQ ID NO: 46 - DNA, 39 bp, synthetic construct
  source 1..39: mol_type = other DNA
aatgcacgtactttcagggtatgatacggcgaccaccga

SEQ ID NO: 47 - DNA, 41 bp, synthetic construct
  source 1..41: mol_type = other DNA
aatgcacgtactttcagggtaatgatacggcgaccaccgag

SEQ ID NO: 48 - DNA, 40 bp, synthetic construct
  source 1..40: mol_type = other DNA
aatgcacgtactttcagggtatgatacggcgaccaccgag

SEQ ID NO: 49 - DNA, 38 bp, synthetic construct
  source 1..38: mol_type = other DNA
aatgcacgtactttcagggtgaccaccgagatctacac

SEQ ID NO: 50 - DNA, 45 bp, synthetic construct
  source 1..45: mol_type = other DNA
catgtaatgcacgtactttcagggtaatgatacggcgaccaccga

SEQ ID NO: 51 - DNA, 44 bp, synthetic construct
  source 1..44: mol_type = other DNA
catgtaatgcacgtactttcagggtatgatacggcgaccaccga

SEQ ID NO: 52 - DNA, 46 bp, synthetic construct
  source 1..46: mol_type = other DNA
catgtaatgcacgtactttcagggtaatgatacggcgaccaccgag

SEQ ID NO: 53 - DNA, 45 bp, synthetic construct
  source 1..45: mol_type = other DNA
catgtaatgcacgtactttcagggtatgatacggcgaccaccgag

SEQ ID NO: 54 - DNA, 43 bp, synthetic construct
  source 1..43: mol_type = other DNA
catgtaatgcacgtactttcagggtgaccaccgagatctacac

SEQ ID NO: 55 - DNA, 46 bp, synthetic construct
  source 1..46: mol_type = other DNA
ccatgtaatgcacgtactttcagggtaatgatacggcgaccaccga

SEQ ID NO: 56 - DNA, 45 bp, synthetic construct
  source 1..45: mol_type = other DNA
ccatgtaatgcacgtactttcagggtatgatacggcgaccaccga

SEQ ID NO: 57 - DNA, 47 bp, synthetic construct
  source 1..47: mol_type = other DNA
ccatgtaatgcacgtactttcagggtaatgatacggcgaccaccgag

SEQ ID NO: 58 - DNA, 46 bp, synthetic construct
  source 1..46: mol_type = other DNA
ccatgtaatgcacgtactttcagggtatgatacggcgaccaccgag

SEQ ID NO: 59 - DNA, 44 bp, synthetic construct
  source 1..44: mol_type = other DNA
ccatgtaatgcacgtactttcagggtgaccaccgagatctacac

SEQ ID NO: 60 - DNA, 45 bp, synthetic construct
  source 1..45: mol_type = other DNA
catgtaatgcacgtactttcagggtgaacgacatggctacgatcc

SEQ ID NO: 61 - DNA, 45 bp, synthetic construct
  source 1..45: mol_type = other DNA
catgtaatgcacgtactttcagggtctctcagtacgtcagcagtt

SEQ ID NO: 62 - DNA, 25 bp, synthetic construct
  source 1..25: mol_type = other DNA
catgtaatgcacgtactttcagggt

SEQ ID NO: 63 - DNA, 26 bp, synthetic construct
  source 1..26: mol_type = other DNA
ccatgtaatgcacgtactttcagggt

SEQ ID NO: 64 - DNA, 20 bp, synthetic construct
  source 1..20: mol_type = other DNA
aatgatacggcgaccaccga

SEQ ID NO: 65 - DNA, 20 bp, synthetic construct
  source 1..20: mol_type = other DNA
ctctcagtacgtcagcagtt

SEQ ID NO: 66 - DNA, 35 bp, synthetic construct
  source 1..35: mol_type = other DNA
cgtgctggattggctcaccagacaccttccgacat

SEQ ID NO: 67 - DNA, 33 bp, synthetic construct
  source 1..33: mol_type = other DNA
acactctttccctacacgacgctcttccgatct

SEQ ID NO: 68 - DNA, 33 bp, synthetic construct
  source 1..33: mol_type = other DNA
tcgtcggcagcgtcagatgtgtataagagacag

SEQ ID NO: 69 - DNA, 32 bp, synthetic construct
  source 1..32: mol_type = other DNA
gctcacagaacgacatggctacgatccgactt

SEQ ID NO: 70 - DNA, 35 bp, synthetic construct
  source 1..35: mol_type = other DNA
atgtcggaaggtgtgcaggctaccgcttgtcaact

SEQ ID NO: 71 - DNA, 34 bp, synthetic construct
  source 1..34: mol_type = other DNA
agatcggaagagcacacgtctgaactccagtcac

SEQ ID NO: 72 - DNA, 34 bp, synthetic construct
  source 1..34: mol_type = other DNA
ctgtctcttatacacatctccgagcccacgagac

SEQ ID NO: 73 - DNA, 32 bp, synthetic construct
  source 1..32: mol_type = other DNA
ttgtcttcctaagaccgcttggcctccgactt

SEQ ID NO: 74 - DNA, 21 bp, synthetic construct
  source 1..21: mol_type = other DNA
tcgtatgccgtcttctgcttg

SEQ ID NO: 75 - DNA, 18 bp, synthetic construct
  source 1..18: mol_type = other DNA
ctgataaggtcgccatgc

SEQ ID NO: 76 - DNA, 23 bp, synthetic construct
  source 1..23: mol_type = other DNA
agtcgtcgcagcctcacctgatc

SEQ ID NO: 77 - DNA, 22 bp, synthetic construct
  source 1..22: mol_type = other DNA
agtcgtcgcagcctcacctgat

SEQ ID NO: 78 - DNA, 56 bp, synthetic construct
  source 1..56: mol_type = other DNA
  modified_base 54..56: mod_base = OTHER; note = uracil
catgtaatgcacgtactttcagggtaaacatgtaatgcacgtactttcagggtttt

SEQ ID NO: 79 - DNA, 52 bp, synthetic construct
  modified_base 50..52: mod_base = OTHER; note = uracil
  source 1..52: mol_type = other DNA
gatcaggtgaggctgcgacgactaaagatcaggtgaggctgcgacgactttt

SEQ ID NO: 80 - DNA, 22 bp, synthetic construct
  source 1..22: mol_type = other DNA
attacatggatcaggtgaggct

SEQ ID NO: 81 - DNA, 15 bp, synthetic construct
  source 1..15: mol_type = other DNA
gaggctgcgacgact

SEQ ID NO: 82 - DNA, 23 bp, synthetic construct
  source 1..23: mol_type = other DNA
gatcaggtgaggctgcgacgact

SEQ ID NO: 83 - DNA, 25 bp, synthetic construct
  source 1..25: mol_type = other DNA
attacatggatcaggtgaggctgcg

SEQ ID NO: 84 - DNA, 26 bp, synthetic construct
  source 1..26: mol_type = other DNA
cattacatggatcaggtgaggctgcg

SEQ ID NO: 85 - DNA, 27 bp, synthetic construct
  source 1..27: mol_type = other DNA
gcattacatggatcaggtgaggctgcg

SEQ ID NO: 86 - DNA, 32 bp, synthetic construct
  source 1..32: mol_type = other DNA
gcattacatggatcaggtgaggctgcgacgac

SEQ ID NO: 87 - DNA, 22 bp, synthetic construct
  source 1..22: mol_type = other DNA
gtatcattcaagcagaagacgg

SEQ ID NO: 88 - DNA, 27 bp, synthetic construct
  source 1..27: mol_type = other DNA
ccgtatcattcaagcagaagacggcat

SEQ ID NO: 89 - DNA, 28 bp, synthetic construct
  source 1..28: mol_type = other DNA
gtatcattcaagcagaagacggcatacg

SEQ ID NO: 90 - DNA, 30 bp, synthetic construct
  source 1..30: mol_type = other DNA
ccgtatcattcaagcagaagacggcatacg

SEQ ID NO: 91 - DNA, 30 bp, synthetic construct
  source 1..30: mol_type = other DNA
gtatcattcaagcagaagacggcatacgag

SEQ ID NO: 92 - DNA, 31 bp, synthetic construct
  source 1..31: mol_type = other DNA
cgtatcattcaagcagaagacggcatacgag

SEQ ID NO: 93 - DNA, 32 bp, synthetic construct
  source 1..32: mol_type = other DNA
ccgtatcattcaagcagaagacggcatacgag

SEQ ID NO: 94 - DNA, 30 bp, synthetic construct
  source 1..30: mol_type = other DNA
tcggtggtcgccgtatcattcaagcagaag

SEQ ID NO: 95 - DNA, 29 bp, synthetic construct
  source 1..29: mol_type = other DNA
tcggtggtcgccgtatcattcaagcagaa

SEQ ID NO: 96 - DNA, 29 bp, synthetic construct
  source 1..29: mol_type = other DNA
tcggtggtcgccgtatcatttcaagcaga

SEQ ID NO: 97 - DNA, 30 bp, synthetic construct
  source 1..30: mol_type = other DNA
tcggtggtcgccgtatcatttcaagcagaa

SEQ ID NO: 98 - DNA, 29 bp, synthetic construct
  source 1..29: mol_type = other DNA
gtatcatttcaagcagaagacggcatacg

SEQ ID NO: 99 - DNA, 31 bp, synthetic construct
  source 1..31: mol_type = other DNA
gtatcatttcaagcagaagacggcatacgag

SEQ ID NO: 100 - DNA, 32 bp, synthetic construct
  source 1..32: mol_type = other DNA
cgtatcatttcaagcagaagacggcatacgag

SEQ ID NO: 101 - DNA, 32 bp, synthetic construct
  source 1..32: mol_type = other DNA
cgtatcattccaagcagaagacggcatacgag

SEQ ID NO: 102 - DNA, 32 bp, synthetic construct
  source 1..32: mol_type = other DNA
gtcgccgtatcaagcagaagacggcatacgag

SEQ ID NO: 103 - DNA, 30 bp, synthetic construct
  source 1..30: mol_type = other DNA
gtcgccgtatcaagcagaagacggcatacg

SEQ ID NO: 104 - DNA, 32 bp, synthetic construct
  source 1..32: mol_type = other DNA
  modified_base 23: mod_base = i
gtcgccgtatcaagcagaagacgncatacgag

SEQ ID NO: 105 - DNA, 32 bp, synthetic construct; identical to SEQ ID NO: 102
  modified_base 27: mod_base = OTHER; note = uracil
  source 1..32: mol_type = other DNA

SEQ ID NO: 106 - DNA, 30 bp, synthetic construct
  source 1..30: mol_type = other DNA
cgccgtatcaagcagaagacggcatacgag

SEQ ID NO: 107 - DNA, 31 bp, synthetic construct
  source 1..31: mol_type = other DNA
cgtatcattaagcagaagacggcatacgaga

SEQ ID NO: 108 - DNA, 30 bp, synthetic construct
  source 1..30: mol_type = other DNA
cgtatcattgcagaagacggcatacgagat

SEQ ID NO: 109 - DNA, 30 bp, synthetic construct
  source 1..30: mol_type = other DNA
gtcgccgtcaagcagaagacggcatacgag

SEQ ID NO: 110 - DNA, 30 bp, synthetic construct; identical to SEQ ID NO: 106
  source 1..30: mol_type = other DNA

SEQ ID NO: 111 - DNA, 31 bp, synthetic construct
  source 1..31: mol_type = other DNA
tcgccgtatcaagcagaagacggcatacgag

SEQ ID NO: 112 - DNA, 32 bp, synthetic construct
  source 1..32: mol_type = other DNA
ttcgccgtatcaagcagaagacggcatacgag

SEQ ID NO: 113 - DNA, 33 bp, synthetic construct
  source 1..33: mol_type = other DNA
gttcgccgtatcaagcagaagacggcatacgag

SEQ ID NO: 114 - DNA, 34 bp, synthetic construct
  source 1..34: mol_type = other DNA
cgttcgccgtatcaagcagaagacggcatacgag

SEQ ID NO: 115 - DNA, 30 bp, synthetic construct
  source 1..30: mol_type = other DNA
agatctcggtggtcgccgtatcaagcagaa

SEQ ID NO: 116 - DNA, 31 bp, synthetic construct
  source 1..31: mol_type = other DNA
agatctcggtggtcgccgtatcaagcagaag

SEQ ID NO: 117 - DNA, 32 bp, synthetic construct
  source 1..32: mol_type = other DNA
agatctcggtggtcgccgtatcaagcagaaga

SEQ ID NO: 118 - DNA, 33 bp, synthetic construct
  source 1..33: mol_type = other DNA
agatctcggtggtcgccgtatcaagcagaagac

SEQ ID NO: 119 - DNA, 20 bp, synthetic construct
  source 1..20: mol_type = other DNA
aatgcacgtactttcagggt

SEQ ID NO: 120 - DNA, 25 bp, synthetic construct
  source 1..25: mol_type = other DNA
catgtaatgcacgtactttcagggt

SEQ ID NO: 121 - DNA, 20 bp, synthetic construct
  source 1..20: mol_type = other DNA
gcgaccaccgagatctacac

SEQ ID NO: 122 - DNA, 40 bp, synthetic construct
  source 1..40: mol_type = other DNA
aatgcacgtactttcagggtgcgaccaccgagatctacac

SEQ ID NO: 123 - DNA, 40 bp, synthetic construct
  source 1..40: mol_type = other DNA
gcgaccaccgagatctacacaatgcacgtactttcagggt

SEQ ID NO: 124 - DNA, 18 bp, synthetic construct
  source 1..18: mol_type = other DNA
agtcgtcgcagcctcacc

SEQ ID NO: 125 - DNA, 20 bp, synthetic construct
  source 1..20: mol_type = other DNA
atctcgtatgccgtcttctg

SEQ ID NO: 126 - DNA, 21 bp, synthetic construct
  source 1..21: mol_type = other DNA
caagcagaagacggcatacga

SEQ ID NO: 127 - DNA, 20 bp, synthetic construct
  source 1..20: mol_type = other DNA
aatgatacggcgaccaccga

SEQ ID NO: 128 - protein, 773 aa, unidentified
MKKTLLDADYITREEKAVVRLFYKTEEGREIQEVADFRPYMYVLPEEHDLKKLQREIKELKNITNVEIKRMIEGDREVEVLKVMVNQPRDVPNLRGLIKELEGCKEVREAHIPFAERYLIDSGLIPMQDCENFDLRIAAFDMEVYNPRGEPKAERDPIIIISYADNRGLRRVWTYKTVENLNLDYMEVLKDEREIIRRFIDTIREREIDIIVTYNGDNFDFPYLKERAEKHRIPVSLGVDGSPLRLERRGMNLGARVTGRPHIDMYPVCRQIFNLSRYTLEDVYLEITGREKKDIRVGEMAGIWDNPEKEKFKELIEYAMSDAESTLEIAITLLPLHYEISRITRELIYQSSRAGSGQRVESLLIKKAFEKSILVPNRPSDRVVNERQRKTYIGAYVVEPKRGIHDNILLFDFRSLYPSIIISHNIDPSTIDCECCPEDSYRSPTGHYFCKKKRGLIPETLNELVQRRIEVKKGLKNEKNPERRRFLDVKQQALKLLANSMYGYFGFPRARWYCRECAESITALGRKYILHTIDIVPKFGFDVIYGDTDSVYLIKPNITDRERVMKNAEHFLDKINSELPEAMELEFEGFYPRGIFITKKRYALIDERGKLIVKGLETKRRDWANIAKDTQEKVLDALLKDKNPEKAASIVKDVIRNIKTGKIPLKDLAINTQITRGMAEYKTEGPHIVAAKKAMKRGLEFKQGNIVTYVVTKKGKSISDKARVIDFVEEGDYDPDYYINNQVLPSVLRILEALGYSEDELKGLGKQMKLGGF

SEQ ID NO: 129 - protein, 806 aa, unidentified
MLLDVDYAEEEEKPSIRLFVKTGSEVLVAIDPDFEEYFYVVSDHPAKASKLIEKVEVEEDGVSIRPKGVEIVKRTFLGNEVEAIKVSFYQAKDSSKLRHKIRELPGVREIYEFDIPPARRYLIDRGLTPMAGVEFDGRIEERQGIKTVILDSPPRPAQVEEPKLNIMSFDIEVYNPTGSVRPDKDPIIMISLADNNGLRKVITWKNFERSQEYVEVVGSEREMIKRFVDLVKERDVDILLGYNTDLFDLPYIRSRAKQLKVKLDLGRDGSELVVRKRRFATASKIRGRIHVDVFAMVDFLATIGSIKLIHYSLADVYRHLLGKEKPDFEFTEMVDAWEKGGDAGRKFLEYSMSDADATLEVGLELLPLFLGLTRVVGQTLFDVQRMTPGQLVEWLLVAEAHRIGELVPGRPVGEEYEERMEETFVGAYVMEPVKGLHENLVVFDFRSLYPSIIVTHNIDPSTLNCKDCKPGEREQVPGLEYYFCRRRKGFIPATLQRIIEERMKLKAELKKLVRGTKEYRALDARQWAMKIVANSFYGMLGYPRARWYSKECAESVTSFGRHYIHKTIDMAREFGLEVVYGDTDSLHCKLNGKTREEALAFLKKVNDSLPGIMELELEGFYPRGIFITKKRYAMIDDEGRMVVKGLEFVRRDWAAIAKKTQEEVLKAILRDGSPEKAAEIIRKTTRDVYEGRVNLEDLIIYTQLKMPIESYKAIGPHVVAAKRLRELGHEIEPGMMIAYVEVKGPGSISERAVPVEDFKGREYDPDYYVGHQVLPAVMRIMEVLGYREIDLKFERQRQVGLDRFMK

SEQ ID NO: 130 - protein, 809 aa, unidentified
MRGLLFDVDIAEEEERPNVRLFVKVASETVVAIDPQFEEYFYVVADHPAKTSKLIEKIELDEGGRPIRPKSVEMVRRTLLGNEVEAIRVSFHQPRDAAKLRHKIRELPGVKEIYEFDIPPARRYLIDRGLTPMAGIEFSGSIEVRDGVKTVVMDGPPKPAPVEETRLNIMSFDIEVYNPTGSVRPDKDPIIMISLADNRGLRKVITWKNFDKKPEYVEVVGSEREMIKKFVELVKERDVDILLGYNTDLFDLPYIRSRAKQLRVKLDLGRDGSELVVRKRRFATASKIRGRVHVDVFAMVDFLATIGSIRLIHYSLADVYRHYAGREKPDFEFSEMINAWERGGDAGRRFLEYSMSDADATLEVGSELLPLFLGLTRVVGQTLFDVQRMTPGQLVEWLLVAEAHRIGELVPPRPVGEEFEERAEGTFTGAYVMEPVKGLHEDLVVFDFRSLYPSIIVTHNIDPSTLNCRDCKPGEREQVPGLSYYFCKRRKGFIPAVLERVIEERTKLKAELKKIGRETREYRALDARQWAMKIVANSFYGMLGYPRARWYSKQCAESVTSFGRHYIHRTIEMAREFGLEVVYGDTDSLHCKLNGKTREEAMVFLRKVNESLPGIMELELEGFYPRGIFITKKRYAMVDDEGRMVVKGLEFVRRDWAAIAKKTQEEVLRAILRDGSPKKAAEIIRKTTRDVYEGRVNLEDLIIYTQLKMPIESYKAIGPHVVAAKRLRELGHEIEPGMMIAYIEVKGPGSISDRAVPVEDFEGKEYDPDYYVGHQILPAVMRIMEVLGYSEEDLKFEREKQIGLDRFMK

SEQ ID NO: 131 - protein, 792 aa, unidentified
MPRILKGFLIDADYETVEGRALIRLFLRGEEGSFVVYDDSFSPYFYALPGDEPERVKERILASGAAEAIQKVEIEEKRLFGTPRVALRITVSHPQDVPRIRERIRRVEGVDLILEHDILFVRRYLIDRGIKPLTWLRLEVEERDGRALLRGVEQLEEEPPELRVAAVDIEVYNPKGAPRSSKDEIIMISVATSDGVEKVLTWREVQGLEQVEVLQDEKEMLLRFAELIKEGDYDVIVGYNTDSFDFPYIRDRLKKLGISLPLGRLDAELEVSRRGALPEARIRGRAHVDLYPIVRRHVKLNSYVLESVVEELLGIKKEKLDGERLFTYWDEGGEKRALLARYALEDARVTLALAEKFLPLYCELSTIVGQSLNDVARMTSGQLVEWLLMRYATPRGELIPNHPAGEEYAARARATYAGGYVREPKRGLVEHIAVFDFRSLYPSIIVSHNIDPSTLIVGNCEENRAPELEYCFSLEREGFIPAILKELIRRRAEIKRELKRSEGDRRRTLSFAEKALKILANSFYGYMGYPRARWYRRECAESVAAFARMYIKQVMRIAEEEFKLEVVYGDTDSLFVVIPPEKRELAQKFLQKVNESMPGIIELEFEGFYRRGLFVTKKRYALLSEDGKMVVKGLEFVRRDWAPIARETQKEVLRILLEEADPEKAARLVRDVIERIRQRRVSLEDITIYTQLTKRIKSYKSLEPHVVAAQKLKERGREVAPGMIIGYIITKGTKGISQRATPVEFARLEDYDPEYYIDNQILPAIQRIFEAIGYTRDYLKEGITQTSLSRWF

SEQ ID NO: 132 - protein, 773 aa, unidentified
MARDLLLDIDYVTVDEKAQVRLFLKDKILFDPGFQPYFYVLAHDGAVEERLRDFGAVEAVQRRMLGREMRFFKLILSHPSEVPKIREEVRSIEGVEGIFEHDILFARRYLIDKGLTPLNYAEYRAEQGFLKGISSAGEGPESLRVMAFDIETYNPKGAPRAEKDPVIMLSLSTNTGLRRLLTYKSGEGLDFVELVEDEKALLHRFKELVNEEGVEVLVGYNSDQFDLPYLVARAKALGVELPLGQDGSQPQIRKGRGLVESVVKGRPHVDLYPIVRRNVRLSSYVLENVVKEVLGREKEKIPHDAMCGYWDRGGRELQRFMAYSMEDADVTLELAERFLPLYIELSRVVGLPLHDVARMTAGQLVEWLLIREAFARGEVVPNKGSGREYLARSEDTYAGGYVMEPVKGIVENIVVFDFRSLYPSIIVTHNIDPATLRPGRGENSPPELDYHFTTEEEGFIPSVLKRVLERRLSAKRRMKEARDPGEKRMLDISQRALKIIANSFYGYMGYPRARWYKKECAESVTSFARMYTKKVMAIAEEEYGFKVVYGDTDSLFIVVQPEEKERAMSFMEDVNRRLPGTVELEYDGFYPRGIFITKKRYALIDEKGNIVVKGLETVRRDWTRLSRDTQQKVLSVILREGDPKKAADIVKDTINRLKERRVDLEDITIYTQLTKGIGRYKNVGPHVKAAQKAIDRGREVNPGMAIGYIIKKGRGLISDRAEPVEDATIEDYDVDYYIENQVLPPVARIMEVLGYSKEHLKEEMVQGSLQRWF

SEQ ID NO: 133 - protein, 687 aa, unidentified
MDGFLLDVDYKTVDEKPVVRLFLRDVIALDPSFRPYVYVACDDPRAVAGEIKDLELDGRRPVTGVEEMERGLLGRPRRFLKVYLGHPQQVPRVRDLLRRLPGVSAVLEDDILFSRRYLIDKGLVPTAWVELQGRVEGSEFWVEEVRRAEGPLPRLKVMSFDIETYNPKGAPRGDQDPIIMVSMATSGGLRKVLSWKAPTAGLEFVETLEDEAAVLRRFEELVRQEDPDILVGYNTDNFDFPYLNQRLKALGIELALGRDGSPHKTSTRMGMSETRMAGRPHMDLYPIVRRSLRLPSYVLEDVVAEVLGEEKEKVPGERMGEIWDKGGEELDRFFRYSLEDAEVTLRIGEKYLPLYIELSRLVGQSIHDVARMTAGQLVEWYLMREAFARGEVIPERPGGREFARRAGDTYEGGYVREPRKGLLEKVFDFRSLYPSVIVTHNIDPSTIRPGPGENQPPGIDYHFTTEKEGFIPALLKRLVARRAELKEEMKKARDPGERKMLDVQQQALKILANSFYGYMGYPRARWYRKECAESVTAFARDYIKKVMEVAEKEFGLEVVYGDTDSLFILVPGGKKERAFAFLEEVNRRLPGTIELEYEGFYRRGIFVTKKRYALIDEKDRIIVKGLEFVRRDWAPIARDTQEKVLKALLKDASPEEAVRIVRKAMDDIRARRVSLEDLTIYTQLTKK

SEQ ID NO: 134 - protein, 773 aa, unidentified
MILDTDYITENGKPVIRIFKKEKGEFKIEYDRNFEPYIYALLEDDESIEDIKKITGERHGKKVRIIRVEKVKKKFLGEPIEVWKLVFEHPQDVPAIRDAIRSHPAVREIFEYDIPFAKRYLIDKGLVPMEGGEELKLLAFDIETFYHEGDEFAEGEILMISYADESGAKVITWKKIDLPYVEVVSTEREAIKRFLQVLREKDPDVLLTYNGDNFDFAYIKKRCEKLGLKFTIGRDGSEPKIQRMGDRFAVEVKGRIHLDLYPVVRHTIRLPTYTLEAVYEAVFGKKKEKVYAEEIAEAWKSEEGLKRVAQYSMEDARATYELGREFFPMEVELAKLIGQSVWDVSRSSTGNLVEWYLLRVAYERNELAPNKPGGEEYQRRMRSSYIGGYVKEPEKGLWESIAYLDFRSLYPSIIVTHNVSPDTLEKECKNYDVAPIVGYRFCKDFKGFIPSILEDLIETRQKVKRKMKATIDPIEKKMLDYRQRALKILANSYYGYQGYPKARWYSKECAESVTAWGRHYIETTIKEAEKFGFKVLYADTDGFFATIPNEKPETIKSKAKKFLKHINEKLPGMLELEYEGFYLRGFFVTKKKYALIDEDGHITTRGLEVVRRDWSEIAKETQAKVLEVILREGSIEKAAGIVKKVVEDLANYRVPVEKLIIHEQITRELKDYKATGPHVAIAKRLQARGIKVKPGTIISYVVLKGSKKISDRVILFDEYDSSRHKYDPDYYIHNQVLPAVLRILEAFGYKEKDLEYQRMKQTGLGAWLKMGKK

SEQ ID NO: 135 - protein, 773 aa, unidentified
MILDTDYITENGKPVIRIFKKEKGEFKIEYDRNFEPYIYALLEDDESIEDIKKITGERHGKKVRIIRVEKVKKKFLGEPIEVWKLVFEHPQDVPAIRDAIRSHPAVREIFEYDIPFAKRYLIDKGLVPMEGGEELKLLAFDIETFYHEGDEFAEGEILMISYADESGAKVITWKKIDLPYVEVVSTEREAIKRFLQVLREKDPDVLLTYNGDNFDFAYIKKRCEKLGLKFTIGREGSEPKIQRMGDRFAVEVKGRIHLDLYPVVRHTIRLPTYTLEAVYEAVFGKKKEKVYAEEIAEAWKSEEGLKRVAQYSMEDARATYELGREFFPMEVELAKLIGQSVWDVSRSSTGNLVEWYLLRVAYERNELAPNKPGGEEYQRRMRSSYIGGYVKEPEKGLWESIAYLDFRSLYPSIIVTHNVSPDTLEKECKNYDVAPIVGYRFCKDFKGFIPSILEDLIETRQKVKRKMKATIDPIEKKMLDYRQRALKILANSYYGYQGYPKARWYSKECAESVTAWGRHYIETTIKEAEKFGFKVLYADTDGFFATIPNEKPETIKSKAKKFLKHINEKLPGMLELEYEGFYLRGFFVTKKKYALIDEDGHITTRGLEVVRRDWSEIAKETQAKVLEVILREGSIEKAAGIVKKVVEDLANYRVPVEKLIIHEQITRELKDYKATGPHVAIAKRLQARGIKVKPGTIISYVVLKGSKKISDRVILFDEYDSSRHKYDPDYYIHNQVLPAVLRILEAFGYKEKDLEYQRMKQTGLGAWLKMGKK

SEQ ID NO: 136 - protein, 774 aa, unidentified
MILDTDYITKDGKPIIRIFKKENGEFKIELDPHFQPYIYALLKDDSAIEEIKAIKGERHGKTVRVLDAVKVRKKFLGREVEVWKLIFEHPQDVPAMRDKIKEHPAVIDIYEYDIPFAKRYLIDKGLIPMEGDEELKLLAFDIETFYHEGDEFGKGEIIMISYADEEEARVITWKNIDLPYVDVVSNEREMIKRFVQVVKEKDPDVIITYNGDNFDLPYLIKRAEKLGIRLVLGRDKENPEPKIQRMGDSFAVEIKGRIHFDLFPVVRRTINLPTYTLEAVYEAVLGKTKSKLGAEEIAAIWETEESMKKLAQYSMEDARATYELGKEFFPMEAELAKLIGQSVWDVSRSSTGNLVEWYLLRVAYARNELAPNKPDEEEYKRRLRTTYLGGYVKEPEKGLWENIIYLDFRSLYPSIIVTHNVSPDTLEKEGCENYDIAPIVGYKFCKDFPGFIPSILGDLIAMRQEIKKKMKATIDPIEKKMLDYRQRAVKLLANSYYGYMGYPKARWYSKECAESVTAWGRHYIEMTIKEIEEKFGFKVLYADTDGFYATIPGEKPEIIKKKAREFLNYINSKLPGLLELEYEGFYLRGFFVTKKRYAVIDEEGRITTRGLEVVRRDWSEIAKETQAKVLEAILKDGSVEKAVEIVRDVVEKIAKYRVPLEKLVIHEQITRDLKDYKAIGPHVSIAKRLATRGIKVKPGTIISYLVLKGGGRISDRVILLTEYDPEKHKYDPDYYIENQVLPAVLRILEAFGYRKEDLRYQSSKQTGLDAWLKR

SEQ ID NO: 137 - protein, 575 aa, unidentified [sequence omitted]

SEQ ID NO: 138 - protein, 876 aa, unidentified
MKKKLVLIDGNSVAYRAFFALPLLHNDKGIHTNAVYGFTMMLNKILAEEQPTHLLVAFDAGKTTFRHETFQEYKGGRQQTPPELSEQFPLLRELLKAYRIPAYELDHYEADDIIGTLAARAEQEGFEVKIISGDRDLTQLASRHVTVDITKKGITDIEPYTPETVREKYGLTPEQIVDLKGLMGDKSDNIPGVPGIGEKTAVKLLKQFGTVENVLASIDEVKGEKLKENLRQHRDLALLSKQLASICRDAPVELSLDDIVYEGQDREKVIALFKELGFQSFLEKMAAPAAEGEKPLEEMEFAIVDVITEEMLADKAALVVEVMEENYHDAPIVGIALVNEHGRFFMRPETALADSQFLAWLADETKKKSMFDAKRAVVALKWKGIELRGVAFDLLLAAYLLNPAQDAGDIAAVAKMKQYEAVRSDEAVYGKGVKRSLPDEQTLAEHLVRKAAAIWALEQPFMDDLRNNEQDQLLTKLEQPLAAILAEMEFTGVNVDTKRLEQMGSELAEQLRAIEQRIYELAGQEFNINSPKQLGVILFEKLQLPVLKKTKTGYSTSADVLEKLAPHHEIVENILHYRQLGKLQSTYIEGLLKVVRPDTGKVHTMFNQALTQTGRLSSAEPNLQNIPIRLEEGRKIRQAFVPSEPDWLIFAADYSQIELRVLAHIADDDNLIEAFQRDLDIHTKTAMDIFHVSEEEVTANMRRQAKAVNFGIVYGISDYGLAQNLNITRKEAAEFIERYFASFPGVKQYMENIVQEAKQKGYVTTLLHRRRYLPDITSRNFNVRSFAERTAMNTPIQGSAADIIKKAMIDLAARLKEEQLQARLLLQVHDELILEAPKEEIERLCELVPEVMEQAVTLRVPLKVDYHYGPTWYDAK

SEQ ID NO: 139 - protein, 775 aa, unidentified
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPAEGDEELTMLAFDIETLYHEGEEFGTGPILMISYADGSEARVITWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSLYPSIIITHNVSPDTLNREGCKEYDVAPEVGHKFCKDFPGFIPSLLGDLLEERQKIKRKMKATVDPLEKKLLDYRQRAIKILANSFYGYYGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDEEGKITTRGLEIVRRDWSEIAKETQARVLEAILKHGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 140 - protein, 775 aa, unidentified
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPMEGDEELTMLAFDIETLYHEGEEFGTGPILMISYADGSEARVITWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSLYPSIIITHNVSPDTLNREGCKEYDVAPEVGHKFCKDFPGFIPSLLGDLLEERQKIKRKMKATVDPLEKKLLDYRQRAIKILANSFYGYYGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDEEGKITTRGLEIVRRDWSEIAKETQARVLEAILKHGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 141 - protein, 1702 aa, unidentified
MILDTDYITKDGKPIIRIFKKENGEFKIELDPHFQPYIYALLKDDSAIEEIKAIKGERHGKTVRVLDAVKVRKKFLGREVEVWKLIFEHPQDVPAMRGKIREHPAVVDIYEYDIPFAKRYLIDKGLIPMEGDEELKLLAFDIETFYHEGDEFGKGEIIMISYADEEEARVITWKNIDLPYVDVVSNEREMIKRFVQVVKEKDPDVIITYNGDNFDLPYLIKRAEKLGVRLVLGRDKEHPEPKIQRMGDSFAVEIKGRIHFDLFPVVRRTINLPTYTLEAVYEAVLGKTKSKLGAEEIAAIWETEESMKKLAQYSMEDARATYELGKEFFPMEAELAKLIGQSVWDVSRSSTGNLVEWYLLRVAYARNELAPNKPDEEEYKRRLRTTYLGGYVKEPEKGLWENIIYLDFRSLYPSIIVTHNVSPDTLEKEGCKNYDVAPIVGYRFCKDFPGFIPSILGDLIAMRQDIKKKMKSTIDPIEKKMLDYRQRAIKLLANSILPNEWLPIIENGEIKFVKIGEFINSYMEKQKENVKTVENTEVLEVNNLFAFSFNKKIKESEVKKVKALIRHKYKGKAYEIQLSSGRKINITAGHSLFTVRNGEIKEVSGDGIKEGDLIVAPKKIKLNEKGVSINIPELISDLSEEETADIVMTISAKGRKNFFKGMLRTLRWMFGEENRRIRTFNRYLFHLEKLGLIKLLPRGYEVTDWERLKKYKQLYEKLAGSVKYNGNKREYLVMFNEIKDFISYFPQKELEEWKIGTLNGFRTNCILKVDEDFGKLLGYYVSEGYAGAQKNKTGGISYSVKLYNEDPNVLESMKNVAEKFFGKVRVDRNCVSISKKMAYLVMKCLCGALAENKRIPSVILTSPEPVRWSFLEAYFTGDGDIHPSKRFRLSTKSELLANQLVFLLNSLGISSVKIGFDSGVYRVYINEDLQFPQTSREKNTYYSNLIPKEILRDVFGKEFQKNMTFKKFKELVDSGKLNREKAKLLEFFINGDIVLDRVKSVKEKDYEGYVYDLSVEDNENFLVGFGLLYAHNSYYGYMGYPKARWYSKECAESVTAWGRHYIEMTIREIEEKFGFKVLYADSVSGESEIIIRQNGKIRFVKIKDLFSKVDYSIGEKEYCILEGVEALTLDDDGKLVWKPVPYVMRHRANKRMFRIWLTNSWYIDVTEDHSLIGYLNTSKTKTAKKIGERLKEVKPFELGKAVKSLICPNAPLKDENTKTSEIAVKFWELVGLIVGDGNWGGDSRWAEYYLGLSTGKDAEEIKQKLLEPLKTYGVISNYYPKNEKGDFNILAKSLVKFMKRHFKDEKGRRKIPEFMYELPVTYIEAFLRGLFSADGTVTIRKGVPEIRLTNIDADFLREVRKLLWIVGISNSIFAETTPNRYNGVSTGTYSKHLRIKNKWRFAERIGFLIERKQKRLLEHLKSARVKRNTIDFGFDLVHVKKVEEIPYEGYVYDIEVEETHRFFANNILVHNTDGFYATIPGEKPELIKKKAKEFLNYINSKLPGLLELEYEGFYLRGFFVTKKRYAVIDEEGRITTRGLEVVRRDWSEIAKETQAKVLEAILKEGSVEKAVEVVRDVVEKIAKYRVPLEKLVIHEQITRDLKDYKAIGPHVAIAKRLAARGIKVKPGTIISYIVLKGSGKISDRVILLTEYDPRKHKYDPDYYIENQVLPAVLRILEAFGYRKEDLRYQSSKQTGLDAWLKR

SEQ ID NO: 142 - protein, 1312 aa, unidentified
MILDADYITEDGKPIIRIFKKENGEFKVEYDRNFRPYIYALLKDDSQIDEVRKITAERHGKIVRIIDAEKVRKKFLGRPIEVWRLYFEHPQDVPAIRDKIREHSAVIDIFEYDIPFAKRYLIDKGLIPMEGDEELKLLAFDIETLYHEGEEFAKGPIIMISYADEEEAKVITWKKIDLPYVEVVSSEREMIKRFLKVIREKDPDVIITYNGDSFDLPYLVKRAEKLGIKLPLGRDGSEPKMQRLGDMTAVEIKGRIHFDLYHVIRRTINLPTYTLEAVYEAIFGKPKEKVYAHEIAEAWETGKGLERVAKYSMEDAKVTYELGREFFPMEAQLSRLVGQPLWDVSRSSTGNLVEWYLLRKAYERNELAPNKPDEREYERRLRESYAGGYVKEPEKGLWEGLVSLDFRSLYPSIIITHNVSPDTLNREGCREYDVAPEVGHKFCKDFPGFIPSLLKRLLDERQEIKRKMKASKDPIEKKMLDYRQRAIKILANSILPEEWVPLIKNGKVKIFRIGDFVDGLMKANQGKVKKTGDTEVLEVAGIHAFSFDRKSKKARVMAVKAVIRHRYSGNVYRIVLNSGRKITITEGHSLFVYRNGDLVEATGEDVKIGDLLAVPRSVNLPEKRERLNIVELLLNLSPEETEDIILTIPVKGRKNFFKGMLRTLRWIFGEEKRVRTASRYLRHLENLGYIRLRKIGYDIIDKEGLEKYRTLYEKLVDVVRYNGNKREYLVEFNAVRDVISLMPEEELKEWRIGTRNGFRMGTFVDIDEDFAKLLGYYVSEGSARKWKNQTGGWSYTVRLYNENDEVLDDMEHLAKKFFGKVKRGKNYVEIPKKMAYIIFESLCGTLAENKRVPEVIFTSSKGVRWAFLEGYFIGDGDVHPSKRVRLSTKSELLVNGLVLLLNSLGVSAIKLGYDSGVYRVYVNEELKFTEYRKKKNVYHSHIVPKDILKETFGKVFQKNISYKKFRELVENGKLDREKAKRIEWLLNGDIVLDRVVEIKREYYDGYVYDLSVDEDENFLAGFGFLYAHNSYYGYYGYAKARWYCKECAESVTAWGREYIEFVRKELEEKFGFKVLYIDTDGLYATIPGAKPEEIKKKALEFVDYINAKLPGLLELEYEGFYVRGFFVTKKKYALIDEEGKIITRGLEIVRRDWSEIAKETQAKVLEAILKHGNVEEAVKIVKEVTEKLSKYEIPPEKLVIYEQITRPLHEYKAIGPHVAVAKRLAARGVKVRPGMVIGYIVLRGDGPISKRAILAEEFDLRKHKYDAEYYIENQVLPAVLRILEAFGYRKEDLRWQKTKQTGLTAWLNIKKK

SEQ ID NO: 143 - protein, 775 aa, unidentified
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPMEGDEELTMLAFAIATLYHEGEEFGTGPILMISYADGSEARVITWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSLYPSIIITHNVSPDTLNREGCKEYDVAPEVGHKFCKDFPGFIPSLLGDLLEERQKIKRKMKATVDPLEKKLLDYRQRLIKILANSFYGYYGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDEEGKITTRGLEIVRRDWSEIAKETQARVLEAILKHGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 144 - protein, 775 aa, unidentified
MILDVDYITEEGKPVIRLFKKENGKFKIEHDRTFRPYIYALLRDDSKIEEVKKITGERHGKIVRIVDVEKVEKKFLGKPITVWKLYLEHPQDVPTIREKVREHPAVVDIFEYDIPFAKRYLIDKGLIPMEGEEELKILAFDIETLYHEGEEFGKGPIIMISYADENEAKVITWKNIDLPYVEVVSSEREMIKRFLRIIREKDPDIIVTYNGDSFDFPYLAKRAEKLGIKLTIGRDGSEPKMQRIGDMTAVEVKGRIHFDLYHVITRTINLPTYTLEAVYEAIFGKPKEKVYADEIAKAWESGENLERVAKYSMEDAKATYELGKEFLPMEIQLSRLVGQPLWDVSRSSTGNLVEWFLLRKAYERNEVAPNKPSEEEYQRRLRESYTGGFVKEPEKGLWENIVYLDFRALYPSIIITHNVSPDTLNLEGCKNYDIAPQVGHKFCKDIPGFIPSLLGHLLEERQKIKTKMKETQDPIEKILLDYRQKAIKLLANSFYGYYGYAKARWYCKECAESVTAWGRKYIELVWKELEEKFGFKVLYIDTDGLYATIPGGESEEIKKKALEFVKYINSKLPGLLELEYEGFYKRGFFVTKKRYAVIDEEGKVITRGLEIVRRDWSEIAKETQARVLETILKHGDVEEAVRIVKEVIQKLANYEIPPEKLAIYEQITRPLHEYKAIGPHVAVAKKLAAKGVKIKPGMVIGYIVLRGDGPISNRAILAEEYDPKKHKYDAEYYIENQVLPAVLRILEGFGYRKEDLRYQKTRQVGLTSWLNIKKS

SEQ ID NO: 145 - protein, 771 aa, unidentified
MIIDADYITEDGKPIIRIFKKEKGEFKVEYDRTFRPYIYALLKDDSAIDEVKKITAERHGKIVRITEVEKVQKKFLGRPIEVWKLYLEHPQDVPAIREKIREHPAVVDIFEYDIPFAKRYLIDKGLTPMEGNEELTFLAVDIETLYHEGEEFGKGPIIMISYADEEGAKVITWKSIDLPYVEVVSSEREMIKRLVKVIREKDPDVIITYNGDNFDFPYLLKRAEKLGIKLPLGRDNSEPKMQRMGDSLAVEIKGRIHFDLFPVIRRTINLPTYTLEAVYEAIFGKSKEKVYAHEIAEAWETGKGLERVAKYSMEDAKVTFELGKEFFPMEAQLARLVGQPVWDVSRSSTGNLVEWFLLRKAYERNELAPNKPDEREYERRLRESYEGGYVKEPEKGLWEGIVSLDFRSLYPSIIITHNVSPDTLNRENCKEYDVAPQVGHRFCKDFPGFIPSLLGNLLEERQKIKKRMKESKDPVEKKLLDYRQRAIKILANSYYGYYGYAKARWYCKECAESVTAWGRQYIDLVRRELESRGFKVLYIDTDGLYATIPGAKHEEIKEKALKFVEYINSKLPGLLELEYEGFYARGFFVTKKKYALIDEEGKIVTRGLEIVRRDWSEIAKETQAKVLEAILKHGNVDEAVKIVKEVTEKLSKYEIPPEKLVIYEQITRPLSEYKAIGPHVAVAKRLAAKGVKVKPGMVIGYIVLRGDGPISKRAIAIEEFDPKKHKYDAEYYIENQVLPAVERILRAFGYRKEDLKYQKTKQVGLGAWLKF

SEQ ID NO: 146 - protein, 903 aa, unidentified
MKEFYLTVEQIGDSIFERYIDSNGRERTREVEYKPSLFAHCPESQATKYFDIYGKPCTRKLFANMRDASQWIKRMEDIGLEALGMDDFKLAYLSDTYNYEIKYDHTKIRVANFDIEVTSPDGFPEPSQAKHPIDAITHYDSIDDRFYVFDLLNSPYGNVEEWSIEIAAKLQEQGGDEVPSEIIDKIIYMPFDNEKELLMEYLNFWQQKTPVILTGWNVESFDIPYVYNRIKNIFGESTAKRLSPHRKTRVKVIENMYGSREIITLFGISVLDYIDLYKKFSFTNQPSYSLDYISEFELNVGKLKYDGPISKLRESNHQRYISYNIIDVYRVLQIDAKRQFINLSLDMGYYAKIQIQSVFSPIKTWDAIIFNSLKEQNKVIPQGRSHPVQPYPGAFVKEPIPNRYKYVMSFDLTSLYPSIIRQVNISPETIAGTFKVAPLHDYINAVAERPSDVYSCSPNGMMYYKDRDGVVPTEITKVFNQRKEHKGYMLAAQRNGEIIKEALHNPNLSVDEPLDVDYRFDFSDEIKEKIKKLSAKSLNEMLFRAQRTEVAGMTAQINRKLLINSLYGALGNVWFRYYDLRNATAITTFGQMALQWIERKVNEYLNEVCGTEGEAFVLYGDTDSIYVSADKIIDKVGESKFRDTNHWVDFLDKFARERMEPAIDRGFREMCEYMNNKQHLMFMDREAIAGPPLGSKGIGGFWTGKKRYALNVWDMEGTRYAEPKLKIMGLETQKSSTPKAVQKALKECIRRMLQEGEESLQEYFKEFEKEFRQLNYISIASVSSANNIAKYDVGGFPGPKCPFHIRGILTYNRAIKGNIDAPQVVEGEKVYVLPLREGNPFGDKCIAWPSGTEITDLIKDDVLHWMDYTVLLEKTFIKPLEGFTSAAKLDYEKKASLFDMFDF

SEQ ID NO: 147 - protein, 346 aa, unidentified
MNIFNTNPFKAVSFVESAVKKALETSGYLIADCKYDGVRGNIVVDNVAEAAWLSRVSKFIPALEHLNGFDKRWQQLLNDDRCIFPDGFMLDGELMVKGVDFNTGSGLLRTKWVKRDNMGFHLTNVPTKLTPKGREVIDGKFEFHLDPKRLSVRLYAVMPIHIAESGEDYDVQNLLMPYHVEAMRSLLVEYFPEIEWLIAETYEVYDMDSLTELYEEKRAEGHEGLIVKDPQGIYKRGKKSGWWKLKPECEADGIIQGVNWGTEGLANEGKVIGFSVLLETGRLVDANNISRALMDEFTSNVKAHGEDFYNGWACQVNYMEATPDGSLRHPSFEKFRGTEDNPQEKM

SEQ ID NO: 148 - protein, 487 aa, unidentified
MILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGL

SEQ ID NO: 149 - protein, 358 aa, unidentified
MNIKTNPFKAVSFVESAIKKALDNAGYLIAEIKYDGVRGNICVDNTANSYWLSRVSKTIPALEHLNGFDVRWKRLLNDDRCFYKDGFMLDGELMVKGVDFNTGSGLLRTKWTDTKNQEFHEELFVEPIRKKDKVPFKLHTGHLHIKLYAILPLHIVESGEDCDVMTLLMQEHVKNMLPLLQEYFPEIEWQAAESYEVYDMVELQQLYEQKRAEGHEGLIVKDPMCIYKRGKKSGWWKMKPENEADGIIQGLVWGTKGLANEGKVIGFEVLLESGRLVNATNISRALMDEFTETVKEATLSQWGFFSPYGIGDNDACTINPYDGWACQISYMEETPDGSLRHPSFVMFRGTEDNPQEKM

SEQ ID NO: 150 - protein, 559 aa, unidentified
MKYSELAGLYRRLEKTTLKTLKTRFVADFLKNVPDELLEIVPYLILGKVFPDWDERELGVGEKLLIKAVSIATGVPEGEIENSIKDTGDLGESIALAVKKKKQKSFFSQPLTIKRVYDTFVKVAESQGEGSQDRKMKYLANLFMDAQPEEAKYIARTVLGTMRTGVAEGILRDAIAEAFKVKAELVERAYMLTSDFGYVTKVAKLEGNEGLSKVRIQVGKPVRPMLAQNAASVKDALLEMGGEAAFEIKYDGARVQVHKDGDRVVIYSRRLENVTRSIPEIVEAVRSQLRPEKAIVEGELVAVGDGGKPRPFQYVLRRFRRKYNIEEMIERIPLELNLFDVLYVDGESLVDTPFMERRKRLEEAVEESERIKLAQQLVTKKAEEAEEFYRRALELGHEGLMAKRLDSVYEPGNRGKKWLKIKPTMEDLDLVIIGAEWGEGRRAHLLGSFLVAAYDQHRGEFVPVGKVGSGFTDEDLAEFTKMLKPLIVREEGKYVEIEPRVVIQVTYQEIQKSPKYESGFALRFPRYVALREDKSPEEADTIERISELYGLQERFKAKR

SEQ ID NO: 151 - protein, 559 aa, Uahuka affinis
MKYSELAELYRRLEKTTLKTLKTKFVADFLKKTPDDLLEIVPYLILGKVFPDWDERELGVGEKLLIRAVSMATGVPEREIENSIKDTGDLGESVALALKKRKQKSFFSQPLTIKRVYSTFVKVAEASGEGSQDRKMKYLANLFMDAQPEEGKYIARTVLGTMRTGVAEGILRDAIAEAFKVKPELVERAYMLTSDFGYVAKVAKLEGNEGLSKVSIQIGKPIRPMLAQNAANVKEALIEMGGEAAFEIKYDGARVQVHRDGDRVIIYSRRLENVTRSIPEIVEAVKASLKPSKVIVEGELVAVGENGRPRPFQYVLRRFRRKYNIEEMIEKIPLELNLFDILYVDGESLIDTKFAERRKKLEESVEESDKIKLAEQLVTKKVEEAEEFYKRALELGHEGLMAKRLDSIYEPGNRGKKWLKIKPTMENLDLVIIGAEWGEGRRAHLLGSFLVAAYDPESGEFVPVGKVGSGFTDEDLVEFTKMLKPLIVREEGKFVEIEPKVVIEVTYQEIQKSPKYKSGFALRFPRYVALREDKSPEEADTIERVAQLYELQERFKAKR

SEQ ID NO: 152-159 - skipped (8 entries)

SEQ ID NO: 160 - DNA, 25 bp, synthetic construct
  source 1..25: mol_type = other DNA
gaacgacatggctacgatccgactt

SEQ ID NO: 161 - DNA, 20 bp, synthetic construct
  source 1..20: mol_type = other DNA
aatgatacggcgaccaccga

SEQ ID NO: 162 - DNA, 21 bp, synthetic construct
  source 1..21: mol_type = other DNA
tcgtatgccgtcttctgcttg